(19) United States
(12) Patent Application Publication
(10) Pub. No.: US 2025/0009893 A1
(43) Pub. Date: Jan. 9, 2025

(54) DENDRIMER CONJUGATES AND METHODS OF USE THEREOF

(71) Applicant: Ashvattha Therapeutics, Inc., Redwood City, CA (US)

(72) Inventors: Jeffrey L. Cleland, Redwood City, CA (US); Rishi Sharma, Redwood City, CA (US)

(73) Assignee: Ashvattha Therapeutics, Inc., Redwood City, CA (US)

(21) Appl. No.: 18/568,516

(22) PCT Filed: Jun. 10, 2022
(86) PCT No.: PCT/US22/33112

§ 371 Date: Dec. 8, 2023

Related U.S. Application Data
(60) Provisional application No. 63/327,301, filed on Apr. 4, 2022.
(60) Provisional application No. 63/209,348, filed on Jun. 10, 2021.

(51) Int. Cl. A61K 47/59; A61K 47/34

(52) U.S. Cl. CPC A61K 47/595; A61K 47/34

(57) ABSTRACT

Aspects of the disclosure provide dendrimer conjugates, compositions comprising dendrimer conjugates, and methods of using dendrimer conjugates and compositions thereof. In some aspects, the disclosure provides dendrimer conjugates comprising a dendrimer conjugated to at least one agent. In some embodiments, a dendrimer conjugate comprises one or more agents useful in therapy, imaging, and/or targeted delivery. In some aspects, provided are methods of synthesizing a functionalized dendrimer, comprising reacting a first dendrimer with one or more amines.

### CROSS-REFERENCE TO RELATED APPLICATIONS

This application is a national stage filing under 35 U.S.C. § 371 of International Patent Application Serial No. PCT/US2022/033112, filed Jun. 10, 2022, entitled “DENDRIMER CONJUGATES AND METHODS OF USE THEREOF”, which claims priority under 35 U.S.C. § 119(e) to U.S. Provisional Patent Application No. 63/327,301, filed Apr. 4, 2022, and U.S. Provisional Patent Application No. 63/209,348, filed Jun. 10, 2021, each of which is hereby incorporated by reference in its entirety.

### BACKGROUND

Advancements in the field of nanotechnology have resulted in the creation of many new materials and devices with a vast range of applications. Nanotechnology manifests in a wide range of materials and particles, such as fullerenes and dendrimers. However, in vivo application of nanoparticles is challenged by the rapid elimination from circulation due to their interactions with biological systems. Alternative strategies in nanoparticle development could allow for the advantageous properties of nanoparticles to be utilized more effectively in the context of clinical applications, diagnostics, and biomedical research.

### SUMMARY

In some aspects, the disclosure provides therapeutic and/or diagnostic compounds comprising a dendrimer conjugated to an agent through a terminal ether or amide bond. In some embodiments, the dendrimer comprises terminal hydroxyl groups optionally substituted with the agent. In some embodiments, the agent is a therapeutic agent or an imaging agent.

In some aspects, the disclosure provides a dendrimer conjugate of Formula (I):

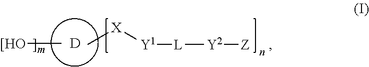

wherein: D is a dendrimer; X is O or NH; Y¹ is optionally substituted alkylene, optionally substituted alkenylene, optionally substituted alkynylene, or a covalent bond; Y² is selected from the group consisting of secondary amides, tertiary amides, sulfonamide, secondary carbamates, tertiary carbamates, carbonates, ureas, carbinols, disulfides, hydrazones, hydrazides, ethers, carbonyls, and combinations thereof; Z is a therapeutic agent or an imaging agent; L is a linker; m is an integer from 16 to 4096, inclusive; and n is an integer from 1 to 100, inclusive. In some embodiments, the ratio of m to (m+n) is at least 0.5. In some embodiments, the ratio of m to (m+n) is between about 0.50 and about 0.99.

In some aspects, the disclosure provides a dendrimer conjugate of Formula (II):

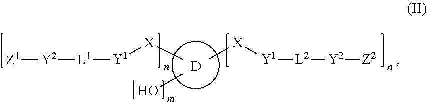

wherein: D is a dendrimer; each instance of X is independently O or NH; each instance of Y¹ is independently optionally substituted alkylene, optionally substituted alkenylene, optionally substituted alkynylene, or a covalent bond; each instance of Y² is independently selected from the group consisting of secondary amides, tertiary amides, sulfonamide, secondary carbamates, tertiary carbamates, carbonates, ureas, carbinols, disulfides, hydrazones, hydrazides, ethers, carbonyls, and combinations thereof; Z¹ and Z² are independently therapeutic agents, targeting agents, or imaging agents, with the proviso that Z¹ and Z² are different; L¹ and L² are independently linkers; m is an integer from 16 to 4096, inclusive; and each instance of n is independently an integer from 1 to 100, inclusive.

In some embodiments, the dendrimer of a therapeutic compound or dendrimer conjugate of the disclosure is selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof.

In some embodiments, the linker of a therapeutic compound or dendrimer conjugate comprises a polymer. In some embodiments, the polymer is a polymeric polyol, a polypeptide, or an unsubstituted alkyl chain. In some embodiments, the polymer is a polymeric polyol selected from the group consisting of polyethylene glycol (PEG), polypropylene glycol, and polyvinyl alcohol. In some embodiments, the polymer is a polypeptide comprising at least 2 and up to 25 amino acids. In some embodiments, the polymer is an unsubstituted C2-30 alkyl chain. In some embodiments, the linker comprises at least one moiety selected from the group consisting of 1,2,3-triazolyl, 4,5-dihydro-1,2,3-triazolyl, isoxazolyl, 4,5-dihydroisoxazolyl, and 1,4-dihydropyridazyl. In some embodiments, the linker comprises the polymer and the at least one moiety. In some embodiments, the linker is non-hydrolyzable under physiological conditions.

In some embodiments, the therapeutic agent of a therapeutic compound or dendrimer conjugate is selected from the group consisting of angiotensin II receptor blockers, Farnesoid X receptor agonists, death receptor 5 agonists, sodium-glucose cotransporter type-2 inhibitors, lysophosphatidic acid 1 receptor antagonists, endothelin-A receptor antagonist, PPAR6 agonists, ATl receptor antagonists, CCR5/CCR2 antagonists, anti-fibrotic agents, anti-inflammatory agents, antioxidant agents, STING agonists, CSF1R inhibitors, AXL inhibitors, c-Met inhibitors, PARP inhibitors, receptor tyrosine kinase inhibitors, MEK inhibitors, group I p21-activated kinase (PAK1) inhibitors, glutaminase inhibitors, TIE II antagonists, CXCR2 inhibitors, CD73 inhibitors, arginase inhibitors, PI3K inhibitors, TLR4 agonists, TLR7 agonists, SHP2 inhibitors, chemotherapeutic agents, STING antagonists, and JAK1 inhibitors.

In some embodiments, the therapeutic agent of a therapeutic compound or dendrimer conjugate is a MEK inhibitor. In some embodiments, the MEK inhibitor is selected from the group consisting of Trametinib, Cobimetinib, Binimetinib, Selumetinib, PD325901, PD035901, PD032901, and TAK-733. In some embodiments, the therapeutic agent of a therapeutic compound or dendrimer conjugate is a receptor tyrosine kinase inhibitor. In some embodiments, the receptor tyrosine kinase inhibitor is selected from the group consisting of sunitinib, sorafenib, pazopanib, vandetanib, axitinib, cediranib, vatalanib, dasatinib, bemcentinib (R428), dubermatinib (TP-0903), nintedanib, cabozantinib, and motesanib. In some embodiments, the therapeutic agent of a therapeutic compound or dendrimer conjugate is a PAK1 inhibitor. In some embodiments, the PAK1 inhibitor is Frax-1036 (6-[2-chloro-4-(6-methyl-2-pyrazinyl)phenyl]-8-ethyl-2-[[2-(1-methyl-4-piperidinyl)ethyl]amino]-pyrido[2,3-d]pyrimidin-7(8H)-one).

In some embodiments, the imaging agent of a therapeutic compound or dendrimer conjugate is selected from the group consisting of dyes, fluorescent dyes, near infrared dyes, single-photon emission computed tomography (SPECT) imaging agents, positron emission tomography (PET) imaging agents, magnetic resonance imaging (MRI) contrast agents, and radionuclides.

In some embodiments, a therapeutic compound or dendrimer conjugate of the disclosure comprises at least one targeting agent conjugated to the dendrimer. In some embodiments, the targeting agent is a triantennary-N-acetylgalactosamine (GalNAc).

In some aspects, the disclosure provides a composition comprising a carrier and functionalized dendrimers of Formula (I-A):

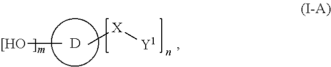

wherein: D is a dendrimer selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof; X is NH; Y¹ is optionally substituted alkylene, optionally substituted alkenylene, optionally substituted alkynylene, or a covalent bond; m is an integer from 16 to 4096, inclusive; and n is an integer from 1 to 100, inclusive, wherein the polydispersity value of the functionalized dendrimers in the composition is less than or equal to 1.10.

In some aspects, the disclosure provides methods of synthesizing a functionalized dendrimer of Formula (I-A):

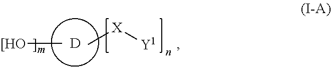

wherein:
D is a dendrimer selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof; X is NH; Y¹ is optionally substituted alkylene, optionally substituted alkenylene, optionally substituted alkynylene, or a covalent bond; m is an integer from 16 to 4096, inclusive; and n is an integer from 1 to 100, inclusive; comprising: reacting a dendrimer of Formula (II-A):

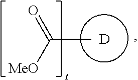

under suitable conditions to form the functionalized dendrimer of Formula (I-A);
wherein: D is a dendrimer selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof; t is an integer from 16 to 4096, inclusive; with one or more amines, wherein each amine is of the formula H₂NR¹, wherein R¹ is optionally substituted alkylene, optionally substituted alkenylene, optionally substituted alkynylene, or a covalent bond. In some embodiments, provided is a functionalized dendrimer described herein, or a salt, solvate, hydrate, stereoisomer, polymorph, tautomer, isotopically enriched form (e.g., isotopically labeled derivative) thereof.

In some embodiments, n is approximately 3 or 10 (e.g., 3 or 10). In some embodiments, m is the value of n subtracted from 64. In some embodiments, n is 3 and m is 61. In some embodiments, n is 10 and m is 54. In some embodiments, m is approximately 61 or 54 (e.g., 61 or 54).

In some embodiments, a dendrimer of Formula (II-A) is of formula:

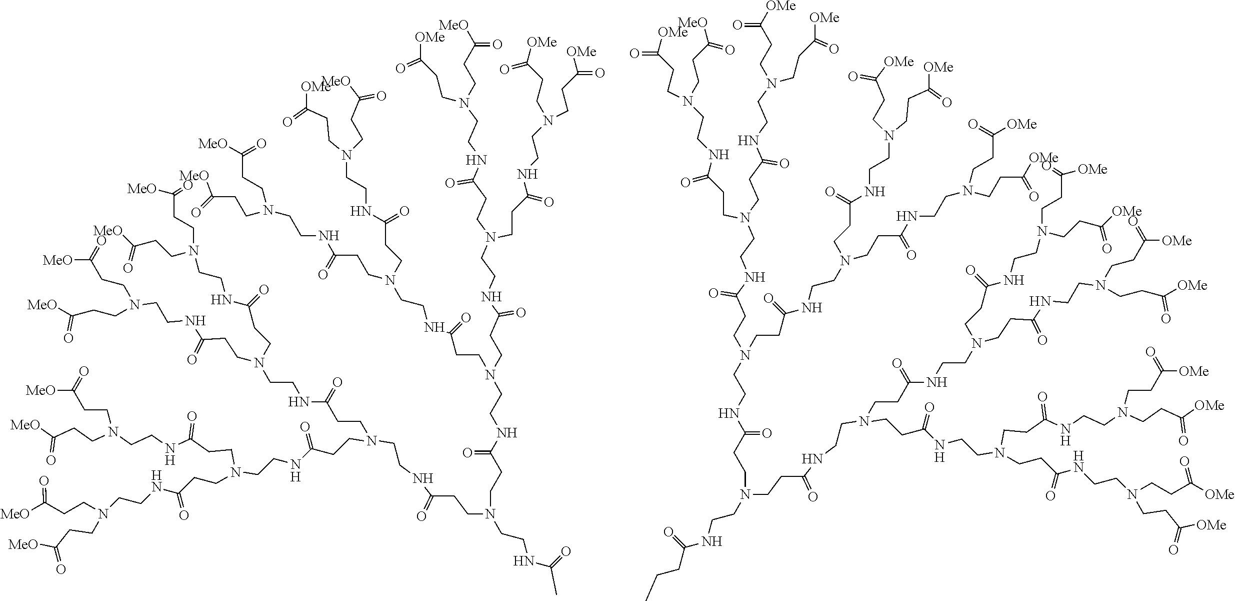

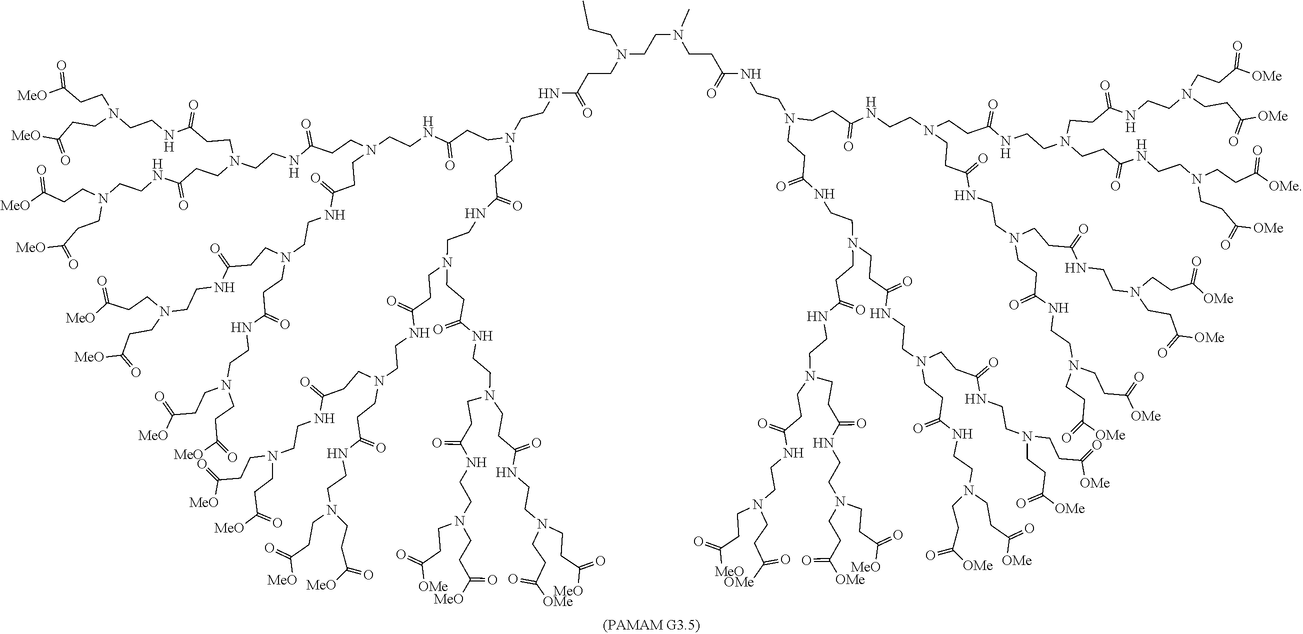

In some embodiments, the functionalized dendrimer of Formula (I-A) is of formula:

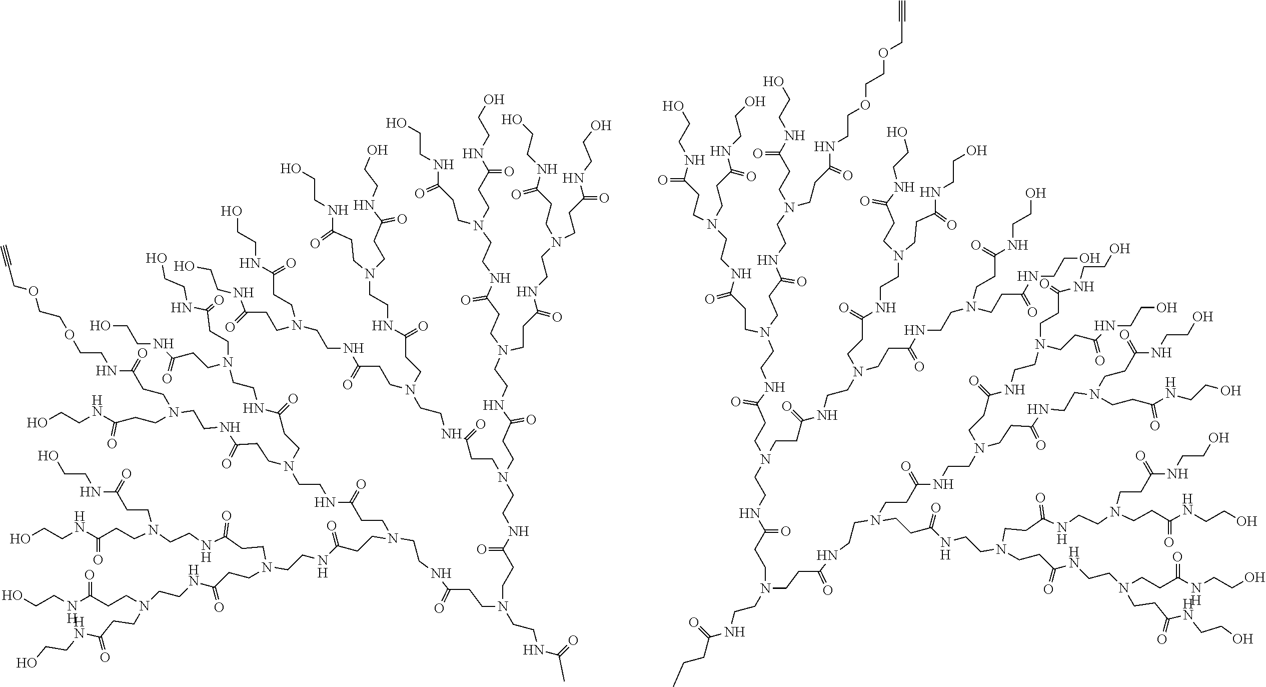

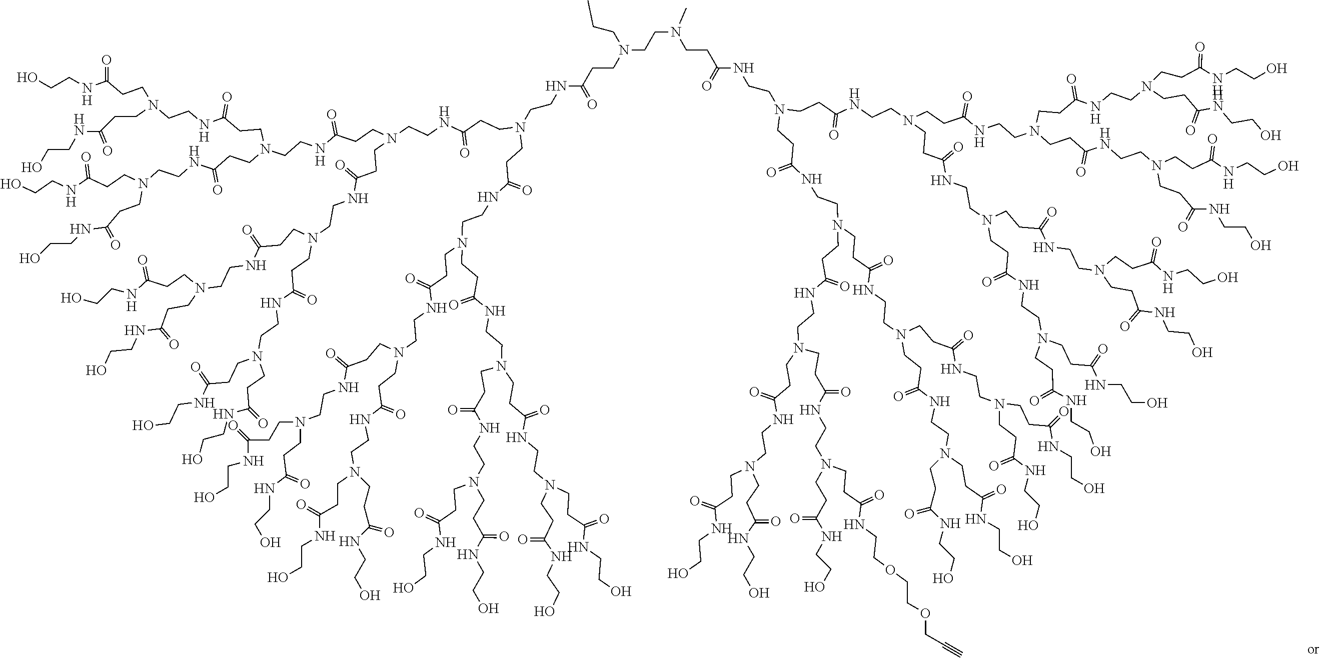

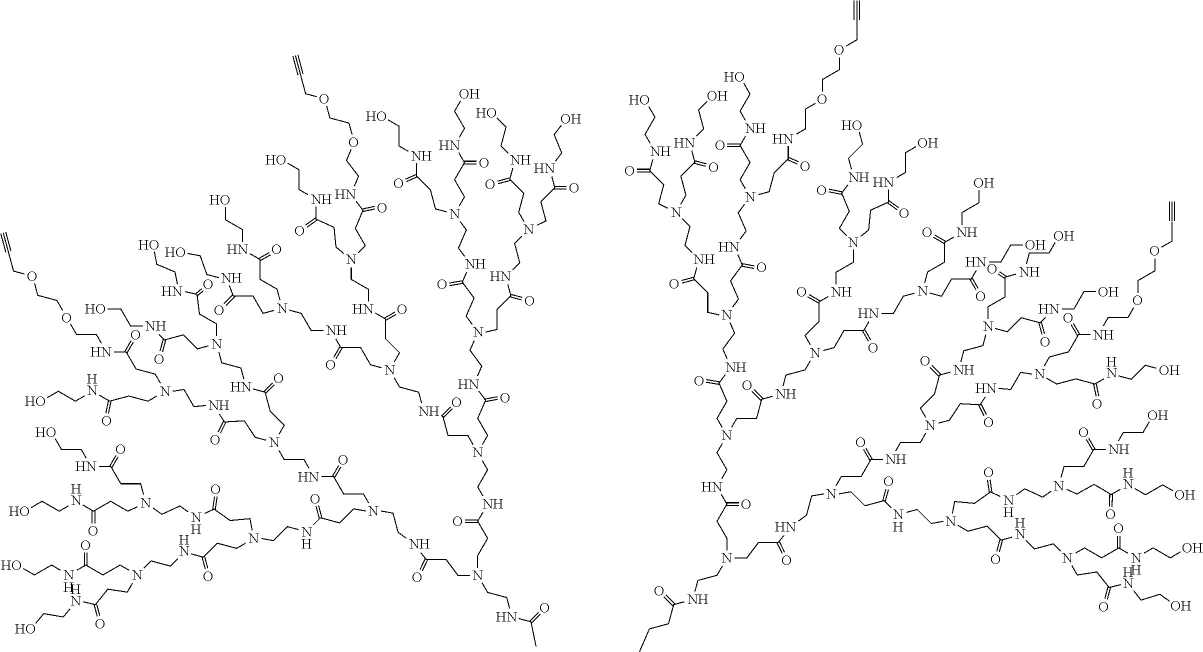

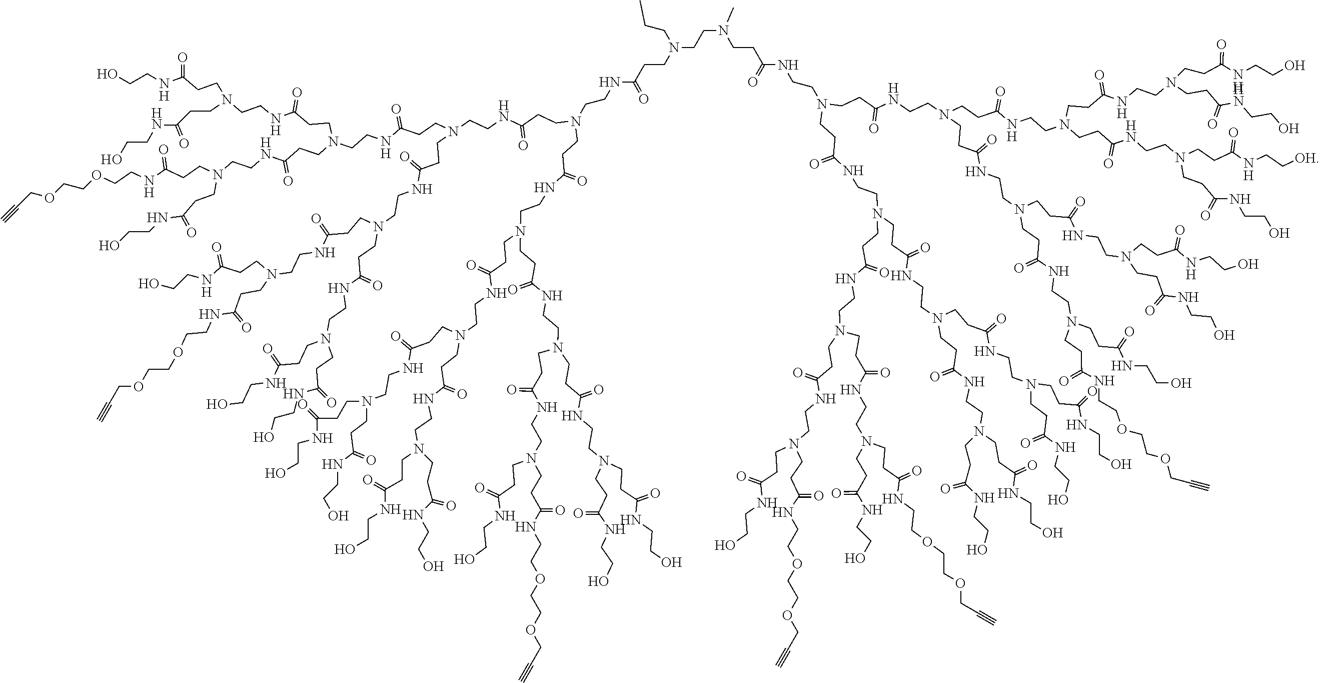

In yet another aspect, the present disclosure provides functionalized dendrimers of Formula (I-A), synthesized by methods described herein. In some aspects, provided is a dendrimer of Formula (II-A) of formula:

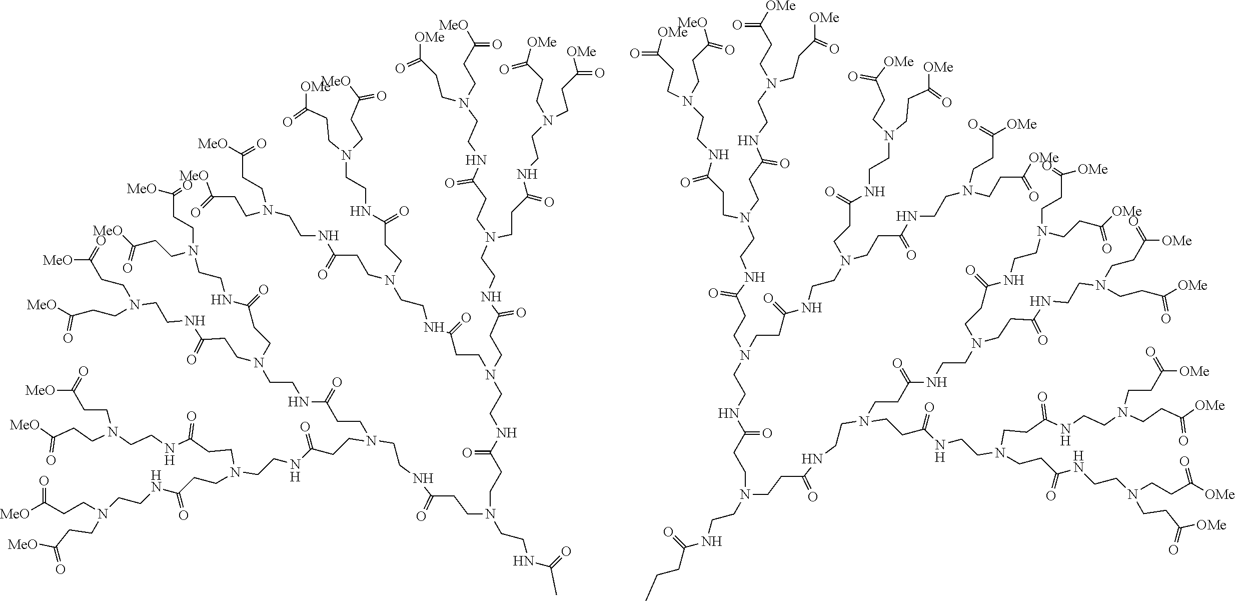

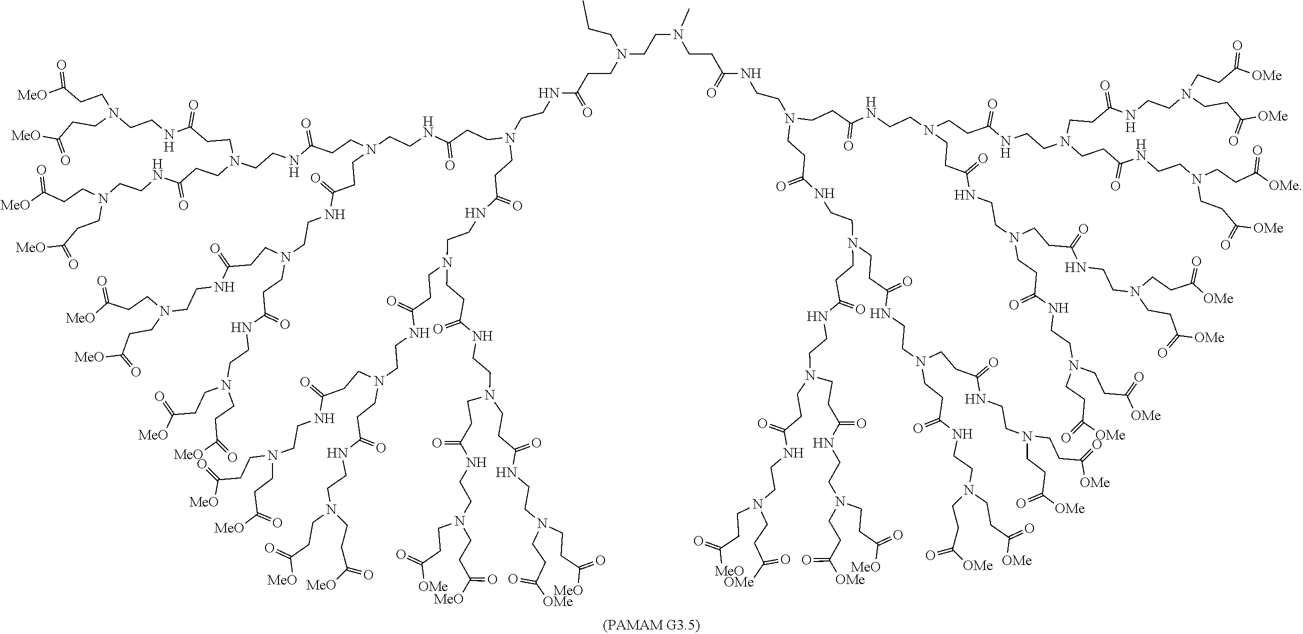

In some aspects, the disclosure provides a composition of a therapeutic compound comprising a dendrimer conjugated to a therapeutic agent through a terminal ether or amide bond. In some embodiments, the dendrimer comprises a high-density of terminal hydroxyl groups optionally substituted with the therapeutic agent. In some embodiments, a therapeutic compound comprising a dendrimer conjugated to a therapeutic agent is 10-20% by mass of therapeutic agent. In some embodiments, the terminal ether or amide bond is conjugated to the therapeutic agent through a linker.

In some embodiments, the therapeutic compound is about 10% to about 15% by mass of therapeutic agent. In some embodiments, the therapeutic compound is about 15% to about 20% by mass of therapeutic agent. In some embodiments, at least 50% of terminal sites on the dendrimer comprise terminal hydroxyl groups. In some embodiments, at least 50% and up to 99% (e.g., 50-95%, 50-90%, 50-80%, 50-70%, 50-60%, 60-80%, 70-90%) of terminal sites on the dendrimer comprise terminal hydroxyl groups.

In some embodiments, the therapeutic agent has an aqueous solubility that is increased relative to an unconjugated compound comprising the therapeutic agent in absence of the dendrimer. In some embodiments, the aqueous solubility is increased by at least 10% relative to the unconjugated compound. In some embodiments, the aqueous solubility is increased by between about 10% and about 100% relative to the unconjugated compound. In some embodiments, the aqueous solubility is increased by at least about a factor of two relative to the unconjugated compound. In some embodiments, the aqueous solubility is increased by between about a factor of two and about a factor of ten relative to the unconjugated compound. In some embodiments, the aqueous solubility is solubility under physiological conditions. In some embodiments, the aqueous solubility is solubility in water having a pH of between about 7.0 and about 8.0. In some embodiments, the therapeutic agent is present at a concentration at which the unconjugated compound is insoluble under physiological conditions.

In some aspects, the disclosure provides methods of treating or imaging a disease or disorder of the brain or central nervous system in a subject in need thereof, the methods comprising administering to the subject a composition comprising a dendrimer conjugate described herein in an amount effective to treat or image the disease or disorder of the brain or central nervous system in the subject.

In some embodiments, the dendrimer conjugate is selectively taken up by activated microglia and/or activated macrophages in the brain or central nervous system of the subject. In some embodiments, the activated macrophages are resident macrophage cells of the central nervous system. In some embodiments, the dendrimer conjugate crosses the blood-brain barrier of the subject. In some embodiments, the disease or disorder is a tumor of the brain or central nervous system. In some embodiments, the tumor is a benign or malignant tumor associated with neurofibromatosis. In some embodiments, the disease or disorder is neurofibromatosis (e.g., neurofibromatosis type 1 (NF1)). Accordingly, in some embodiments, the subject has or is suspected of having neurofibromatosis (e.g., NF1). In some embodiments, the dendrimer conjugate is selectively taken up by tumor-associated macrophages of the tumor in the subject. In some embodiments, the tumor is a brain cancer or a cancer of the central nervous system. In some embodiments, the tumor is a brain cancer selected from the group consisting of neoplasias and hyperplasias. In some embodiments, the tumor is a cancer of the central nervous system selected from the group consisting of gliomas, glioblastoma, astrocytoma, oligodendroglioma, meningioma, medulloblastoma, ganglioma, and Schwannoma. In some embodiments, the dendrimer conjugate is not conjugated to a targeting moiety. In some embodiments, the composition is administered to the subject systemically, intravenously, or orally. In some embodiments, the dendrimer conjugate comprises a therapeutic agent, and the composition has a therapeutic index that is increased relative to a composition comprising the therapeutic agent in absence of the dendrimer. In some embodiments, the therapeutic agent is a MEK inhibitor (e.g., Trametinib, Cobimetinib, Binimetinib, Selumetinib, PD325901, PD035901, PD032901, or TAK-733). In some embodiments, the therapeutic agent is a receptor tyrosine kinase inhibitor (e.g., sunitinib, sorafenib, pazopanib, vandetanib, axitinib, cediranib, vatalanib, dasatinib, bemcentinib (R428), dubermatinib (TP-0903), nintedanib, cabozantinib, or motesanib). In some embodiments, the therapeutic agent is a PAK1 inhibitor (e.g., Frax-1036 (6-[2-chloro-4-(6-methyl-2-pyrazinyl)phenyl]-8-ethyl-2-1[[2-(1-methyl-4-piperidinyl)ethyl]amino]-pyrido[2,3-d]pyrimidin-7(8H)-one)).

In some aspects, the disclosure provides methods of treating or imaging a disease or disorder of the eye in a subject in need thereof, the methods comprising administering to the subject a composition comprising a dendrimer conjugate described herein in an amount effective to treat or image the disease or disorder of the eye in the subject.

In some embodiments, the dendrimer conjugate is selectively taken up by activated microglia and/or activated macrophages in the eye of the subject. In some embodiments, the dendrimer conjugate crosses the blood-retinal barrier of the subject. In some embodiments, the dendrimer conjugate is not conjugated to a targeting moiety. In some embodiments, the composition is administered to the subject systemically, intravenously, or orally. In some embodiments, the dendrimer conjugate comprises a therapeutic agent, and the composition has a therapeutic index that is increased relative to a composition comprising the therapeutic agent in absence of the dendrimer.

In some aspects, the disclosure provides methods of treating or imaging a proliferative disease in a subject in need thereof, the methods comprising administering to the subject a composition comprising a dendrimer conjugate described herein in an amount effective to treat or image the proliferative disease in the subject.

In some embodiments, the proliferative disease is neurofibromatosis. In some embodiments, the proliferative disease is selected from neurofibromatosis type 1 (NF1), neurofibromatosis type 2 (NF2), and schwannomatosis. In some embodiments, the proliferative disease is NF1. In some embodiments, the dendrimer conjugate is not conjugated to a targeting moiety. In some embodiments, the composition is administered to the subject systemically, intravenously, or orally. In some embodiments, the dendrimer conjugate comprises a therapeutic agent, and the composition has a therapeutic index that is increased relative to a composition comprising the therapeutic agent in absence of the dendrimer. In some embodiments, the therapeutic agent is a MEK inhibitor (e.g., Trametinib, Cobimetinib, Binimetinib, Selumetinib, PD325901, PD035901, PD032901, or TAK-733). In some embodiments, the therapeutic agent is a receptor tyrosine kinase inhibitor (e.g., sunitinib, sorafenib, pazopanib, vandetanib, axitinib, cediranib, vatalanib, dasatinib, bemcentinib (R428), dubermatinib (TP-0903), nintedanib, cabozantinib, or motesanib). In some embodiments, the therapeutic agent is a PAK1 inhibitor (e.g., Frax-1036 (6-[2-chloro-4-(6-methyl-2-pyrazinyl)phenyl]-8-ethyl-2-[[2-(1-methyl-4-piperidinyl)ethyl]amino]-pyrido[2,3-d]pyrimidin-7(8H)-one)).

The details of certain embodiments of the disclosure are set forth in the Detailed Description, as described below. Other features, objects, and advantages of the disclosure will be apparent from the Examples, Drawings, and Claims.

### BRIEF DESCRIPTION OF THE DRAWINGS

The accompanying drawings, which constitute a part of this specification, illustrate several embodiments of the invention and together with the description, serve to explain the principles of the invention.

FIG. 1 is a schematic showing the chemical structure of a dendrimer conjugate (compound D-4517.2).

FIG. 2 is a reaction scheme showing one synthesis strategy of N, N-didesethyl sunitinib azide with an amide linkage.

FIG. 3 is a reaction scheme showing the synthesis of a dendrimer conjugate (D-4517.2) in which N, N-didesethyl sunitinib is conjugated to a dendrimer with ether linkages for enhanced in vivo stability.

FIG. 4 is a bar graph showing drug release percentage by weight (0.0%-0.50%) of D-didesethyl sunitinib conjugate, D-4517.2, in human, mouse, and rat plasma conditions over time points for each of 4, 24 and 48 hours, respectively.

FIG. 5 is a reaction scheme showing the synthesis of a Dendrimer-N-Acetyl-L-cysteine methyl ester conjugate.

FIG. 6 is a reaction scheme showing the synthesis of β-GalNAc-triantennary-PEG3-Azide building block for conjugation to dendrimer.

FIG. 7 is a chromatograph showing an exemplary integration performed using method A. Example calculation:

$$Average \# PEG - Alkyne arms = 60 * \frac{1}{1 + \frac{{H}_{e , k , l} - ( {H}_{k} + {H}_{l} )}{{H}_{m}}} = 64 * \frac{1}{1 + \frac{{H}_{e , k , l} - 3 * {H}_{m}}{{H}_{m}}} = 64 * \frac{1}{1 + \frac{165.36 - 41.19}{13.73}} = 6.37$$

FIG. 8 is a chromatograph showing an exemplary integration performed using method A. Example calculation: Hₖ+H₁=3*Hₘ

$$Average \# PEG - Alkyne arms = 64 * \frac{{H}_{m}}{128} = 64 * \frac{13.73}{128} = 6.87$$

FIG. 9 is a calibration data plot for FID vs Ethanolamine (mg/mL) and FID vs PEG-Alkyne (mg/mL)

FIG. 10 is a plot showing average alkyne arms vs. PEG-Alkyne loading for experiment DP07-55-1.

FIG. 11 is a plot showing average alkyne arms vs. PEG-Alkyne loading for experiment AA08-85, AA08-88 and DP07-55-1.

FIG. 12 is a plot showing average alkyne arms vs. PEG-Alkyne loading for experiment DP07-51 and DP07-60.

FIG. 13 is a calibration data plot for FID vs Ethanolamine (mg/mL) and FID vs PEG-Alkyne (mg/mL)

FIGS. 14A-14C is the ASTRA report for PAMAM G3.5 dendrimer. Concentration: 7.080 mg/mL.

FIGS. 15A-15C is the ASTRA report for PAMAM G4. Concentration: 7.080 mg/mL.

FIGS. 16A-16C is the ASTRA report for PAMAM G3.5. Concentration: 7.080 mg/mL.

FIG. 17 is the ¹H-NMR data for DP07-51-1. 1H NMR (300 MHz, METHANOL-d4) δ ppm 2.38 (br t, J=5.78 Hz, 248H) 3.50-3.74 (m, 191H) 4.20 (d, J=2.49 Hz, 27H) 4.18-4.20 (m, 1H).

FIG. 18 is the ¹H-NMR data for DP07-51-2. ¹H NMR (300 MHz, METHANOL-d4) δ ppm 2.23-2.49 (m, 248H) 3.61 (s, 194H) 4.16-4.24 (m, 31H).

FIG. 19 is the ¹H-NMR data for DP07-51-3. ¹H NMR (300 MHz, METHANOL-d4) δ ppm 2.38 (br t, J=5.78 Hz, 248H) 3.50-3.74 (m, 191H) 4.20 (d, J=2.49 Hz, 27H) 4.18-4.20 (m, 1H).

FIG. 20 is the ¹H-NMR data for DP07-51-4. 1H NMR (300 MHz, METHANOL-d4) δ ppm 2.23-2.49 (m, 248H) 3.61 (s, 194H) 4.16-4.24 (m, 31H).

FIGS. 21A-21C is the ASTRA report for DP07-51-1.

FIGS. 22A-22C is the ASTRA report for DP07-51-2.

FIGS. 23A-23C is the ASTRA report for DP07-51-3.

FIGS. 24A-24C is the ASTRA report for DP07-51-4.

FIG. 25 is the ¹H-NMR data for DP07-60-1. ¹H NMR (300 MHz, METHANOL-d4) δ ppm 2.25-2.49 (m, 248H) 3.50-3.74 (m, 145H) 4.20 (d, J=2.34 Hz, 6H).

FIG. 26 is the ¹H-NMR data for DP07-60-2. ¹H NMR (300 MHz, METHANOL-d4) δ ppm 2.26-2.50 (m, 248H) 3.61 (t, J=5.63 Hz, 150H) 4.20 (d, J=2.49 Hz, 6H).

FIG. 27 is the ¹H-NMR data for DP07-60-3. ¹H NMR (300 MHz, METHANOL-d4) δ ppm 2.26-2.49 (m, 248H) 3.61 (t, J=5.63 Hz, 143H) 4.18-4.22 (m, 6H).

FIG. 28 is the ¹H-NMR data for DP07-60-4. ¹H NMR (300 MHz, METHANOL-d4) δ ppm 2.28-2.48 (m, 248H) 3.61 (t, J=5.63 Hz, 148H) 4.18-4.22 (m, 7H).

FIGS. 29A-29C is the ASTRA report for DP07-60-1.

FIGS. 30A-30C is the ASTRA report for DP07-60-2.

FIGS. 31A-31C is the ASTRA report for DP07-60-3.

FIGS. 32A-32C is the ASTRA report for DP07-60-4.

FIGS. 33A-33C is the ASTRA report for DP07-74-1.

FIG. 34 is the ¹H-NMR data for DP07-74-1. ¹H NMR (300 MHz, METHANOL-d4) δ ppm 2.27-2.48 (m, 243H) 2.53-2.67 (m, 121H) 2.68-2.95 (m, 255H) 3.31 (s, 46H) 3.61 (t, J=5.71 Hz, 143H) 4.20 (d, J=2.34 Hz, 6H).

FIGS. 35A-35B is the GC data for DP07-74-1.

FIGS. 36A-36B is the GC data for the IPA blank.

FIGS. 37A-37C is the ASTRA report for DP07-68-1.

FIGS. 38A-38B is the ¹H-NMR data for DP07-68-1. ¹H NMR (300 MHz, METHANOL-d4) δ ppm 2.38 (br t, J=6.07 Hz, 248H) 2.50-2.67 (m, 138H) 2.71-2.87 (m, 241H) 2.94 (s, 36H) 3.18-3.44 (m, 39H) 3.49-3.73 (m, 178H) 4.20 (d, J=2.34 Hz, 21H).

FIGS. 39A-39B is the GC data for DP07-68-1.

FIG. 40 is the ¹H-NMR data for DP07-82-1. ¹H NMR (300 MHz, METHANOL-d4) δ ppm 2.37 (br s, 248H) 3.71-3.74 (m, 84H) 6.05-6.08 (m, 198H).

FIGS. 41A-41C is the ASTRA report for DP07-82-1.

FIG. 42 is the ¹H-NMR data for DP07-82-1 Alkyne. ¹H NMR (300 MHz, METHANOL-d4) δ ppm 2.25-2.48 (m, 247H) 2.36-2.36 (m, 5H) 3.50-3.71 (m, 140H) 3.60-3.61 (m, 3H) 4.20 (d, J=2.49 Hz, 5H) 4.20-4.20 (m, 1H).

FIG. 43 is the ¹³C-NMR data for DP07-82-1. ¹³C NMR (75 MHz, METHANOL-d4) δ ppm 33.32 (s, 1C) 41.61 (s, 1C) 47.87 (s, 1C) 49.80 (s, 1C) 60.23 (s, 1C) 173.75 (s, 1C).

FIG. 44 is the ¹H-NMR data for DP07-85-1. ¹H NMR (300 MHz, METHANOL-d4) δ ppm 2.38 (br s, 242H) 6.04-6.08 (m, 218H).

FIGS. 45A-45C is the ASTRA report for DP07-85-1.

FIG. 46 is the ¹H-NMR data for DP07-85-1 Alkyne. ¹H NMR (300 MHz, METHANOL-d4) δ ppm 2.38 (br t, J=6.14 Hz, 244H) 2.36-2.36 (m, 9H) 3.49-3.73 (m, 167H) 4.16-4.22 (m, 20H).

FIGS. 47A-47B is the ¹³C-NMR data for DP07-85-1. ¹³C NMR (75 MHz, METHANOL-d4) δ ppm 33.31 (s, 1C) 41.63 (s, 1C) 47.87 (s, 1C) 60.23 (s, 1C) 173.74 (s, 1C).

FIG. 48 is the ¹H-NMR data for DP07-82-1 (LOT #DP07-82-2). ¹H NMR (300 MHz, METHANOL-d4) δ ppm 2.37 (br s, 248H) 3.71-3.74 (m, 84H) 6.05-6.08 (m, 198H).

FIGS. 49A-49C is the ASTRA report for DP07-82-1 (LOT #DP07-82-2).

FIG. 50 is the ¹H-NMR data for DP07-82-1 (LOT #DP07-82-2) Alkyne. ¹H NMR (300 MHz, METHANOL-d4) δ ppm 2.25-2.48 (m, 247H) 2.36-2.36 (m, 5H) 3.50-3.71 (m, 140H) 3.60-3.61 (m, 3H) 4.20 (d, J=2.49 Hz, 5H) 4.20-4.20 (m, 1H).

FIGS. 51A-51B is the ¹³C-NMR data for DP07-82-1 (LOT #DP07-82-2). ¹³C NMR (75 MHz, METHANOL-d4) δ ppm 33.32 (s, 1C) 41.61 (s, 1C) 47.87 (s, 1C) 49.80 (s, 1C) 60.23 (s, 1C) 173.75 (s, 1C).

FIG. 52 is the ¹H-NMR data for DP07-85-1 (LOT #DP07-85-3). ¹H NMR (300 MHz, METHANOL-d4) δ ppm 2.38 (br s, 242H) 6.04-6.08 (m, 218H).

FIGS. 53A-53C is the ASTRA report for DP07-85-1 (LOT #DP07-85-3).

FIG. 54 is the ¹H-NMR data for DP07-85-1 (LOT #DP07-85-3) Alkyne. ¹H NMR (300 MHz, METHANOL-d4) δ ppm 2.38 (br t, J=6.14 Hz, 244H) 2.36-2.36 (m, 9H) 3.49-3.73 (m, 167H) 4.16-4.22 (m, 20H).

FIGS. 55A-55B is the ¹³C-NMR data for DP07-85-1 (LOT #DP07-85-3). ¹³C NMR (75 MHz, METHANOL-d4) δ ppm 33.31 (s, 1C) 41.63 (s, 1C) 47.87 (s, 1C) 60.23 (s, 1C) 173.74 (s, 1C).

FIGS. 56A-56C show results from synthesis and characterization of dendrimers conjugated to the following PAK1 inhibitor: Frax-1036.

FIGS. 57A-57T show results from synthesis and characterization of dendrimers conjugated to the following MEK inhibitors: selumetinib (FIGS. 57A-57D), trametinib (FIGS. 57E-57P), and cobimetinib (FIGS. 57Q-57T).

FIGS. 58A-58T show results from synthesis and characterization of dendrimers conjugated to the following receptor tyrosine kinase inhibitors: dasatinib (FIGS. 58A-58H), bemcentinib (FIGS. 58I-58N), dubermatinib (FIGS. 58O-58P), and cabozantinib (FIGS. 58Q-58T).

### DETAILED DESCRIPTION

Among other aspects, the disclosure provides dendrimer conjugates, compositions comprising dendrimer conjugates, and methods of using dendrimer conjugates and compositions thereof. In some embodiments, a dendrimer conjugate comprises a dendrimer conjugated to at least one agent. In some embodiments, a dendrimer conjugate comprises one or more agents useful in therapy, imaging, and/or targeted delivery.

In some aspects, the disclosure relates to a therapeutic compound comprising a dendrimer conjugated to a therapeutic agent. The inventors have recognized and appreciated that certain therapeutics with unfavorable in vivo profiles can be modified by conjugation to dendrimers to provide a therapeutic compound that shows reduced off-target toxicity, more highly selective uptake, and sustained intracellular effects. The inventors have further recognized and appreciated that such therapeutic compounds are highly tunable in the dendrimer portion, such that the hydrophilicity of a therapeutic compound can be tailored to allow for the targeted delivery of certain therapeutics to biological targets which would otherwise be poorly accessible by the therapeutic. In other aspects, provided are methods of synthesizing a functionalized dendrimer of Formula (I-A), comprising: reacting a dendrimer of Formula (II-A) with one or more amines, wherein each amine is of the formula H₂NR¹, under suitable conditions to form the functionalized dendrimer of Formula (I-A). In other aspects, provided are functionalized dendrimers of Formula (I-A), synthesized by methods described herein.

In some aspects, the disclosure provides methods of treating or imaging a disease or disorder in a subject in need thereof, comprising administering to the subject a composition comprising a dendrimer conjugate described herein. In some embodiments, dendrimer conjugates of the disclosure are capable of targeting reactive immune cells in absence of any targeting moieties. For example, in some embodiments, dendrimer conjugates described herein are capable of crossing the blood-brain barrier in the central nervous system of a subject, where the dendrimer conjugate is selectively taken up by activated microglia and/or activated macrophages. In some embodiments, dendrimer conjugates described herein are capable of crossing the blood-retinal barrier in the eye of a subject, where the dendrimer conjugate is selectively taken up by activated microglia and/or activated macrophages.

### Dendrimer Conjugates

In some embodiments, a dendrimer conjugate refers to a dendrimer that is conjugated to at least one agent as described herein. In some embodiments, a dendrimer is covalently conjugated (e.g., covalently attached) to at least one agent. In some embodiments, a dendrimer conjugate comprises a dendrimer, which can be described as having a molecular architecture with an interior core and layers (or “generations”) of repeating units which are attached to and extend from this interior core, each layer having one or more branching points, and the outermost generation having terminal functional groups.

In some embodiments, terminal functional groups of a dendrimer include one or more hydroxyl groups, one or more amine groups, and/or one or more carboxyl groups. In some embodiments, the terminal functional groups of a dendrimer provide attachment sites through which the at least one agent is conjugated to form the dendrimer conjugate. Accordingly, in some embodiments, the at least one agent is conjugated to the dendrimer through an ether bond, an amide bond, or an ester bond formed by conjugation to a terminal functional group of the dendrimer. In some embodiments, the at least one agent is conjugated to the dendrimer through an ether bond or an amide bond. In some embodiments, the at least one agent is conjugated to the dendrimer through an ether bond.

In some embodiments, the number of terminal sites on a dendrimer can depend on the particular dendrimeric scaffold and its generation. For example, in some embodiments, a dendrimer is based on a generation 0, 1, 2, 3, 4, 5, 6, 7, 8, 9, or 10 PAMAM dendrimeric scaffold, which have 4, 8, 16, 32, 64, 128, 256, 512, 1024, 2048, and 4096 terminal sites, respectively. However, it should be appreciated that different dendrimeric scaffolds having a different number of terminal sites at each generation can be used in accordance with the disclosure.

In some embodiments, all terminal sites of a dendrimer comprise hydroxyl groups. In some embodiments, each terminal site of a dendrimer comprises either a hydroxyl group or an amine group. In some embodiments, each terminal site of a dendrimer conjugate comprises a hydroxyl group, an amine group, or an agent conjugated to the dendrimer through an ether or amide bond. In some embodiments, each terminal site of a dendrimer conjugate comprises either a hydroxyl group or an agent conjugated to the dendrimer through an ether bond.

In some embodiments, at least 50% of terminal sites on a dendrimer conjugate comprise hydroxyl groups (e.g., at least 50% of terminal sites do not comprise either an amine group or an agent). For example, in some embodiments, at least 60%, at least 70%, at least 80%, at least 90%, at least 95%, at least 98%, or at least 99% of terminal sites on a dendrimer conjugate comprise hydroxyl groups. In some embodiments, about 50-99%, about 60-99%, about 70-99%, about 80-99%, about 90-99%, about 95-99%, about 98-99%, about 70-95%, about 70-90%, about 80-95%, or about 80-90% of terminal sites on a dendrimer conjugate comprise hydroxyl groups.

In some embodiments, one or more terminal sites on a dendrimer conjugate comprise an agent. In some embodiments, at least 2, at least 3, at least 4, at least 5, at least 10, at least 15, at least 20, or more, terminal sites on a dendrimer conjugate comprise an agent. In some embodiments, at least 1% of terminal sites on a dendrimer conjugate comprise an agent. For example, in some embodiments, at least 2%, at least 5%, at least 10%, at least 15%, at least 20%, at least 25%, or at least 30% of terminal sites on a dendrimer conjugate comprise an agent. In some embodiments, about 1-50%, about 1-40%, about 1-25%, about 1-10%, about 5-50%, about 5-40%, about 5-25%, about 5-10%, about 10-50%, about 10-40%, or about 10-25% of terminal sites on a dendrimer conjugate comprise an agent. In some embodiments, about 1%, about 2%, about 3%, about 4%, or about 5% of terminal sites on a dendrimer comprise an agent. In some embodiments, less than 5%, less than 10%, less than 15%, less than 20%, less than 25%, less than 30%, less than 35%, less than 40%, less than 45%, less than 50%, less than 55%, less than 60%, less than 65%, less than 70%, less than 75% of terminal sites on a dendrimer comprise an agent. In some embodiments, a dendrimer conjugate has an effective amount of terminal functional groups (e.g., terminal hydroxyl groups) for targeting to a specific cell type, while having to an effective amount of agent for treating and/or imaging as described herein. In some embodiments, terminal sites of a dendrimer conjugate can be evaluated using proton nuclear magnetic resonance (¹H NMR), or other analytical methods known in the art, to determine a percentage of terminal sites having an agent and/or terminal functional group.

In some embodiments, a desired agent loading can depend on certain factors, including the choice of agent, dendrimer structure and size, and cell or tissue to be treated. In some embodiments, a dendrimer conjugate (e.g., a therapeutic compound) is about 0.01% to about 45% by mass (m/m) of agent (e.g., therapeutic agent). In some embodiments, a dendrimer conjugate (e.g., a therapeutic compound) is about 10% to about 20% by mass of agent (e.g., therapeutic agent). In some embodiments, a dendrimer conjugate is about 0.1% to about 30%, about 0.1% to about 20%, about 0.1% to about 10%, about 1% to about 10%, about 1% to about 5%, about 3% to about 20%, about 3% to about 10% by mass of agent.

As described herein, in some embodiments, a dendrimer conjugate can be characterized in terms of mass percentage (e.g., % by mass (m/m)) of agent. In some embodiments, mass percentage refers to a molecular weight (Da) percentage of agent in a dendrimer conjugate. In some embodiments, mass percentage can be determined by the general formula of: (agent Mw)/(conjugate Mw)×100. For example, in some embodiments, (agent Mw) can be determined by calculating or approximating the molecular weight of an agent as a single molecule or compound (conjugated or unconjugated), and multiplying this value by the number of terminal sites at which the agent is present in a dendrimer conjugate. In some embodiments, (agent Mw) can be determined by calculating or approximating the sum of the atomic mass of all atoms which form the agent in a dendrimer conjugate. The value for (agent Mw) can be taken as a fraction of total molecular weight of the dendrimer conjugate (conjugate Mw), and multiplied by 100 to provide a mass percentage. In some embodiments, mass percentage can be determined by experimental or empirical means. For example, in some embodiments, mass percentage can be determined using proton nuclear magnetic resonance (¹H NMR) or other analytical methods known in the art.

In some embodiments, a dendrimer has a diameter of between about 1 nm and about 50 nm. For example, in some embodiments, the diameter is between about 1 nm and about 20 nm, between about 1 nm and about 10 nm, or between about 1 nm and about 5 nm. In some embodiments, the diameter is between about 1 nm and about 2 nm. In some embodiments, a dendrimer that is conjugated to a relatively large agent (e.g., a large protein, such as an antibody) can have a diameter that increases these values by approximately 5-15 nm relative to the unconjugated dendrimer. In some embodiments, a dendrimer has a molecular weight of between about 500 Daltons (Da) and about 100,000 Da (e.g., between about 500 Da and about 50,000 Da, or between about 1,000 Da and about 20,000 Da).

In some embodiments, a dendrimer of a conjugate described herein is a poly(amidoamine) (PAMAM) dendrimer, a polypropylamine (POPAM) dendrimer, a 2,2-bis(hydroxymethyl)propionic acid (bis-MPA) dendrimer, a polyethylenimine dendrimer, a polylysine dendrimer, a polyester dendrimer, an iptycene dendrimer, aliphatic poly(ether) dendrimer, an aromatic polyether dendrimer, or a combination thereof.

In some embodiments, a dendrimer conjugate comprises a PAMAM dendrimer. In some embodiments, a PAMAM dendrimer comprises different cores with amidoamine building blocks. In some embodiments, a PAMAM dendrimer comprises carboxylic, amine, and/or hydroxyl terminal groups of any generation including, but not limited to, generation 1, generation 2, generation 3, generation 4, generation 5, generation 6, generation 7, generation 8, generation 9, or generation 10 PAMAM dendrimers. In some embodiments, a PAMAM dendrimer is a generation 4, generation 5, generation 6, generation 7, or generation 8 hydroxyl-terminated PAMAM dendrimer.

In some embodiments, the dendrimers include a plurality of hydroxyl groups. Some exemplary high-density hydroxyl groups-containing dendrimers include commercially available polyester dendritic polymer such as hyperbranched 2,2-Bis(hydroxyl-methyl)propionic acid polyester polymer (for example, hyperbranched bis-MPA polyester-64-hydroxyl, generation 4), dendritic polyglycerols. In some embodiments, the high-density hydroxyl groups-containing dendrimers are oligo ethylene glycol (OEG)-like dendrimers. For example, a generation 2 OEG dendrimer (D2-OH-60) can be synthesized using highly efficient, robust and atom economical chemical reactions such as Cu (I) catalyzed alkyne-azide click and photo catalyzed thiol-ene click chemistry. Highly dense polyol dendrimer at very low generation in minimum reaction steps can be achieved by using an orthogonal hypermonomer and hypercore strategy, for example as described in WO 2019094952. In some embodiments, dendrimer backbone has non-cleavable polyether bonds throughout the structure to avoid the disintegration of dendrimer in vivo, and to allow the elimination of such dendrimers as a single entity from the body (e.g., non-biodegradable).

In some embodiments, a dendrimer conjugate comprises a dendrimer that is conjugated to one or more therapeutic agents, one or more imaging agents, and/or one or more targeting agents. It should be appreciated that, in some embodiments, “at least one” agent, “one or more” agents, and similar terminology refer to a particular agent and not necessarily the amount of the particular agent that is conjugated to a dendrimer. For example, in some embodiments, a dendrimer conjugate comprising two agents refers to a dendrimer having a first agent at one or more terminal positions and a second agent at one or more different terminal positions, where the first and second agents are different (e.g., chemically different). In some embodiments, the first and second agents may be useful for a similar purpose (e.g., both agents are therapeutic agents), or the first and second agents may be useful for different purposes (e.g., the first agent is a therapeutic agent, and the second agent is a targeting agent). When used for a similar purpose, the first and second agents are chemically different and can therefore provide different functionalities—for example, different therapeutic agents targeting different receptors or biological pathways, or different imaging agents having different spectral properties.

In some embodiments, an agent (e.g., a therapeutic agent, an imaging agent, a targeting agent) of a dendrimer conjugate is a peptide, a protein, a sugar, a carbohydrate, an oligonucleotide, a nucleic acid, a lipid, a small-molecule compound, or a combination thereof. In some embodiments, an agent is an antibody or an antigen-binding fragment of an antibody. In some embodiments, an agent is a nucleic acid or oligonucleotide that encodes a protein, such as a DNA expression vector or an mRNA. In some embodiments, an agent is an RNA-silencing agent, such as an siRNA, shRNA, or a microRNA.

In some embodiments, an agent is a small-molecule compound, such as a small-molecule organic, organometallic, or inorganic compound. In some embodiments, an agent is a small-molecule compound having a molecular weight of less than 2,000 daltons (Da), less than 1,500 Da, less than 1,000 Da, or less than 500 Da. In some embodiments, an agent is a small-molecule compound having a molecular weight of between about 100 and about 2,000 Da. For example, in some embodiments, the small-molecule compound has a molecular weight of between about 100 and about 1,500 Da, between about 100 and about 1,000 Da, between about 500 and about 2,000 Da, or between about 300 and about 700 Da.

In some aspects, a non-releasable form of a dendrimer conjugate described herein provides enhanced therapeutic efficacy as compared to a releasable form of the same conjugate. Accordingly, in some embodiments, an agent is conjugated to a dendrimer through a linker, which is attached to the dendrimer and to the agent in a non-releasable manner (e.g., by ether and/or amide bonds). In some embodiments, a linker has a composition that is minimally releasable (e.g., minimally cleavable) under physiological conditions.

In some embodiments, a dendrimer is conjugated to an agent through covalent bonds that are stable under in vivo conditions. In some embodiments, the covalent bonds are minimally cleavable when administered to a subject and/or excreted intact from the body. For example, in some embodiments, less than 10%, 5%, 4%, 3%, 2%, 1%, 0.5%, 0.4%, 0.3%, 0.2%, 0.1%, or less than 0.1% of the total dendrimer conjugates have agent cleaved within 24 hours, or 48 hours, or 72 hours after in vivo administration to a subject. In some embodiments, the covalent bonds comprise ether bonds. In some embodiments, the covalent bonds between dendrimer and agent are not hydrolytically or enzymatically cleavable bonds, such as ester bonds.

In some aspects, the disclosure provides a dendrimer conjugate of Formula (I):

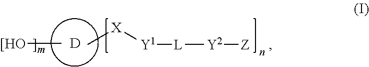

wherein: D is a dendrimer; X is O or NH; Y¹ is a first group; Y² is a second group; Z is an agent; L is a linker; m is an integer from 16 to 4096, inclusive; and n is an integer from 1 to 100, inclusive.

In some embodiments, D is a dendrimer selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof.

In some embodiments, Y¹ is non-hydrolyzable under physiological conditions. In some embodiments, Y¹ is optionally substituted alkylene, optionally substituted alkenylene, optionally substituted alkynylene, or a covalent bond. In some embodiments, Y¹ is optionally substituted C₁₋₂₀ alkylene. In some embodiments, Y¹ is unsubstituted C₁₋₁₀ alkylene.

In some embodiments, Y¹ is optionally substituted alkylene, optionally substituted alkenylene, optionally substituted alkynylene, or a covalent bond. In some embodiments, Y¹ is optionally substituted alkylene (e.g., optionally substituted C₁₋₂₀ alkylene). In some embodiments, Y¹ is optionally substituted ethylene. In some embodiments, Y¹ is optionally substituted methylene. In some embodiments, Y¹ is of the formula:

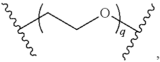

wherein q is 1, 2, 3, 4, 5, or 6 (e.g., wherein q is 1, 2, or 3). In some embodiments, q is 2. In some embodiments, q is 1. In some embodiments, Y¹ is unsubstituted alkylene (e.g., unsubstituted C₁₋₁₀alkylene). In some embodiments, Y¹ is of the formula:

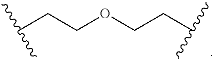 [SMILES: *CCOCC*]

In some embodiments, Y² is selected from the group consisting of secondary amides, tertiary amides, sulfonamide, secondary carbamates, tertiary carbamates, carbonates, ureas, carbinols, disulfides, hydrazones, hydrazides, ethers, carbonyls, and combinations thereof. In some embodiments, Y² is selected from the group consisting of —CONH—, —CONR^{A}—, —SO₂NR^{A}—, —OCONH—, —NHCOO—, —OCONR^{A}—, —NR^{A}COO—, —OC(═O)O—, —NHCONH—, —NR^{A}CONH—, —NHCONR^{A}—, —NRCONR^{A}—, —CHOH—, —CR^{A}OH—, —C(═O)—, and —C(═O)R^{A}—, wherein R^{A} is an optionally substituted alkyl group, an optionally substituted aryl group, or an optionally substituted heterocyclic group.

In some embodiments, Y² comprises a polymer. In some embodiments, Y² comprises an alkylene chain (e.g., a C_{1-100,000} alkylene), wherein the chain is the shortest path between L and Z, excluding hydrogen atoms and substituents. In some embodiments, the chain of Y² comprises up to 5,000-7,000; 7,000-9,000; 9,000-10,000; 10,000-12,000; 100,000-120,000; 120,000-150,000; or 150,000-200,000 consecutive covalently bonded atoms or in length, excluding hydrogen atoms and substituents. In some embodiments, Y² is an all-carbon, substituted or unsubstituted C_{1-200,000} hydrocarbon chain as the shortest path between L and Z, excluding hydrogen atoms and substituents. In some embodiments, any of the atoms in Y² can be substituted. In some embodiments, none of the atoms in Y² are substituted. In some embodiments, none of the carbon atoms in Y² are substituted. In some embodiments, at least one chain atom of the hydrocarbon chain of Y² is independently replaced with —C(═O)—, —O—, —NR—, —S—, or a cyclic moiety, wherein R^{b} is independently hydrogen, substituted or unsubstituted C₁₋₆ alkyl, or a nitrogen protecting group. In some embodiments, at least one chain atom of the hydrocarbon chain of Y² is independently replaced with amide, hydroxamate, ether, n-alkyl, carbonate, carbamate, hydrazone, thioether, thioester, disulfide, orthoester, urethane, oxime, optionally substituted carbocyclylene, optionally substituted heterocyclylene, optionally substituted arylene, and/or optionally substituted heteroarylene. In some embodiments, at least one chain atom of the hydrocarbon chain of Y² is independently replaced with amide, hydroxamate, ether, carbonate, carbamate, hydrazone, thioether, thioester, disulfide, orthoester, urethane, and/or oxime. In some embodiments, Y² comprises an alkylene moiety (e.g., of formula

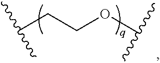

wherein q is an integer between 1-100, inclusive). In some embodiments, Y² comprises a polyethylene glycol moiety (e.g., of formula

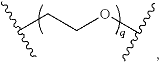

wherein q is an integer between 1-100, inclusive). In some embodiments, Y² comprises an alkylene moiety or a polyethylene glycol moiety, and a hydrocarbon chain. In some embodiments, at least one chain atom of the hydrocarbon chain of Y² is independently replaced with amide, hydroxamate, ether, carbonate, carbamate, hydrazone, thioether, thioester, disulfide, orthoester, urethane, and/or oxime.

In some embodiments, Z is a therapeutic agent, an imaging agent, or a targeting agent as described herein. In some embodiments, Z is a therapeutic agent or an imaging agent. In some embodiments, the dendrimer conjugate of Formula (I) further comprises at least one targeting agent conjugated to the dendrimer.

In some embodiments, Z comprises a PAK1 inhibitor. In some embodiments, Z comprises Frax-1036. In some embodiments, Z is of the formula:

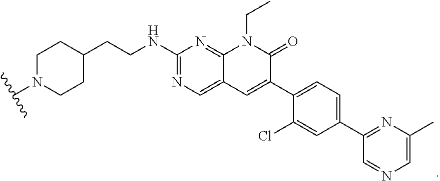 [SMILES: *N1CCC(CCNc2ncc3cc(-c4ccc(-c5cncc(C)n5)cc4Cl)c(=O)n(CC)c3n2)CC1]

In some embodiments, Z comprises a MEK inhibitor. In some embodiments, Z comprises selumetinib. In some embodiments, Z is of the formula:

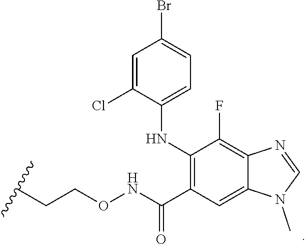 [SMILES: *CCONC(=O)c1cc2c(ncn2C)c(F)c1Nc1ccc(Br)cc1Cl]

In some embodiments, Z comprises trametinib. In some embodiments, Z is of the formula:

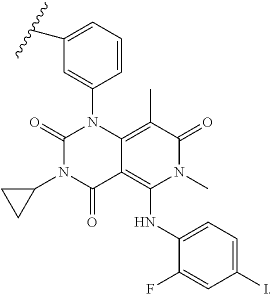 [SMILES: *c1cccc(-n2c(=O)n(C3CC3)c(=O)c3c(Nc4ccc(I)cc4F)n(C)c(=O)c(C)c32)c1]

In some embodiments, Z comprises cobimetinib. In some embodiments, Z is of the formula:

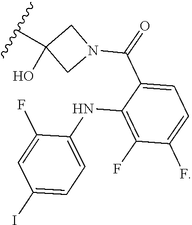 [SMILES: *C1(O)CN(C(=O)c2ccc(F)c(F)c2Nc2ccc(I)cc2F)C1]

In some embodiments, Z is of the formula:

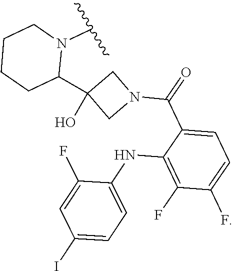 [SMILES: *N1CCCCC1C1(O)CN(C(=O)c2ccc(F)c(F)c2Nc2ccc(I)cc2F)C1]

In some embodiments, Z is of the formula:

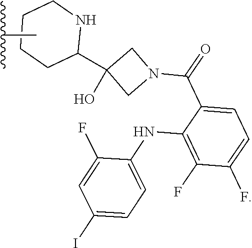

In some embodiments, Z comprises a receptor tyrosine kinase inhibitor. In some embodiments, Z comprises dasatinib. In some embodiments, Z is of the formula:

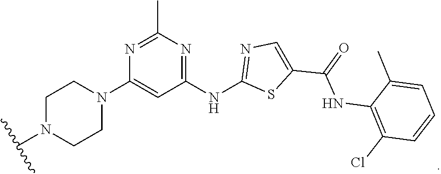 [SMILES: *N1CCN(c2cc(Nc3ncc(C(=O)Nc4c(C)cccc4Cl)s3)nc(C)n2)CC1]

In some embodiments, Z comprises bemcentinib (R428). In some embodiments, Z is of the formula:

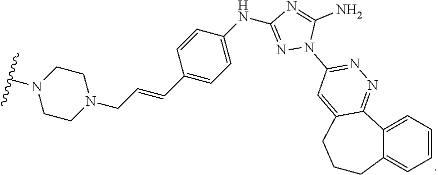 [SMILES: *N1CCN(C/C=C/c2ccc(Nc3nc(N)n(-c4cc5c(nn4)-c4ccccc4CCC5)n3)cc2)CC1]

In some embodiments, Z comprises dubermatinib (TP-0903). In some embodiments, Z is of the formula:

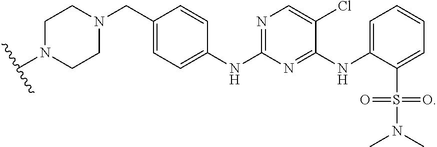 [SMILES: *N1CCN(Cc2ccc(Nc3ncc(Cl)c(Nc4ccccc4S(=O)(=O)N(C)C)n3)cc2)CC1]

In some embodiments, Z comprises cabozantinib. In some embodiments, Z is of the formula:

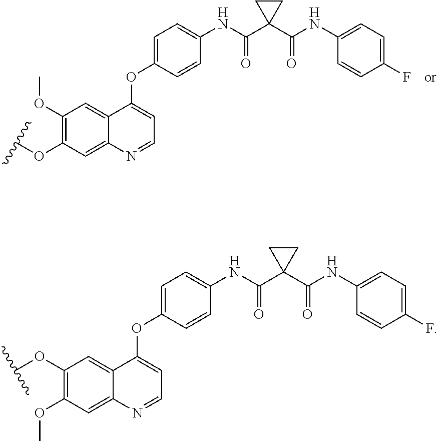 [SMILES: *Oc1cc2nccc(Oc3ccc(NC(=O)C4(C(=O)Nc5ccc(F)cc5)CC4)cc3)c2cc1OC ; *Oc1cc2c(Oc3ccc(NC(=O)C4(C(=O)Nc5ccc(F)cc5)CC4)cc3)ccnc2cc1OC]

In some embodiments, L is a linker comprising a polymer and at least one moiety. In some embodiments, the polymer is a polymeric polyol, a polypeptide, or an unsubstituted alkyl chain. In some embodiments, the polymer is a polymeric polyol selected from the group consisting of polyethylene glycol (PEG), polypropylene glycol, and polyvinyl alcohol. In some embodiments, the polymer is a polypeptide having at least 2 amino acids. In some embodiments, the polymer is a polypeptide having between about 2 and about 40 amino acids (e.g., 2-25, 5-30, 10-25, or 5-15 amino acids). In some embodiments, the polymer is an unsubstituted alkyl chain. In some embodiments, the polymer is an unsubstituted C₂₋₅₀alkyl chain. In some embodiments, the polymer is an unsubstituted C₂₋₃₀ alkyl chain. In some embodiments, the polymer is an unsubstituted C₅₋₂₅ alkyl chain. In some embodiments, the polymer is a polymer as described elsewhere herein.

In some embodiments, the at least one moiety of L is a moiety resulting from a click reaction. In some embodiments, the at least one moiety is a 5-membered heterocyclic ring resulting from an electrocyclic reaction (e.g., 3+2 cycloaddition, or 4+2 cycloaddition) between reactive click chemistry handles (e.g., azides and terminal or strained alkynes, dienes and dienophiles, thiols and alkenes) used to produce the conjugate. In some embodiments, the at least one moiety is a diradical comprising 1,2,3-triazolyl, 4,5-dihydro-1,2,3-triazolyl, isoxazolyl, 4,5-dihydroisoxazolyl, or 1,4-dihydropyridazyl.

In some embodiments, L comprises a polyethylene glycol moiety of the formula:

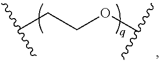

wherein q is an integer between 1-100, inclusive, and a hydrocarbon chain. In some embodiments, at least one chain atom of the hydrocarbon chain of L is independently replaced with amide, hydroxamate, ether, carbonate, carbamate, hydrazone, thioether, thioester, disulfide, orthoester, urethane, and/or oxime. In some embodiments, q is an integer between 1-50, inclusive. In some embodiments, q is an integer between 1-10, inclusive. In some embodiments, q is an integer between 1-8, inclusive. In some embodiments, L is of the formula

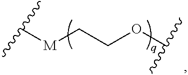

wherein M is a diradical comprising 1,2,3-triazolyl, 4,5-dihydro-1,2,3-triazolyl, isoxazolyl, 4,5-dihydroisoxazolyl, or 1,4-dihydropyridazyl, and q is an integer between 1-100, inclusive. In some embodiments, q is an integer between 1-50, inclusive. In some embodiments, q is an integer between 1-10, inclusive. In some embodiments, q is an integer between 1-8, inclusive. In some embodiments, L is of the formula

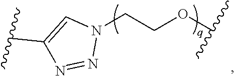

wherein q is an integer between 1-100, inclusive. In some embodiments, q is an integer between 1-50, inclusive. In some embodiments, q is an integer between 1-10, inclusive. In some embodiments, q is an integer between 1-8, inclusive.

In some aspects, the disclosure provides a dendrimer conjugate of Formula (II):

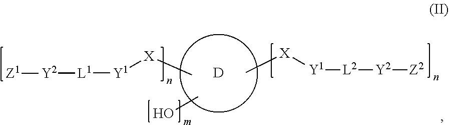

wherein: D, m, each instance of n, each instance of X, each instance of Y¹, and each instance of Y² is independently as defined with respect to Formula (I); L¹ and L² are independently linkers as defined with respect to Formula (I); and Z¹ and Z² are different agents.

In some embodiments, Z¹ and Z² are independently therapeutic agents, targeting agents, or imaging agents, with the proviso that Z¹ and Z² are different (e.g., chemically different). In some embodiments, Z¹ and Z² are different therapeutic agents. In some embodiments, Z¹ and Z² are different therapeutic agents targeting different biological pathways implicated in a common pathology. In some embodiments, Z¹ and Z² are different therapeutic agents, and the dendrimer conjugate of Formula (II) further comprises at least one targeting agent conjugated to the dendrimer. In some embodiments, Z¹ and Z² are different imaging agents. In some embodiments, Z¹ is a therapeutic agent, and Z² is a targeting agent. In some embodiments, Z¹ is an imaging agent, and Z² is a targeting agent.

### Methods of Synthesis

In some aspects, the disclosure relates to the discovery of new techniques for synthesizing dendrimers (e.g., functionalized dendrimers, for example, dendrimers functionalized with a specified number of PEG-alkynes, to allow for further installation of an agent via click conjugation chemistry, e.g., azide-alkyne cycloaddition). Advantageously, methods and compositions provided herein can allow for large multigram-scale synthesis (e.g., over 10 grams, over 40-50 grams, over 100 grams) of the dendrimers (e.g., dendrimers functionalized with a specified number of PEG-alkynes) with high product uniformity (low polydispersity, e.g., less than 1.05) using milder conditions with tuned stoichiometry relative to previous techniques.

In some aspects, the disclosure provides methods of synthesizing a functionalized dendrimer of Formula (I-A):

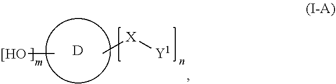

wherein:
D is a dendrimer selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof;
X is NH;
Y¹ is optionally substituted alkylene, optionally substituted alkenylene, optionally substituted alkynylene, or a covalent bond;
m is an integer from 16 to 4096, inclusive; and
n is an integer from 1 to 100, inclusive;
comprising:
reacting a dendrimer of Formula (II-A):

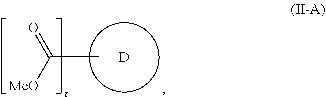

under suitable conditions to form the functionalized dendrimer of Formula (I-A);
   wherein:
D is a dendrimer selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof;
t is an integer from 16 to 4096, inclusive;
with one or more amines, wherein each amine is of the formula H₂NR¹,
wherein R¹ is optionally substituted alkylene, optionally substituted alkenylene, optionally substituted alkynylene, or a covalent bond.

In some embodiments, in a dendrimer of Formula (I-A) or (II-A), D is a dendrimer selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof.

In some embodiments, Y¹ is optionally substituted alkylene, optionally substituted alkenylene, optionally substituted alkynylene, or a covalent bond. In some embodiments, Y¹ is optionally substituted alkylene (e.g., optionally substituted C₁₋₂₀ alkylene). In some embodiments, X is —NH and Y¹ is optionally substituted ethyl. In some embodiments, X is —NH and Y¹ is ethyl substituted with a PEG-alkyne group. In some embodiments, X is —NH and Y¹ is of the formula:

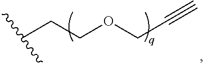

wherein q is 1, 2, 3, 4, 5, or 6 (e.g., wherein q is 1, 2, or 3). In some embodiments, X is —NH and Y¹ is of the formula:

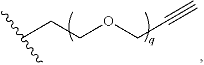

q, wherein q is 2. In some embodiments, Y¹ is unsubstituted alkylene (e.g., unsubstituted C₁₋₁₀ alkylene).

In some embodiments, m is an integer from 16 to 4096, inclusive. In some embodiments, m is an integer from 16 to 20, 20-40, 40-50, 50-60, 60-70, 70-80, 80-90, 90-100, 100-110, 110-120, 120-130, 130-140, 140-160, 160-180, 180-200, 200-250, 250-300, 300-350, 350-400, 400-450, 450-500, 500-550, 550-600, 600-650, 650-700, 700-750, 750-800, 800-850, 850-900, 900-950, 950-1000, 1000-1500, 1500-2000, 2000-2500, 2500-3000, 3000-3500, 3500-4000, 4000-4100, or 4100-4200, inclusive. In some embodiments, m is approximately 61 or 54 (e.g., 61 or 54).

In some embodiments, n is an integer from 1 to 100, inclusive. In some embodiments, n is an integer from 1 to 3, 3 to 5, 1 to 5, 5-7, 7-9, 9-10, 10-12, 12-14, 14-16, 16-18, 18-20, 20-22, 22-24, 24-26, 26-28, 28-30, 30-32, 32-34, 34-36, 36-38, 38-40, 40-42, 42-44, 44-46, 46-48, 48-50, 50-52, 52-54, 54-56, 56-58, 58-60, 60-62, 62-64, 64-66, 66-68, 68-70, 70-72, 72-74, 74-76, 76-78, 78-80, 80-82, 82-84, 84-86, 86-88, 88-90, 90-92, 92-94, 94-96, 96-98, or 98-100, inclusive. In some embodiments, n is approximately 3 or 10 (e.g., 3 or 10). In some embodiments, m is the value of n subtracted from 64. In some embodiments, n is 1. In some embodiments, n is 2. In some embodiments, n is 3. In some embodiments, n is 4. In some embodiments, n is 5. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75. In some embodiments, n is between approximately 1-2, 2-4, 4-5, 5-6, 6-8, 8-10, or 10-12; or between approximately 2-11 (e.g., 3, 4, 5, 8, 10). In some embodiments, n is approximately 3 or 10 (e.g., 3 or 10). In some embodiments, n is 3 and m is 61. In some embodiments, n is 10 and m is 54. In some embodiments, m is approximately 61 or 54 (e.g., 61 or 54).

In some embodiments, in the methods of synthesis described herein, provided is a dendrimer of Formula (II-A):

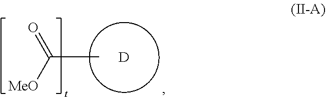

wherein D is a dendrimer selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof;
t is an integer from 16 to 4096, inclusive;
with one or more amines, wherein each amine is of the formula H₂NR¹,
wherein R¹ is optionally substituted alkylene, optionally substituted alkenylene, optionally substituted alkynylene, or a covalent bond.

In some embodiments, in Formula (II-A), D is a dendrimer as described herein. In some embodiments, the method of synthesis comprises reacting a dendrimer of Formula (II-A), or a salt, solvate, hydrate, stereoisomer, polymorph, tautomer, isotopically enriched form (e.g., isotopically labeled derivative) thereof. Formula (II-A) includes substituent t. In some embodiments, t is an integer from 16 to 4096, inclusive. In some embodiments, t is an integer from 16 to 20, 20-40, 40-50, 50-60, 60-70, 70-80, 80-90, 90-100, 100-110, 110-120, 120-130, 130-140, 140-160, 160-180, 180-200, 200-250, 250-300, 300-350, 350-400, 400-450, 450-500, 500-550, 550-600, 600-650, 650-700, 700-750, 750-800, 800-850, 850-900, 900-950, 950-1000, 1000-1500, 1500-2000, 2000-2500, 2500-3000, 3000-3500, 3500-4000, 4000-4100, or 4100-4200, inclusive. In some embodiments, n is t. In some embodiments, t is n. In some embodiments, in the dendrimer of Formula (II-A), t is the same as n. In some embodiments, in the dendrimer of Formula (II-A), t is 64. In some embodiments, t is an integer from 20-40, 40-50, 50-60, 60-70, 70-80, inclusive.

In some embodiments, the method of synthesis comprises reacting a dendrimer of Formula (II-A) with one or more amines. In some embodiments, the method of synthesis comprises reacting a dendrimer of Formula (II-A) with one or more amines, wherein at least one instance of the one or more amines is a compound of Formula (Z),

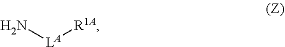

or a salt, solvate, hydrate, stereoisomer, polymorph, tautomer, isotopically enriched form (e.g., isotopically labeled derivative) thereof, wherein L^{A} is a linker, and R^{1A} is halogen, optionally substituted acyl, optionally substituted alkenylene, optionally substituted alkynylene, optionally substituted acetylene, optionally substituted carbocyclyl, optionally substituted heterocyclyl, optionally substituted aryl, optionally substituted heteroaryl, —CH(═N)(OH)R^{D1}, —CN, —NO₂, —OR^{D1}, —N(R^{D1a})₂, —SO₂OR^{D1}, or —SR^{D1} and R^{D1} and R^{D1a} are as defined herein. In some embodiments, linker L^{A} Comprises a polymer. In some embodiments, linker L^{A} comprises an alkylene chain (e.g., a C_{1,100,000} alkylene), wherein the chain is the shortest path between —NH₂ and R^{1A}, excluding hydrogen atoms and substituents. In certain embodiments, the chain of linker L^{A} Comprises up to 5,000-7,000; 7,000-9,000; 9,000-10,000; 10,000-12,000; 100,000-120,000; 120,000-150,000; or 150,000-200,000 consecutive covalently bonded atoms or in length, excluding hydrogen atoms and substituents. In certain embodiments, L^{A} is an all-carbon, substituted or unsubstituted C_{1-200,000} hydrocarbon chain as the shortest path between —NH₂ and R^{1A} excluding hydrogen atoms and substituents. In certain embodiments, any of the atoms in L^{A} can be substituted. In certain embodiments, none of the atoms in the linker L^{A} are substituted. In certain embodiments, none of the carbon atoms in the linker are substituted. In certain embodiments, at least one chain atom of the hydrocarbon chain of L^{A} is independently replaced with —C(═O)—, —O—, —NR—, —S—, or a cyclic moiety, wherein R^{b} is independently hydrogen, substituted or unsubstituted C₁₋₆ alkyl, or a nitrogen protecting group. In certain embodiments, at least one chain atom of the hydrocarbon chain of L^{A} is independently replaced with amide, hydroxamate, ether, n-alkyl, carbonate, carbamate, hydrazone, thioether, thioester, disulfide, orthoester, urethane, oxime, optionally substituted carbocyclylene, optionally substituted heterocyclylene, optionally substituted arylene, and/or optionally substituted heteroarylene. In certain embodiments, at least one chain atom of the hydrocarbon chain of L^{A} is independently replaced with amide, hydroxamate, ether, carbonate, carbamate, hydrazone, thioether, thioester, disulfide, orthoester, urethane, and/or oxime. In certain embodiments, the linker L^{A} comprises a polyethylene glycol moiety (e.g., of formula

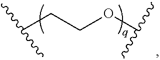

wherein q is an integer between 1-100,000, inclusive), and at least one chain atom of the hydrocarbon chain of L^{A} is independently replaced with amide, hydroxamate, ether, carbonate, carbamate, hydrazone, thioether, thioester, disulfide, orthoester, urethane, and/or oxime. In certain embodiments, the linker L^{A} comprises a moiety resulting from a click reaction. In some embodiments, the at least one moiety resulting from a click reaction is a 5-membered heterocyclic ring resulting from an electrocyclic reaction (e.g., 3+2 cycloaddition, or 4+2 cycloaddition) between reactive click chemistry handles (e.g., azides and terminal or strained alkynes, dienes and dienophiles, thiols and alkenes) used to produce the conjugate. In some embodiments, the at least one moiety resulting from a click reaction is a diradical comprising 1,2,3-triazolyl, 4,5-dihydro-1,2,3-triazolyl, isoxazolyl, 4,5-dihydroisoxazolyl, or 1,4-dihydropyridazyl. In certain embodiments, the linker L^{A} is of formula:

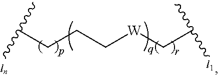

wherein 1, indicates the attachment to —NH₂ in a compound of Formula (Z), and hi indicates the attachment to R^{1A}. In some embodiments, the method of synthesis comprises reacting a dendrimer of Formula (II-A) with one or more amines, wherein at least one instance of the one or more amines is a compound of Formula (A):

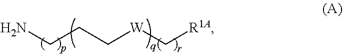

or a salt, solvate, hydrate, stereoisomer, polymorph, tautomer, isotopically enriched form (e.g., isotopically labeled derivative) thereof, wherein p, q, r, W, and R^{1A} are as defined herein. In some embodiments, the method of synthesis comprises reacting a dendrimer of Formula (II-A) with one or more amines, wherein at least one instance of the one or more amines is a compound of Formula (A):

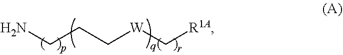

wherein: R^{1A} is halogen, optionally substituted acyl, optionally substituted alkenylene, optionally substituted alkynylene, optionally substituted acetylene, optionally substituted carbocyclyl, optionally substituted heterocyclyl, optionally substituted aryl, optionally substituted heteroaryl, —CH(═N)(OH)R^{D1}, —CN, —NO₂, —OR^{D1}, —N(R^{D1a})₂, —SO₂OR^{D1}, or —SR^{D1}, wherein R^{D1} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group when attached to an oxygen atom, or a sulfur protecting group when attached to a sulfur atom; wherein each occurrence of R^{D1a} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring; W is —O— or —CH₂—, as valency permits; p is 0, 1, 2, or 3; q is an integer between 1-100,000, inclusive; and r is 0, 1, 2, 3, 4, 5, or 6. In some embodiments, R^{1A} is halogen (e.g., F, Cl, Br, or I). In certain embodiments, R^{1A} is optionally substituted acyl (e.g., —C(═O)Me). In certain embodiments, R^{1A} is optionally substituted alkyl (e.g., substituted or unsubstituted C₁₋₆ alkyl). In certain embodiments, R^{1A} is substituted or unsubstituted methyl. In certain embodiments, R^{1A} is substituted or unsubstituted ethyl. In certain embodiments, R^{1A} is substituted or unsubstituted propyl. In certain embodiments, R^{1A} is optionally substituted alkenyl (e.g., substituted or unsubstituted C₂₋₆ alkenyl). In certain embodiments, R^{1A} is optionally substituted alkynyl (e.g., substituted or unsubstituted C₂₋₆ alkynyl). In certain embodiments, R^{1A} is optionally substituted carbocyclyl (e.g., substituted or unsubstituted, 3- to 7-membered, monocyclic carbocyclyl comprising zero, one, or two double bonds in the carbocyclic ring system). In certain embodiments, R^{1A} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R^{1A} is optionally substituted aryl (e.g., substituted or unsubstituted, 6- to 10-membered aryl). In certain embodiments, R^{1A} is benzyl. In certain embodiments, R^{1A} is substituted or unsubstituted phenyl. In certain embodiments, R^{1A} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R^{1A} is —CH(═N)(OH)R^{D1}, —CN, —NO₂, —OR^{D1}, —N(R^{D1a})₂, —SO₂OR^{D1} or —SR^{D1}, wherein R^{D1} and R^{D1a} are as defined herein. In certain embodiments, R^{1A} is —CH(═N)(OH)R^{D1} (e.g., —CH(═N)(OH)(optionally substituted C₁₋₆ alkyl)). In certain embodiments, R^{1A} is —CH(═N)(OH)(C₁₋₆ alkyl optionally substituted with a polyethylene glycol linker). In certain embodiments, R^{1A} is —CN. In certain embodiments, R^{1A} is —NO₂. In certain embodiments, R^{1A} is —OR^{D1} (e.g., —OH or —OMe). In certain embodiments, R^{1A} is —N(R^{D1a})₂ (e.g., —NMe₂). In certain embodiments, R^{1A} is —SO₂OR^{D1} (e.g., —SO₂O (optionally substituted alkyl)). In certain embodiments, R^{1A} is —SR^{D1} (e.g., —SMe). In certain embodiments, R^{1A} is —CH(═N)(OH)R^{D1}, —CN, —NO₂, —OR^{D1}, —N(R^{D1a})₂, —SO₂OR^{D1}, or —SR^{D1} wherein R^{D1} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group when attached to an oxygen atom, or a sulfur protecting group when attached to a sulfur atom; and wherein each occurrence of R^{D1a} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring. In certain embodiments, R^{1A} is bromo, alkyne, acetylene, alkene, aldehyde, amine, COOH, hydroxyl, carboxyl (e.g., dibenzocyclooctyne or DBCO), thiol, sulphonate, or —CN. In certain embodiments, R^{1A} is a click reaction handle partner (e.g., a click chemistry handle in Table A, or shown in Schemes 1-19 of Example 6). In certain embodiments, click handle, click partner, and click reaction handle partner are used interchangeably. In certain embodiments, the click reaction is: Cu (I) catalyzed alkyne-azide click and photo catalyzed thiol-ene click chemistry; or a electrocyclic click reaction (e.g., 3+2 cycloaddition, or 4+2 cycloaddition).

TABLE A
Exemplary click chemistry reactions and click chemistry handles

| | |
|---|---|
| (a) | 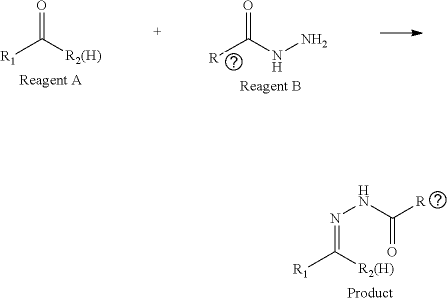 |
| (b) | 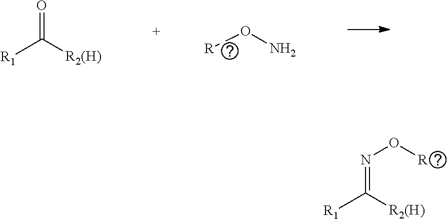 |
| (c) | 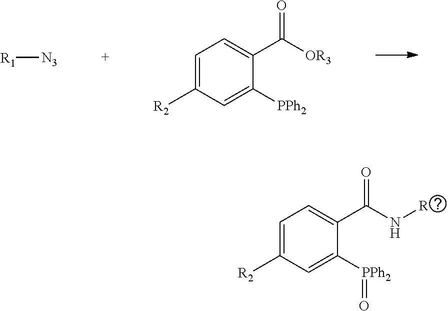 |
| (d) | 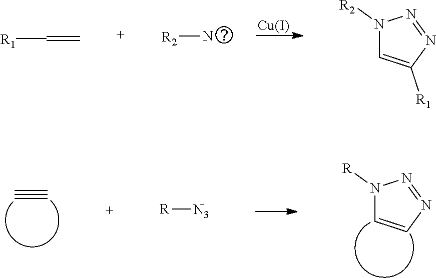 |
| | 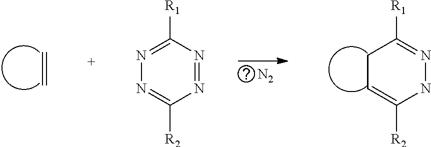 |

(a) ketone/aldehyde condensation;
(b) Staudinger reaction;
(c) 1,3 bipolar cycloaddition (top: Copper-catalyzed alkyne-azide cycloaddition (CuAAC); bottom: strain-promoted [3 + 2] azide-alkyne cycloaddition (SPAAC));
(d) inverse electron demand Diels-Alder reaction.

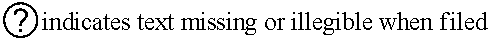

In certain embodiments, one of R^{1A} and R² is —N₃ and the other of R^{1A} and R² is dibenzocyclooctyne. In certain embodiments, one of R^{1A} and R² is

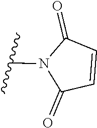 [SMILES: *N1C(=O)C=CC1=O]

and the other of R^{1A} and R² is —SH. In certain embodiments, one of R^{1A} and R² is tetrazine and the other of R^{1A} and R² is trans-cyclooctene. In certain embodiments, one of R^{1A} and R² is

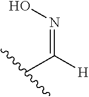 [SMILES: */C=N\O]

and the other of R^{1A} and R² is

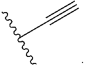 [SMILES: *C#C]

In certain embodiments, one of R^{1A} and R² is —SH and the other of R^{1A} and R² is

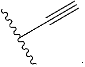 [SMILES: *C#C]

In certain embodiments, one of R^{1A} and R² is —SH and the other of R^{1A} and R² is

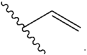 [SMILES: *C=C]

In certain embodiments, one of R^{1A} and R²—SH and the other of R^{1A} and R² is

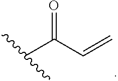 [SMILES: *C(=O)C=C]

In certain embodiments, R^{D1} is hydrogen. In certain embodiments, R^{D1} is optionally substituted acyl (e.g., —C(═O)Me). In certain embodiments, R^{D1} is optionally substituted alkyl (e.g., substituted or unsubstituted C₁₋₆ alkyl). In certain embodiments, R^{D1} is substituted or unsubstituted methyl. In certain embodiments, R^{D1} is substituted or unsubstituted ethyl. In certain embodiments, R^{D1} is substituted or unsubstituted propyl. In certain embodiments, R^{D1} is optionally substituted alkenyl (e.g., substituted or unsubstituted C₂₋₆ alkenyl). In certain embodiments, R^{D1} is optionally substituted alkynyl (e.g., substituted or unsubstituted C₂₋₆ alkynyl). In certain embodiments, R^{D1} is optionally substituted carbocyclyl (e.g., substituted or unsubstituted, 3- to 7-membered, monocyclic carbocyclyl comprising zero, one, or two double bonds in the carbocyclic ring system). In certain embodiments, R^{D1} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R^{D1} is optionally substituted aryl (e.g., substituted or unsubstituted, 6- to 10-membered aryl). In certain embodiments, R^{D1} is benzyl. In certain embodiments, R^{D1} is substituted or unsubstituted phenyl. In certain embodiments, R^{D1} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R^{D1} is an oxygen protecting group when attached to an oxygen atom. In certain embodiments, R^{D1} is a sulfur protecting group when attached to a sulfur atom.

In certain embodiments, at least one instance of R^{D1a} is hydrogen. In certain embodiments, at least one instance of R^{D1a} is optionally substituted acyl (e.g., —C(═O)Me). In certain embodiments, at least one R^{D1a} is optionally substituted alkyl (e.g., substituted or unsubstituted C₁₋₆ alkyl). In certain embodiments, at least one instance of R^{D1a} is substituted or unsubstituted methyl. In certain embodiments, at least one instance of R^{D1a} is substituted or unsubstituted ethyl. In certain embodiments, at least one instance of R^{D1a} is substituted or unsubstituted propyl. In certain embodiments, at least one instance of R^{D1a} is optionally substituted alkenyl (e.g., substituted or unsubstituted C₂₋₆ alkenyl). In certain embodiments, at least one instance of R^{D1a} is optionally substituted alkynyl (e.g., substituted or unsubstituted C₂₋₆ alkynyl). In certain embodiments, at least one instance of R^{D1a} is optionally substituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{D1a} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{D1a} is optionally substituted aryl (e.g., substituted or unsubstituted, 6- to 10-membered aryl). In certain embodiments, at least one instance of R^{D1a} is benzyl. In certain embodiments, at least one instance of R^{D1a} is substituted or unsubstituted phenyl. In certain embodiments, at least one instance of R^{D1a} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{D1a} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, two instances of R^{D1}a are taken together with their intervening atoms to form a optionally substituted heterocyclic ring (e.g., substituted or unsubstituted, 5- to 10-membered monocyclic or bicyclic heterocyclic ring, wherein one or two atoms in the heterocyclic ring are independently nitrogen, oxygen, or sulfur) or optionally substituted heteroaryl ring (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur).

In some embodiments, p is 0. In some embodiments, p is 1. In some embodiments, p is 2. In some embodiments, p is 3.

In some embodiments, W is —O—. In some embodiments, W is —O— and q is an integer between 1-100,000, inclusive. In some embodiments, W is —CH₂—. In some embodiments, W is —CH₂— and q is an integer between 1-10,000, inclusive.

In some embodiments, q is an integer between 1-50, inclusive. In some embodiments, q is an integer between 1-100, inclusive. In some embodiments, q is an integer between 1-5,000, inclusive. In some embodiments, q is an integer between 1-10,000, inclusive. In some embodiments, q is an integer between 1-50,000, inclusive. In some embodiments, q is an integer between 1-100,000, inclusive. In some embodiments, q is an integer between 1-25, 25-50, 50-75, 75-100, 100-125, 125-150, 150-175, 175-200, 200-225, 225-250, 250-275, 275-300, 300-325, 325-350, 350-375, 375-400, 400-425, 400-450, 450-500, 500-550, 550-600, 600-650, 650-700, 700-750, 750-800, 800-850, 850-900, 900-1000, 1000-1500, 1500-2000, 2000-2500, 2500-3000, 3000-3500, 3500-4000, 4000-4500, 4500-5000, 5000-6000, 6000-7000, 7000-8000, 8000-9000; 9000-10,000; 10,000-12,000; 100,000-120,000; 120,000-150,000; 150,000-175,000; or 175,000-200,000.

In some embodiments, the method of synthesis comprises reacting a dendrimer of Formula (II-A) with one or more amines, wherein each amine is of the formula H₂NR¹, and R¹ is as defined herein. In some embodiments, at least one instance of R¹ is optionally substituted alkylene, optionally substituted alkenylene, optionally substituted alkynylene, or a covalent bond. In some embodiments, at least one instance of R¹ is optionally substituted alkylene (e.g., optionally substituted C₁₋₂₀ alkylene). In some embodiments, each instance of R¹ is Y¹ in the one or more amines, wherein each amine is of the formula H₂NR¹. In some embodiments, at least one instance of R¹ is optionally substituted ethyl. In some embodiments, at least one instance of R¹ is ethyl substituted with a PEG-alkyne group. In some embodiments, at least one instance of R¹ is of the formula:

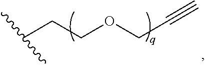

wherein q is 1, 2, 3, 4, 5, or 6 (e.g., wherein q is 1, 2, or 3). In some embodiments, at least one instance of R¹ is of the formula:

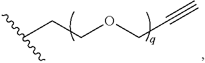

wherein q is 2.

In some embodiments, at least one instance of R¹ is unsubstituted alkylene (e.g., unsubstituted C₁₋₁₀ alkylene).

In some embodiments, the amine is a PEG-alkyne of formula:

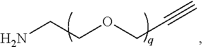

wherein q is 1, 2, 3, 4, 5 or 6. In some embodiments, the amine is a PEG-alkyne of formula:

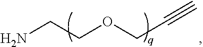

wherein q is 1, 2, or 3. In some embodiments, q is 1. In some embodiments, q is 2. In some embodiments, q is 3. In some embodiments, q is 4. In some embodiments, q is 5. In some embodiments, q is 6. In some embodiments, at least one instance of the one or
more amines is a PEG-alkyne of formula:

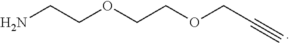

In some embodiments, at least one instance of the one or more amines is ethanolamine of formula:

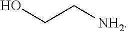

In some embodiments, the methods of synthesis described herein comprise two different amines. In some embodiments, one of the one or more amines is a PEG-alkyne of formula:

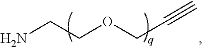

wherein q is 1, 2, 3, 4, 5, or 6; and the other amine is ethanolamine (e.g.,

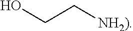

In some embodiments, one of the one or more amines is a PEG-alkyne of formula:

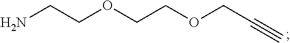

and the other amine is ethanolamine (e.g.,

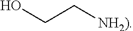

In some embodiments, in the methods of synthesis described herein, the dendrimer of Formula (II-A) is reacted with both ethanolamine and the PEG-alkyne of formula:

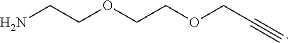

In some embodiments, a ratio of a first amine (e.g., ethanolamine) to a second amine (e.g., PEG-alkyne of formula:

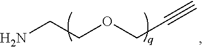

wherein q is 1, 2, 3, 4, 5, or 6) is approximately 10:1, 9.8: 1, 9.5: 1, 9.4: 1, 9.3: 1, 9.2: 1, 9.1: 1, or 9:1. In some embodiments, a ratio of the ethanolamine to the PEG-Alkyne of formula:

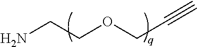

(e.g.,

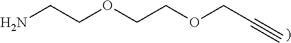

is 9.4:1. In some embodiments, a ratio of the ethanolamine to the PEG-Alkyne of formula:

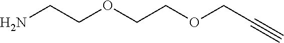

In some embodiments, a ratio of a first amine (e.g., ethanolamine) to a second amine (e.g., PEG-alkyne of formula:

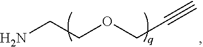

wherein q is 1, 2, 3, 4, 5, or 6) is approximately 3:1, 3.0: 1, 2.8: 1, 2.6: 1, 2.4: 1, 2.3: 1, 2.2: 1, or 2.1:1, or 2.0:1. In some embodiments, a ratio of the ethanolamine to the PEG-Alkyne of formula:

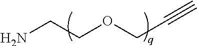

(e.g.,

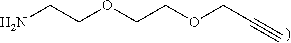

is 2.2:1. In some embodiments, a ratio of the ethanolamine to the PEG-Alkyne of formula:

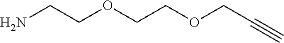

is 2.23:1. In some embodiments, a ratio of the ethanolamine to the PEG-Alkyne of formula:

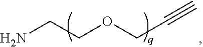

wherein q is 1, 2, 3, 4, 5, or 6 (e.g., the PEG-Alkyne of

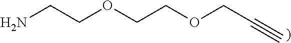

is 2.2:1 or 9.4:1.

In some embodiments, a ratio of one amine (e.g., the PEG-Alkyne of formula:

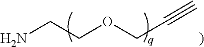

to the dendrimer of Formula (II-A) is approximately 150:1. In some embodiments, a ratio of one amine (e.g., the PEG-Alkyne of formula:

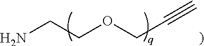

to the dendrimer of Formula (II-A)

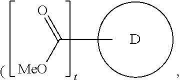

wherein D is PAMAM, and t is an integer of approximately 50-60, 60-70, or 70-80, inclusive) is approximately 150:1. In some embodiments, a ratio of one amine (e.g., the PEG-Alkyne of formula:

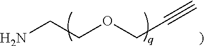

to the dendrimer of Formula (II-A) is approximately 150:1; the ratio of the ethanolamine to the PEG-Alkyne is 9.42:1; and n is 3.

In some embodiments, a ratio of one amine (e.g., the PEG-Alkyne of formula:

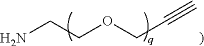

to the dendrimer of Formula (II-A)

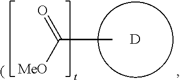

wherein D is PAMAM, and t is an integer of approximately 50-60, 60-70, or 70-80, inclusive) is approximately 152:1, 150:1, 149:1, 148:1, 145:1, 142:1, 140:1, or 138:1. In some embodiments, a ratio of one amine (e.g., the PEG-Alkyne of formula:

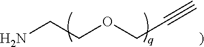

to the dendrimer of Formula (II-A)

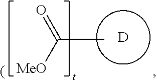

wherein D is PAMAM, and t is an integer of approximately 50-60, 60-70, or 70-80, inclusive) is approximately 152:1, 150:1, 149:1, 148:1, 145:1, 142:1, 140:1, or 138:1; and n is 3. In some embodiments, a ratio of one amine (e.g., the PEG-Alkyne of formula:

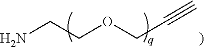

to the dendrimer of Formula (II-A)

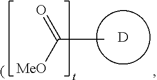

wherein D is PAMAM, and t is an integer of approximately 50-60, 60-70, or 70-80, inclusive) is approximately 152:1, 150:1, 149:1, 148:1, 145:1, 142:1, 140:1, or 138:1; the ratio of the ethanolamine to the PEG-Alkyne is 9.42:1; and n is 3. In some embodiments, a ratio of one amine (e.g., the PEG-Alkyne of formula:

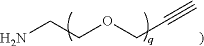

to the dendrimer of Formula (II-A)

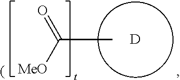

wherein D is PAMAM, and t is an integer of approximately 50-60, 60-70, or 70-80, inclusive) is approximately 150:1; the ratio of the ethanolamine to the PEG-Alkyne is 9.42:1; and n is 3.

In some embodiments, D is PAMAM. In some embodiments, D is PAMAM from generation 3.5, 4.5, 5.5, 6.5, 7.5, 8.5 or 9.5 where the PAMAM dendrimers are carrying A carboxy methyl group. In some embodiments, a ratio of one amine (e.g., the PEG-Alkyne of formula:

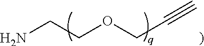

to the dendrimer of Formula (II-A)

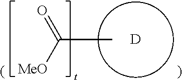

is approximately 500:1. In some embodiments, a ratio of one amine (e.g., the PEG-Alkyne of formula:

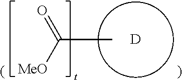

to the dendrimer of Formula (II-A)

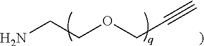

is approximately 500:1; the ratio of the ethanolamine to the PEG-Alkyne is 2.2:1; and n is 10. In some embodiments, a ratio of one amine (e.g., the PEG-Alkyne of formula:

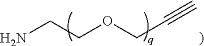

to the dendrimer of Formula (II-A)

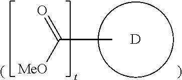

is 495:1. In some embodiments, a ratio of one amine (e.g., the PEG-Alkyne of formula:

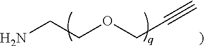

to the dendrimer of Formula (II-A)

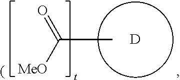

wherein D is PAMAM, and t is an integer of approximately 50-60, 60-70, or 70-80, inclusive) is approximately 505:1, 503:1, 502:1, 501:1, 500:1, 499:1, 498:1, 497:1, 496:1, 495:1, 494:1, 493:1, 492:1, 491:1, or 490:1. In some H₂NO embodiments, a ratio of one amine (e.g., the PEG-Alkyne of formula:

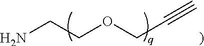

to the dendrimer of Formula (II-A)

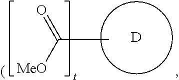

wherein D is PAMAM, and t is an integer of approximately 50-60, 60-70, or 70-80, inclusive) is approximately 495:1. In some embodiments, a ratio of one amine (e.g., the PEG-Alkyne of formula:

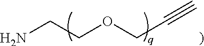

to the dendrimer of Formula (II-A)

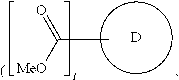

wherein D is PAMAM, and t is an integer of approximately 50-60, 60-70, or 70-80, inclusive) is approximately 505:1, 503:1, 502:1, 501:1, 500:1, 499:1, 498:1, 497:1, 496:1, 495:1, 494:1, 493:1, 492:1, 491:1, or 490:1 (e.g., approximately 495:1); and n is 10. In some embodiments, a ratio of one amine (e.g., the PEG-Alkyne of formula:

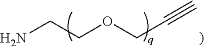

to the dendrimer of Formula (II-A) (

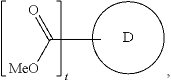

wherein D is PAMAM, and t is an integer of approximately 50-60, 60-70, or 70-80, inclusive) is approximately 495:1; and n is 10. In some embodiments, a ratio of one amine (e.g., the PEG-Alkyne of formula:

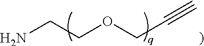

to the dendrimer of Formula (II-A) (

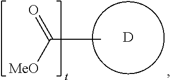

wherein D is PAMAM, and t is an integer of approximately 50-60, 60-70, or 70-80, inclusive) is approximately 500:1; the ratio of the ethanolamine to the PEG-Alkyne is 2.2:1; and n is 10.

In some embodiments, the molecular weight of the dendrimer of Formula (II-A) is approximately 12,500 g/mol, approximately 12,425 g/mol, approximately 12,420 g/mol, or approximately 12,418 g/mol, or approximately 12,415 g/mol. In some embodiments, the molecular weight of the dendrimer of Formula (II-A) is between approximately 12,000-12,500 g/mol.

In some embodiments, the dendrimer of Formula (II-A) is of formula:

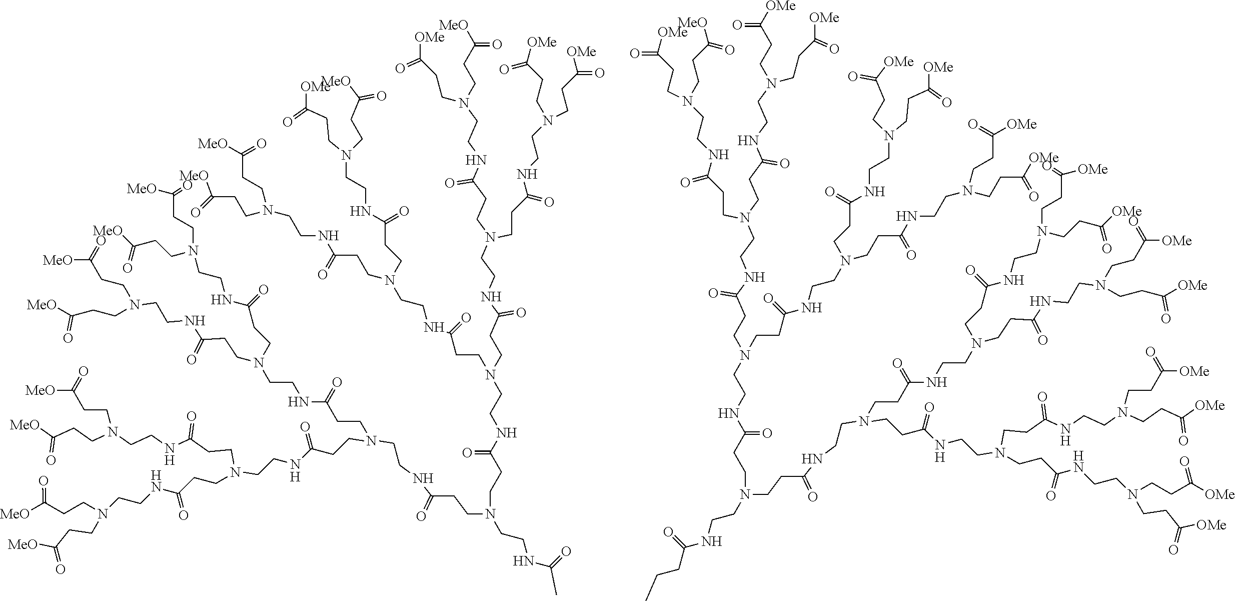

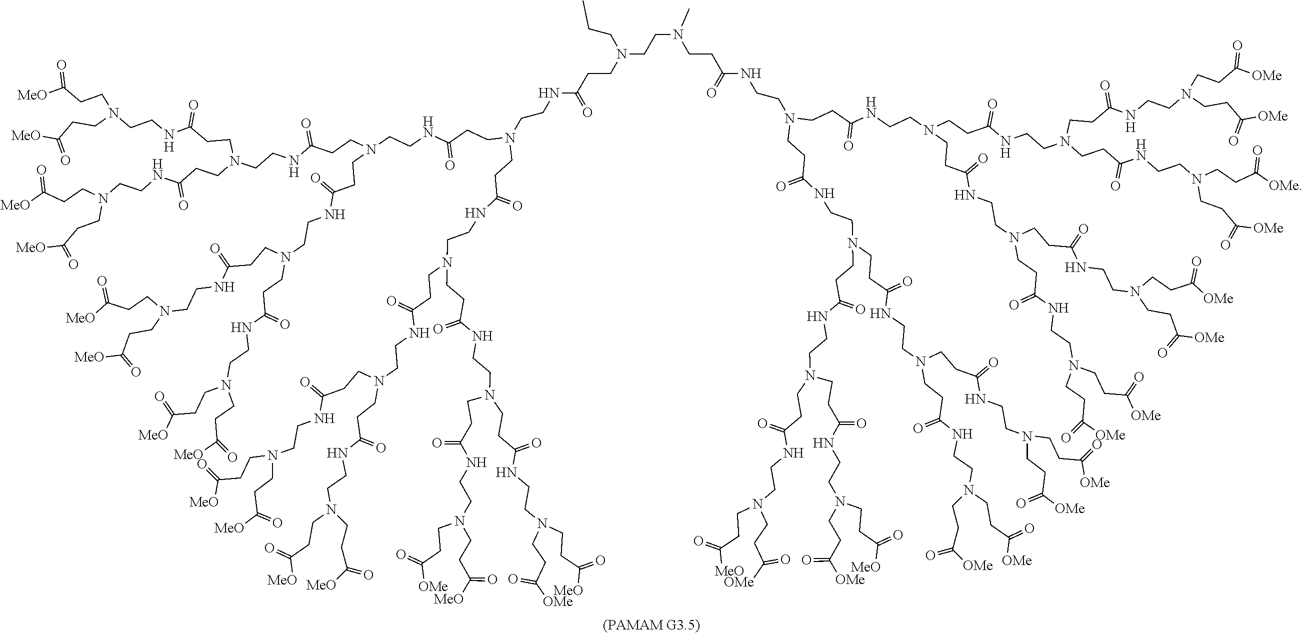

In some embodiments, the polydispersity value of the functionalized dendrimer of Formula (I-A) is low, for example, about 1.00 to about 1.05 (e.g., about 1.03). In some embodiments, the polydispersity value of the functionalized dendrimer of Formula (I-A) is about 1.03.

In some embodiments, over 10-20 grams, 20-30 grams, 30-40 grams, 40-50 grams, 50-60 grams, 60-70 grams, 70-80 grams, 80-90 grams, 90-100 grams, 100-110 grams, 110-120 grams, 120-130 grams, 130-140 grams, 150-160 grams, 160-180 grams, 180-200 grams, of the functionalized dendrimer of Formula (I-A) are synthesized by the methods of synthesis described herein. In some embodiments, over 30 grams, or over 50-100 grams (e.g., 40 grams, 160 grams) of the functionalized dendrimer of Formula (I-A) are synthesized by the methods of synthesis described herein.

In some embodiments, in the methods of synthesizing a functionalized dendrimer of Formula (I-A), wherein the method comprises reacting a dendrimer of Formula

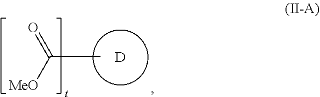

with one or more amines, wherein each amine is of the formula H₂NR¹; under suitable conditions to form the functionalized dendrimer of Formula (I-A); the suitable conditions comprise a reaction solvent and reacting the reactants at room temperature (e.g., approximately 19° C. to approximately 23° C.). In certain embodiments, the suitable conditions comprise a reaction solvent that is a protic solvent. For example, the reaction solvent may be a protic solvent, or a mixture of protic and aprotic solvents. In certain embodiments, the protic solvent is an alcohol, e.g., methanol, ethanol, or isopropanol. In certain embodiments, the protic solvent is methanol. In certain embodiments, the alcohol is anhydrous, or it may contain water. In certain embodiments, the reaction solvent may include dichloromethane.

In certain embodiments, the suitable conditions for synthesizing a functionalized dendrimer of Formula (I-A) comprise reacting the reactants at approximately 19° C. to approximately 23° C., for example, approximately 20° C. to approximately 22° C. In certain embodiments, the suitable conditions comprise reacting the reactants at approximately 18-19° C., 19-20° C., 20-21° C., 21-22° C., or 22-23° C. the suitable conditions comprise a reaction solvent of methanol and reacting the reactants at approximately 20° C.

In certain embodiments, the suitable conditions for synthesizing a functionalized dendrimer of Formula (I-A) comprise a first step of stirring for approximately 1-5 hours (e.g., 2 hours) at 0° C. the one or more amines, wherein each amine is of the formula H₂NR¹, together with the dendrimer of Formula (II-A), and reaction solvent (e.g., alcohol, for example, methanol); followed by a second step of stirring for approximately 3-8 days (e.g., 6 days) at approximately 19° C. to approximately 23° C. (e.g., 20° C.). In certain embodiments, the synthesized functionalized dendrimer of Formula (I-A) is of formula:

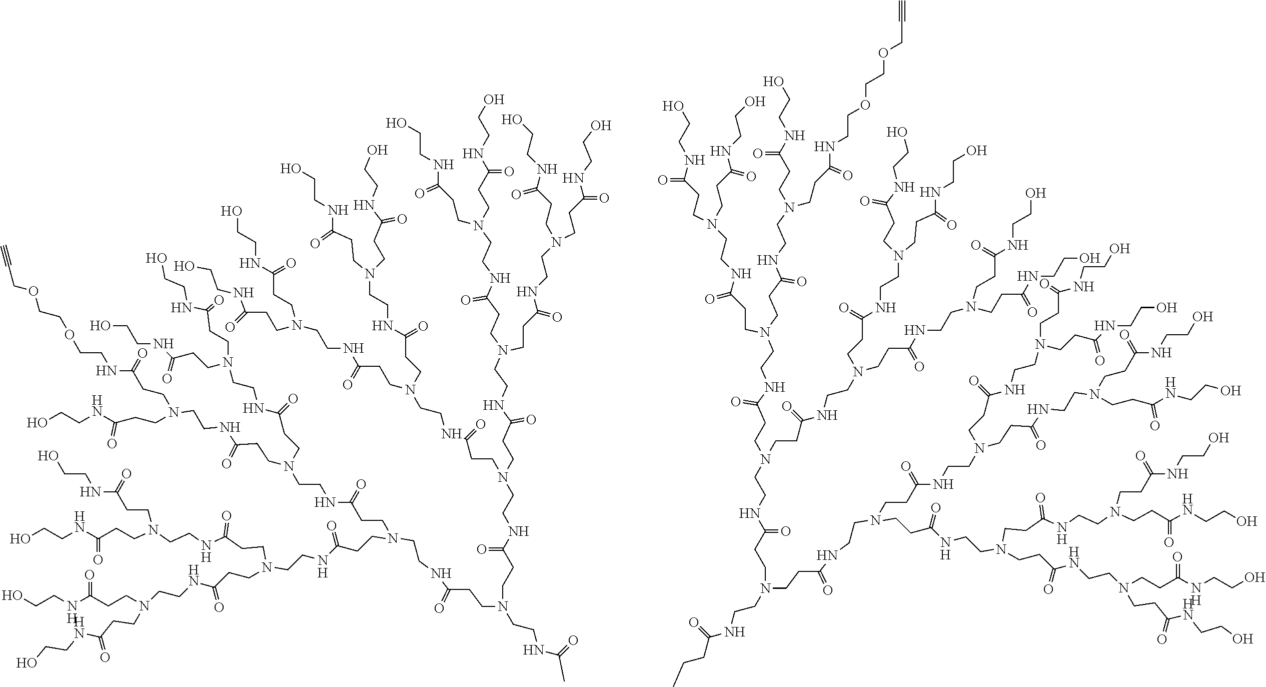

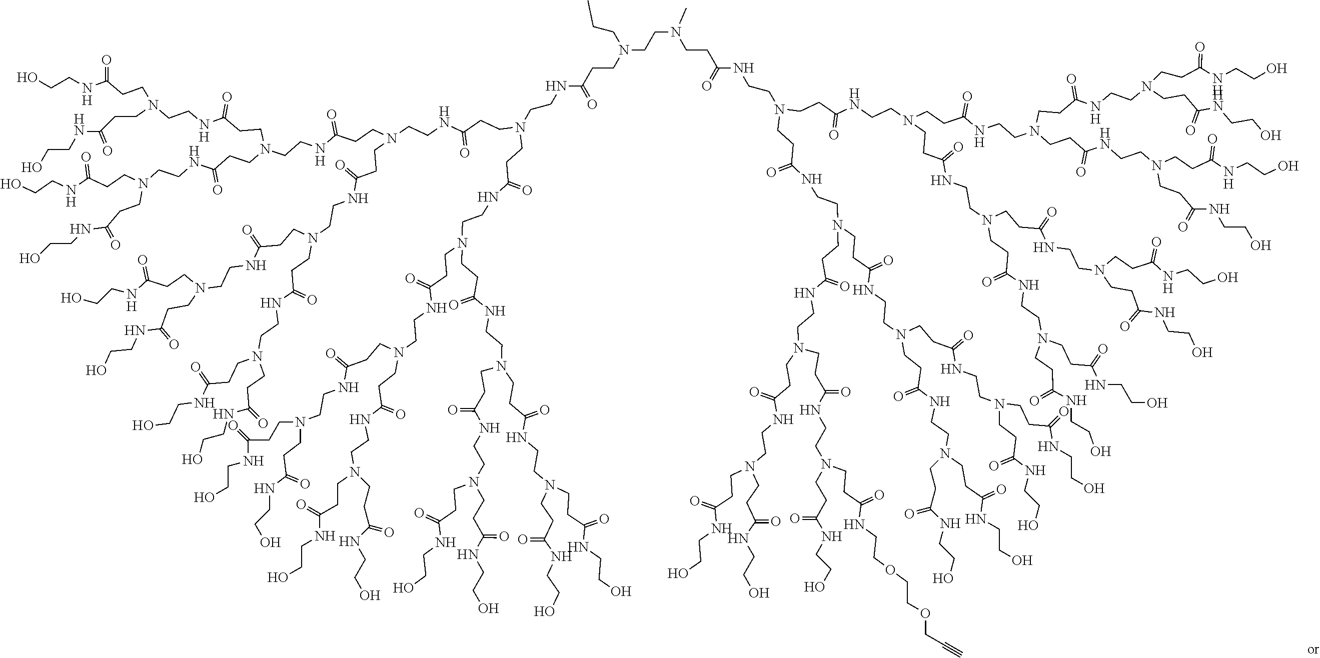

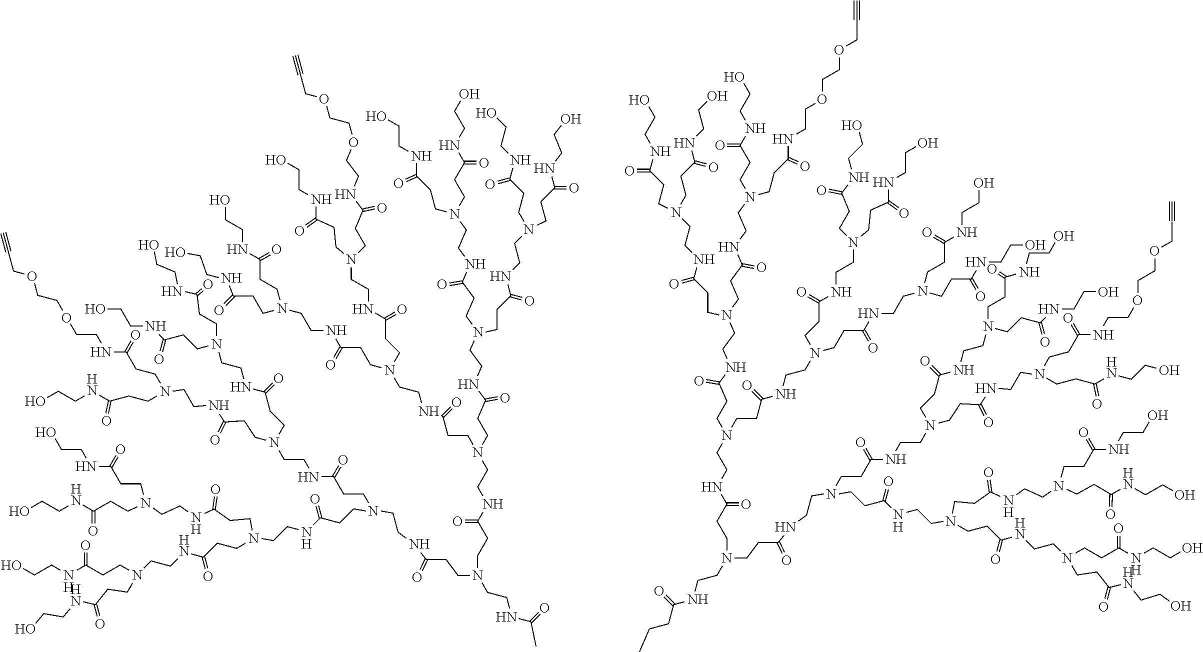

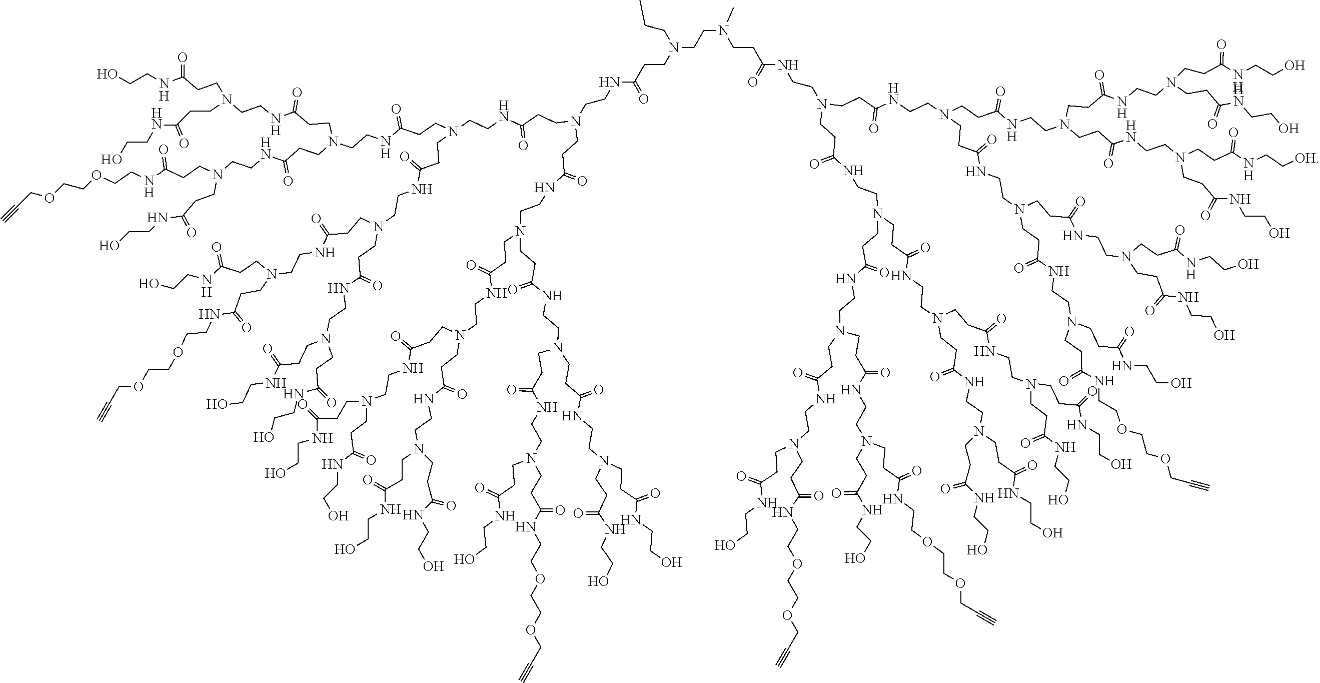

In some embodiments, the method for synthesizing a functionalized dendrimer of Formula (I-A) further comprises reacting with a compound of Formula (B):

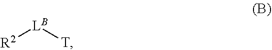

wherein: R² is halogen, optionally substituted acyl, optionally substituted alkenyl, optionally substituted alkynyl, optionally substituted acetyl, optionally substituted carbocyclyl, optionally substituted heterocyclyl, optionally substituted aryl, optionally substituted heteroaryl, —N₃, —CH(═N)(OH)R^{D1}, —CN, —NO₂, —OR^{D1}, —N(R^{D1a})₂, —SO₂OR^{D1}, or —SR^{D1} wherein R^{D1} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group when attached to an oxygen atom, or a sulfur protecting group when attached to a sulfur atom; wherein each occurrence of R^{D1a} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{D1} are taken together with their intervening atoms to form a substituted or unsubstituted heterocyclic or substituted or unsubstituted heteroaryl ring; provided that R^{1A} and R² are reaction partners; L^{B} is an alkylene linker, wherein one or more chain atoms of the hydrocarbon chain are independently replaced with amide, ester, hydroxamate, ether, carbonate, carbamate, hydrazone, thioether, thioester, disulfide, orthoester, urethane, or oxime moiety, or a cyclic moiety; and T is a therapeutic agent.

In some embodiments, linker L^{B} comprises a polymer. In some embodiments, linker L^{B} comprises an alkylene chain (e.g., a C_{1,100,000} alkylene), wherein the chain is the shortest path between R² and T, excluding hydrogen atoms and substituents. In certain embodiments, the chain of linker L^{B} comprises up to 5,000-7,000; 7,000-9,000; 9,000-10,000; 10,000-12,000; 100,000-120,000; 120,000-150,000; or 150,000-200,000 consecutive covalently bonded atoms or in length, excluding hydrogen atoms and substituents. In certain embodiments, L^{B} is an all-carbon, substituted or unsubstituted C₁-200,000 hydrocarbon chain as the shortest path between R² and T, excluding hydrogen atoms and substituents. In certain embodiments, any of the atoms in L^{B} can be substituted. In certain embodiments, none of the atoms in the linker L^{B} are substituted. In certain embodiments, none of the carbon atoms in the linker are substituted. In certain embodiments, at least one chain atom of the hydrocarbon chain of L^{B} is independently replaced with —C(═O)—, —O—, —NR—, —S—, or a cyclic moiety, wherein R^{b} is independently hydrogen, substituted or unsubstituted C₁₋₆ alkyl, or a nitrogen protecting group. In certain embodiments, at least one chain atom of the hydrocarbon chain of L^{B} is independently replaced with amide, hydroxamate, ether, n-alkyl, carbonate, carbamate, hydrazone, thioether, thioester, disulfide, orthoester, urethane, oxime, optionally substituted carbocyclylene, optionally substituted heterocyclylene, optionally substituted arylene, and/or optionally substituted heteroarylene. In certain embodiments, at least one chain atom of the hydrocarbon chain of L^{B} is independently replaced with amide, hydroxamate, ether, carbonate, carbamate, hydrazone, thioether, thioester, disulfide, orthoester, urethane, and/or oxime. In certain embodiments, the linker L^{B} comprises an alkylene moiety (e.g., of formula

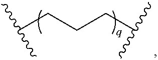

wherein q is an integer between 1-10,000 or 1-100,000, inclusive). In certain embodiments, the linker L^{B} comprises a polyethylene glycol moiety (e.g., of formula

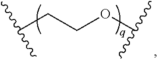

wherein q is an integer between 1-100,000, inclusive), and at least one chain atom of the hydrocarbon chain of L^{B} is independently replaced with amide, hydroxamate, ether, carbonate, carbamate, hydrazone, thioether, thioester, disulfide, orthoester, urethane, and/or oxime. In certain embodiments, the linker L^{B} comprises a moiety resulting from a click reaction. In some embodiments, the at least one moiety resulting from a click reaction is a 5-membered heterocyclic ring resulting from an electrocyclic reaction (e.g., 3+2 cycloaddition, or 4+2 cycloaddition) between reactive click chemistry handles (e.g., azides and terminal or strained alkynes, dienes and dienophiles, thiols and alkenes) used to produce the conjugate. In some embodiments, the at least one moiety resulting from a click reaction is a diradical comprising 1,2,3-triazolyl, 4,5-dihydro-1,2,3-triazolyl, isoxazolyl, 4,5-dihydroisoxazolyl, or 1,4-dihydropyridazyl.

In certain embodiments, R² is halogen (e.g., F, Cl, Br, or I). In certain embodiments, R² is optionally substituted acyl (e.g., —C(═O)Me). In certain embodiments, R² is optionally substituted alkyl (e.g., substituted or unsubstituted C₁₋₆ alkyl). In certain embodiments, R² is substituted or unsubstituted methyl. In certain embodiments, R² is substituted or unsubstituted ethyl. In certain embodiments, R² is substituted or unsubstituted propyl. In certain embodiments, R² is optionally substituted alkenyl (e.g., substituted or unsubstituted C₂₋₆ alkenyl). In certain embodiments, R² is optionally substituted alkynyl (e.g., substituted or unsubstituted C₂₋₆ alkynyl). In certain embodiments, R² is optionally substituted acetyl. In certain embodiments, R² is optionally substituted carbocyclyl (e.g., substituted or unsubstituted, 3- to 7-membered, monocyclic carbocyclyl comprising zero, one, or two double bonds in the carbocyclic ring system). In certain embodiments, R² is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R² is optionally substituted aryl (e.g., substituted or unsubstituted, 6- to 10-membered aryl). In certain embodiments, R^{1A} is benzyl. In certain embodiments, R² is substituted or unsubstituted phenyl. In certain embodiments, R² is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R² is —CH(═N)(OH)R^{D1}, —CN, —NO₂, —OR^{D1}, —N(R^{D1a})₂, —SO₂OR^{D1} or —SR^{D1}, wherein R^{D1} and R^{D1a} are as defined herein. In certain embodiments, R² is —CH(═N)(OH)R^{D1} (e.g., —CH(═N)(OH)(optionally substituted C₁₋₆ alkyl). In certain embodiments, R² is —CH(═N)(OH)(C₁₋₆ alkyl optionally substituted with a polyethylene glycol linker). In certain embodiments, R² is —CN. In certain embodiments, R² is —NO₂. In certain embodiments, R² is —OR^{D1} (e.g., —OH or —OMe). In certain embodiments, R^{1A} is —N(R^{D1a})₂ (e.g., —NMe₂). In certain embodiments, R² is —SO₂OR^{D1} (e.g., —SO₂O (optionally substituted alkyl)). In certain embodiments, R² is —SR^{D1} (e.g., —SMe). In certain embodiments, R² is —CH(═N)(OH)R^{D1}, —CN, —NO₂, —OR^{D1}, —N(R^{D1a})₂, —SO₂OR^{D1}, or —SR^{D1} wherein R^{D1} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group when attached to an oxygen atom, or a sulfur protecting group when attached to a sulfur atom; and wherein each occurrence of R^{D1a} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring. In certain embodiments, R² is bromo, alkyne, acetylene, alkene, aldehyde, amine, COOH, hydroxyl, carboxyl (e.g., dibenzocyclooctyne or DBCO), thiol, sulphonate, or —CN. In certain embodiments, R² is a click reaction handle (e.g., a click chemistry handle in Table A, or shown in Schemes 1-19 of Example 6). In certain embodiments, R^{1A} and R² are reaction partners (e.g., bioconjugation reaction partners). In certain embodiments, R^{1A} and R² are bioconjugation reaction partners (e.g., click reaction partners). In certain embodiments, R^{1A} and R² are click reaction partners from Table A. In certain embodiments, L^{B} is an alkylene linker, wherein one or more chain atoms of the hydrocarbon chain are independently replaced with amide, ester, hydroxamate, ether, carbonate, carbamate, hydrazone, thioether, thioester, disulfide, orthoester, urethane, or oxime moiety, or a cyclic moiety.

In certain embodiments, at least one instance of T is a therapeutic agent, targeting agent, or imaging agent as defined herein. In certain embodiments, at least one instance of T is a therapeutic agent as defined herein. In certain embodiments, at least one instance of T is a biomolecule. In certain embodiments, at least one instance of T is a proteolysis targeting chimera (PROTAC) drug. In certain embodiments, at least one instance of T is a biologic therapeutic agent (e.g., protein, peptide, a nucleic acid, or an antibody). In certain embodiments, at least one instance of T is a peptide, a nucleic acid, or an antibody. In certain embodiments, the nucleic acid is an oligonucleotide, DNA, or RNA (e.g., siRNA, mRNA). In certain embodiments, at least one instance of T is a gene, protein, peptide, oligonucleotide, carbohydrate, DNA, or RNA. In certain embodiments, each instance of T is different. In certain embodiments, each instance of T is the same.

In certain embodiments, provided herein is an intermediate dendrimer of Formula (II-A):

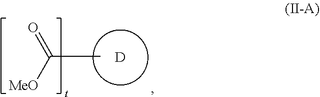

wherein D is a dendrimer selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof; and
t is an integer from 16 to 4096, inclusive.

In certain embodiments, provided herein is an intermediate dendrimer in the methods of synthesis described herein, wherein the intermediate dendrimer is of the formula:

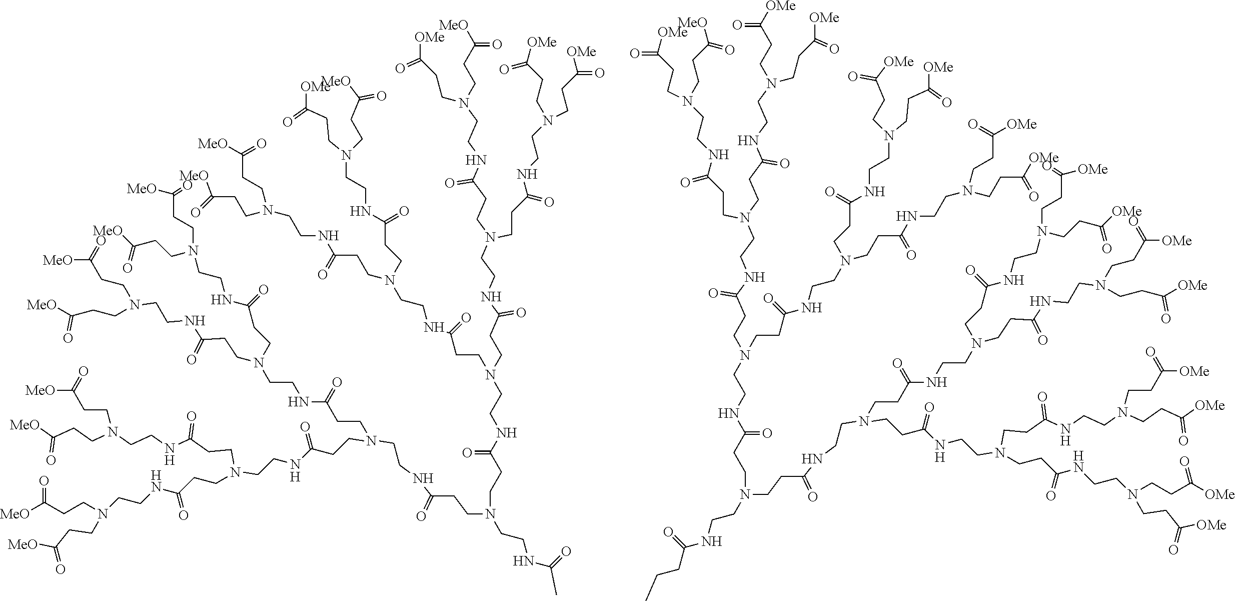

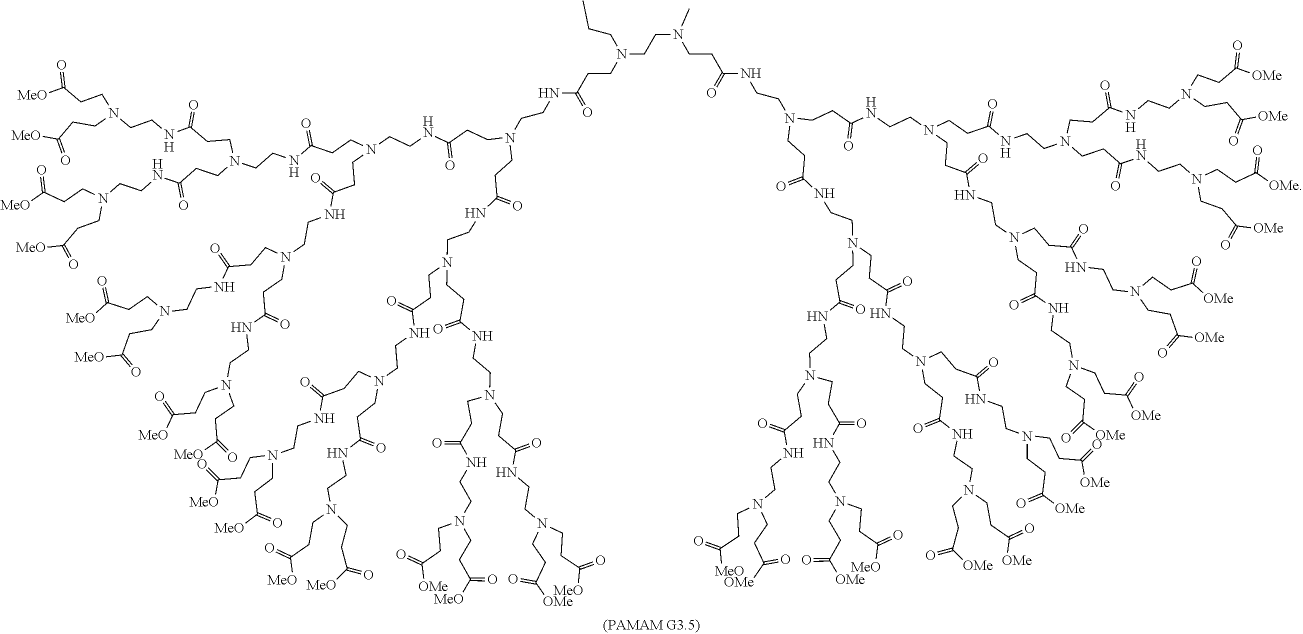

### Compositions Comprising Functionalized Dendrimer

In some aspects, the disclosure provides a composition comprising functionalized dendrimers of Formula (I-A):

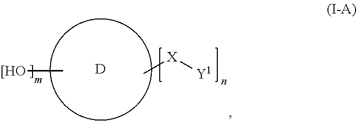

wherein:
D is a dendrimer selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof;
X is NH;
Y¹ is optionally substituted alkylene, optionally substituted alkenylene, optionally substituted alkynylene, or a covalent bond;
m is an integer from 16 to 4096, inclusive; and
n is an integer from 1 to 100, inclusive.

In some embodiments, the polydispersity value of the functionalized dendrimers in the composition is less than or equal to 1.10. In some aspects, the disclosure provides a composition comprising a carrier and functionalized dendrimers of Formula (I-A):

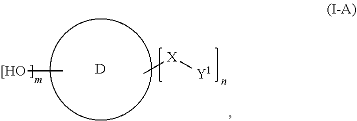

wherein:
D is a dendrimer selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof;
X is NH;
Y¹ is optionally substituted alkylene, optionally substituted alkenylene, optionally substituted alkynylene, or a covalent bond;
m is an integer from 16 to 4096, inclusive; and
n is an integer from 1 to 100, inclusive.

In some embodiments, the polydispersity value of the functionalized dendrimers in the composition is less than or equal to 1.10. In certain embodiments, the definitions for substituents D, X, Y¹, m, and n in the functionalized dendrimers of Formula (I-A) are as described above. In certain embodiments, polydispersity value of the functionalized dendrimers in the composition is less than or equal to 1.10 (e.g., less than or equal to 1.09, less than or equal to 1.08, less than or equal to 1.07, less than or equal to 1.06, less than or equal to 1.05, less than or equal to 1.04, less than or equal to 1.03, less than or equal to 1.02). In certain embodiments, polydispersity value of the functionalized dendrimers in the composition is less than or equal to 1.05, less than or equal to 1.04, less than or equal to 1.03, less than or equal to 1.02, less than or equal to 1.01, or less than or equal to 1.00. In certain embodiments, the composition comprises at least 10 grams of the functionalized dendrimer. In certain embodiments, the composition comprises at least 10 grams, at least 25 grams, at least 50 grams, at least 150 grams, or between 10-100 grams, 100-150 grams, 150-200 grams, 20-200 grams, 50-200 grams, or 100-200 grams of the functionalized dendrimer (e.g., the functionalized dendrimer of Formula (I-A)). In some embodiments, the composition comprises over 10-20 grams, 20-30 grams, 30-40 grams, 40-50 grams, 50-60 grams, 60-70 grams, 70-80 grams, 80-90 grams, 90-100 grams, 100-110 grams, 110-120 grams, 120-130 grams, 130-140 grams, 150-160 grams, 160-180 grams, 180-200 grams, of the functionalized dendrimer (e.g., the functionalized dendrimer of Formula (I-A)). In some embodiments, the composition comprises over 30 grams, or over 50-100 grams (e.g., 40 grams, 160 grams) of the functionalized dendrimer (e.g., the functionalized dendrimer of Formula (I-A)).

In certain embodiments, the composition comprises a suitable carrier (e.g., solid or liquid carrier) as described herein.

### Therapeutic Agents

In some embodiments, the disclosure provides a dendrimer conjugate comprising a dendrimer having at least one therapeutic agent at one or more terminal positions of the dendrimer. In some embodiments, such dendrimer-drug conjugates have a therapeutic index that is increased relative to the unconjugated drug (e.g., therapeutic agent in absence of the dendrimer). In some embodiments, the dendrimer-drug conjugate has a therapeutic index of greater than 10% of the therapeutic index of the unconjugated drug. In some embodiments, the dendrimer-drug conjugate has a therapeutic index of greater than 20%, 30%, 40%, 50%, 60%, 70%, 80%, or 90% of the therapeutic index of the unconjugated drug.

The therapeutic index (TI) of a therapeutic agent is a comparison of the amount of the therapeutic agent that causes the therapeutic effect to the amount that causes toxicity. In some embodiments, a therapeutic index can be expressed as the ratio, LD₅₀/ED₅₀, where ED₅₀ corresponds to a dose that is therapeutically effective in 50% of the population, and LD₅₀ corresponds to the dose that is lethal to 50% of the population. In some embodiments, therapeutic efficacy and toxicity of drugs and dendrimer-drug conjugates can be determined by standard pharmaceutical procedures in cell cultures or experimental animals

In some embodiments, a dendrimer is complexed with or conjugated to two or more different classes of therapeutic agents, providing simultaneous delivery with different or independent release kinetics at the target site. For example, in some embodiments, a STING agonist and a CSF1R inhibitor are conjugated to a dendrimer for delivery to target cells or tissues. In some embodiments, dendrimer conjugates, each carrying different classes of therapeutic agents, are administered simultaneously for a combination treatment. In some embodiments, a generation 4 or generation 6 PAMAM dendrimer is conjugated to sunitinib and a CXCR2 inhibitor, or analogs thereof. In some embodiments, a generation 4 or generation 6 PAMAM dendrimer is conjugated to vincristine and sunitinib, or analogs thereof.

In some embodiments, a therapeutic agent is any of the compounds described below or a pharmaceutically acceptable derivative, analog, or prodrug of any of the compounds described below. Prodrugs are compounds that, when metabolized in vivo, undergo conversion to compounds having the desired pharmacological activity. Prodrugs can be prepared by replacing appropriate functionalities present in a therapeutic agent with “pro-moieties” as described in the art (see, e.g., H. Bundgaar, Design of Prodrugs (1985)). Examples of prodrugs include ester, ether or amide derivatives of a therapeutic agent described herein, polyethylene glycol derivatives of a therapeutic agent described herein, N-acyl amine derivatives, dihydropyridine pyridine derivatives, amino-containing derivatives conjugated to polypeptides, 2-hydroxybenzamide derivatives, carbamate derivatives, N-oxides derivatives that are biologically reduced to the active amines, and N-Mannich base derivatives. For further discussion of prodrugs, see, for example, Rautio, J. et al. Nature Reviews Drug Discovery. 7:255-270 (2008).

In some embodiments, a dendrimer conjugate of the disclosure comprises a therapeutic agent selected from the group consisting of angiotensin II receptor blockers, Farnesoid X receptor (FXR) agonists, death receptor 5 agonists, sodium-glucose cotransporter type-2 (SGLT2) inhibitors, lysophosphatidic acid 1 receptor antagonists, endothelin-A receptor antagonist, peroxisome proliferator-activated receptor delta (PPAR6) agonists, ATl receptor antagonists, CCR5/CCR2 antagonists, anti-fibrotic agents, anti-inflammatory agents, antioxidant agents, stimulator of interferon genes (STING) agonists, colony-stimulating factor 1 receptor (CSF1R) inhibitors, AXL inhibitors, c-Met inhibitors, poly(ADP-ribose) polymerase (PARP) inhibitors, receptor tyrosine kinase inhibitors, MEK inhibitors, PAK1 inhibitors, glutaminase inhibitors, TIE II antagonists, chemokine receptor 2 (CXCR2) inhibitors, CD73 inhibitors, arginase inhibitors, phosphatidylinositol-3-kinase (PI3K) inhibitors, toll-like receptor 4 (TLR4) agonists, toll-like receptor 7 (TLR7) agonists, Src homology-2 domain-containing protein tyrosine phosphatase-2 (SHP2) inhibitors, chemotherapeutic agents, STING antagonists, and JAK1 inhibitors.

In some embodiments, a therapeutic agent is an immunomodulatory agent. In some embodiments, an immunomodulatory agent refers to an agent that elicits a specific effect upon the immune system of the recipient. Immunomodulation can include, in some embodiments, suppression, reduction, enhancement, prolonging, or stimulation of one or more physiological processes of the innate or adaptive immune response to antigen, as compared to a control. In some embodiments, immunomodulatory agents can modulate immune microenvironment for a desired immunological response (e.g., increasing anti-tumor activity, or increasing anti-inflammatory activities sites in need thereof in autoimmune diseases) by targeting one or more immune cells or cell types at a target site. In some embodiments, immunomodulatory agents are delivered to kill, inhibit, or reduce activity or quantity of suppressive immune cells such as tumor-associated macrophages for an enhanced anti-tumor response at a tumor site. In other embodiments, immunomodulatory agents are delivered to kill, inhibit, or reduce activity or quantity of pro-inflammatory immune cells (e.g., M1-type macrophages) for reducing pro-inflammatory immune environment at pathogenic sites associated with autoimmune diseases.

Examples of immunomodulatory agents for use in accordance with the disclosure include, without limitation, STING agonists, STING antagonists, Janus kinase 1 (JAK1) inhibitors, CSF1R inhibitors, AXL inhibitors, c-Met inhibitors, PARP inhibitors, receptor tyrosine kinase inhibitors, MEK inhibitors, PAK1 inhibitors, glutaminase inhibitors, TIE II antagonists, CXCR2 inhibitors, CD73 inhibitors, arginase inhibitors, PI3K inhibitors, TLR4 agonists, TLR7 agonists, SHP2 inhibitors, anti-inflammatory agents, and combinations thereof.

In some embodiments, a therapeutic agent is a STING agonist selected from a cyclic dinucleotide GMP-AMP and DMXAA. In some embodiments, a therapeutic agent is a STING antagonist selected from C-178, C-176, C18, Astin C, NO₂-CLA, H-151, and alpha-mangostin. In some embodiments, a therapeutic agent is a JAK1 inhibitor selected from tofacitinib, ruxolitinib, baricitinib, peficitinib, decernotiniba, filgotinib, solcitinibb, itacitinib, SHR0302, upadacitinib, PF-04965842, Target-007, and Target-006. In some embodiments, a therapeutic agent is a CSF1R inhibitor selected from PLX3397, PLX108-01, ARRY-382, PLX7486, BLZ945, JNJ-40346527, and GW 2580. In some embodiments, a therapeutic agent is a PARP inhibitor selected from Olaparib, Veliparib, Niraparib, and Rucaparib. In some embodiments, a therapeutic agent is a receptor tyrosine kinase inhibitor of vascular endothelial growth factor receptors (VEGFR) or epidermal growth factor receptor (EGFR). In some embodiments, a therapeutic agent is an AXL inhibitor (e.g., bemcentinib (R428), dubermatinib (TP-0903)). In some embodiments, a therapeutic agent is a c-Met inhibitor (e.g., cabozantinib). In some embodiments, a therapeutic agent is a receptor tyrosine kinase (e.g., VEGFR, CSF1R, AXL, and/or c-Met) inhibitor selected from sunitinib, sorafenib, pazopanib, vandetanib, axitinib, cediranib, vatalanib, dasatinib, bemcentinib (R428), dubermatinib (TP-0903), nintedanib, cabozantinib, and motesanib. In some embodiments, a therapeutic agent is a MEK inhibitor selected from Trametinib, Cobimetinib, Binimetinib, Selumetinib, PD325901, PD035901, PD032901, and TAK-733. In some embodiments, a therapeutic agent is a PAK1 inhibitor. In some embodiments, the PAK1 inhibitor is Frax-1036 (6-[2-chloro-4-(6-methyl-2-pyrazinyl)phenyl]-8-ethyl-2-[[2-(1-methyl-4-piperidinyl)ethyl]amino]-pyrido[2,3-d]pyrimidin-7(8H)-one). In some embodiments, a therapeutic agent is a glutaminase inhibitor selected from Bis-2-(5-phenylacetimido-1,2,4-thiadiazol-2-yl)ethyl sulfide (BPTES), azaserine, acivicin, and CB-839. In some embodiments, a therapeutic agent is a CXCR2 inhibitor selected from Navarixin, SB225002, and SB332235. In some embodiments, a therapeutic agent is a CD73 inhibitor selected from APCP, quercetin, and tenofovir. In some embodiments, a therapeutic agent is an arginase inhibitor, such as 2-(S)-amino-6-boronohexanoic acid. In some embodiments, a therapeutic agent is a PI3K inhibitor selected from alpelisib, serabelisib, pilaralisib, WX-037, dactolisib, prexasertib, voxtalisib, PX-866, ZSTK474, buparlisib, pictilisib, and copanlisib.

In some embodiments, a therapeutic agent is an anti-inflammatory agent. In some embodiments, an anti-inflammatory agent reduces inflammation and can include steroidal and non-steroidal drugs. Examples of steroidal drugs include, without limitation, glucocorticoids, progestins, mineralocorticoids, and corticosteroids. Examples of non-steroidal anti-inflammatory drugs (NSAIDs) include, without limitation, mefenamic acid, aspirin, Diflunisal, Salsalate, Ibuprofen, Naproxen, Fenoprofen, Ketoprofen, Deacketoprofen, Flurbiprofen, Oxaprozin, Loxoprofen, Indomethacin, Sulindac, Etodolac, Ketorolac, Diclofenac, Nabumetone, Piroxicam, Meloxicam, Tenoxicam, Droxicam, Lornoxicam, Isoxicam, Meclofenamic acid, Flufenamic acid, Tolfenamic acid, elecoxib, Rofecoxib, Valdecoxib, Parecoxib, Lumiracoxib, Etoricoxib, Firocoxib, Sulphonanilides, Nimesulide, Niflumic acid, and Licofelone. Additional examples of anti-inflammatory agents include, without limitation, triamcinolone acetonide, fluocinolone acetonide, methylprednisolone, prednisolone, prednisone, dexamethasone, loteprendol, fluorometholone, ibuprofen, aspirin, naproxen, cyclosporine, tacrolimus, rapamycin, and metformin. In some embodiments, a therapeutic agent is triamcinolone acetonide, prednisone, or dexamethasone.

In some embodiments, a therapeutic agent is a cytotoxic agent. In some embodiments, a therapeutic agent is a chemotherapeutic agent. Examples of cytotoxic agents for use in accordance with the disclosure include, without limitation, amsacrine, bevacizumab, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, daunorubicin, lomustine, mechlorethamine, melphalan, mercaptopurine, mertansine, mesna, methotrexate, mitomycin, mitoxantrone, monomethyl auristatin E, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A, trastuzumab, cetuximab, and rituximab.

In some embodiments, a therapeutic agent is an anti-cancer agent, such as a cytotoxic agent described herein. In some embodiments, a therapeutic agent is a histone deacetylase (HDAC) inhibitor, such as vorinostat. In some embodiments, a therapeutic agent is a topoisomerase I and/or II inhibitor, such as etoposide or camptothecin. Additional examples of anti-cancer agents include, without limitation, irinotecan, exemestane, octreotide, carmofur, clarithromycin, zinostatin, tamoxifen, tegafur, toremifene, doxifluridine, nimustine, vindensine, nedaplatin, pirarubicin, flutamide, fadrozole, prednisone, medroxyprogesterone, mitotane, mycophenolate mofetil, and mizoribine.

In some embodiments, a therapeutic agent is an anti-angiogenesis agent. Examples of anti-angiogenesis agents include, without limitation, bevacizumab (AVASTIN®), rhuFAb V2 (ranibizumab, LUCENTIS®), aflibercept (EYLEA®), MACUGEN® (pegaptanim sodium, anti-VEGF aptamer or EYE001), pigment epithelium derived factor(s) (PEDF), celecoxib (CELEBREX®), rofecoxib (VIOXX®), interferon alpha, interleukin-12 (IL-12), thalidomide (THALOMID®), lenalidomide (REVLIMID®), squalamine, endostatin, angiostatin, ANGIOZYME® (Sirna Therapeutics), NEOVASTAT® (AE-941) (Aeterna Laboratories, Quebec City, Canada), sunitinib (SUTENT®), sorafenib (Nexavar®), erlotinib (Tarceva®), panitumumab (VECTIBIX®), and cetuximab (ERBITUX®). Additional examples of anti-angiogenesis agents include agents targeting members of the platelet-derived growth factor family, epidermal growth factor family, fibroblast growth factor family, transforming growth factor-β superfamily (TGF-β1, activins, follistatin and bone morphogenetic proteins), angiopoietin-like family, galectins family, integrin superfamily, as well as pigment epithelium derived factor, hepatocyte growth factor, angiopoietins, endothelins, hypoxia-inducible factors, insulin-like growth factors, cytokines, matrix metalloproteinases and their inhibitors, and glycosylation proteins.

In some embodiments, a therapeutic agent is an agent that can be used to treat one or more conditions or diseases associated with the liver and/or associated diseases or conditions such as infections, sepsis, diabetic complications, hypertension, obesity, high blood pressure, heart failure, kidney diseases, and cancers. Examples of such therapeutic agents include, without limitation, angiotensin II receptor blockers, FXR agonists, death receptor 5 agonists, SGLT2 inhibitors, lysophosphatidic acid 1 receptor antagonists, endothelin-A receptor antagonist, PPARS agonists, ATl receptor antagonists, CCR5/CCR2 antagonists, insulin sensitizer, pioglitazone, anti-fibrotic agents, antioxidant agents, antiangiogenic agents, anti-excitotoxic agents (e.g., valproic acid, D-aminophosphonovalerate, D-aminophosphonoheptanoate), inhibitors of glutamate formation/release (e.g., baclofen, NMDA receptor antagonists, ranibizumab, anti-VEGF agents), and immunomodulatory and cytotoxic agents described herein.

In some embodiments, a therapeutic agent is an angiotensin II receptor blocker, such as telmisartan, a telmisartan-amide derivative, or a telmisartan-ester derivative. In some embodiments, a therapeutic agent is an FXR agonist, such as chenodeoxycholic acid, a chenodeoxycholic acid-amide derivative, or a chenodeoxycholic acid-ester derivative. In some embodiments, a therapeutic agent is an SGLT2 inhibitor selected from phlorizin, T-1095, canagliflozin, dapagliflozin, ipragliflozin, tofogliflozin, empagliflozin, luseogliflozin, ertugliflozin, and remogliflozin etabonate. In some embodiments, a therapeutic agent is a PPARS agonist, such as GW0742, a GW0742-amide derivative, or a GW0742-ester derivative. In some embodiments, a therapeutic agent is an antioxidant agent, such as vitamin E.

In some embodiments, a therapeutic agent is N-acetyl-L-cysteine. In some embodiments, N-acetyl-L-cysteine is conjugated to a hydroxyl-terminated dendrimer via non-cleavable linkage for minimal release of free N-acetyl-cysteine in vivo after administration. In some embodiments, a non-cleavable form of a dendrimer/N-acetyl-cysteine conjugate provides enhanced therapeutic efficacy as compared to a releasable or cleavable form of the dendrimer/N-acetyl-cysteine complex.

In some embodiments, a therapeutic agent is polysialic acid (e.g., low molecular weight polySia with an average degree of polymerization 20 (polySia avDP20)), Translocator Protein Ligands (e.g., Diazepam binding inhibitor (DBI)), Interferon-β (IFN-β), or minocycline.

In some embodiments, a therapeutic agent is an anti-infective agent. Examples of anti-infective agents include, without limitation, antiviral agents, antibacterial agents, antiparasitic agents, and anti-fungal agents. In some embodiments, a therapeutic agent is selected from moxifloxacin, ciprofloxacin, erythromycin, levofloxacin, cefazolin, vancomycin, tigecycline, gentamycin, tobramycin, ceftazidime, ofloxacin, gatifloxacin, amphotericin, voriconazole, and natamycin.

Therapeutic agents suitable for use in accordance with the disclosure are described in additional detail in co-pending International Application Numbers PCT/US2020/063332, PCT/US2020/063347, PCT/US2020/063342, and PCT/US2021/029139, the relevant contents of each of which are incorporated by reference herein in their entireties.

### Imaging Agents

In some embodiments, the disclosure provides a dendrimer conjugate comprising a dendrimer having at least one imaging agent at one or more terminal positions of the dendrimer. In some embodiments, a dendrimer conjugate comprising an imaging agent can be used for diagnostic, therapeutic, or labeling purposes. In some embodiments, an imaging agent is a paramagnetic molecule, a fluorescent compound, a magnetic molecule, a radionuclide, an x-ray imaging agents, or a contrast agent. In some embodiments, a contrast agent is a gas or gas-emitting compound, which is radioopaque. In some embodiments, a dendrimer conjugate comprising an imaging agent can be used for determining the location of administered compositions. Imaging agents useful for this purpose include, without limitation, fluorescent tags, radionuclides, and contrast agents. Examples of imaging agents useful for diagnostic purposes include, without limitation, dyes, fluorescent dyes, near infrared dyes, SPECT imaging agents, PET imaging agents, and radioisotopes. Examples of dyes include, without limitation, carbocyanine, indocarbocyanine, oxacarbocyanine, thiocarbocyanine and merocyanine, polymethine, coumarin, rhodamine, xanthene, fluorescein, boron-dipyrromethane (BODIPY), Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS.

In some embodiments, a dendrimer conjugate comprises a radionuclide reporter appropriate for imaging by scintigraphy, single-photon emission computed tomography (SPECT), or positron emission tomography (PET). In some embodiments, a dendrimer conjugate comprises a radionuclide appropriate for radiotherapy. In some embodiments, a dendrimer conjugate comprises a contrast agent for imaging by magnetic resonance imaging (MRI). In some embodiments, a dendrimer conjugate comprises a chelator for a radionuclide or an MRI contrast agent useful for diagnostic imaging, and a chelator useful for radiotherapy. Accordingly, in some embodiments, a single dendrimer/imaging agent conjugate can simultaneously treat and diagnose a disease or a condition at one or more locations in the body. In some embodiments, a dendrimer conjugate comprises a radioactively labeled SPECT, or scintigraphic imaging agents that have a suitable amount of radioactivity.

Suitable imaging agents can be selected based on a particular imaging methodology. For example, in some embodiments, an imaging agent is a near infrared fluorescent dye for optical imaging, a Gadolinium chelate for MRI imaging, a radionuclide for PET or SPECT imaging, or a gold nanoparticle for CT imaging.

In some embodiments, a dendrimer conjugate comprises one or more imaging agents for PET imaging, such as one or more radionuclides. PET is a technique that uses a special camera and a computer to detect small amounts of radioactive radiotracers or radiopharmaceuticals in vivo, to evaluate organ and tissue functions (e.g., to detect early onset of a disease).

PET involves the detection of gamma rays in the form of annihilation photons from short-lived positron emitting radioactive isotopes including, but not limited to, ¹⁸F with a half-life of approximately 110 minutes, ¹¹C with a half-life of approximately twenty minutes, ¹³N with a half-life of approximately ten minutes, and ¹⁵O with a half-life of approximately two minutes, using coincidence detection. Accordingly, in some embodiments, examples of imaging agents for use in PET imaging include, without limitation, one or more of the various positron emitting metal ions, such as ⁵¹Mn, ⁵²Fe, ⁶⁰Cu, ⁶⁸Ga, ⁷²As, ⁹⁴mTc, or ¹¹⁰In. In some embodiments, an imaging agent is a radionuclide selected from ¹⁸F, ¹²⁴I, ¹²⁵I, ¹³¹I, ⁷⁷Br, and ⁷⁶Br. Examples of metal radionuclides for scintigraphy or radiotherapy include, without limitation, ^{99m}Tc, ⁵¹Cr, ⁶⁷Ga, ⁶⁸Ga, ⁴⁷Sc, ⁵¹Cr, ¹⁶⁷Tm, ¹⁴¹Ce, ¹¹¹In, ¹⁶⁸Yb, ¹⁷⁵Yb, ¹⁴⁰La, ⁹⁰Y, ⁸⁸Y, ¹⁵³Sm, ¹⁶⁶Ho, ¹⁶⁵Dy, ¹⁶⁶Dy, ⁶²Cu, 6Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ¹¹⁷mSn, ¹⁴⁹Pm, ¹⁶¹Tb, ¹⁷⁷Lu, ²²⁵Ac, ¹⁹⁸Au and ¹⁹⁹Au. The choice of metal will be determined based on the desired therapeutic or diagnostic application. For example, for diagnostic purposes, in some embodiments, useful radionuclides include ⁶Cu, ⁶⁷Ga, ⁶⁸Ga, ^{99m}Tc, and ¹¹¹In. For therapeutic purposes, in some embodiments, useful radionuclides include ⁶⁴Cu, ⁹⁰Y, ¹⁰⁵Rh, ¹¹¹In, ¹¹⁷mSn, ¹⁴⁹Pm, ¹⁵³Sm, ¹⁶¹Tb, ¹⁶⁶T, ¹⁶⁶Dy, ¹⁶⁶Ho, ¹⁷⁵Yb, ¹⁷⁷Lu, ²²⁵Ac, ^{186/188}Re, and ¹⁹⁹Au.

In some embodiments, an imaging agent is technetium-99m (^{99m}Tc). In some embodiments, ^{99m}Tc is useful for diagnostic applications because of its low cost, availability, imaging properties, and high specific activity. The nuclear and radioactive properties of ^{99m}Tc make this isotope useful for scintigraphic imaging. This isotope has a single photon energy of 140 keV and a radioactive half-life of about 6 hours, and is readily available from a ⁹⁹Mo-^{99m}Tc generator. In some embodiments, radionuclides useful for PET imaging include ¹⁸F, 4-[¹⁸F]fluorobenzaldehyde (¹⁸FB), Al[¹⁸F]-NOTA, ⁶⁸Ga-DOTA, and ⁶⁸Ga-NOTA. In some embodiments, ¹⁵³Sm can be used as an imaging agent with chelators such as ethylenediaminetetramethylenephosphonic acid (EDTMP) or 1,4,7,10-tetraazacyclododecanetetramethylenephosphonic acid (DOTMP).

MRI can be used to assess brain disease, spinal disorder, angiography, cardiac function, and musculoskeletal damage, among other uses. MRI does not require the use of ionizing radiation, and scans can be performed at any chosen orientation. MRI provides full three-dimensional capabilities, high soft-tissue contrast, high spatial resolution, and is adept at morphological and functional imaging. Accordingly, in some embodiments, a dendrimer comprises one or more imaging agents for MRI, such as one or more MRI contrast agents. Examples of MRI contrast agents are known in the art and include, without limitation, Gd, Mn, BaSO4, iron oxides, and iron platinum.

### Targeting Agents

In some embodiments, the dendrimer includes one or more tissue targeting or tissue binding moieties, for targeting the dendrimer to a specific location in vivo, and/or for enhancing the in vivo residence time at a desired location within the body. For example, in some embodiments, the dendrimer is sequestered or bound to one or more distinct tissues or organs following local or systemic administration into the body. Therefore, the presence of a targeting or binding moiety can enhance the delivery of an agent to a target site relative to the dendrimer and agent in the absence of a targeting or binding moiety. Conjugation of the dendrimer to one or more targeting or binding moieties can be via a spacer, and the linkage between the spacer and dendrimer, and/or the spacer and targeting agent can be designed to provide releasable or non-releasable forms of the dendrimer-targeting agent complex.

An exemplary targeting agent is alendronic acid (alendronate), which binds to hypoxyapetite at the surface of bones, and enhances the residence tine of the dendrimer complex to bones. Alendronate is a small molecule targeting moiety, which selectively binds to hydroxyapatite, a component of bone. Therefore, in some embodiments, the dendrimer is conjugated to alendronate, for selective targeting of the dendrimer to bone. In some embodiments, the conjugation between the alendronate and the dendrimer is via a reversible (non-covalent) linker. In other embodiments, the conjugation between the alendronate and the dendrimer is via a non-cleavable or a minimally cleavable linker. In some embodiments, the targeting agent also has a therapeutic effect at the targeted site. In some embodiments, the dendrimer is conjugated to alendronate, for targeting the dendrimer complex to bone and for providing a therapeutic effect at the site of bone inflammation. In some embodiments, alendronate-bound dendrimers are conjugated to one or more agents for selective delivery of the agents to sites of bone inflammation.

It has been established that dendrimers conjugated or complexed with the carbohydrate triantennary N-Acetylgalactosamine (GalNAc) selectively accumulate within hepatocyte cells. Compositions of dendrimers modified by addition of triantennary N-Acetylgalactosamine (GalNAc) to the dendrimer surface are described.

The abundantly expressed asialoglycoprotein receptor (ASGPR) on hepatocytes can selectively recognize galactose and N-acetylgalactosamine (GalNAc) through carbohydrate recognition domain (CRD) and binds to the receptor tightly. The efficient binding of carbohydrate moieties to the ASGPR receptors allows selective internalization within the hepatocyte via receptor-mediated endocytosis. The low pH in the endosomes results in the disruption of the tetravalent calcium-chelation between the sugar ligand and the ASGPR receptor, which releases the ligand in the hepatocytes. Once the ligand is released, the receptor complex recycles allowing large amounts of ligand to be internalized into hepatocytes without saturation effects. GalNAc binding to ASGPR occurs at the sinusoidal surface of the hepatocyte, which contains ˜500,000 ASGPR receptors per cell, of which about 5%-10% are present at the cell surface at any one time. Previous studies have shown that the binding of ligands to ASGPR is dependent upon the type of sugar (GalNAc >Gal) and number of sugars with 4>3>2>1. X-ray crystal structures of the extracellular domain of ASGPR revealed a shallow carbohydrate-binding pocket, explaining the requirement for multivalency. Multivalent binding has therefore been explored, and the binding affinity of trivalent and tetravalent carbohydrate constructs to ASGPR is 100-1000 folds stronger compared to monovalent ligands due to the glyco-cluster effect.

Bi- and triantennary GalNAc ligands conjugated to SiRNAs demonstrated significantly higher levels of GalNAc-siRNA in the livers of C57BL/6 mice from subcutaneous administration with 94% of the GalNAc-siRNA localized in hepatocytes. Further, these siRNA conjugates mediated efficient gene silencing. Further studies reported that anti-sense oligonucleotides (ASOs) linked to triantennary GalNAc were up to 10-fold more potent than the parent ASOs in mouse models.

Carbohydrate-protein interactions play an important role in biological processes such as receptor-mediated endocytosis and have been applied to cell recognition studies as well as designs for biomedical materials. Carbohydrate-terminating dendrimers (glycodendrimers) are endowed with enhanced binding affinities with allied receptors, which enables them to interact with specific cell types with avidity and selectivity for targeted drug delivery. Introduction of carbohydrate moieties in the drug delivery platform also provides biocompatibility, as well as increases water solubility of the dendrimer complexes.

Triantennary-GalNAc provides effective multivalent binding to ASGPR on hepatocytes. Therefore, in some embodiments, the dendrimers are modified at one or more surface terminal groups (e.g., —OH) with one or more triantennary-GalNAc groups.

Triantennary GalNAc modification of a dendrimer gives rise to a set of three GalNAc at each surface terminal group. In some embodiments, three β-GalNAc molecules are grafted via one or more linkers onto a building block to yield an AB3 building block (i.e., triantennary GalNAc dendron) suitable for conjugation to the surface functional groups of the dendrimers.

In some embodiments, three β-GalNAc molecules are grafted via one or more linkers onto a propargylated pentaerythritol building block to yield an AB3 building block suitable for conjugation to the surface functional groups of the dendrimers as shown below.

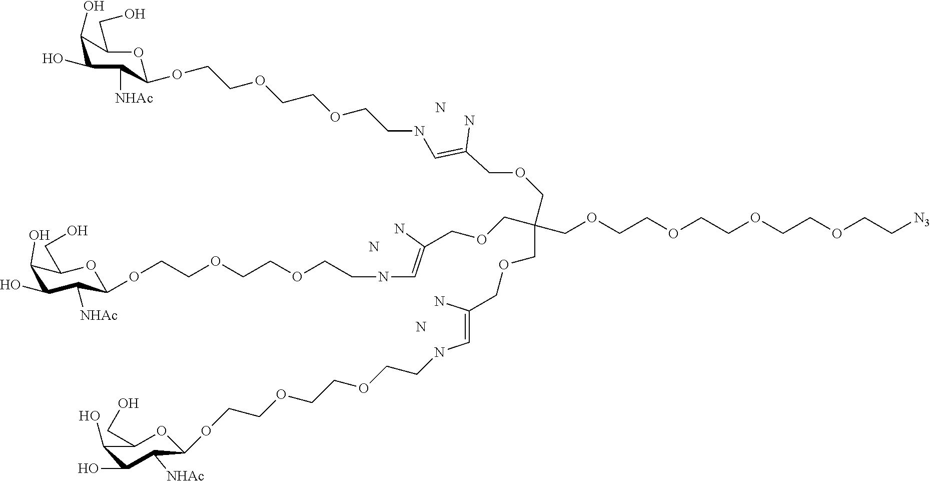

In some embodiments, conjugation of triantennary-GalNAc through one or more surface groups occurs via about 1%, 2%, 3%, 4%, 5%, 6%, 7%, 8%, 9%, 10%, 15%, 20%, 25%, or 30% of the total available surface functional groups, such as hydroxyl groups, of the dendrimers prior to the conjugation. In other embodiments, the conjugation of triantennary-β-GalNAc occurs on less than 5%, less than 10%, less than 15%, less than 20%, less than 25%, less than 30%, less than 35%, less than 40%, less than 45%, less than 50% of total available surface functional groups of the dendrimers prior to the conjugation. In some embodiments, dendrimers are conjugated to an effective amount of triantennary-β-GalNAc for binding to ASGPR and/or targeting and on hepatocytes, whilst conjugated to an effective amount of agents to treat, prevent, and/or image the liver disease or disorder.

### Compositions

In some aspects, the disclosure provides a composition comprising one or more dendrimer conjugates described herein. In some embodiments, the composition is a pharmaceutical composition. Pharmaceutical compositions including one or more dendrimer conjugates can be formulated in a conventional manner using one or more physiologically acceptable carriers, including excipients and auxiliaries which facilitate processing of the active compounds into preparations which can be used pharmaceutically. Proper formulation is dependent upon the route of administration chosen. In some embodiments, the compositions are formulated for parenteral delivery. In some embodiments, the compositions are formulated for intratumoral injection. In some embodiments, the compositions can be formulated in sterile saline or buffered solution for injection into the tissues or cells to be treated. The compositions can be stored lyophilized in single use vials for rehydration immediately before use.

In some embodiments, a pharmaceutical composition comprises one or more dendrimer conjugates and one or more pharmaceutically acceptable excipients. Examples of excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients may be selected from materials which are generally recognized as safe (GRAS), and may be administered to a subject without causing undesirable biological side effects or unwanted interactions.

In some embodiments, pharmaceutically acceptable salts can be prepared by reaction of the free acid or base forms of a compound with a stoichiometric amount of the appropriate base or acid in water or in an organic solvent (e.g., non-aqueous media, such as ether, ethyl acetate, ethanol, isopropanol, or acetonitrile). Pharmaceutically acceptable salts can include salts of a compound derived from inorganic acids, organic acids, alkali metal salts, and alkaline earth metal salts, as well as salts formed by reaction of the compound with a suitable organic ligand (e.g., quaternary ammonium salts). Lists of suitable salts are found, for example, in Remington's Pharmaceutical Sciences, 20th ed., Lippincott Williams & Wilkins, Baltimore, MD, 2000, p. 704.

In some embodiments, a composition is formulated in dosage unit form for ease of administration and uniformity of dosage. In some embodiments, a dosage unit form refers to a physically discrete unit of conjugate appropriate for a subject to be treated. A therapeutically effective dose can be estimated initially either in cell culture assays or in animal models, such as mice, rabbits, dogs, or pigs. An animal model can also be used to achieve a desirable concentration range and route of administration. Such information may be used to determine useful doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index and it can be expressed as the ratio, LD50/ED50.

In some embodiments, a composition is administered locally, for example, by injection directly into a site to be treated. In some embodiments, a composition is injected, topically applied, or otherwise administered directly into the vasculature onto vascular tissue at or adjacent to a site of injury, surgery, or implantation. For example, in some embodiments, a composition is topically applied to vascular tissue that is exposed, during a surgical or implantation, or transplantation procedure. Pharmaceutical compositions formulated for administration by parenteral (intramuscular, intraperitoneal, intravenous (IV) or subcutaneous injection), enteral, and topical routes of administration are described.

In some embodiments, a dendrimer conjugate is formulated to be administered parenterally. The phrases “parenteral administration” and “administered parenterally” are art-recognized terms, and include modes of administration other than enteral and topical administration, such as injections, and can include intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intraarticular, subcapsular, subarachnoid, intraspinal, and intrasternal injection and infusion. In some embodiments, a composition is administered parenterally, for example, by subdural, intravenous, intrathecal, intraventricular, intraarterial, intra-articular, intra-synovial, intra-amniotic, intraperitoneal, or subcutaneous routes.

For liquid formulations, pharmaceutically acceptable carriers can be, for example, aqueous or non-aqueous solutions, suspensions, emulsions, or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents include propylene glycol, polyethylene glycol, and injectable organic esters such as ethyl oleate. Aqueous carriers include, for example, water, alcoholic/aqueous solutions, cyclodextrins, emulsions, or suspensions, including saline and buffered media. A composition can also be administered in an emulsion, for example, water in oil. Examples of oils include those of petroleum, animal, vegetable, or synthetic origin, for example, peanut oil, soybean oil, mineral oil, olive oil, sunflower oil, fish-liver oil, sesame oil, cottonseed oil, and corn oil. Suitable fatty acids for use in parenteral formulations include, for example, oleic acid, stearic acid, isostearic acid, ethyl oleate, and isopropyl myristate.

In some embodiments, compositions suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, and electrolyte replenishers, such as those based on Ringer's dextrose. In general, water, saline, aqueous dextrose and related sugar solutions, and glycols such as propylene glycols or polyethylene glycol are preferred liquid carriers, particularly for injectable solutions. Injectable pharmaceutical carriers for injectable compositions are known in the art (see, e.g., Pharmaceutics and Pharmacy Practice, J.B. Lippincott Company, Philadelphia, PA, Banker and Chalmers, eds., pages 238 250 (1982), and ASHP Handbook on Injectable Drugs, Trissel, 15th ed., pages 622 630 (2009)).

In some embodiments, a dendrimer conjugate is formulated to be administered enterally. The carriers or diluents may be solid carriers or diluents for solid formulations, liquid carriers or diluents for liquid formulations, or mixtures thereof. For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. Examples of oils and fatty acids are as described for formulating compositions for parenteral administration.

Vehicles include, for example, sodium chloride solution, Ringer's dextrose, dextrose and sodium chloride, lactated Ringer's, and fixed oils. Formulations include, for example, aqueous and non aqueous, isotonic sterile injection solutions, which can contain antioxidants, buffers, bacteriostats, and solutes that render the formulation isotonic with the blood of the intended recipient, and aqueous and non aqueous sterile suspensions that can include suspending agents, solubilizers, thickening agents, stabilizers, and preservatives. In general, water, saline, aqueous dextrose, and related sugar solutions can be used as liquid carriers. These can also be formulated with proteins, fats, saccharides and other components of infant formulas.

In some embodiments, a dendrimer conjugate is formulated for oral administration. Oral formulations may be in the form of chewing gum, gel strips, tablets, capsules or lozenges. Encapsulating substances for the preparation of enteric-coated oral formulations include cellulose acetate phthalate, polyvinyl acetate phthalate, hydroxypropyl methylcellulose phthalate, and methacrylic acid ester copolymers. Solid oral formulations such as capsules or tablets are preferred. Elixirs and syrups also are well known oral formulations.

In some embodiments, a dendrimer conjugate is formulated to be administered topically. Topical administration can include application directly to exposed tissue, vasculature, mucosa or to tissues or prostheses, for example, during surgery. The preferred tissue for topical administration is tumor.

### Therapeutic Uses

In some embodiments, the dendrimer complexes are used to treat cancer. In other embodiments, the dendrimer complexes are used to treat autoimmune diseases. The methods typically include administering to a subject in a need thereof an effective amount of a composition including dendrimer and one or more therapeutic agents to modulate the immune microenvironment, either to decrease an autoimmune response or increase and anti-tumor response.

In general, the compositions and methods of treatment thereof are useful in the context of cancer, including tumor therapy. The compositions can also be used for treatment of other diseases, disorders and injury including inflammatory diseases, including, but not limited to, ulcerative colitis, Crohn's disease, and rheumatoid arthritis.

In some embodiments, a subject to be treated is a human. All the methods described can include the step of identifying and selecting a subject in need of treatment, or a subject who would benefit from administration with the compositions. Therefore, in some embodiments, compositions of dendrimers conjugated or complexed with one or more immunomodulatory agents and/or additional therapeutic or diagnostic agents are administered to a subject in need of immunomodulation in the context of treatment for cancer, or treatment of other diseases, disorders and injury including inflammatory diseases such as ulcerative colitis, Crohn's disease, rheumatoid arthritis, and bone diseases.

In some embodiments, compositions of dendrimers conjugated or complexed with one or more immunomodulatory agents and/or additional therapeutic or diagnostic agents are administered to a subject having a proliferative disease, such as a benign or malignant tumor. In some embodiments, the subjects to be treated have been diagnosed with stage I, stage II, stage III, or stage IV cancer. In some embodiments, the proliferative disease is neurofibromatosis. Neurofibromatosis refers to a group of genetic disorders that cause tumors to form on nerve tissue. These tumors can develop anywhere in the nervous system, including the brain, spinal cord and nerves. There are three types of neurofibromatosis: neurofibromatosis type 1 (NF1), neurofibromatosis type 2 (NF2), and schwannomatosis. NF1 is usually diagnosed in childhood, while NF2 and schwannomatosis are usually diagnosed in early adulthood. The tumors in these disorders are usually noncancerous (benign), but sometimes can become cancerous (malignant). Accordingly, in some embodiments, a subject to be treated has or is suspected of having a proliferative disease, such as NF1, NF2, and/or schwannomatosis. In some embodiments, a subject to be treated has or is suspected of having NF1.

Neurofibromatosis Type 1 (NF1) is a common cancer predisposition syndrome characterized by the progressive development of slow growing tumors called plexiform neurofibromas. These tumors involve the cranial and large peripheral nerves, are initiated by loss of NF1 heterozygosity in Schwann cells, contain high levels of collagen, and are infiltrated by inflammatory cells. Due to the high inoperability of neurofibromas, pharmacological therapeutics are the main strategy in targeting these neoplasms. Selumetinib (Koselugo™), a mitogen-activated protein kinase (MAPK) kinase (MEK) inhibitor, is the only FDA-approved drug for NF1, and though this important inhibitor promotes partial responses in both children and adults, there is still a need for greater shrinkage and durability.

However, global toxicity of combinatorial agents is a limiting factor in treating neurofibromas, thereby reducing the ability of drugs to attenuate tumor shrinkage or progression.

The compositions and methods are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth. For example, in some embodiments, the disclosure provides compositions and methods for treating a subject having a tumor that is associated with (e.g., caused by) a proliferative disease, such as neurofibromatosis (e.g., NF1).

The types of cancer that can be treated with the compositions and methods include, but are not limited to, cancers such as vascular cancer such as multiple myeloma, adenocarcinomas and sarcomas, of bone, bladder, brain, breast, cervical, colorectal, esophageal, kidney, liver, lung, nasopharangeal, pancreatic, prostate, skin, stomach, and uterine. In some embodiments, the compositions are used to treat multiple cancer types concurrently. The compositions can also be used to treat metastases or tumors at multiple locations.

In some embodiments, compositions of dendrimers conjugated or complexed with one or more immunomodulatory agents and/or additional therapeutic or diagnostic agents are administered to a subject with an autoimmune or inflammatory disease or disorder. Autoimmune disease happens when the body's natural defense system cannot effectively differentiate between the body's own cells and foreign cells, causing the body to mistakenly attack normal cells. There are more than 80 types of autoimmune diseases that affect a wide range of body parts. Common autoimmune diseases include rheumatoid arthritis, psoriasis, psoriatic arthritis, systemic lupus erythematosus (SLE), type 1 diabetes, inflammatory bowel disease, and thyroid diseases.

In some embodiments, the compositions can also be used for treatment of autoimmune or inflammatory disease or disorder such as rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Bechet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis-juvenile, discoid lupus, essential mixed cryoglobulinemia, fibromyalgia-fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

In some embodiments, the compositions and methods can also be used for treatment of autoimmune or inflammatory diseases or disorders involving bones and joints, including infections and immunologically-mediated local and systemic diseases.

The compositions and methods are suitable for treatment one or more diseases or disorders of the eye. The compositions and methods are suitable for alleviating one or more symptoms associated with one or more diseases or disorder of the eye, for example, discomfort, pain, dryness, excessive tearing, injuries, infections, burns, and gradual loss of vision.

In some embodiments, the eye disorder to be treated is a back of the eye disease such as diabetic eye disease, symptomatic vitreomacular adhesion/vitreomacular traction (sVMA/VMT), and wet (neovascular) or dry AMD (age-related macular degeneration). In some embodiments, the eye disorder to be treated is one or more retinal and choroidal vascular diseases (e.g., AMD, retinopathy of prematurity, diabetic macular edema, retinal vein occlusion, retinopathy associated with toxicity of chemotherapy e.g., MEK retinopathy). In some embodiments, the eye disorder to be treated is age-related macular degeneration (AMD). Age-related macular degeneration (AMD) is a neurodegenerative, neuroinflammatory disease of the macula, which is responsible for central vision loss. The pathogenesis of age-related macular degeneration involves chronic neuroinflammation in the choroid (a blood vessel layer under the retina), the retinal pigment epithelium (RPE), a cell layer under the neurosensory retina, Bruch's membrane and the neurosensory retina, itself.

In other embodiments, the eye disorder to be treated is an inflammatory disease of the eye, i.e., diseases of the eye associated with inflammation of the tissues of the eye, including, for example, AMD, retinitis pigmentosa, optic neuritis, sarcoid, retinal detachment, temporal arteritis, retinal ischemia, arteriosclerotic retinopathy, hypertensive retinopathy, retinal artery blockage, retinal vein blockage, diabetic retinopathy, macular edema, Stargardt disease (also known as Stargardt macular dystrophy or juvenile macular degeneration), geographic atrophy, neuromyelitis optica, and also including angiogenic diseases including, for example, retinal neovascularization and choroidal neovascularization. Other conditions can also result in inflammation and/or angiogenesis in the eye, for example, infection, sickle cell disease, hypotension, etc.

Further examples of eye disorders that may be treated include amoebic keratitis, fungal keratitis, bacterial keratitis, viral keratitis, onchorcercal keratitis, bacterial keratoconjunctivitis, viral keratoconjunctivitis, corneal dystrophic diseases, Fuchs' endothelial dystrophy, meibomian gland dysfunction, anterior and posterior blepharitis, conjunctival hyperemia, conjunctival necrosis, cicatrical scaring and fibrosis, punctate epithelial keratopathy, filamentary keratitis, corneal erosions, thinning, ulcerations and perforations, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, anterior uveitis, posterior uveitis (including toxoplasmosis), pan-uveitis, inflammatory disease of the vitreous or retina, endophthalmitis prophylaxis and treatment, macular edema, macular degeneration, age-related macular degeneration, proliferative and non-proliferative diabetic retinopathy, hypertensive retinopathy, an autoimmune disease of the retina, primary and metastatic intraocular melanoma, other intraocular metastatic tumors, open angle glaucoma, closed angle glaucoma, pigmentary glaucoma and combinations thereof. Other disorders include injury, burn, or abrasion of the cornea, cataracts and age related degeneration of the eye or vision associated therewith.

In some embodiments, triantennary-GalNAc modified dendrimers complexed with or conjugated to one or more agents to treat, prevent, and/or diagnose one or more liver disorders and/or diseases are administered to a subject to treat, prevent, and/or diagnose one or more symptoms of one or more liver disorders and/or diseases in the subject.

Dendrimer-triantennary-β-GalNAc compositions are effective for treating or ameliorating one or more symptoms of a liver disease, or disorder, such as acute or chronic liver diseases. Exemplary indications that can be treated include, but are not limited to, acute liver failure (acute hepatitis, fulminant hepatitis), e.g., resulting from neoplastic infiltration, acute Budd-Chiari syndrome, heatstroke, mushroom ingestion, metabolic diseases such as Wilson's disease, or associated with viral liver disease such as caused by herpes simplex viruses, cytomegalovirus, Epstein-Barr virus, parvoviruses, hepatitis viruses (e.g., hepatitis A, hepatitis E, hepatitis D+B infections), or drug-induced liver injury, including rifampicin-induced hepatotoxicity, acetaminophen-induced hepatotoxicity, recreational-drug induced toxicity such as by 3,4-methylenedioxy-N-methylamphetamine (MDMA, also known as ecstasy), or cocaine-induced toxicity, acute ischemic hepatocellular injury, or hypoxic hepatitis, or resulting from traumatic liver injury. The methods can treat and prevent any hyperacute, acute and subacute liver disease defined by the occurrence of encephalopathy, coagulopathy and jaundice in an individual with a previously normal liver.

Symptoms and clinical manifestations of acute liver disease include jaundice and encephalopathy, and impaired liver function (e.g., loss of metabolic function, decreased gluconeogenesis leading to hypoglycemia, decreased lactate clearance leading to lactic acidosis, decreased ammonia clearance leading to hyperammonemia, and reduced synthetic capacity leading to coagulopathy). Acute liver diseases and disorders are often associated with multiple systemic manifestations, including immunoparesis contributing to high risk of sepsis; systemic inflammatory responses, with high energy expenditure or rate of catabolism; portal hypertension; kidney dysfunction; myocardial injury; pancreatitis (particularly in acetaminophen-related disease); inadequate glucocorticoid production in the adrenal gland contributing to hypotension; and acute lung injury, leading to acute respiratory distress syndrome.

All the methods described can also include the step of identifying and selecting a subject in need of treatment, or a subject who would benefit from administration with the compositions. In some embodiments, the subject has been medically diagnosed as having an acute liver disease or disorder by exhibiting clinical (e.g., physical) symptoms of the disease. In other embodiments, the subject has been medically diagnosed as having a subacute or chronic liver disease or disorder by exhibiting clinical (e.g., physical) symptoms, which are indicative of an increased risk or likelihood of developing acute liver disease. Therefore, in some embodiments, formulations of the disclosed dendrimer compositions are administered to a subject prior to a clinical diagnosis of acute liver disease.

In some embodiments, the methods treat or prevent non-alcoholic steatohepatitis, liver fibrosis associated with non-alcoholic steatohepatitis, primary biliary cholangitis.

In some embodiments, a dendrimer conjugate can be administered in combination with one or more additional therapeutically active agents, which are known to be capable of treating conditions or diseases discussed above.

### EXAMPLES

### Example 1. Conjugation of Didesethyl Sunitinib Via a Non-Cleavable Linkage

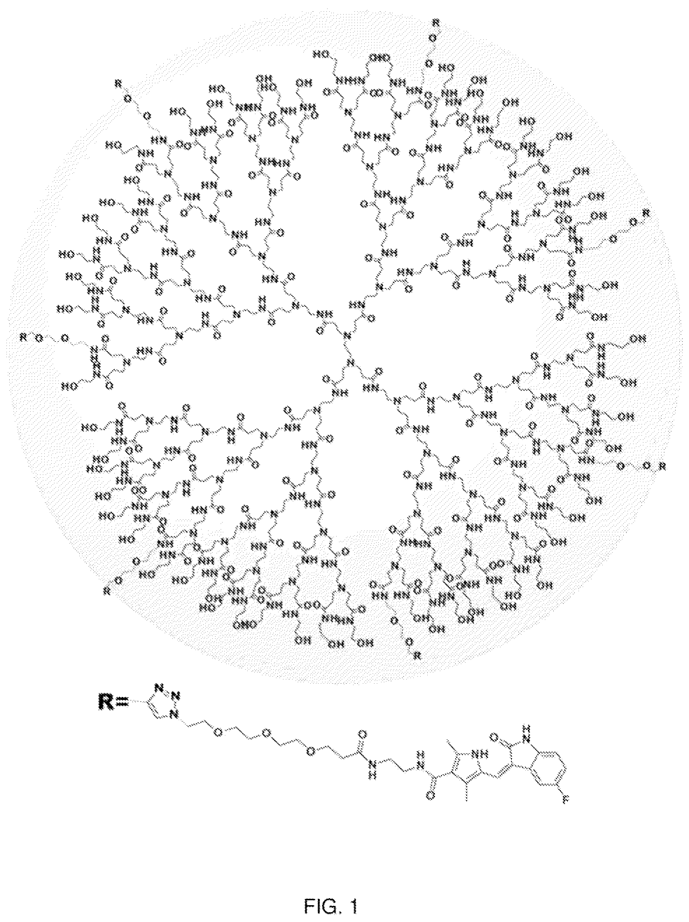

Overexpression of vascular endothelial growth factor (VEGF) has been implicated in a number of diseases associated with angiogenesis. Sunitinib is a receptor tyrosine kinase inhibitor that blocks VEGF receptors and has excellent antiangiogenic activity and is approved by the FDA for use in different types of cancers. Didesethyl sunitinib is an active metabolite of sunitinib. Despite the excellent therapeutic value of sunitinib and its analogues, their clinical development is hampered by the associated toxicity. The dendrimer-didesethyl sunitinib conjugates aim to overcome the dose related toxicities of sunitinib by attaching it to a hydroxyl terminated dendrimer. The chemical structure of the dendrimer conjugate synthesized in this example is shown in FIG. 1.

### Synthesis and Characterization of N, N-Didesethyl Sunitinib Amide Azide

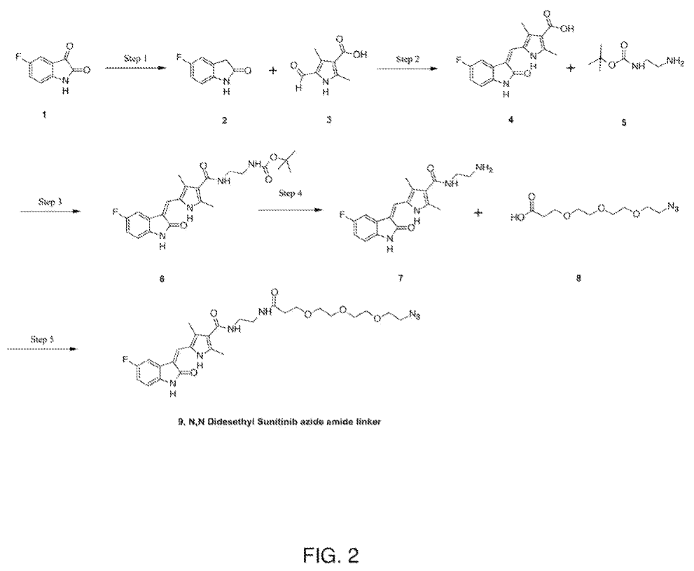

The reaction scheme for synthesis of N, N-didesethyl sunitinib azide with an amide linkage is shown in FIG. 2.

### Step 1: Synthesis of 5-fluoro-2,3-dihydro-1H-indol-2-one (Compound 2)

To a stirred solution of 5-fluoro-2,3-dihydro-1H-indole-2,3-dione (6.0 gm, 1.0 eq.) in n-butanol (10V) was added triethyl amine (6.12 mL, 1.2 eq.) and followed by hydrazine hydrate (3.56 mL, 2.0 eq.) was added at room temperature. The resulting solution was stirred for 16 hours at 100° C. Reaction progress was monitored by TLC (50% ethylacetate in Hexanes). Once the reaction was judged to completion, reaction mass was as such evaporated to dryness under vacuum at 45° C. to obtain dark brown solid. The obtained solid was quenched with water (20 V) and extracted with ethyl acetate (30V) and organic layer was given water wash. Organic layer was concentrated to dryness on rotary evaporator. The crude product was purified by recrystallization using ethyl acetate to get grey color fluffy solid (4.0 g, 72% yield). The compound 2 shown in FIG. 2 was confirmed by ¹H NMR, liquid chromatography, and mass spectrometry.

### Step 2: Synthesis of 5-{[(3Z)-5-fluoro-2-oxo-2,3-dihydro-1H-indol-3-ylidene]methyl}-2,4-dimethyl-1H-pyrrole-3-carboxylic acid (Compound 4)

To a stirred solution of 5-fluoro-2,3-dihydro-1H-indol-2-one (compound 2) (4.0 gm, 1.0 eq.) and 5-formyl-2,4-dimethyl-1H-pyrrole-3-carboxylic acid (compound 3) (4.41 gm, 1.0 eq.) in ethanol (10V) was added pyrrolidine (4.42 mL, 2.0 eq.) at room temperature. The resulting solution was stirred for 3 hours at 80° C. Reaction progress was monitored by TLC (10% Methanol in DCM). Once the reaction was judged to completion, reaction mass was cooled to room temperature added 2M HCl solution to pH=3. A brownish-red precipitate was formed and filtered. The obtained solid was washed with ethanol (20 V) followed by hexanes (30 V) and filtered to get reddish-orange solid. (6.6 g, 82% yield.) The compound 4 shown in FIG. 2 was confirmed by ¹H NMR.

### Step 3: Synthesis of tert-butyl N-{2-[(5-{[(3Z)-5-fluoro-2-oxo-2,3-dihydro-1H-indol-3-ylidene]methyl}-2,4-dimethyl-1H-pyrrol-3-yl)formamido]ethyl}carbamate (Compound 6)

To a solution of 5-{[(3Z)-5-fluoro-2-oxo-2,3-dihydro-1H-indol-3-ylidene]methyl}-2,4-dimethyl-1H-pyrrole-3-carboxylic acid (compound 4) (6.5 g, 1.0 eq.) in DMF were added triethyl amine (6.08 mL, 2.0 eq.) EDC·HCl ((8.68 g, 2.1 eq.), HOBT (3.94 g, 1.35 eq.) and tert-butyl N-(2-aminoethyl) carbamate (4.16 g, 1.2 eq.) and at 0° C. Reaction was stirred at room temperature for 16 hours. Reaction mixture was diluted with water (20.0 V), stirred for 10 min. to precipitate and filtered to get brown solid. The obtained solid was washed with ethyl acetate (15.0 V), followed by hexanes (15.0V), filtered and dried to get brownish-orange solid as a tert-butyl N-{2-[(5-{[(3Z)-5-fluoro-2-oxo-2,3-dihydro-1H-indol-3-ylidene]methyl}-2,4-dimethyl-1H-pyrrol-3-yl)formamido]ethyl}carbamate (compound 6) (7.5 g, 78% yield). The compound 6 shown in FIG. 2 was confirmed by ¹H NMR.

### Step 4: Synthesis of N-(2-aminoethyl)-5-[{(3Z)-5-fluoro-2-oxo-2,3-dihydro-1H-indol-3-ylidene]methyl}-2,4-dimethyl-1H-pyrrole-3-carboxamide (Compound 7)

To a solution of tert-butyl N-{2-[(5-{[(3Z)-5-fluoro-2-oxo-2,3-dihydro-1H-indol-3-ylidene]methyl}-2,4-dimethyl-1H-pyrrol-3-yl)formamido]ethyl}carbamate (compound 6) (9.0 g, 1.0 eq.) in DCM (10.0 V) was added trifluoro acetic acid (3.0 V) at 0-5° C. Reaction was stirred at room temperature for 12 hours. Reaction mass was as such evaporated to dryness under vacuum at 45° C. to obtain dark brown solid. The obtained solid was washed with diethyl ether (15.0 V) filtered and dried to get orange-yellow solid (6.0 g crude). The compound 7 shown in FIG. 2 was confirmed by ¹H NMR, liquid chromatography, and mass spectrometry.

### Step 5: Synthesis of N-{2-[(5-{[(3Z)-5-fluoro-2-oxo-2,3-dihydro-1H-indol-3-ylidene]methyl}-2,4-dimethyl-1H-pyrrol-3-yl)formamido]ethyl}-3-[2-(2-propoxyethoxy)ethoxy]propanamide (Compound 9)

To a solution of 3-[2-(2-propoxyethoxy)ethoxy]propanoic acid (8) (5.95 g, 1.0 eq.) in DMF (10.0 V) were added DIPEA (8.40 mL, 2.0 eq.) EDC·HCl (6.90 g, 1.5 eq.), HOBT (0.65 g, 0.2 eq.), N-(2-aminoethyl)-5-{[(3Z)-5-fluoro-2-oxo-2,3-dihydro-1H-indol-3-ylidene]methyl}-2,4-dimethyl-1H-pyrrole-3-carboxamide (compound 7) (11.0 g, 1.0 eq.) and DMAP (0.294 g, 0.1 eq.) at 0-5° C. Reaction was stirred at room temperature for 3 hours. Reaction progress was monitored by TLC (10% MeOH in DCM). Reaction mixture was diluted with water (20.0 V) stirred for 10 min. to form brown precipitate and filtered. The obtained solid purified by reverse phase column chromatography to obtain N, N-didesethyl sunitinib amide azide as an orange solid (5.2 g, 37% yield). The compound 9 was confirmed by ¹H NMR, liquid chromatography, and mass spectrometry.

### Synthesis of Dendrimer Conjugate Via a Non-Cleavable Ether Linkage on Dendrimer

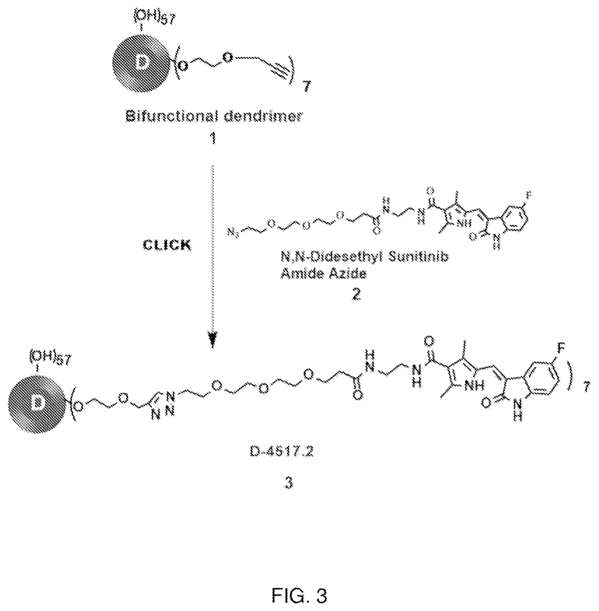

The synthesis began by the construction of a bifunctional dendrimer. At dendrimer generation 3.5, 7 alkyne functional groups were introduced using a polyethyl glycol (PEG) linker with an amine at one end and a hexyne at the other end to produce a generation 4 bifunctional dendrimer (compound 1 in FIG. 3) with 7 alkyne arms and 57 hydroxyl groups on the surface. The structure of the dendrimer was confirmed by 1H NMR spectroscopy.

The clickable didesethyl sunitinib analog (compound 2 in FIG. 3, AVT-4517), including didesethyl sunitinib, a three ethylene glycol (PEG3) spacer and a terminal azide, was synthesized to participate in the click reaction with alkyne groups on the surface of the dendrimer. The active agent, compound 2, is manufactured using a 5-step synthesis as shown in FIG. 2 and described above.

AVT-4517 (compound 2 in FIG. 3) is finally reacted with the bifunctional dendrimer (compound 1 in FIG. 3) with hexyne groups by copper (I) catalyzed alkyne-azide click chemistry to yield D-4517.2 (compound 3 in FIG. 3) with the full structure shown in FIG. 1. After conjugation of the analog to the dendrimer, the D-4517.2 is purified by tangential flow filtration (TFF) to remove any impurities and enable purification into the final formulation.

### ¹H-NMR Analysis of D-4517.2 Conjugates

The formation of product D-4517.2 is confirmed by ¹H NMR. The ¹H NMR spectrum of the conjugate clearly shows the peaks corresponding to the dendrimer, drug and linkers attached to it, and the drug loading was calculated by comparing these peaks with the help of proton integration method. The internal amide protons from the dendrimer are present in between δ 8.5-7.5 ppm when spectrum is recorded in deuterated DMSO. These amide peaks are a reference standard for the rest of the peaks. The —NH peaks from drug appear at δ 13.6 and 10.8 ppm. There are 4 protons from the drug and one triazole proton which is formed after the click reaction merged with internal amide peaks and comes in between δ 8.5-7.5 ppm. Additionally, 2 aromatic protons from sunitinib situated next to the fluorine group appear at δ 6.95-6.85 ppm. A sharp triazole peak at δ 7.7 ppm which is a signature peak for the click transformation is observed when the NMR solvent is switched from deuterated DMSO to CD₃OD. After the click, the CH₂ present next to the azide down shielded and can be observed at δ 4.4 ppm. NMR is also used to quantitate the number of drug molecules conjugated to the hydroxyl dendrimer. The drug loading was calculated by proton integration method by comparing the protons of dendrimer internal amide protons to drug protons.

### HPLC Analysis for Assessment of Purity of D-4517.2

The purity of the dendrimer drug conjugate, intermediate and drug linker was evaluated using HPLC. The final conjugate is >99% pure by HPLC. The dendrimer G4-OH and dendrimer hexyne intermediate is visible at 210 nm channel and the didesethyl suntinib is visible at 430 nm in HPLC. The retention time of the compound 2 is around 16.9 minutes but once the hydrophobic drug molecules are attached to the dendrimer, the peak of the final conjugate shifts towards the right and comes around 27 minutes, which confirms the attachment of hydrophobic drugs to the dendrimer construct. Once the drug is attached to dendrimer the peak corresponding to it can be observed at both 210 nm (dendrimer absorption wavelength) and 430 nm (drug absorption wavelength) channels, which further confirms the formation of product. The drug loading of the dendrimer conjugate is around 12.6% wt/wt which corresponds to 7 molecules of drug attached per dendrimer molecule.

### Size and Zeta Potential

The size and the zeta potential distribution of the D-4517.2 are determined using a Zetasizer Nano ZS instrument. For the size measurement, the sample was prepared by dissolving the dendrimer in deionized water (18.2Ω) to make a solution with a final concentration of 0.5 mg/mL. The solution was then filtered through 0.2 m syringe filters (Pall Corporation, 0.2 m HT Tuffryn membrane) directly into the cell (UV transparent disposable cuvette, Dimensions: 12.5×12.5×45 mm). For zeta potential measurement, the sample was prepared at a concentration of 0.2 mg/mL in 10 mM NaCl using above mentioned procedure. Malvern Zetasizer Nanoseries disposable folded capillary cell was used for the measurements. The size of D-4517 was 5.5±0.5 nm and zeta potential was slightly positive (+5.4±0.4 mV).

### Size Exclusion Chromatography Multiple-Angle Laser Scattering (SEC-MALS)

The molar mass of D-4517.2 will be determined by size exclusion chromatography multiple-angle laser scattering (SEC-MALS).

### Results

D-4517 has nanomolar affinity for VEGFR2 and does not require the release of the active drug, AVT-4517. To further increase the stability of the conjugate under physiological conditions and further reduce the release of the drug from the conjugate as observed in D-4517 buffer and plasma stability studies, the cleavable ester linkages on the dendrimer surface were replaced with non-cleavable linkages as demonstrated in the structure of D-4517.2 (FIG. 1). There are no cleavable bonds in the structure of D-4517.2.

D-4517.2 is a covalent conjugate of generation-4, hydroxyl-terminated PAMAM dendrimers, containing an ethylene diamine (EDA) core, amidoamine repeating units [CH₂CH₂CONHCH₂CH₂N]), and 64 hydroxyl end groups (chemical formula: C₆₂₂H₁₁₈₄N₁₈₆O₁₈₈) with didesethyl sunitinib analog (AVT-4517) conjugated to the dendrimer by a highly efficient click chemistry approach. The hydroxyl, generation-4, PAMAM dendrimers are mono-disperse and produced with high compositional purity (>95%). For the preparation of D-4517.2, seven of the 64 hydroxyl groups on the dendrimer are modified to attach AVT-4517 (˜12.6% of total mass).

### Stability Studies in Human, Mouse and Rat Plasma

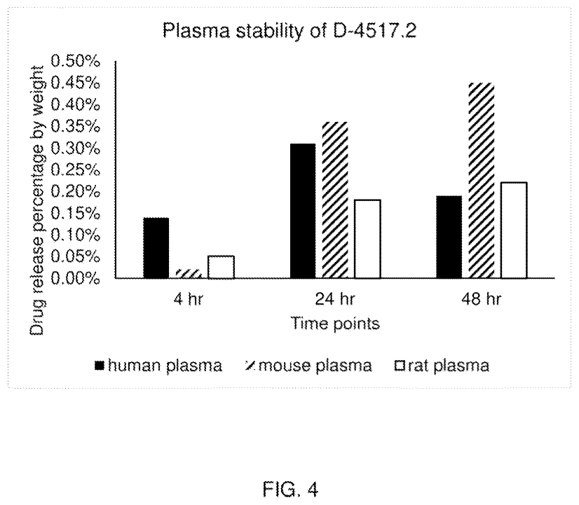

In vitro stability of dendrimer didesethyl sunitinib conjugates, D-4517 and D-4517.2, in human, mouse and rat plasma was evaluated at physiological conditions. The results presented in FIG. 4. Compared with D4517 (2% (weight percentage) release in human plasma, and 4% (weight percentage) release in rat plasma), the plasma stability of D4517.2 is improved significantly. At 48 hours, in all three plasma, less than 0.5% drug (by weight) was released from dendrimer drug conjugates.

### Binding Affinity

The kinase comparative binding affinity of D-4517 and D-4517.2 was evaluated, and the results are presented in Table 1.

TABLE 1
Dendrimer conjugates, D-4517 and D-4517.2 binding assay study

| Compound Name | Gene Symbol | Modifier | Kd (nM) |
|---|---|---|---|
| D4517 | VEGFR2 | = | 27 |
| D4517 | VEGFR1 | = | 1100 |
| D4517 | CSF1R | = | 82 |
| D4517 | KIT | = | 3.4 |
| D4517 | PDGFRA | = | 16 |
| D4517 | PDGFRB | = | 11 |
| D-4517.2 | CSF1R | = | 41 |
| D-4517.2 | VEGFR1 | = | 890 |
| D-4517.2 | KIT | = | 3 |
| D-4517.2 | PDGFRA | = | 11 |
| D-4517.2 | PDGFRB | = | 7.5 |
| D-4517.2 | VEGFR2 | = | 14 |

The IC50 results of D-4517.2 is lower than D-4517 on all tested assay, which indicates the stronger binding between D4517.2 and tyrosine kinase receptor.

### Example 2. Conjugation of N-Acetyl-L-Cysteine Via a Non-Cleavable Linkage

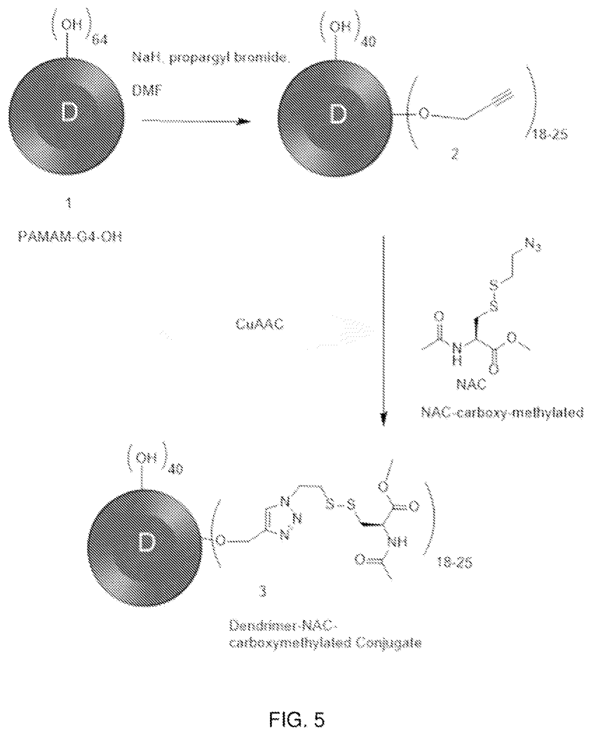

A dendrimer conjugated with N-acetyl-L-cysteine via a non-cleavable linker was synthesized. The synthesis route for a non-releasable (or non-cleavable) form of the dendrimer/N-acetyl-cysteine conjugate is shown in FIG. 5. As shown, N-acetyl-L-cysteine was conjugated to a hydroxyl-terminated PAMAM dendrimer via non-cleavable linkage for minimal release of free N-acetyl-cysteine in vivo after administration. The non-releasable form of the dendrimer/N-acetyl-cysteine complex provides enhanced therapeutic efficacy as compared to a releasable or cleavable form of the dendrimer/N-acetyl-cysteine complex.

### Example 3. Conjugation of Targeting Agent Via a Non-Cleavable Linkage

Tri-antennary GalNAc-based hydroxyl dendrimers were assessed for targeting and delivering drugs to hepatocytes in a site-specific manner. It has been shown that the surface GalNAc sugars create a multivalent binding effect to ASGPR, allowing the dendrimers to selectively target and internalize in hepatocytes in vivo in STAM model of nonalcoholic steatohepatitis.

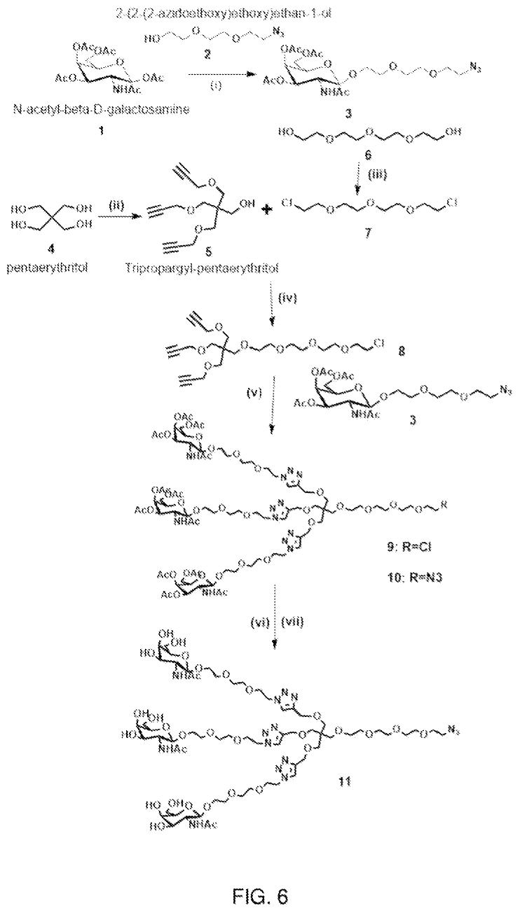

Synthesis scheme of β-GalNAc-triantennary-PEG3-Azide (AB3 building block) is shown in FIG. 6. Reagents and conditions: (i) scandium triflate, DCE, 3 h, 80° C., (ii) propargyl bromide, toluene, sodium hydroxide, water, TBAB, (iii) pyridine, thionyl chloride, chloroform, 65° C., 2 h; (iv) tetrabutylammonium hydrogen sulfate, 50% NaOH, 16 h, rt; (v) (iii) CuSO₄·5H₂O, Na ascorbate, THF, water, 10 h; (vi) DMF, tetrabutylammonium iodide, NaN₃, 80° C., 5 h; (vii) sodium methoxide, dry methanol, 30° C., 3 h.

A triantenary building block was prepared where three molecules of beta-GALNAc-PEG3 azide are grafted on a propargylated pentaerythritol building block to yield AB₃ type orthogonal building block. Synthesis was started with the glycosylation reaction of β-D-GalNAc pentacetate (1, FIG. 6) with 2-[2-(2-azidoethoxy)ethoxy]ethan-1-ol (2) in the presence of scandium triflate in dichloroethane to yield peracetylated β-GalNAc-PEG3-azide (3). On the other hand, pentaerythritol 4 was selectively modified with 3 propargyl arms according to the literature method in the presence of sodium hydroxide and tetrabutylammonium bromide in DMSO to yield tripropargyl pentaerythritol (5). The remaining one hydroxyl group on the compound (5) was reacted with bis-chlorotetraethylene glycol (7) using sodium hydroxide and TBAB in DMSO to afford intermediate compound (8). During the next synthetic step, peracetylated β-GalNAc-PEG3-Azide was clicked with AB₃ building block (8) using conventional CuAAC click reaction conditions (Copper (II) sulphate pentahydrate and sodium ascorbate in THF:Water) to yield compound (9). The success of click reaction is confirmed by ¹H NMR, HRMS and HPLC. In the ¹H NMR, a signature sharp singlet of triazole at δ 7.9 ppm was observed. The other characteristic peaks are the acetate peaks between δ 2.0-1.74 ppm, GalNAc protons from δ 5.2-3.2 ppm and NH of GALNAC at δ7.78 ppm. In the next synthetic step, terminal chloride group of compound (9) was exchanged to azide by nucleophilic substitution in presence of sodium azide and tetrabutyl ammonium iodide in DMF to yield compound (10). The last step is the transesterification using zemplen conditions where the reaction was performed in methanol using sodium methoxide to afford deacetylated β-GalNAc-triantetennary-PEG3 azide (11) building block.

The successful completion of the reaction is confirmed by ¹H NMR where the peaks corresponding to 0-acetates completely disappeared and all the sugar protons shifted upfield. The whole synthetic sequence was characterized using ¹H NMR, HPLC, and HRMS to confirm the desired compounds.

The dendrimer-β-GalNAc conjugate is prepared as described in Examples 1 and 2.

### Example 4. Preparation of PEG-Alkyne Functionalized PAMAM-G4-OH

PAMAM G4-OH-Alkyne_{7_8} was produced in a multi-step process. This process was successfully scaled to provide lots of 1 kg and 0.5 kg of this material.

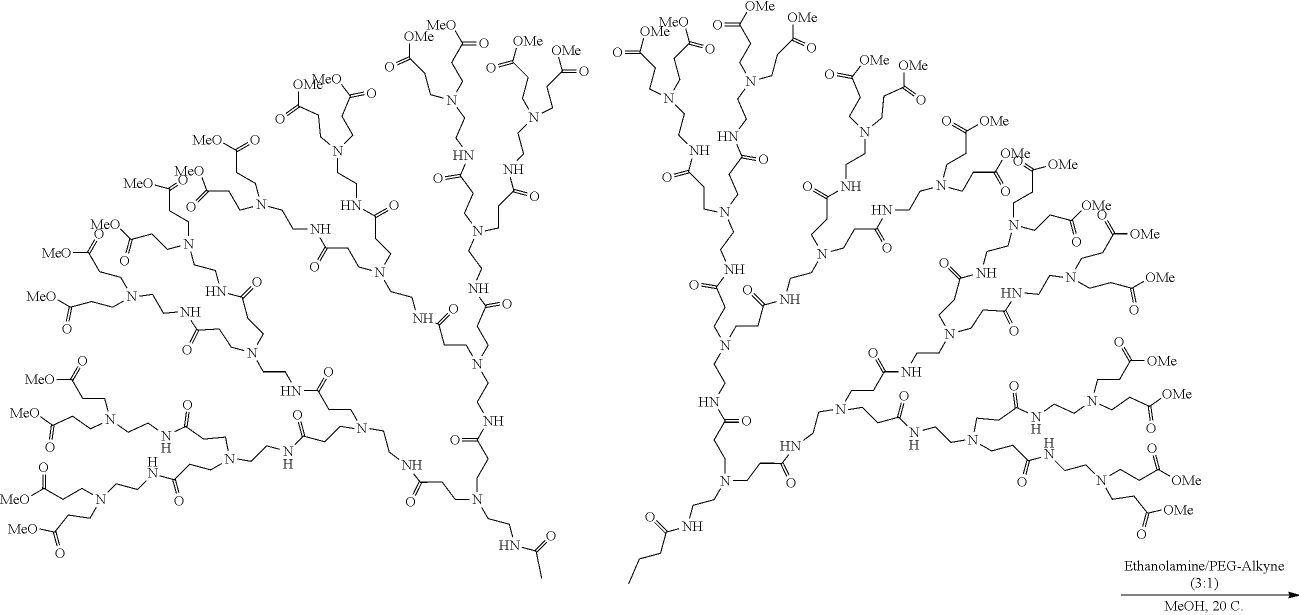

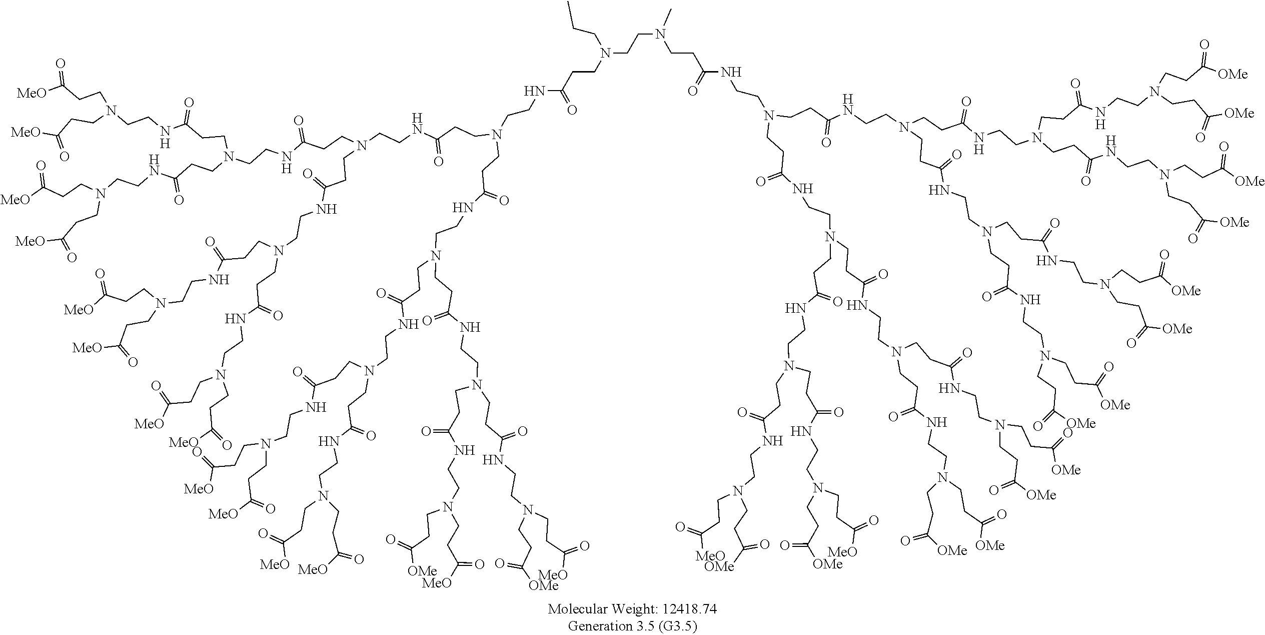

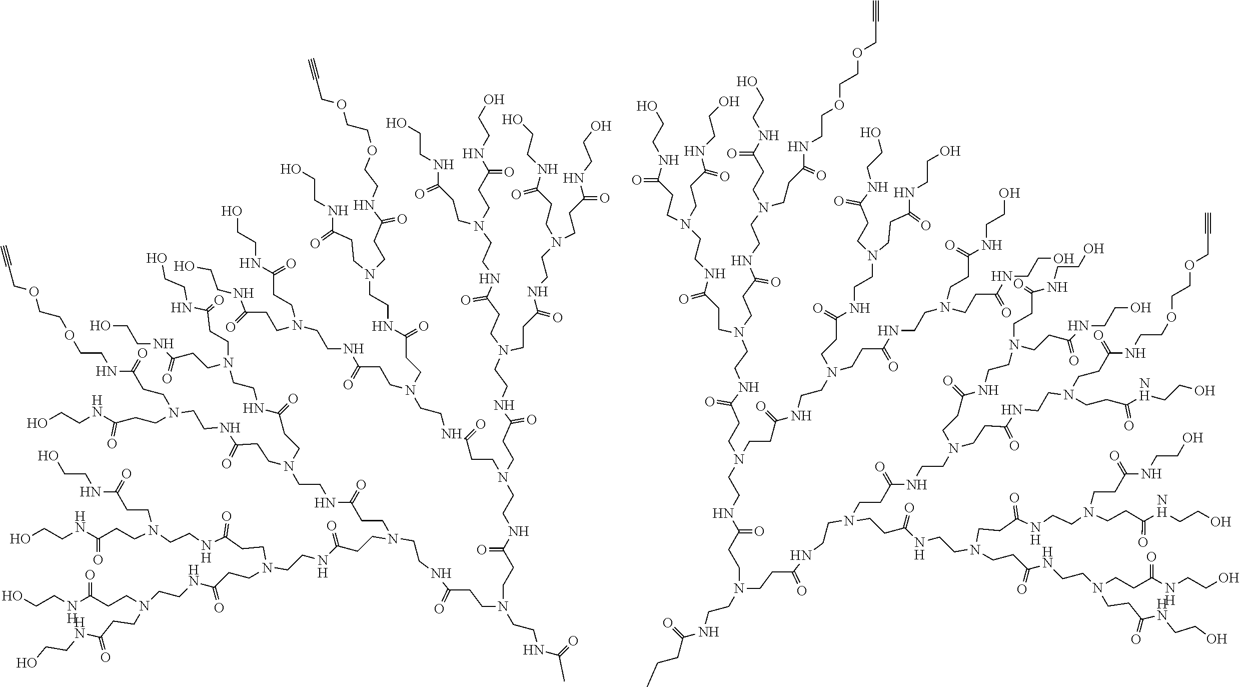

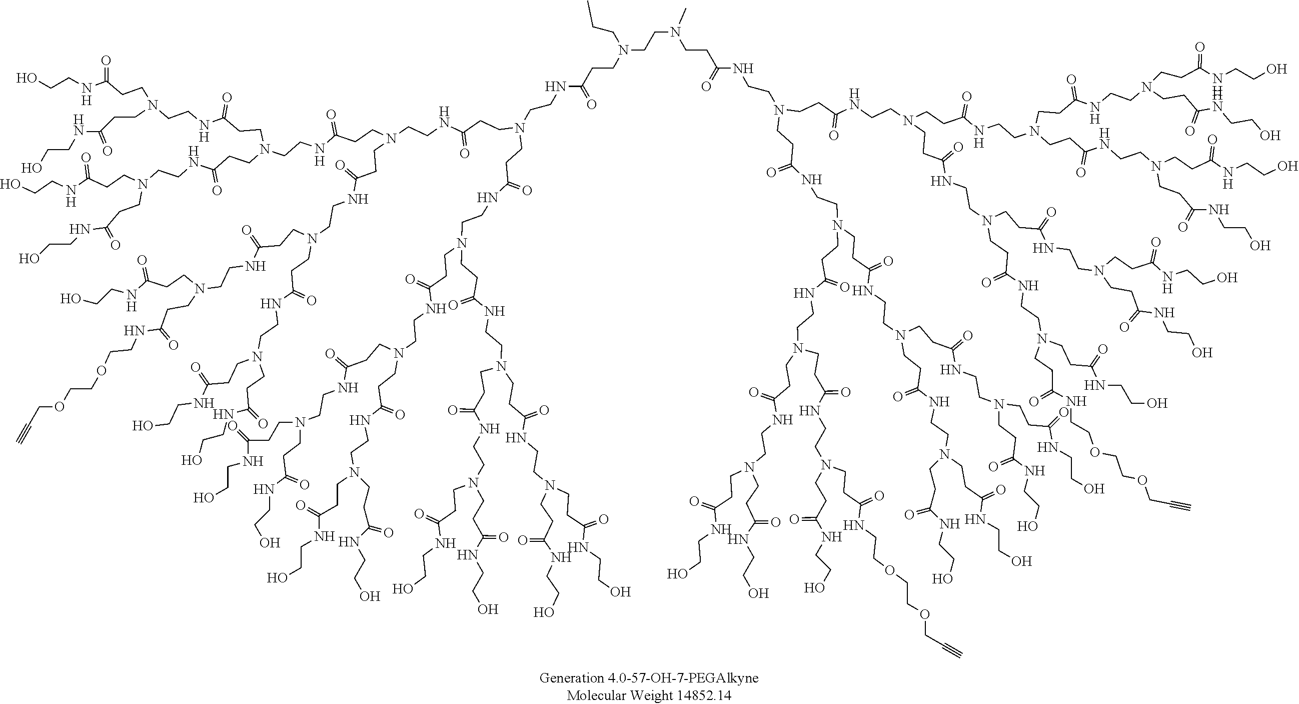

A manufacturing process was developed for a poly(amidoamine) generation 4 hydroxyl terminated dendrimer (PAMAM-G4-OH) for a targeted agent/drug delivery application. An azide modification of the PAMAM G4-OH was used to enable conjugation of the agent using azide-alkyne cycloaddition (click) conjugation technology. One PAMAM G4-OH synthetic route follows a divergent strategy for the preparation of PAMAM dendrimer. The final step of the process is the addition of ethanolamine and a PEG-Alkyne amine resulting in the G4 dendrimer with 56-57 alcohol functional groups and 7-8 PEG-Alkyne functional groups.

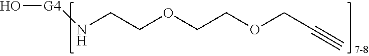

### Analytical Methods

For higher generation PAMAM dendrimers (>G4), development of multiple analytical methods was conducted. A summary of each analytical method is described below.

### UPLC Method (ASHV0010)

A reverse phase UPLC method for resolution each PAMAM dendrimer generation and starting materials was developed using a modification of literature methods (Table 1-1). See Cason, C. A., et al. Journal of Nanomaterials. 2008, 1-7.

### DOI:10.1155/2008/456082.

TABLE 1-1
Summary of UPLC conditions

| Reverse phase column | ACE Excel 3 Super C18, LC column, | |
|---|---|---|
| | 50 × 3 × 3 μm | |
| Column temperature | 40° | C. |
| Mobile phase A | H₂O | (0.05% TFA) |
| Mobile phase B | MeCN | (0.05% TFA) |
| Flowrate | 1.27 | mL/min |
| Observation | 210 | nm |

wavelength

This method was developed using PAMAM core obtained by synthesis and samples of G0-G4 obtained from Sigma Aldrich, Inc. A summary of sample retention times and UPLC reports for the final two generations are provided below in Table 2.

TABLE 2
Retention times and UPLC reports for PAMAM
generations and starting materials

| | Retention time | Relative retention |
|---|---|---|
| Compound | (min) | time (min) |
| PAMAM G3.5 | 2.805 | 1.462 |
| PAMAM G4-OH-Alkyne_{7.8} | 1.918 | 1 |

### SEC-MALLS Method

A size-exclusion chromatography method with multi-angle laser light scattering (MALLS) and differential refractive index (dRI) detectors was developed adopting methods described in the literature. Mullen, D. G., et. al. Macromolecules. 2012, 45, 5316-5320. A summary of this method is provided in the Appendix. Each PAMAM generation peak was able to be resolved using this method. A summary of elution times and measured polydispersities for the final 2 generations is provided below in Table 3.

TABLE 3
SEC elution times for PAMAM dendrimers

| | Elution time | |
|---|---|---|
| Compound | (min) | Ð (M_{w}/Mₙ)^{a} |
| PAMAM G3.5 | 17.9 | 1.036 |
| PAMAM G4-OH-Alkyne_{7.8} | 17.8 | 1.031 |

### Quantitative ¹H NMR

Quantitative ¹H NMR spectroscopy was performed referencing to Sigma Aldrich TraceCERT® grade internal standards. These methods have been used to quantify the potency of PAMAM dendrimer solutions and isolated products. This method has further been used to assay the quality of PEG-Alkyne material (2-[2-(Propargyloxy)ethoxy]ethylamine). 1,3,5-trimethoxybenzene was used as an internal standard in d⁴-methanol as a solvent.

### Protocol for Analysis of Average Number of PEG-Alkyne Arms

Determination of the degree of functionalization of PAMAM G4-OH with PEG-Alkyne was determined via ¹H-NMR spectroscopy using the average value provided by two methods (denoted method A and method B). Each method makes use of the integrations observed with PAMAM-G4-64-OH and the theoretical integrations that would be observed with 100% PEG-Alkyne functionalized PAMAM-G4-64-PEG-Alkyne. The respective integrations for these two materials are provided below in Table 4 and Table 5.

TABLE 4
Proton chemical shift and integrations for PAMAM-G4-64-OH

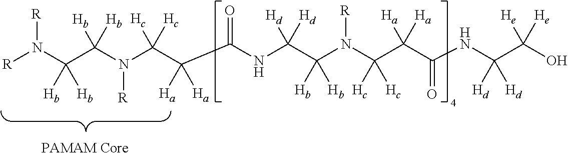

| Proton | Chemical Shift (ppm) | | # of protons |
|---|---|---|---|
| Hₐ | 2.38 | ppm | 248 H |
| H_{b} | 2.58 | ppm | 124 H |
| H_{c} | 2.79 | ppm | 248 H |
| H_{d} | 3.26-3.33 | ppm | 248 H |
| Hₑ | 3.61 | ppm | 128 H |

TABLE 5
Proton chemical shift and integrations for PAMAM-G4-64-PEG-Alkyne (theoretical)

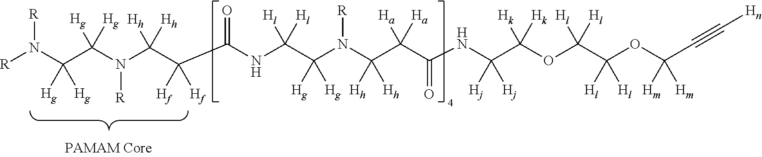

| Proton | Chemical Shift (ppm) | # of protons |
|---|---|---|
| H_{f} | 2.38 ppm | 258 H |
| H_{g} | 2.58 ppm | 124 H |
| Hₕ | 2.79 ppm | 248 H |
| Hᵢ | 3.27 ppm | 120 H |
| Hⱼ | 3.37 ppm | 128 H |
| Hₖ | 3.55 ppm | 128 H |
| Hₗ | 3.65 ppm (multiplet) | 256 H |
| Hₘ | 4.2 ppm | |

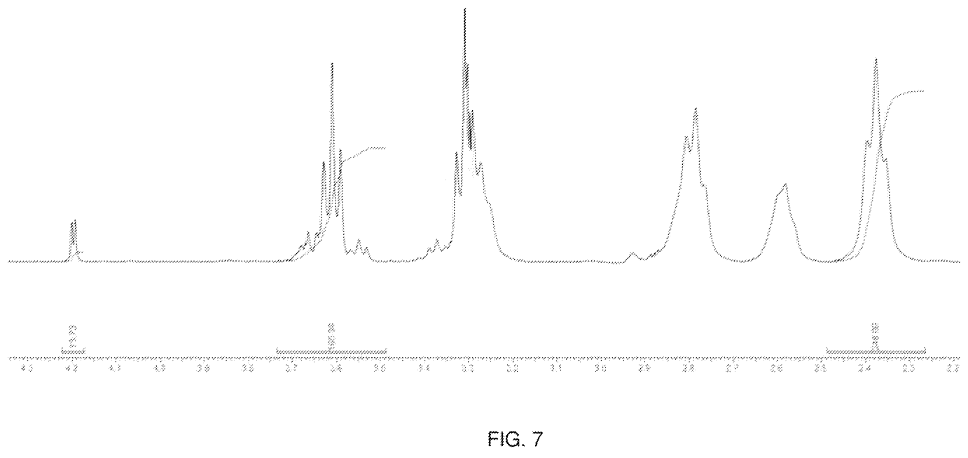

Method A. Method A was based on a calculation of the percentage of the actual integration of protons Hₘ when compared to the integration of Hₘ for 100% alkyne functionalized PAMAM-G4. This was determined by setting the integration for the signal corresponding to protons Hₐ and H_{f} to its theoretical value of 248 (this signal is expected to be 248 for both PAMAM-G4-64-OH and PAMAM-G4-64-PEG-Alkyne). An example calculation of this method is referenced in FIG. 7.

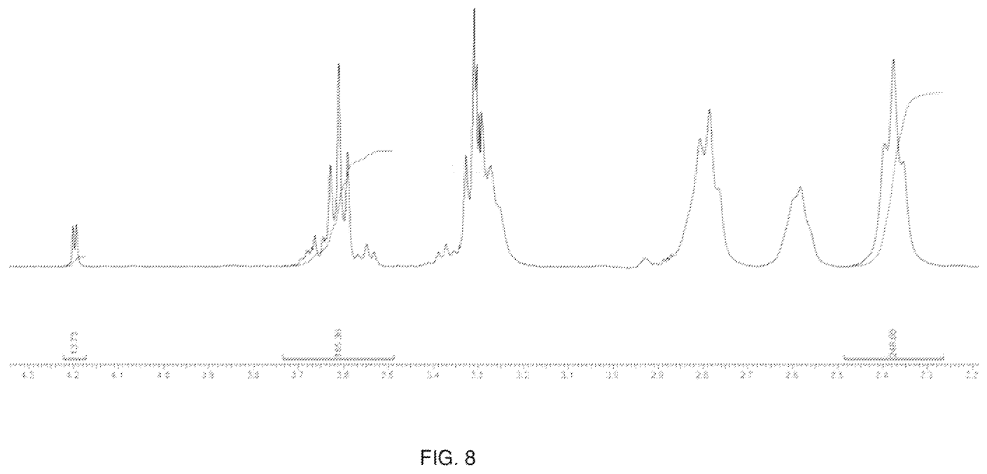

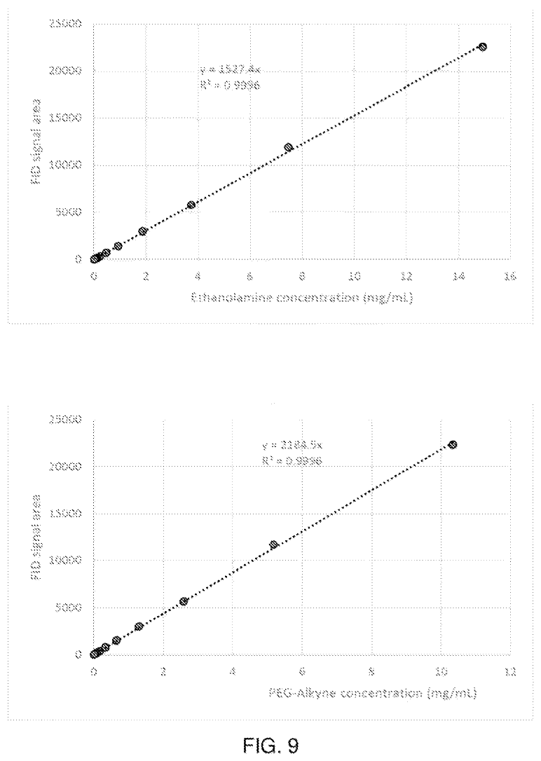

Method B. Method B was based on a calculation of the ratio of protons Hₑ from the ethanolamine terminal arms relative to protons Hₘ from PEG-Alkyne terminal arms. Due to the overlap of protons Hₑ of the ethanolamine arms with Hₖ and Hₗ protons of PEG-Alkyne terminal arms, the integration of these PEG-Alkyne protons must be subtracted out (determination of integration using integration of Hₘ protons). This ratio can then be used to calculate the average number of alkyne functionalized arms. An example calculation is referenced in FIG. 8.

### Manufacturing Summary

### PAMAM Core Synthesis

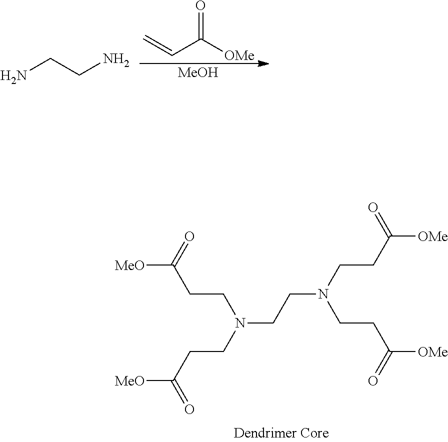

| | MW | Density | Amount | Amount | | | Concentration |
|---|---|---|---|---|---|---|---|
| Reagent | (g/mol) | (g/mL) | (g) | (mL) | mmol | Eq/vol | (Wt %) |
| Methyl Acrylate | | | 602.4699 | | | | |
| solution | | | | | | | |
| Methyl Acrylate | 86.09 | 0.95 | 409.6795 | 431.24 | 4758.74 | 11 | 68 |
| MeOH | | 0.792 | 192.7904 | 243.42 | | | 32 |
| Ethylene Diamine | | | 173.3333 | | | | |
| solution | | | | | | | |
| Ethylene Diamine | 60.1 | 0.9 | 26.0000 | 28.89 | 432.61 | 1 | 15 |
| MeOH | | 0.792 | 147.3333 | 186.03 | | | 85 |

The PAMAM dendrimer core synthesis was carried out as per described previously in the literature, as shown in the scheme above. A methanolic solution of ethylene diamine was added to a methanolic solution of excess methyl acrylate dropwise at 0° C. After 1 hour the solution was warmed to room temperature and stirred for 24 hours. The solution was then concentrated under reduced pressure at 20° C. The resulting oil was then diluted with MeOH (1 L) and concentrated again under reduced pressure. This was repeated once more to provide the PAMAM core as a colorless oil in quantitative yields (17.5 grams). ¹H-NMR and GC analysis showed the PAMAM core to be produced in high purity. A summary of the metrics of the process are shown below in Table 6.

TABLE 6
Summary of metrics for experiment AA08-055

| Crude product (g) | 304.5 | g |
|---|---|---|
| Product potency (% w/w) | 55.12% | |
| Assay yield (g) | 167.8 | grams |

| | |
|---|---|
| Assay Yield (%) | 97% |

### Synthesis of Full Generation Amine Terminated PAMAM Dendrimers

Full generations of PAMAM dendrimers were prepared following literature conditions. Under these conditions a solution of ethylenediamine (86% w/w in methanol, 25 equivalents per ester on PAMAM starting material) was cooled to 0° C. under a nitrogen atmosphere. A solution of half generation PAMAM dendrimer or PAMAM core (10% w/w in methanol) was then added over the course of 2 hours. After addition, the reaction mixture was stirred for 1 hour at 0° C. After this time, the reaction mixture was warmed to 20° C. and allowed to stir for 5 days. The crude product was then concentrated by vacuum distillation maintaining the temperature of the crude product <25° C. Residual ethylenediamine was then removed by the following 1 of 2 methods: Method 1: 1. Dissolving the crude product residue in methanol (2.5 volumes). 2. Dilution with toluene (24 volumes). 3. Concentration by vacuum distillation. This process was repeated 4 times or until residual ethylenediamine was no longer detected in the crude product or distillate by GC and ¹H-NMR analysis. Method 2: 1. Dissolving the crude product residue in pentanol to generate a 10 wt % solution of dendrimer. 2. Concentration by distillation using wiped film evaporator. This process was repeated >4× or until residual ethylenediamine was no longer detected in the crude product or distillate by GC and ¹H-NMR analysis. Method 1 was used for PAMAM G0 and G1, Method 2 was used for PAMAM G2 and G3. A summary of results during the synthesis of each full generation amine-terminated PAMAM dendrimer is provided below in Table 7.

TABLE 7
Summary of results for full generation
amine-terminated PAMAM dendrimers

| | PAMAM | Yield | Yield | Polydispersity |
|---|---|---|---|---|
| Experiment | Generation | (g) | (%) | (M_{w}/Mₙ) |
| AA08-063 | PAMAM G0 | 213.80 | 99.7 | 1.015 |
| HA04-029 | PAMAM G1 | 563 | 95 | 1.012 |
| HA04-036 | PAMAM G2.0 | 652 | —* | 1.056 |
| AA08-086 | PAMAM G3.0 | 1322 | 95 | 1.14 |

### Synthesis of Half-Generation Ester Terminated PAMAM Dendrimers

Half generations of PAMAM dendrimers were prepared following literature conditions. Under these conditions a solution of methyl acrylate (55% w/w in methanol, 3 equivalents per terminal amine on dendrimer starting material) was cooled to 0° C. under a nitrogen atmosphere. A solution of full generation PAMAM dendrimer (10% w/w in methanol) was then added over the course of 2 hours. After addition, the reaction mixture was stirred for 1 hour at 0° C. After this time, the reactions mixture was warmed to 20° C. and allowed to stir for 3-4 days until ¹H-NMR analysis of the crude product indicates complete conversion of the starting material. The crude product was then concentrated by vacuum distillation maintaining the temperature of the crude product <25° C. Residual methyl acrylate is then removed by 3× azeotropic removal with methanol by vacuum distillation (removal of methyl acrylate indicated by ¹H-NMR, GC, and/or UPLC analysis). A summary of results during the synthesis of each half-generation PAMAM dendrimer is shown below in Table 8.

TABLE 8
Summary of results for half generation
ester terminated PAMAM dendrimers

| | PAMAM | | | Polydispersity |
|---|---|---|---|---|
| Experiment | Generation | Yield (g) | Yield (%) | (M_{w}/Mₙ) |
| HA04-024 | PAMAM G0.5 | 499.4 | Quantitative | 1.020 |
| HA04-33 | PAMAM G1.5 | 1025 | 91 | 1.020 |
| HA04-64 | PAMAM G2.5 | 1215 | Quantitative | 1.034 |
| AA10-062 | PAMAM G3.5 | 2360 | 99 | 1.1 |

### PAMAM G4-OH-Alkyne₇₋₈ Production

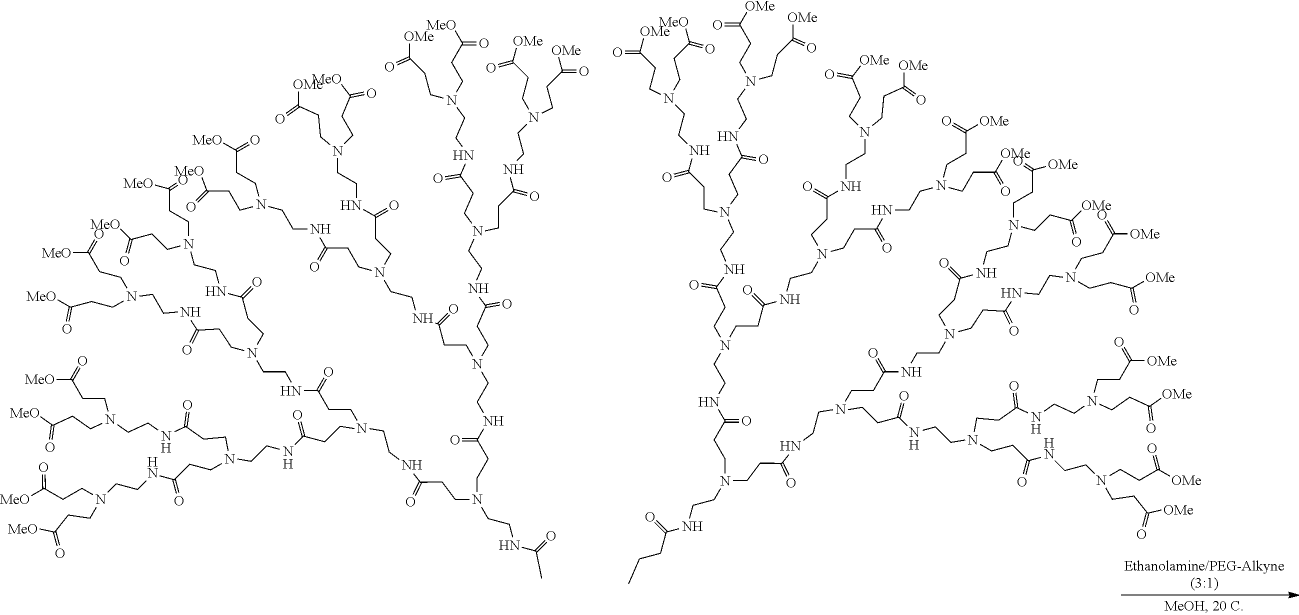

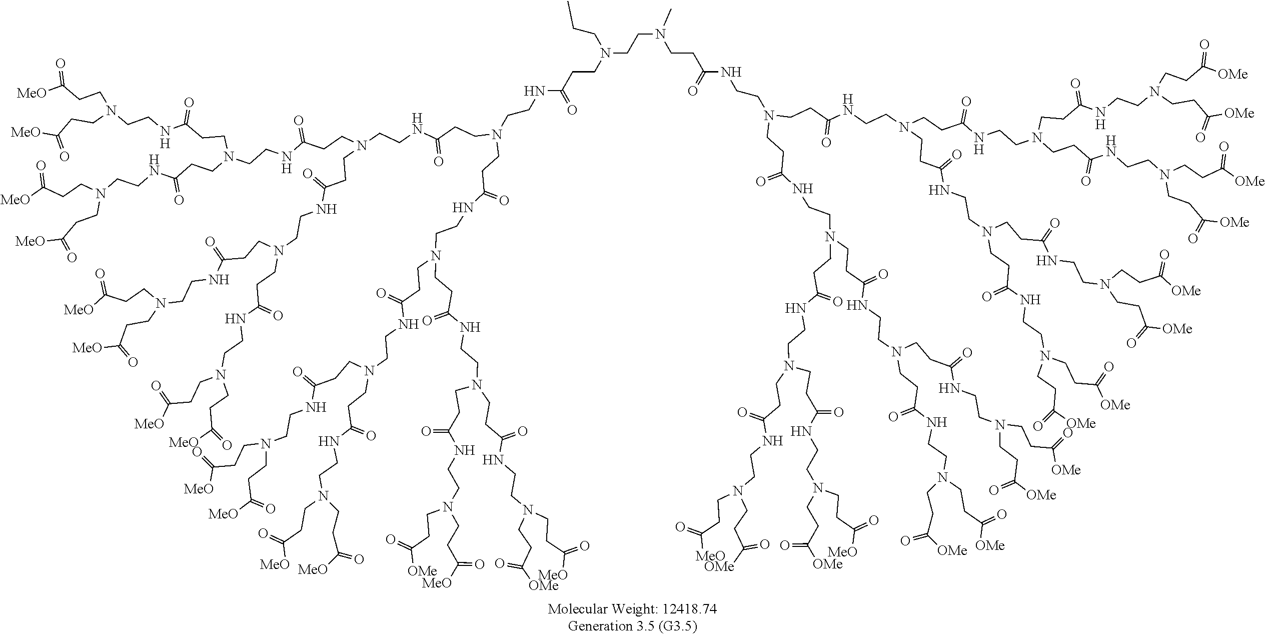

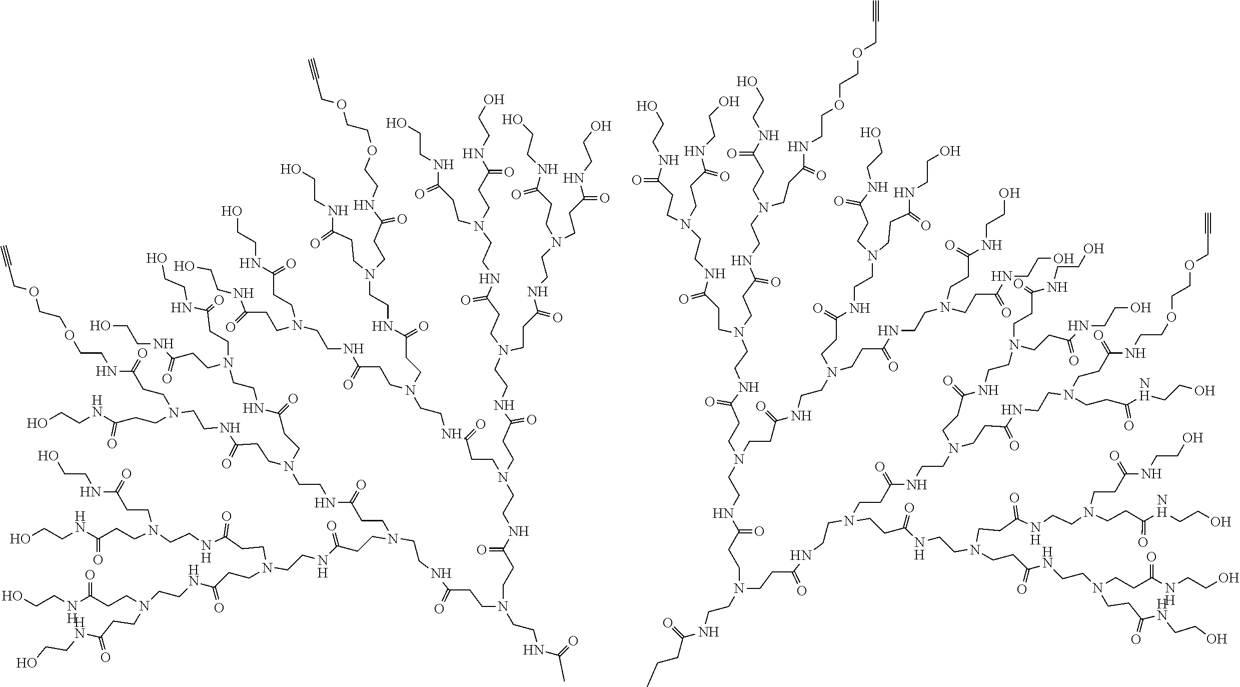

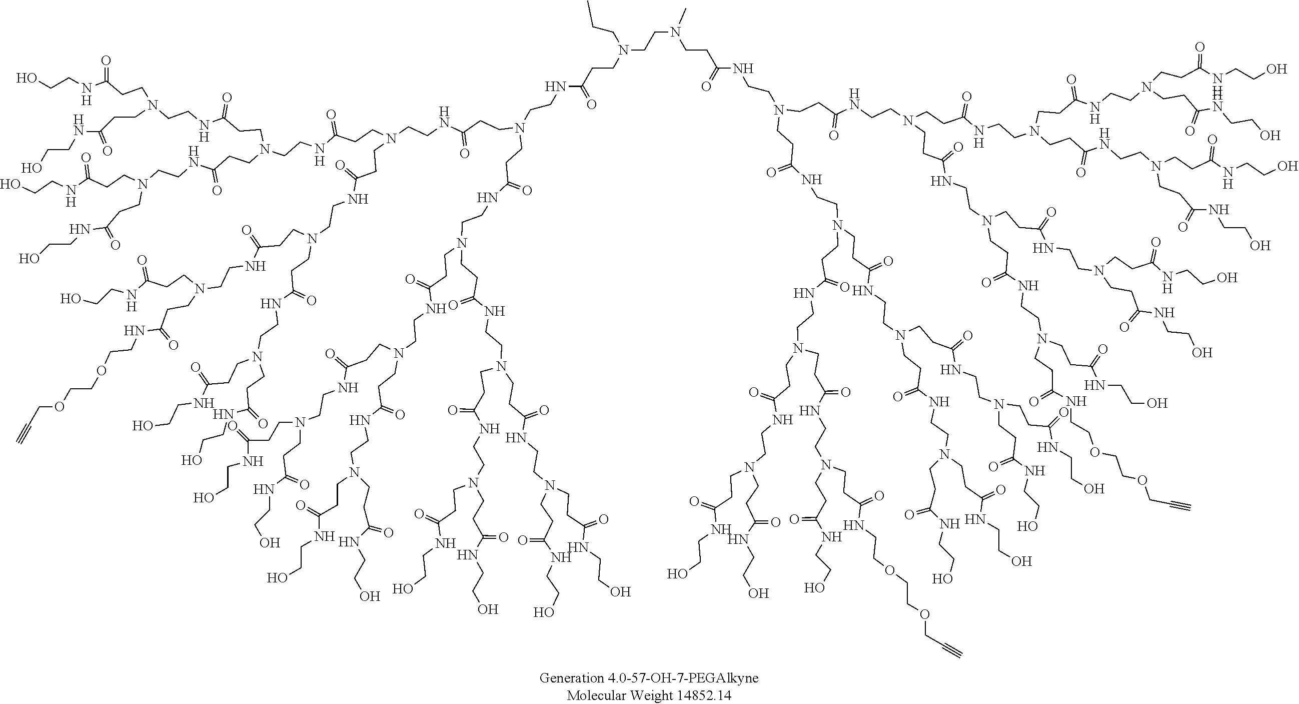

A jacketed vessel is charged with a 3:1 molar ratio of ethanolamine and PEG-Alkyne followed by methanol. The vessel was purged with N₂(g) and cooled to 0° C. A 10 wt % of PAMAM G3.5 in methanol was then added over 2 hours to the reactor. The solution was then stirred 4 hours at 0° C. before warming to 20° C. The solution was then allowed to stir at 20° C. for 6 days. After this time, the solution was diluted to half the concentration with water and subjected to purification by ultrafiltration providing the product as an aqueous solution. For production batch AA10-065, the aqueous solution was then subjected to purification using a larger pore size membrane to remove dimeric impurities. The solvent was then swapped over to methanol by ultrafiltration using 10 diavolumes of methanol. A summary of results for each scale-up is provided below in Table 9.

TABLE 9
Summary of results for experiment AA10-007

| | PAMAM G4-OH-Alkyne₇₋₈ lot | | | |
|---|---|---|---|---|
| Lot number | AA10-064 | | AA10-065 | |
| PAMAM G3.5 source | Dendritech | | Snapdragon (Lot AA10-062) | |
| Methanolic solution | 3.25 | | 8.21 | kg |
| mass (kg) | | | | |
| Potency (% w/w) | 16.6% | w/w | 19.5% | w/w |
| Yield | 539.5 | g | 1.6 | kg |

| | | |
|---|---|---|
| Yield (%) | 93% | 71.8% |
| Polydispersity (M_{w}/Mₙ) | 1.030 | 1.045 |
| Average # of | 8.1 | 7.8 |
| PEG-alkyne arms | | |
| —OH/—NH ratio | 0.39 | ND |
| Residual ethanolamine | 0.2% | 0.4% |
| (dry basis, GC) | | |
| Residual PEG-Alkyne | 0.02% | 0.01% |
| (dry basis, GC) | | |

### Conclusion

A process for producing PAMAM G4-OH-Alkyne₇-8 in >1 kg quantities was successfully scaled and developed. This process was carried out on 500-gram and 2-kg scales producing the product in reproducible quality and yield. This demonstrates that the product quality can be increased and that dimeric impurities can be removed during downstream purification by ultrafiltration. This allowed for the removal of a substantial amount of dimeric impurity that was produced upstream in route to Lot AA10-065.

### Experimental

### PAMAM Core Synthesis

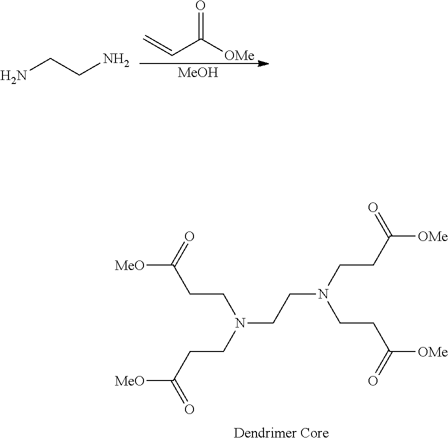

| | MW | Density | Amount | Amount | | | Concentration |
|---|---|---|---|---|---|---|---|
| Reagent | (g/mol) | (g/mL) | (g) | (mL) | mmol | Eq/vol | (Wt %) |
| Methyl Acrylate | | | 602.4699 | | | | |
| solution | | | | | | | |
| Methyl Acrylate | 86.09 | 0.95 | 409.6795 | 431.24 | 4758.74 | 11 | 68 |
| MeOH | | 0.792 | 192.7904 | 243.42 | | | 32 |
| Ethylene Diamine | | | 173.3333 | | | | |
| solution | | | | | | | |
| Ethylene Diamine | 60.1 | 0.9 | 26.0000 | 28.89 | 432.61 | 1 | 15 |
| MeOH | | 0.792 | 147.3333 | 186.03 | | | 85 |

PAMAM dendrimer core synthesis was carried out as per described previously in the literature. A methanolic solution of ethylene diamine was added to a methanolic solution of excess methyl acrylate dropwise at 0° C. After 1 hour the solution was warmed to room temperature and stirred for 24 hours. The solution was then concentrated under reduced pressure at 20° C. The resulting oil was then diluted with MeOH (1 L) and concentrated again under reduced pressure. This was repeated once more to provide the PAMAM core as a colorless oil in quantitative yields (17.5 grams). ¹H-NMR and GC analysis showed the PAMAM core to be produced in high purity. A summary of metrics of the process are shown in Table 10.

TABLE 10
Summary of metrics for experiment AA08-055

| Crude product (g) | 304.5 | g |
|---|---|---|
| Product potency (% w/w) | 55.12% | |
| Assay yield (g) | 167.8 | grams |

| | |
|---|---|
| Assay Yield (%) | 97% |

### PAMAM G0.0 Synthesis

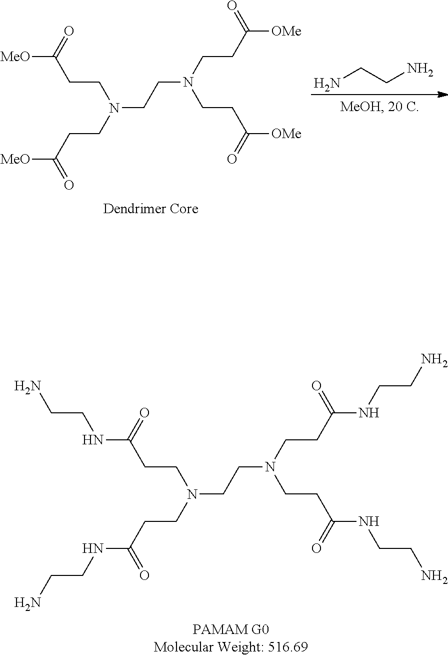

| | MW | Density | Amount | Amount | | |
|---|---|---|---|---|---|---|
| Reagent | (g/mol) | (g/mL) | (g) | (mL) | mmol | Eq/vol |
| Ethylene diamine | | | 1501.9406 | | | |
| solution | | | | | | |
| Ethylene diamine | 60.1 | 0.9 | 1296.5647 | 1440.63 | 21573.46 | 52 |
| MeOH | | 0.792 | 205.3758 | 259.31 | | 0.2 |
| Core solution | | | 1762.5712 | | | |
| Core | 404.46 | | 167.8000 | | 414.87 | 1 |
| MeOH | | 0.792 | 1594.7712 | 2013.60 | | 12 |

A 5 L jacketed vessel was charged with a methanol solution of ethylenediamine under a nitrogen atmosphere. The reaction solution was cooled to 0° C. and a solution of PAMAM core was added over the course of 2 hours. After addition the reaction mixture was allowed to stir for 1 hour at this temperature. After this time the reaction mixture was warmed to room temperature and stirred for 5 days. The methanol and a substantial portion of ethylenediamine were then removed by vacuum distillation while maintaining the reactor contents at below 25° C. Residual ethylenediamine was then removed by 5× azeotropic distillation with 21 volumes of 1:9 methanol/toluene. Complete removal was confirmed by ¹H-NMR analysis. After complete removal of ethylenediamine, residual toluene was removed by 3× azeotropic distillation away from 3 volumes of methanol providing 600 grams of PAMAM G0.0 as a 35.61% w/w solution in methanol.

### PAMAM G0.5 Synthesis

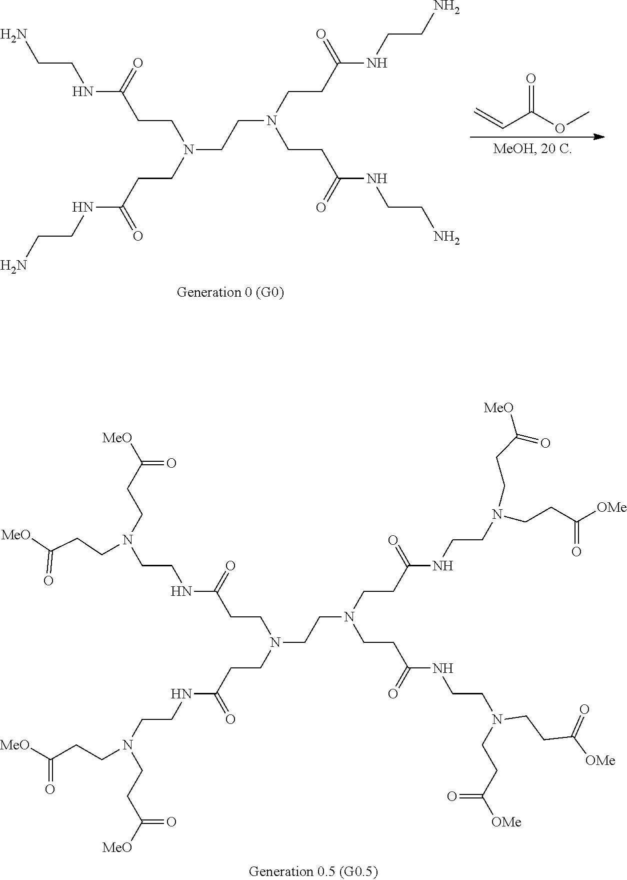

TABLE 11

| | MW | Density | Amount | Amount | | Eq/ | Potency | Concentration |
|---|---|---|---|---|---|---|---|---|
| Reagent | (g/mol) | (g/mL) | (g) | (mL) | mmol | vol | (wt. %) | (wt. %) |
| Methyl Acrylate | | | 2316.2707 | | | | | |
| solution | | | | | | | | |
| Methyl Acrylate | 86.09 | 0.95 | 534.3509 | 562.47 | 6206.89 | 15 | | |
| MeOH | | 0.792 | 1781.9197 | 2249.90 | | 4 | | |
| PAMAM Core | | | 2138.0244 | | | | | |
| solution | | | | | | | | |
| PAMAM Core | 516.69 | 0.9 | 600.4000 | 667.11 | 413.79 | 1 | 35.61 | 10 |
| MeOH | | 0.792 | 1537.6244 | 1941.44 | | | | 90 |

A 10 L reactor was charge with methanolic solution of methylacrylate and a methanolic solution of PAMAM core was added at 0° C. over a period of 2 hours. The solution was stirred for one more hour at 0° C. and then warmed to 20° C. and stirred for 3 days. Reaction completion was determined by Kaiser test for residual primary amines and further validated by ¹H-NMR spectroscopy. Volatiles were then removed by distillation under reduced pressure with the reactor jacket controlled at 20° C. Residual methyl acrylate was then removed by 2× azeotropic removal with methanol (3 vol.) after which the GC and NMR of crude material showed complete removal of methyl acrylate. The product was obtained as 2359.9 g of a viscous oil. Quantitative ¹H-NMR analysis showed the oil to be 21.16 wt. % PAMAM G0.5 providing as assay yield of 499.35 g (100.11% yield). A summary of metrics of the process are shown in Table 12.

TABLE 12
Process metrics for experiment HA04-24

| | | |
|---|---|---|
| Reaction time | 72 | h |
| Crude yield | 2359.9 | g |
| Product potency | 21.16% | w/w |
| Assay yield (g) | 499.35 | g |

| | |
|---|---|
| Assay yield (%) | Quantitative |
| Polydispersity (Ð (M_{w}/Mₙ)) | 1.025 |

### PAMAM G1.0 Synthesis

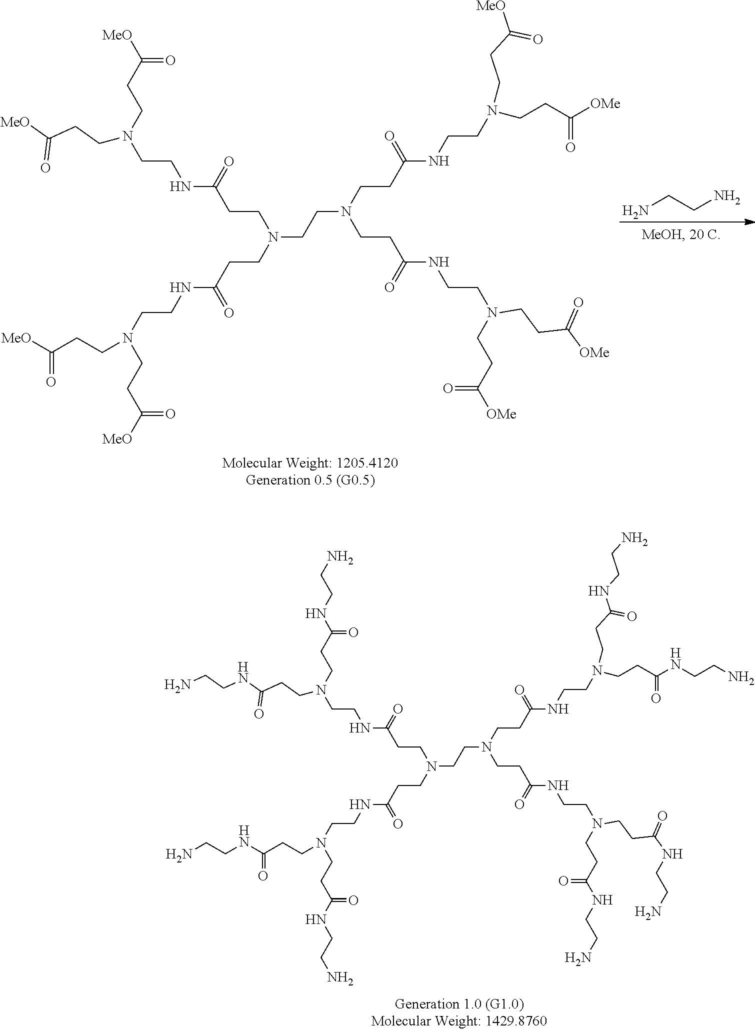

TABLE 13

| | MW | Potency | Density | Amount | Amount | | | Concentration |
|---|---|---|---|---|---|---|---|---|
| Reagent | (g/mol) | (% w/w) | (g/mL) | (g) | (mL) | mmol | Eq/vol | (Wt %) |
| Ethylene Diamine solution | | | | 6086.83 | 6930.93 | | | |
| Ethylene Diamine | 60.1 | 100 | 0.9 | 4979.46 | 5532.74 | 82852.97 | 200 | — |
| MeOH | | 100 | 0.792 | 1107.37 | 1398.19 | | 2.8 | — |
| PAMAM G0.5 solution | | | | 4993.55 | | | | |
| PAMAM G0.5 | 1205.4 | 21.16 | | 2359.9 | | 414.26 | 1 | 10 |
| MeOH | | 100 | 0.792 | 2633.65 | 3325.31 | | | 90 |

A 20 L jacketed reactor was purged with N₂(g) and charged with ethylenediamine solution. PAMAM G0.5 solution was added dropwise at 0° C. over a period of ˜2 hours. After stirring for ˜4 more hours at 0° C. the solution was warmed to 20° C. and stirred 5 days after which ¹H-NMR analysis showed complete consumption of the methyl ester of PAMAM G0.5. Volatiles were removed under reduced pressure with reactor jacket controlled at 20° C. Residual ethylenediamine was then removed by 7× azeotropic distillation with 1:9 MeOH/toluene (23 vol.). Removal of ethylenediamine was monitored by NMR of crude reaction mixture. Residual toluene was then removed by 3× concentration from methanol under reduced pressure. The product was obtained as an oil. Quantitative H-NMR analysis showed the oil to be 23.5 wt. % PAMAM G1.0 providing as assay yield of 563.03 g (95% yield). A summary of metrics of the process are shown in Table 14.

TABLE 14
Process metrics for experiment HA04-29

| | | |
|---|---|---|
| Reaction time | 120 | h |
| Crude yield | 2395. | g |
| Product potency | 23.5% | w/w |
| Assay yield (g) | 563 | g |

| | |
|---|---|
| Assay yield (%) | 95% |
| Polydispersity (Ð (M_{w}/Mₙ)) | 1.012 |

### PAMAM G1.5 Synthesis

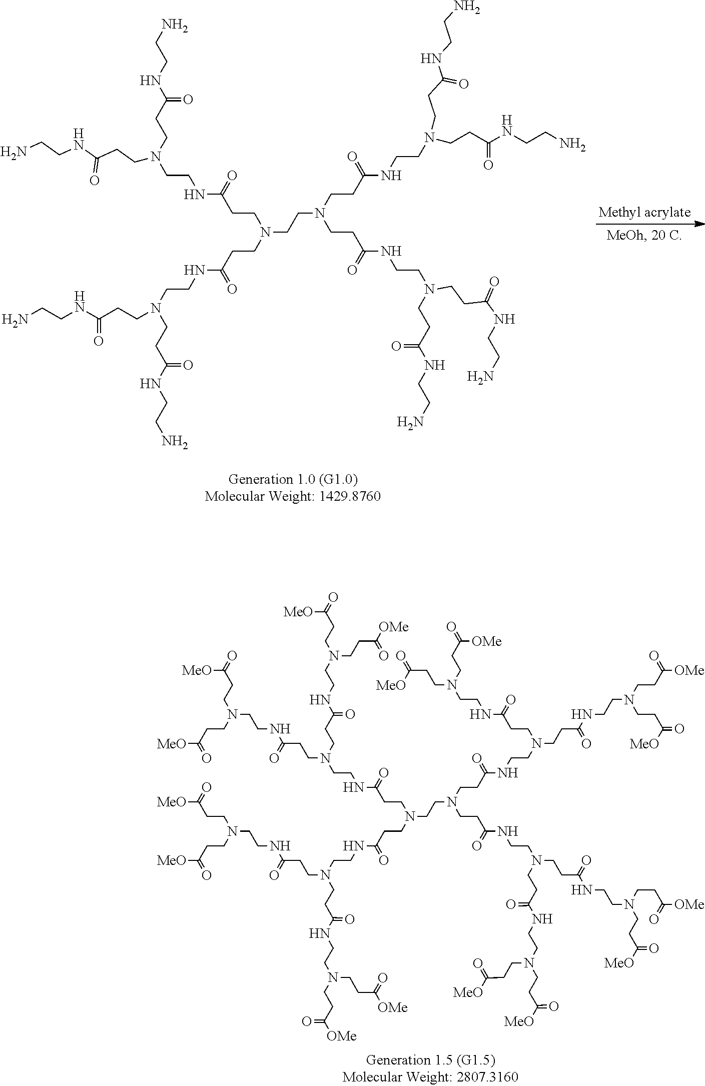 [SMILES: NCCNC(=O)CCN(CCNC(=O)CCN(CCC(=O)NCCN(CCC(=O)NCCN)CCC(=O)NCCN)CCN(CCC(=O)NCCN(CCC(=O)NCCN)CCC(=O)NCCN)CCC(=O)NCCN(CCC(=O)NCCN)CCC(=O)NCCN)CCC(=O)NCCN ; COC(=O)CCN(CCNC(=O)CCN(CCNC(=O)CCN(CCC(=O)NCCN(CCC(=O)NCCN(CCC(=O)OC)CCC(=O)OC)CCC(=O)NCCN(CCC(=O)OC)CCC(=O)OC)CCN(CCC(=O)NCCN(CCC(=O)NCCN(CCC(=O)OC)CCC(=O)OC)CCC(=O)NCCN(CCC(=O)OC)CCC(=O)OC)CCC(=O)NCCN(CCC(=O)NCCN(CCC(=O)OC)CCC(=O)OC)CCC(=O)NCCN(CCC(=O)OC)CCC(=O)OC)CCC(=O)NCCN(CCC(=O)OC)CCC(=O)OC)CCC(=O)OC]

TABLE 15

| | MW | Potency | Density | Amount | Amount | | | Concentration |
|---|---|---|---|---|---|---|---|---|
| Reagent | (g/mol) | (wt %) | (g/mL) | (g) | (mL) | mmol | Eq/vol | (Wt %) |
| Methyl Acrylate | | | | 1523.60 | | | | |
| solution | | | | | | | | |
| Methyl Acrylate | 86.09 | | 0.95 | 830.89 | 874.63 | 9561.46 | 24 | |
| MeOH | | | 0.792 | 692.70 | 874.63 | | 1.5 | |
| Ethylene Diamine | | | | 5750.16 | | | | |
| solution | | | | | | | | |
| PAMAM G1.0 | 1429.876 | 24 | 0.9 | 2395.90 | 2662.11 | 402.14 | 1 | 10 |
| MeOH | | | 0.792 | 3354.26 | 4235.18 | | | 90 |

A 10 L reactor was charge with methanolic solution of methylacrylate and a methanolic solution of PAMAM G1.0 was added at 0° C. over a period of 2 hours. The solution for stirred for two more hour at 0° C. and then warmed to 20° C. and stirred for 3 days. Reaction completion was validated by ¹H-NMR spectroscopy. Volatiles were then removed by distillation under reduced pressure with the reactor jacket controlled at 20° C. Residual methyl acrylate was then removed by 3× azeotropic removal with methanol (3 vol.) after which the GC and NMR of crude material showed complete removal of methyl acrylate. The product was obtained as 2359.9 g of a viscous oil. Quantitative ¹H-NMR analysis showed the oil to be 32.89 wt. % PAMAM G1.5 providing as assay yield of 1025 g (91% yield). A summary of metrics of the process are shown in below in Table 16.

TABLE 16
Process metrics for experiment HA04-33

| Reaction time | 72 | h |
|---|---|---|
| Crude yield | 3116.1 | |
| Product potency | 32.89% | w/w |
| Assay yield (g) | 1024.9 | g |

| | |
|---|---|
| Assay yield (%) | 91% |
| Polydispersity (Ð (M_{w}/Mₙ)) | 1.020 |

### PAMAM G2.0 Synthesis

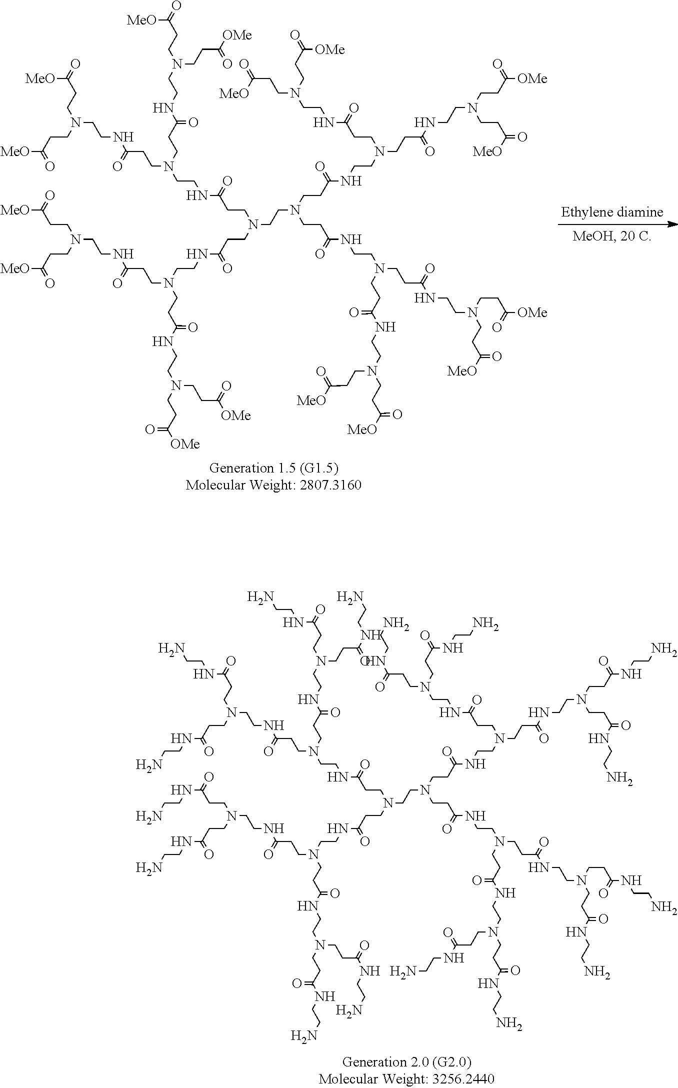

TABLE 17

| | MW | Potency | Density | Amount | Amount | | | Concentration |
|---|---|---|---|---|---|---|---|---|
| Reagent | (g/mol) | (% w/w) | (g/mL) | (g) | (mL) | mmol | Eq/vol | (Wt. %) |
| Ethylene Diamine | | | | 10708.31 | 12190.83 | | | |
| solution | | | | | | | | |
| Ethylene Diamine | 60.1 | 100 | 0.9 | 8776.44 | 9751.60 | 146030.63 | 400 | — |
| MeOH | | 100 | 0.792 | 1931.87 | 2439.23 | | 2.38 | — |
| PAMAM G1.5 | | | | 10248.85 | | | | |
| solution | | | | | | | | |
| PAMAM G1.5 | 2807.316 | 32.89 | | 3116.10 | | 365.08 | 1 | 10 |
| MeOH | | 100 | 0.792 | 7132.75 | 9006.00 | | | 90 |

Jacketed reactor was purged with N₂(g) and charged with ethylenediamine solution. PAMAM G1.5 solution was added dropwise at 0° C. over a period of ˜2 hours. After stirring for ˜4 more hours at 0° C. the solution was warmed to 20° C. and stirred 5 days after which ¹H-NMR analysis showed complete consumption of the methyl ester of PAMAM G1.5. Volatiles were removed under reduced pressure, Residual ethylenediamine was then removed by distillation. Removal of ethylenediamine was monitored by NMR of crude reaction mixture. The product was obtained as an oil. Quantitative H-NMR analysis showed the oil to be 9.5 wt. % PAMAM G2.0. A summary of metrics of the process are shown in Table 18.

TABLE 18
Process metrics for experiment HA04-36

| Reaction time | 120 | h |
|---|---|---|
| Crude yield | 6832.5 | |
| Product potency | 9.55% | w/w |
| Assay yield (g) | 652.5 | g |

| | |
|---|---|
| Assay yield (%) | — |
| Polydispersity (Ð (M_{w}/Mₙ)) | 1.056 |

### PAMAM G2.5 Synthesis

TABLE 19

| | MW | Potency | Density | Amount | Amount | | | Concentration |
|---|---|---|---|---|---|---|---|---|
| Reagent | (g/mol) | (Wt. %) | (g/mL) | (g) | (mL) | mmol | Eq/vol | (Wt. %) |
| Methyl Acrylate | | | | 1518.3962 | | | | |
| solution | | | | | | | | |
| Methyl Acrylate | 86.09 | | 0.95 | 828.0576 | 871.64 | 9618.51 | 48 | |
| MeOH | 32.04 | | 0.792 | 690.3386 | 871.64 | | 1.34 | |
| PAMAM G2.0 | | | | 6832.5000 | | | | |
| solution | | | | | | | | |
| PAMAM G2.0 | 3256.24 | 9.55 | 0.9 | 6832.5000 | 7591.67 | 200.39 | 1 | 9.55 |
| MeOH | | | 0.792 | 0.0000 | 0.00 | | | |

A 20 L jacketed reactor was charge with methanolic solution of methylacrylate and a methanolic solution of PAMAM G2.0 was added at 0° C. over a period of 2 hours. The solution was stirred for two more hour at 0° C. and then warmed to 20° C. and stirred for 4 days. Reaction completion was validated by ¹H-NMR spectroscopy. Volatiles were then removed by distillation under reduced pressure with the reactor jacket controlled at 20° C. Residual methyl acrylate was then removed by 3× azeotropic removal with methanol (3 vol.) after which the GC and NMR of crude material showed complete removal of methyl acrylate. The product was obtained as 7610 g of a viscous oil. Quantitative ¹H-NMR analysis showed the oil to be 15.97 wt. % PAMAM G2.5 providing as assay yield of 1215.3 g (100% yield). A summary of metrics of the process are shown in Table 20.

TABLE 20
Process metrics for experiment HA04-64

| | | |
|---|---|---|
| Reaction time | 96 | h |
| Crude yield | 7610 | g |
| Product potency | 16% | w/w |
| Assay yield (g) | 1215.3 | g |

| | |
|---|---|
| Assay yield (%) | Quantitative |
| Polydispersity (Ð (M_{w}/Mₙ)) | 1.024 |

### PAMAM G3.0 Synthesis

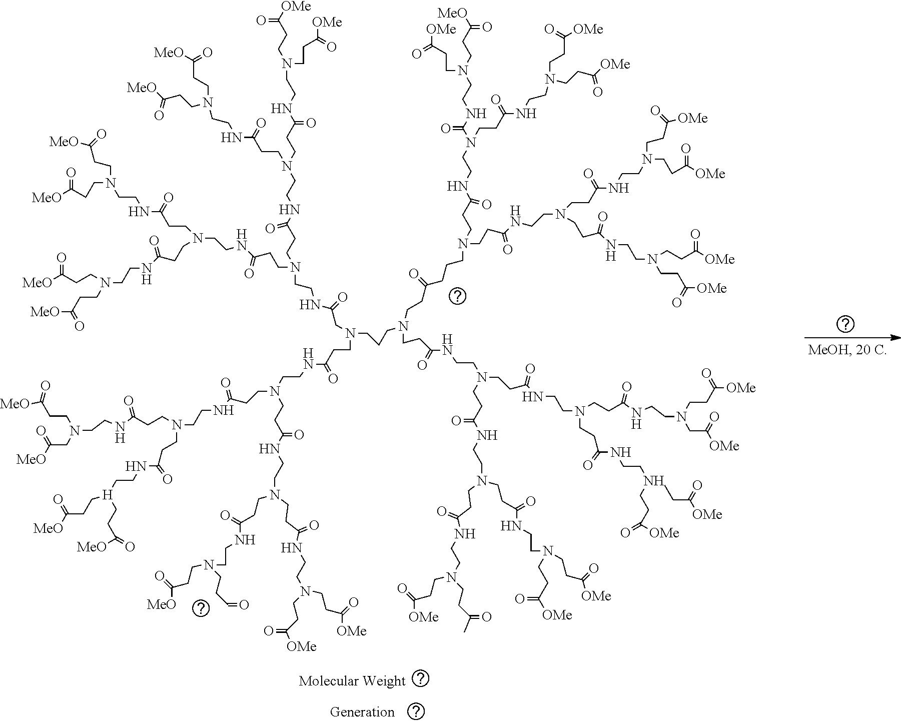

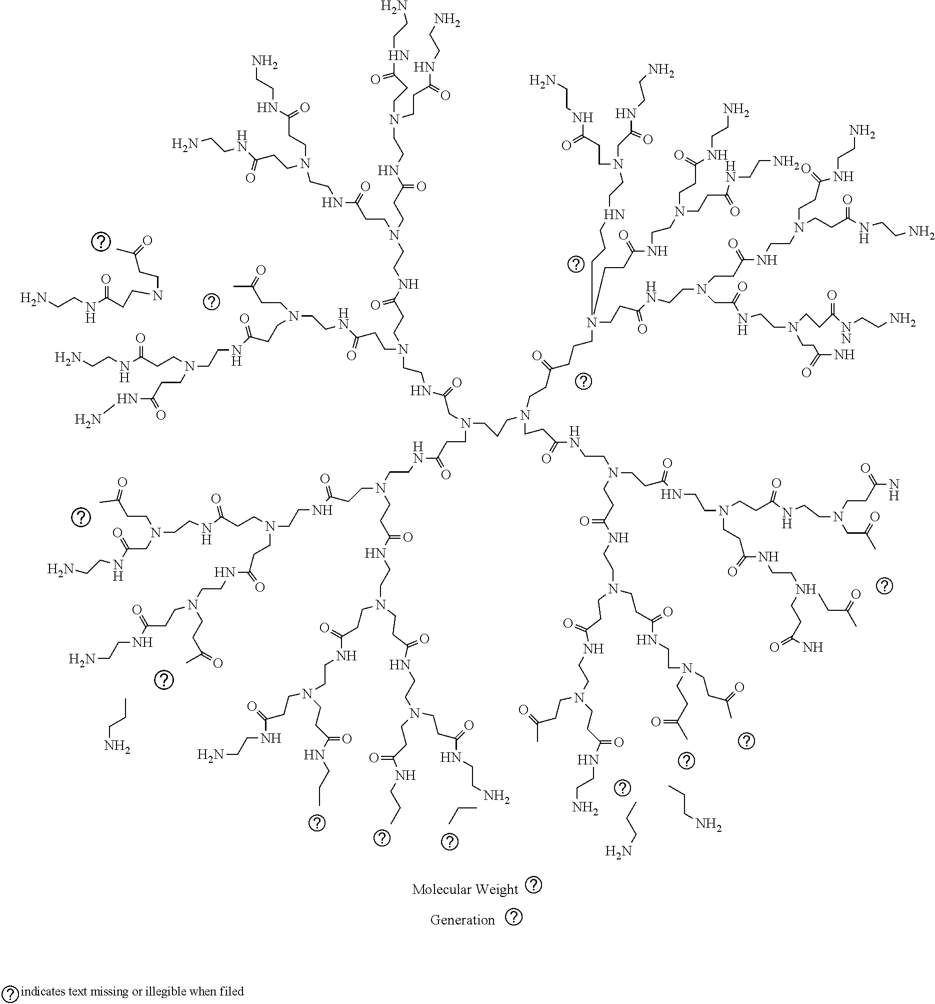

TABLE 21

| | MW | Potency | Density | Amount | Amount | | |
|---|---|---|---|---|---|---|---|
| Reagent | (g/mol) | (% w/w) | (g/mL) | (g) | (mL) | mmol | Eq/vol |
| Ethylene Diamine | | | | 10708.31 | 12190.83 | | |
| solution | | | | | | | |
| Ethylene Diamine | 60.1 | 100 | 0.9 | 9739 | 10821 | 162.044 | 800 |
| MeOH | | 100 | 0.792 | 2143 | 2705 | | 0.36 |
| PAMAM G2.5 solution | | | | 12176 | | | |
| PAMAM G2.5 | 6011.2 | 16 | | 7610 | | 202.56 | 1 |
| MeOH | | 100 | 0.792 | 4566 | 5765 | | 11.37 |

Jacketed reactor was purged with N₂(g) and charged with ethylenediamine solution. PAMAM G2.5 solution (HA04-064) was added via diaphragm pump at 0° C. over a period of ˜2 hours. After stirring for ˜4 more hours at 0° C. the solution was warmed to 20° C. and stirred 5 days after which ¹H-NMR analysis showed complete consumption of the methyl ester of PAMAM G2.5. Volatiles were removed under reduced pressure. Residual ethylenediamine was then removed by distillation using wiped film evaporator. Removal of ethylenediamine was monitored by NMR of crude reaction mixture. The product was obtained as an oil. Quantitative H-NMR analysis showed the oil to be 21.5 wt. % PAMAM G3.0. A summary of metrics of the process are shown in Table 22.

TABLE 22
Process metrics for experiment AA08-086

| | | |
|---|---|---|
| Reaction time | 120 | h |
| Crude yield | 6162.5 | g |
| Product potency | 21.5% | w/w |
| Assay yield (g) | 1322 | g |

| | |
|---|---|
| Assay yield (%) | 95% |
| Polydispersity (Ð (M_{w}/Mₙ)) | 1.14 |

### PAMAM G3.5 Synthesis

TABLE 23

| | MW | Potency | Density | Amount | Amount | | |
|---|---|---|---|---|---|---|---|
| Reagent | (g/mol) | (Wt. %) | (g/mL) | (g) | (mL) | mmol | Eq/vol |
| Methyl Acrylate solution | | | | 1518.3962 | | | |
| Methyl Acrylate | 86.09 | | 0.95 | 1740.1 | 1831.7 | 20212.81 | 96 |
| MeOH | 32.04 | | 0.792 | 1450.71 | 1831.7 | | 0.30 |
| PAMAM G2.0 solution | | | | 13219 | | | |
| PAMAM G2.0 | 3256.24 | 21.45 | 0.9 | 6162.5 | | 191.32 | 1 |
| MeOH | | | 0.792 | 7056 | 8909 | | |

A 20 L jacketed reactor was charge with methanolic solution of methylacrylate and a methanolic solution of PAMAM G3.0 (AA8-086) was added at 0° C. over a period of 2 hours. The solution for stirred for two more hour at 0° C. and then warmed to 20° C. and stirred for 4 days. Reaction completion was validated by ¹H-NMR spectroscopy. Volatiles were then removed by distillation under reduced pressure with the reactor jacket controlled at 20° C. Residual methyl acrylate was then removed by 3× azeotropic removal with methanol (3 vol.) after which the GC and NMR of crude material showed complete removal of methyl acrylate. The product was obtained as 7610 g of a viscous oil. Quantitative ¹H-NMR analysis showed the oil to be 15.97 wt. % PAMAM G2.5 providing as assay yield of 2360 g (99% yield). A summary of metrics of the process are shown in Table 24.

TABLE 24
Process metrics for experiment AA10-062

| | | |
|---|---|---|
| Reaction time | 96 | h |
| Crude yield | 13800 | g |
| Product potency | 17.1% | w/w |
| Assay yield (g) | 2359.8 | g |

| | |
|---|---|
| Assay yield (%) | 99% |
| Polydispersity (Ð (M_{w}/Mₙ)) | 1.1 |

### PAMAM G4-OH-Alkyne₇₋₈ Synthesis—Lot AA10-064

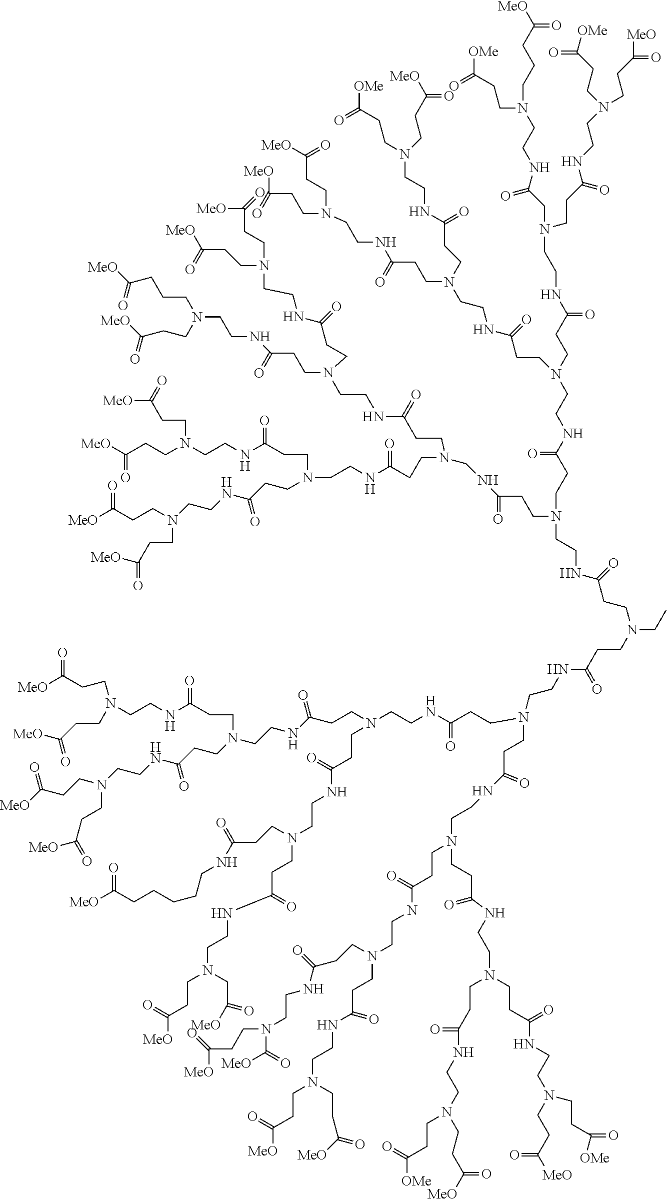

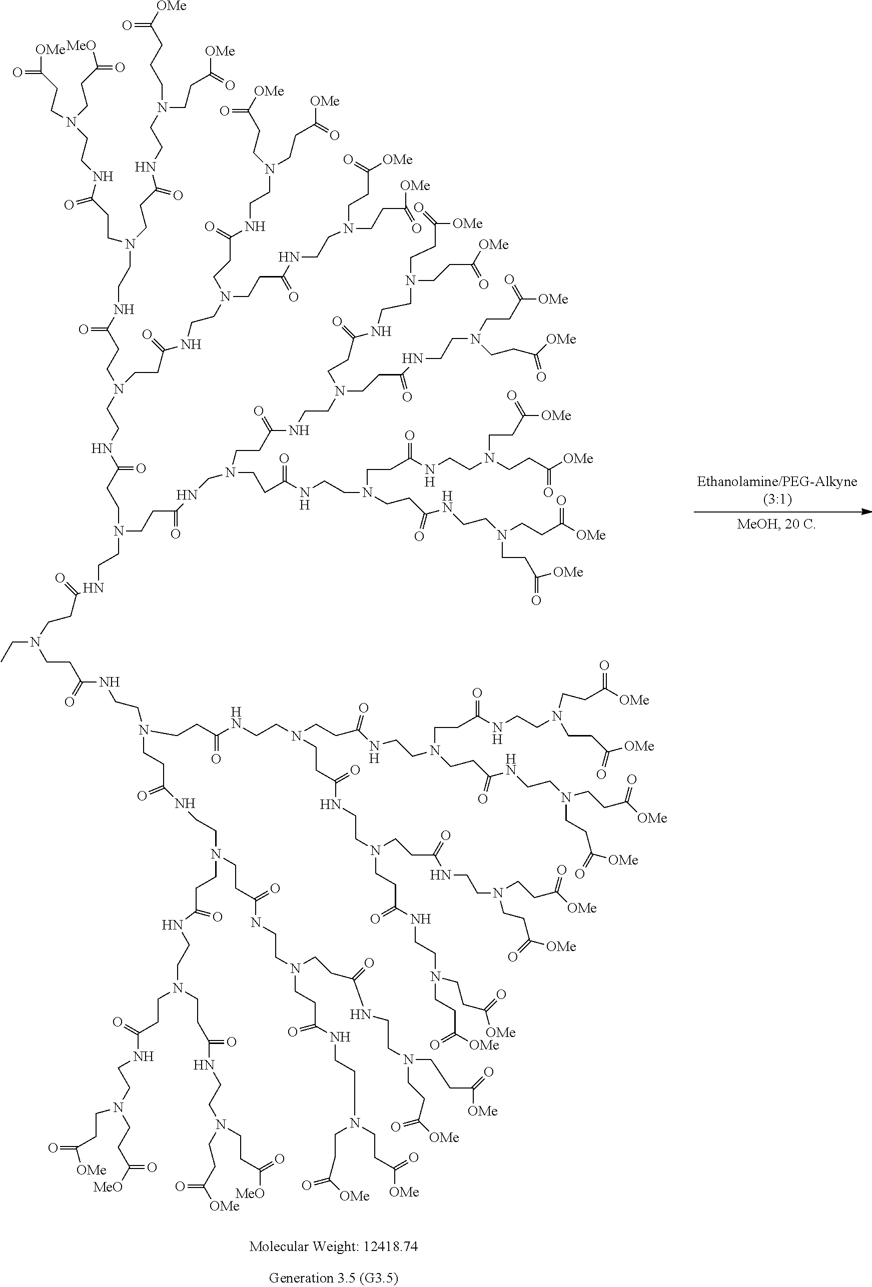

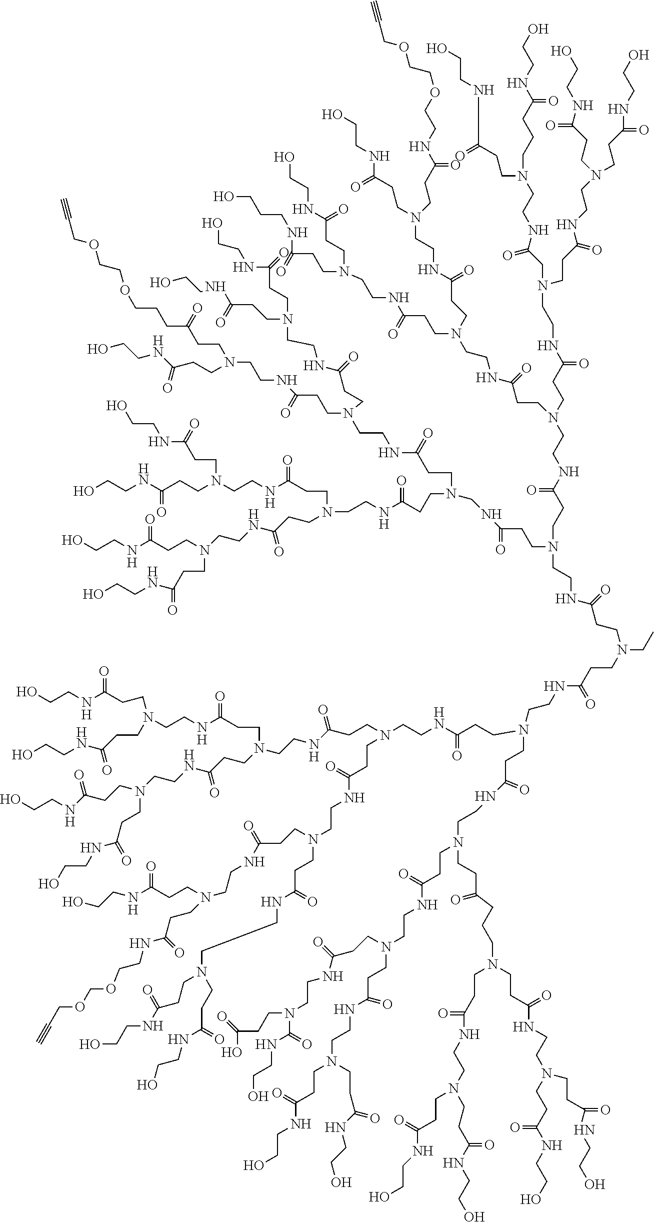

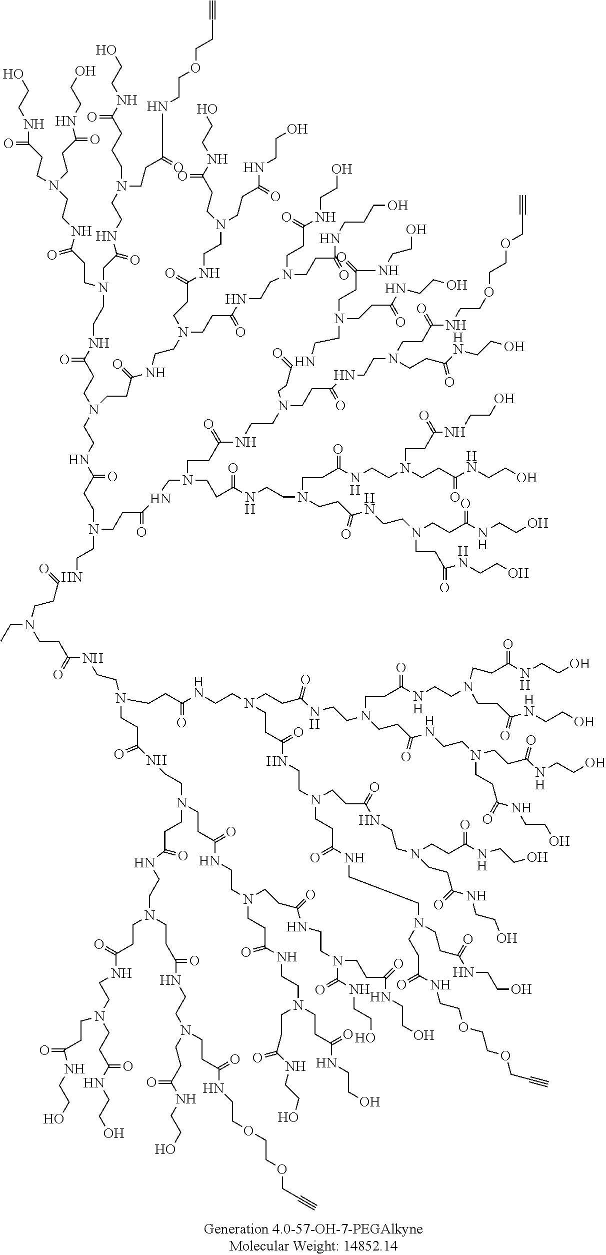

TABLE 25

| | MW | Potency | Density | Amount | Amount | | |
|---|---|---|---|---|---|---|---|
| Reagent | (g/mol) | (% w/w) | (g/mL) | (g) | (mL) | mmol | Eq/vol |
| Amine solution | | | | 6233 | | | |
| Ethanolamine | 61.08 | 100 | 1.012 | 2853 | 2821 | 46623 | 1200 |
| PEG-Alkyne | 143.19 | 100 | | 2355 | | 15541 | 400 |
| MeOH | | 100 | 0.792 | 1025 | 1294 | | 2.59 |
| PAMAM G3.5 solution | | | | 4825 | | | |
| PAMAM G2.5 | 6011.2 | 96.5 | | 500 | | 38.85 | 1 |
| MeOH | | 100 | 0.792 | 4325 | 5461 | | 11.32 |

Jacketed reactor was purged with N₂(g) and charged with ethylenediamine solution. PAMAM G3.5 solution was added via diaphragm pump at 0° C. over a period of ˜2 hours. After stirring for ˜4 more hours at 0° C. the solution was warmed to 20° C. and stirred 6 days. After this time the solution was diluted with H₂O (11 kg). The solution was then subjected to purification by ultrafiltration with a regenerated cellulose membrane with 5 kDa MWCO providing the product as an aqueous solution which showed complete removal of ethanolamine and PEG-Alkyne by ¹H-NMR. The solvent was this exchanged with methanol via continuous diafiltration to provide the product PAMAM G4 as a methanolic solution (3230.9 g). Quantitative H-NMR analysis showed the solution to be 16.6 wt. % PAMAM G4-OH-Alkyne₇_s (93% yield). A summary of metrics of the process are shown in Table 26.

TABLE 26
Summary of results AA10-064

| Lot number | AA10-064 | |
|---|---|---|
| PAMAM G3.5 source | Dendritech | |
| Methanolic solution mass (kg) | 3.25 | |
| Potency (% w/w) | 16.6% | w/w |
| Yield | 539.5 | g |

| | |
|---|---|
| Yield (%) | 93% |
| Polydispersity (M_{w}/Mₙ) | 1.030 |
| Average # of PEG-alkyne arms | 7.8 |
| —OH/—NH ratio | 0.39 |
| Residual ethanolamine (dry basis, | 0.2% |
| GC) | |
| Residual PEG-Alkyne (dry basis, | 0.02% |
| GC) | |

### PAMAM G4-OH-Alkyne₇₋₈ Synthesis—Lot AA10-065

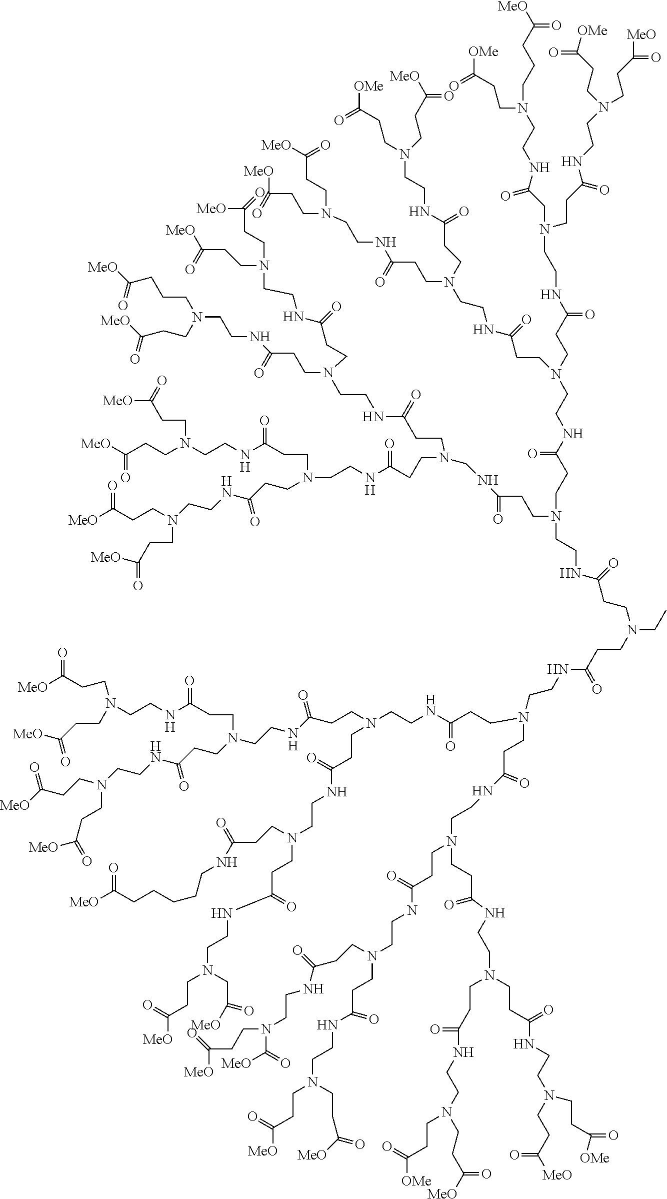

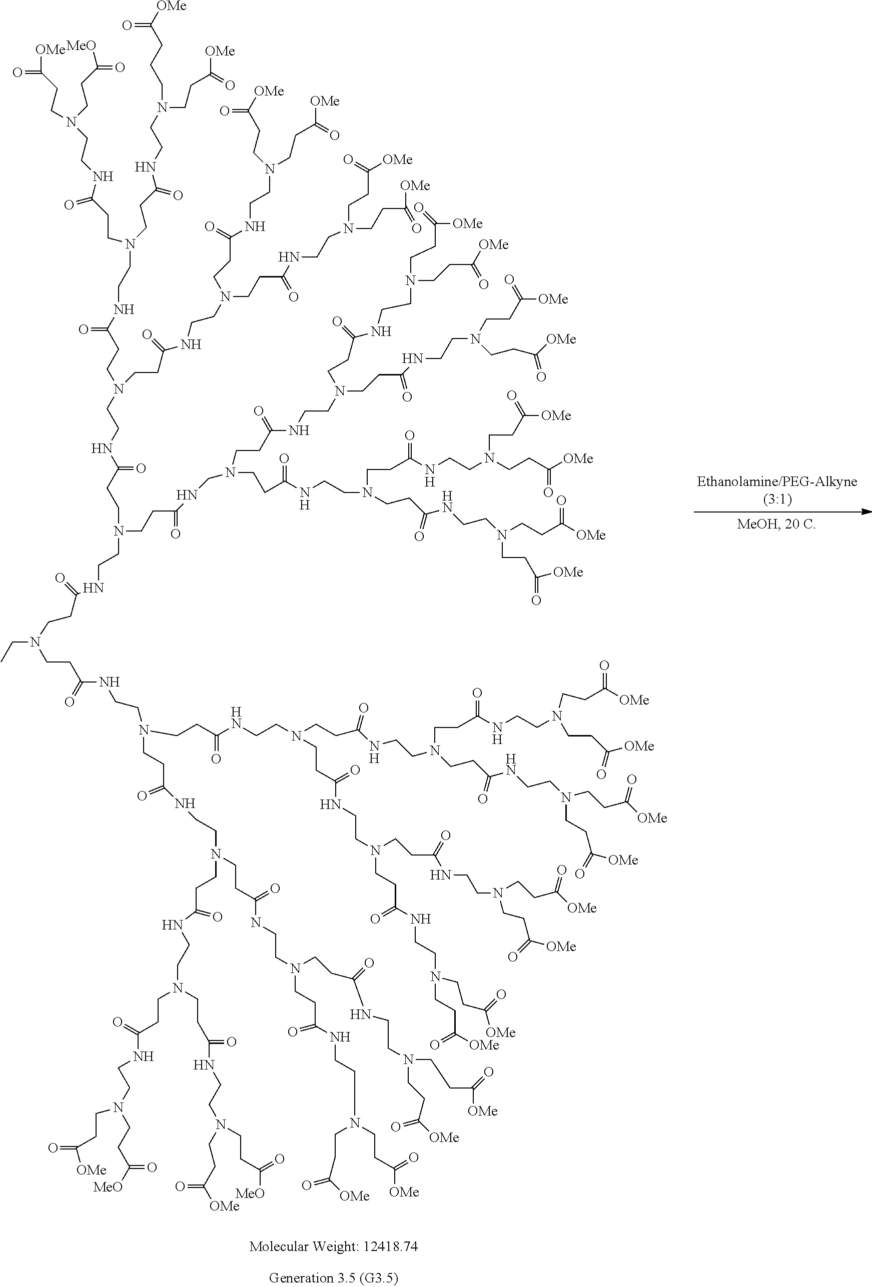

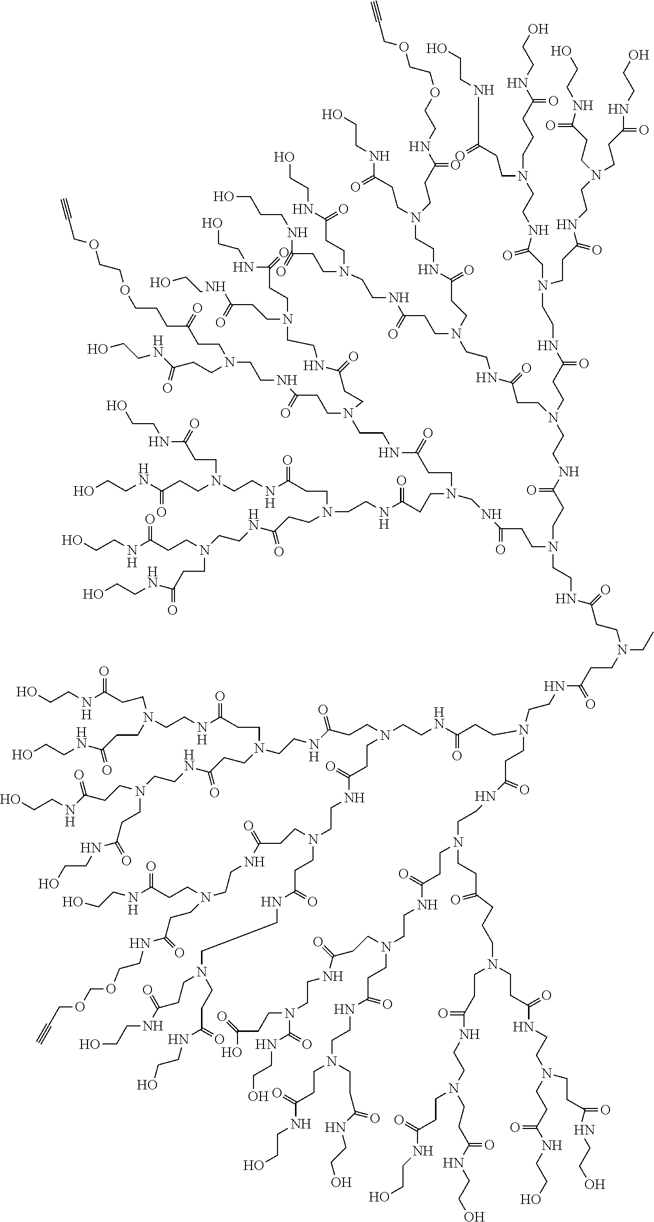

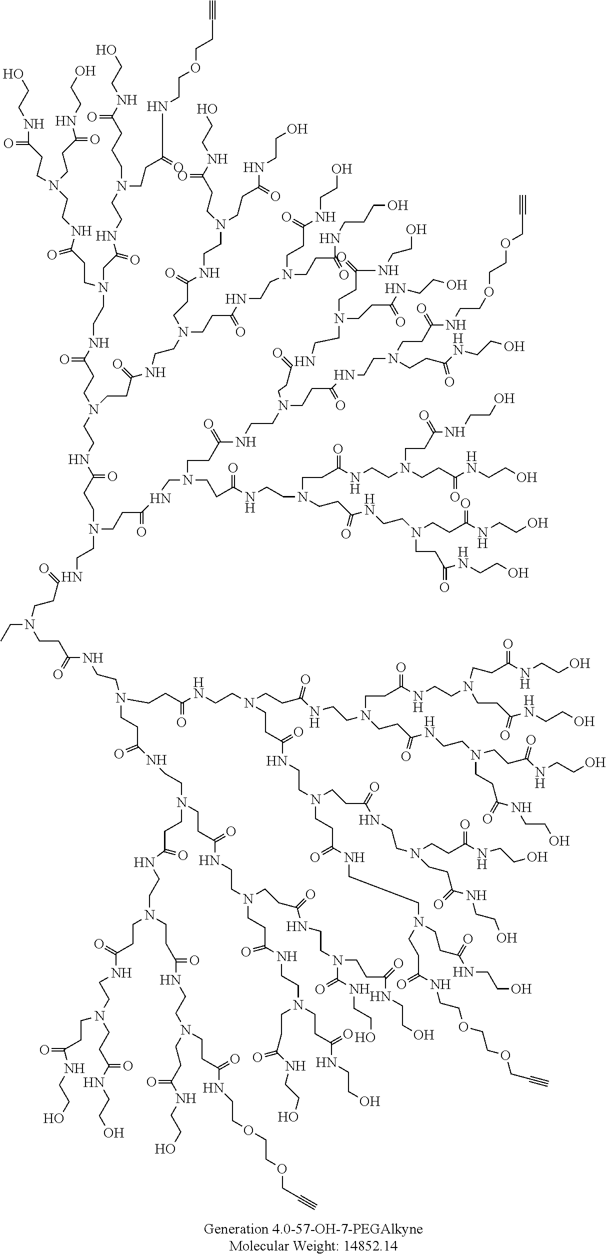

| | MW | Potency | Density | Amount | Amount | | |
|---|---|---|---|---|---|---|---|
| Reagent | (g/mol) | (% w/w) | (g/mL) | (g) | (mL) | mmol | Eq/vol |
| Amine solution | | | | 23457 | | | |
| Ethanolamine | 61.08 | 100 | 1.012 | 11002 | 10876 | 180139 | 1200 |
| PEG-Alkyne | 143.19 | 100 | | 8598 | | 60046 | 400 |
| MeOH | | 100 | 0.792 | 3856 | 4868 | | 2.61 |
| PAMAM G3.5 solution | | | | 18642 | | | |
| PAMAM G2.5 | 6011.2 | 25.9 | | 10902 | | 150.12 | 1 |
| MeOH | | 100 | 0.792 | 7740 | 9773 | | 5.24 |

A jacketed reactor was purged with N₂ (g) and charged with ethylenediamine solution. PAMAM G3.5 solution was added via diaphragm pump at 0° C. over a period of ˜2 hours. After stirring for ˜4 more hours at 0° C. the solution was warmed to 20° C. and stirred 6 days. After this time the solution was diluted with H₂ (42 kg). The solution was then subjected to purification by ultrafiltration with a regenerated cellulose membrane with 5 kDa MWCO providing the product as an aqueous solution which showed complete removal of ethanolamine and PEG-Alkyne by ¹H-NMR. Dimeric impurities were then removed by ultrafiltration using a regenerated cellulose membrane with 3% kDa MWCO with desired product passing through to permeate. The solvent was then exchanged with methanol via continuous diafiltration to provide the product PAMAM G4 as a methanolic solution (8205 g). Quantitative H-NMR analysis showed the solution to be 19.5 wt. % PAMAM G4-OH-Alkyne₇₋₈ (71.8% yield). A summary of metrics of the process are shown below in Table 27.

TABLE 27
Summary of results for AA10-065

| Lot number | AA10-065 | |
|---|---|---|
| PAMAM G3.5 source | Snapdragon (Lot AA10-062) | |
| Methanolic solution mass (kg) | 8.21 | kg |
| Potency (% w/w) | 19.5% | w/w |
| Yield | 1.6 | kg |

| | |
|---|---|
| Yield (%) | 71.8% |
| Polydispersity (M_{w}/Mₙ) | 1.045 |
| Average # of PEG-alkyne arms | 7.8 |
| —OH/—NH ratio | ND |
| Residual ethanolamine (dry basis, | 0.4% |
| GC) | |
| Residual PEG-Alkyne (dry basis, | 0.01% |
| GC) | |

### Analytical Methods

UPLC method (ASHV001O)

### A. Reagents

1. Water: HPLC grade
2. Acetonitrile: HPLC grade
3. Trifluoroacetic acid: HPLC grade

### B. Solutions

1. Diluent: MiliQ water
2. Mobile phase:
   A. 0.05% Trifluoroacetic acid in Water
   B. 0.05% Trifluoroacetic acid in Acetonitrile
3. Sample Solutions: the sample solution at a target concentration of approximately 2 mg/mL.
4. Blank: Diluent.

### C. Chromatographic Conditions

Column: ACE Excel 3 Super C18, LC column, 50×3×3 μm
Temperature: 40° C.
Flow Rate: 1.27 mL/min
Detection: A: UV at 210 nm; 4.8 nm bandwidth
   B: UV at 220 nm; 4.8 nm bandwidth
Injection Volume: 10 μL
Run Time: 12.3 min

Gradient

| Time (min) | % A | % B |
|---|---|---|
| 0 | 98 | 2 |
| 10.00 | 0 | 100 |
| 12.02 | 98 | 2 |
| 12.30 | 98 | 2 |

### D. Calculation

$$Peak Area \% = \frac{{A}_{c}}{{T}_{a} - {A}_{b}} \times 100$$

$${A}_{b} = Total area counts from the blank \\ {A}_{c} = Area counts of component in sample injection \\ {T}_{a} = Total area from sample injection$$

This method may also be used for weight percent of assay analysis with calibration standards. Typical standard concentrations 0.025 to 1.0 mg/mL.

### GC Method (ASV003F)

### A. Chromatographic Conditions

Column: Agilent CP-Volamine (Part number CP7447)
Inlet Temperature: 250° C.
Inlet Pressure: 14.3 psi
Inlet Total flow: 37.3 mL/min
Inlet Split Ratio: 10:1
Injection Volume: 5 μL
Column Flowrate: 3.1 mL/min
FID Temperature: 300° C.
FID H₂ Flow: 40.0 mL/min
FID Air Flow: 360.0 mL/min
FID Makeup Flow: 25 mL/min
FID Lit Offset: 0.5 pA
Run Time: 20.33 min

Oven Temperature Gradient

| Rate (° C./min) | Temperature (° C.) | Hold time | Run time |
|---|---|---|---|
| N/A | 65 | 4 | 4 |
| 15 | 100 | 3 | 9.33 |
| 30 | 250 | 6 | 20.33 |

### D. Calculation

$$Weight \% = ( \frac{{A}_{c} - {A}_{b}}{{R}_{a}} ) / {C}_{s} \times 100$$

$${A}_{b} = Total area counts from the blank \\ {A}_{c} = Area counts of component in sample injection \\ {R}_{a} = Response factor of component \\ {C}_{s} = Concentration of sample$$

For determining impurities using this method, standards of 14.928 and 10.332 mg/mL of ethanolamine and PEG alkyne in isopropyl alcohol respectively were prepared and injected onto the GC. This gave a retention time of ethanolamine around 6.88 minutes while the PEG alkyne had a retention time of 14.1 minutes. These solutions were combined in a 1:1 ratio. This combined solution was used as a stock solution for ½ serial dilutions using isopropyl alcohol reaching concentrations of 0.007 and 0.005 mg for Ethanolamine and PEG alkyne respectively. The results of the calibration are shown below.

GC Calibration data for ethanolamine and PEG-Alkyne

| Concentration of | Concentration of PEG | ethanolamine | PEG-alkyne |
|---|---|---|---|
| Ethanolamine | alkyne | signal | signal |
| (mg/mL) | (mg/mL) | area | area |
| 14.928 | 10.332 | 22543.5 | 22341 |
| 7.4640 | 5.1660 | 11862.6 | 11688.7 |
| 3.7320 | 2.5830 | 5755.5 | 5656.6 |
| 1.8660 | 1.2915 | 2962 | 2966 |
| 0.93300 | 0.64575 | 1423.5 | 1486.3 |
| 0.46650 | 0.32288 | 708.2 | 758.3 |
| 0.23325 | 0.16144 | 323.2 | 360.1 |
| 0.11663 | 0.080719 | 153.4 | 177.7 |
| 0.058313 | 0.040359 | 72.9 | 91 |
| 0.029156 | 0.020180 | 30.5 | 42.3 |
| 0.014578 | 0.010090 | 13 | 21 |
| 0.0072891 | 0.0050449 | 4.7 | 11.5 |

The calibration curve for ethanolamine was used to calculate the concentration of ethanolamine in the sample. The signal for PEG-Alkyne was below the LOD so the lowest value reached in the calibration was reported as the concentration.

### SEC-MALLS Method

### A. Reagents

1. Water: HPLC grade
2. Citric acid: ACS reagent, >99.5%

### B. Solutions

1. Diluent: Water, 0.1 M Citric acid, pH 2.7
2. Mobile phase:
   A. Water, 0.1 M Citric acid, pH 2.7
3. Sample Solutions: the sample solution at a target concentration of approximately 2 mg/mL.
4. Blank: Diluent.

### C. Chromatographic Conditions

Column: TosoHaas TSK-Gel Guard PHW 0662 (75 mm×7.5 mm, 12 μm), G2000 PW 05761 (300 mm×7.5 mm, 10 μm, 125 Å), G 3000 PW 05762 (300 mm×7.5 mm, 10 μm, 200 Å), G4000 PW (300 mm×7.5 mm, 17 μm, 500 Å) Temperature: 25° C.
Flow Rate: 1 mL/min
Detection: A: Wyatt Optilab differential refractive index detector operating at 658 nm
   B: Wyatt miniDAWN multi-angle laser light scattering detector with 120 mW laser operating at 658 nm
Injection Volume: 100 μL
Run Time: 40 min

### EXAMPLE 4 REFERENCES

1. Cason, C. A, et al. Journal of Nanomaterials. 2008, 1-7. DOI:10.1155/2008/456082
2. Mullen, D. G., et. al. Macromolecules. 2012, 45, 5316-5320.

### Example 5. Preparation of PEG-Alkyne Functionalized PAMAM-G4-OH

A process was developed to install G4.0 PAMAM dendrimer with 3 PEG-alkyne (PAMAM G4-OH-Alkyne₃) and 10 PEG-alkyne (PAMAM G4-OH-Alkyne₃) linker arms. These functionalized dendrimers were produced by tuning the stoichiometry of PEG-alkyne relative to ethanolamine to account for differences in reactivity. This process was successfully scaled up to provide 40 g of PAMAM G4-OH-Alkyne₃ and 160 g of the PAMAM G4.0-50-OH-10-PEG-Alkyne. In both cases, the products were obtained with low polydispersity of about 1.03.

In addition, a manufacturing process was developed for a poly(amidoamine) generation 4 hydroxyl terminated dendrimer (PAMAM-G4-OH) for a targeted drug delivery application. An azide modification of the PAMAM G4-OH was used to enable conjugation of the active using azide-alkyne cycloaddition (click) conjugation technology. A process was developed for the preparation of a PAMAM G4 with an average of seven alkyne functional groups. The current PAMAM G4-OH synthetic route follows divergent strategy for the preparation of PAMAM dendrimer. The final step of the process was the addition of ethanolamine resulting in the G4 dendrimer with 64 alcohol functional groups. For conjugation, a process was developed to prepare two G4-OH analogs where n approximately equal to 3 and 10, respectively.

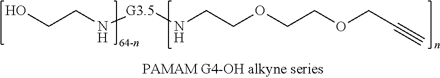

### Analytical Methods

Due to the complexity of the PAMAM dendrimers, the quality attributes of the product were characterized with a number of analytical methods including SEC-MALLS (polydispersity), ¹H NMR (identity, potency, average linker loading), ¹³C NMR (identity), GC (residual ethanolamine and PEG alkyne monomer).

### SEC-MALLS Method

A size-exclusion chromatography method with multi-angle laser light scattering (MALLS) and differential refractive index (dRI) detectors was developed adopting methods described in the literature. See Mullen, D. G., et. al. Macromolecules. 2012, 45, 5316-5320. Each PAMAM generation peak was able to be resolved using this method. A summary of elution times and measured polydispersities for each generation is provided in Table 28.

TABLE 28
SEC elution times for PAMAM dendrimers

| | | Elution time | Ð (M_{w}/ |
|---|---|---|---|
| Compound | Lot # | (min) | Mₙ)^{a} |
| PAMAM G3.5 | — | 17.9 | 1.036 |
| PAMAM G4 | MKCL7410 | 17.8 | 1.031 |

### Quantitative ¹H NMR

Quantitative ¹H NMR spectroscopy was performed referencing to Sigma Aldrich TraceCERT® grade internal standards. These methods have been used to quantify the potency of PAMAM dendrimer solutions and isolated products. This method has further been used to assay the quality of PEG-Alkyne material (2-[2-(propargyloxy)ethoxy]ethylamine). 1,3,5-trimethoxybenzene was used as an internal standard in d⁴-methanol as a solvent.

### Process Development

### PAMAM Generation 3.5 Source Material

A methanolic solution (29.4% w/w) of PAMAM Generation 3.5 was generated prior to use. Quality attributes for this input material are provided in Table 29.

TABLE 29
PAMAM G3.5 reagent solution product information

| PAMAM G3.5 Manufacturer | Dentritech | |
|---|---|---|
| Manufacturer lot# | 0121-02-E3.5-LD-A | |
| Total solution mass | 1648 | g |
| Solution potency (% w/w) | 29.4 | |
| PAMAM G3.5 (g) | 494.3 | g |

| | |
|---|---|
| Polydispersity (M_{w}/Mₙ) | 1.036 |

Reaction of PAMAM G3.5 with PEG-Alkyne/Ethanolamine Under High Amine Excess Conditions

A series of experiments were carried out exploring variations of the PEG-Alkyne/ethanolamine molar ratio under high amine excess conditions (1600 equivalents total amine, 25 equivalents per terminal ester). First, methanolic solution of PEG-Alkyne and ethanol amine (˜1600 equivalents) was cooled to 0° C. Then, a methanolic solution of PAMAM G3.5 (10% w/w) was added dropwise over 2 hours. After stirring for an additional 2 hours at 0° C., the solution was warmed to 20° C. and stirred for 6 days. For purification, the material was processed by TFF providing an aqueous solution of the product. For analysis, a portion of the solution was taken, and water was then removed via vacuum which was analyzed by ¹H-NMR. Following the same protocol three other reactions were set up to explore the PEG-Alkyne/ethanolamine ratio to reach 10 and 14 alkyne chains. A similar study is currently underway to optimize conditions for an average 3 alkyne arm degree of functionalization.

### Protocol for Analysis of Average Number of PEG-Alkyne Arms

Determination of the degree of functionalization of PAMAM G4-OH with PEG-Alkyne was determined via ¹H-NMR spectroscopy using the average value provided by two methods (denoted method A and method B). Each method makes use of the integrations observed with PAMAM-G4-64-OH and the theoretical integrations that would be observed with 100% PEG-Alkyne functionalized PAMAM-G4-64-PEG-Alkyne. The respective integrations for these two materials are provided in Table 30 and Table 31.

TABLE 30
Proton chemical shift and integrations for PAMAM-G4-64-OH

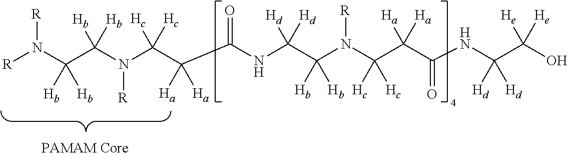

| Proton | Chemical Shift (ppm) | | # of protons |
|---|---|---|---|
| Hₐ | 2.38 | ppm | 248 H |
| H_{b} | 2.58 | ppm | 124 H |
| H_{c} | 2.79 | ppm | 248 H |
| H_{d} | 3.26-3.33 | ppm | 248 H |
| Hₑ | 3.61 | ppm | 128 H |

TABLE 31
Proton chemical shift and integrations for PAMAM-G4-64-PEG-Alkyne (theoretical)

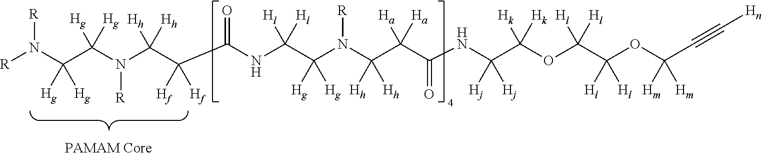

| Proton | Chemical Shift (ppm) | # of protons |
|---|---|---|
| H_{f} | 2.38 ppm | 258 H |
| H_{g} | 2.58 ppm | 124 H |
| Hₕ | 2.79 ppm | 248 H |
| Hᵢ | 3.27 ppm | 120 H |
| Hⱼ | 3.37 ppm | 128 H |
| Hₖ | 3.55 ppm | 128 H |
| Hₗ | 3.65 ppm (multiplet) | 256 H |
| Hₘ | 4.2 ppm | 128 H |

### Results for Experiments Varying Ratio of PEG-Alkyne/Ethanolamine

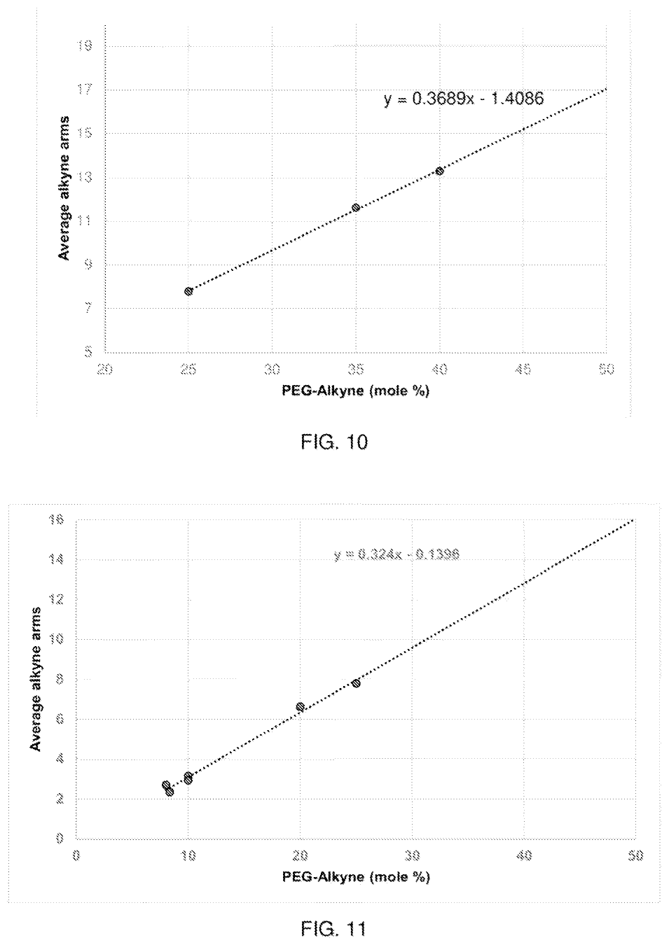

Methods A and B were used for calculation of the average number of PEG-Alkyne arms on the resulting dendrimers in experiments DP07-51 with 1600 equivalents of total amine relative to dendrimer (25 equivalents relative to terminal groups). These results are summarized in Table 32. The relationship between average number of PEG-Alkyne arms vs. mol % of PEG-Alkyne with respect to total amine content is shown in FIG. 10.

TABLE 32
Summary of results for PEG-Alkyne functionalization
of PAMAM G-4 using 1600 equivalents of amine

| | PEG- | | | | |
|---|---|---|---|---|---|
| | Alkyne | | | | |
| | (mol % | | | | |
| | relative | | | | |
| | to total | Average # PEG-Alkyne arms | | | Polydispersity |
| | amine | Method | Method | | (M_{w}/ |
| Sample | content) | A | B | Average | Mₙ) |
| DP07-51-1 | 25 mol % | 7.80 | 7.96 | 7.88 | 1.034 |
| DP07-51-2 | 35 mol % | 11.84 | 11.36 | 11.60 | 1.053 |
| DP07-51-3 | 40 mol % | 14.01 | 13.28 | 13.65 | 1.030 |
| DP07-51-4 | 45 mol % | 15.61 | 15.15 | 15.38 | 1.056 |

The data was combined with previous development data obtained and listed in Table 33 and plotted in FIG. 11. These data further validate the reproducible linear correlation between average number of PEG-Alkyne arms vs. mol % of PEG-Alkyne with respect to total amine content.

TABLE 33
Summary of results for PEG-Alkyne functionalization of PAMAM
G-4 using 1600 equivalents of amine from all the experiments

| | Mole % | Average alkyne |
|---|---|---|
| LNB# | Alkyne | arms |
| AA08-88-2 | 8.33 | 2.33 |
| AA08-88-1 | 10.00 | 2.93 |
| AA08-88-3 | 20.00 | 6.62 |
| DP07-51-1 | 25.00 | 7.78 |
| AA08-88-3 | 26.30 | 8.03 |
| DP07-51-2 | 35.00 | 11.6 |
| DP07-51-3 | 40.00 | 13.28 |
| DP07-51-4 | 45.00 | 15.38 |

Results for Experiments Varying Ratio of PEG-Alkyne/Ethanolamine for n=3

A series of experiments were carried out to fine-tune the PEG-Alkyne/ethanolamine molar ratio to dial in the target average linker n=3 (1600 equivalents total amine, 25 equivalents per terminal ester). First, methanolic solution of PEG-Alkyne and ethanol amine (˜1600 equivalents) was cooled to 0° C. Then, a methanolic solution of PAMAM G3.5 (10% w/w) was added dropwise over 2 hours. After stirring for an additional 2 hours at 0° C., the solution was warmed to 20° C. and stirred for 6 days. For purification, the material was processed by TFF providing an aqueous solution of the product. For analysis, a portion of the solution was taken, and water was then removed via vacuum which was analyzed by ¹H-NMR.

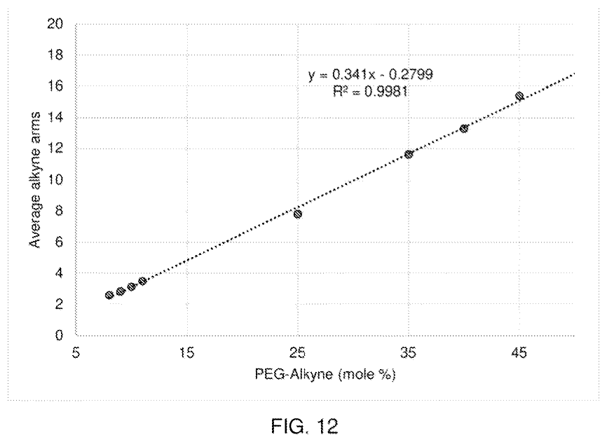

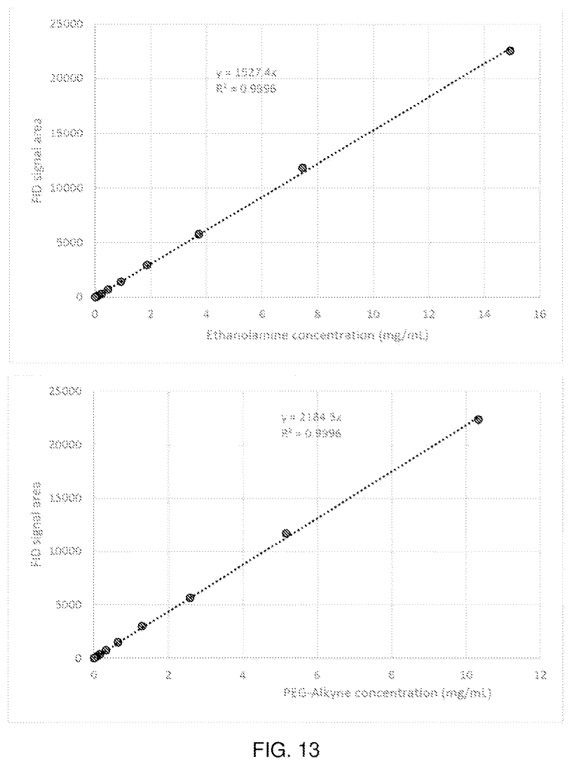

These results are summarized in Table 34. The relationship between average number of PEG-Alkyne arms vs. mol % of PEG-Alkyne with respect to total amine content is shown below in FIG. 12. Based on the linear equation, for n=3, 9.6 mol % PEG-alkyne is required and for n=10, 31 mol % is required.

TABLE 34
Summary of results for PEG-Alkyne functionalization
of PAMAM G-4 using 1600 equivalents of amine

| | PEG-Alkyne (mol % | Average # PEG-Alkyne arms | | | Polydispersity |
|---|---|---|---|---|---|
| | relative to total | Method | Method | | (M_{w}/ |
| Sample | amine content) | A | B | Average | Mₙ) |
| DP07-60-1 | 8 mol % | 2.67 | 2.54 | 2.60 | 1.026 |
| DP07-60-2 | 9 mol % | 2.95 | 2.74 | 2.85 | 1.029 |
| DP07-60-3 | 10 mol % | 3.18 | 3.12 | 3.15 | 1.025 |
| DP07-60-4 | 11 mol % | 3.58 | 3.44 | 3.51 | 1.026 |

TABLE 35
Summary of results for PEG-Alkyne functionalization of PAMAM
G-4 using 1600 equivalents of amine for DP07-60 and DP07-51

| Experiment | Mole % Alkyne | Average alkyne arms |
|---|---|---|
| DP07-60-1 | 8 | 2.61 |
| DP07-60-2 | 9 | 2.85 |
| DP07-60-3 | 10 | 3.15 |
| DP07-60-4 | 11 | 3.51 |

5 g Validation Run to Produce PAMAM G4.0 with n=3 Alkyne Arms (DP07-74-1)

Experiment DP07-74-1 was carried out on 5-gram scale to allow for further validation of the ratio of PEG-Alkyne/ethanolamine previously established via small scale reaction screening. Reaction was set up with 9.6 mol % of PEG-Alkyne as explained before and was stirred for 6 days at 20° C. The crude reaction mass was diluted 2× with water and purified via TFF using 5 kDa ultrafiltration membranes to allow for the removal of impurities. After this purification, water was removed by lyophilization. Quantitative ¹H-NMR analysis indicated this material to be 97% w/w of PAMAM G-4 material for an adjusted isolated yield of 74%. A summary of the analysis for this process is provided below in Table 36. Reduced isolated yields as compared to previous scopes of work are not currently understood and may simply be due to mechanical losses during the TFF purification.

TABLE 36
Summary of analysis for DP07-74-1,
PAMAM G4.0 with 3 alkyne arms

| Parameter | Value |
|---|---|
| Isolated yield (g) | 4.5 g |
| Potency (¹H-NMR, % w/w) | 97% |
| Adjusted yield (g) | 4.34 g |
| Isolated yield (%) | 74% |
| Polydispersity (M_{w}/Mₙ) | 1.029 |
| Average # alkyne arms | 3.18 (Method A: 3.20 and Method B: 3.15) |
| GC data | GC impurity calibration revealed: 0.21% |
| | ethanolamine and 0.14% PEG-Alkyne |
| | (assuming 100 wt %) |
| | GC impurity calibration revealed: 0.21% |
| | ethanolamine and 0.14% PEG-Alkyne |
| | (assuming 100 wt %) |

5 g Validation to Produce PAMAM G4.0 with n=10 Alkyne Arms (DP07-68-1)

Experiment DP07-68-1 was carried out on 5-gram scale to allow for further validation of the ratio of PEG-Alkyne/ethanolamine obtained from reaction screening to synthesize n=10 alkyne arms. Reaction was set up with 31 mol % of PEG-Alkyne as explained before and was stirred for 6 days at 20° C. The crude reaction mass was diluted 2× with water and purified via TFF using 5 kDa ultrafiltration membranes to allow for the removal of impurities. After this purification, water was removed by lyophilization. A summary of the analysis for this process is provided in Table 37. As above, reduced isolated yields compared to previous development work are believed to be due to mechanical losses in the filtration equipment.

TABLE 37
Summary of analysis for DP07-68-1,
PAMAM G4.0 with 10 alkyne arms

| Parameter | Value |
|---|---|
| Isolated yield (g) | 3.8 g |
| Potency (¹H-NMR, % w/w) | 100% |
| Isolated yield (%) | 63% |
| Polydispersity (M_{w}/Mₙ) | 1.038 |
| Average # alkyne arms | 10.03 (Method A: 10.34 |
| | and Method B: 9.72) |

Scale-Up to Produce 40-Gram PAMAM G4.0 with 3.0 Alkyne Arms (DP07-82-1)

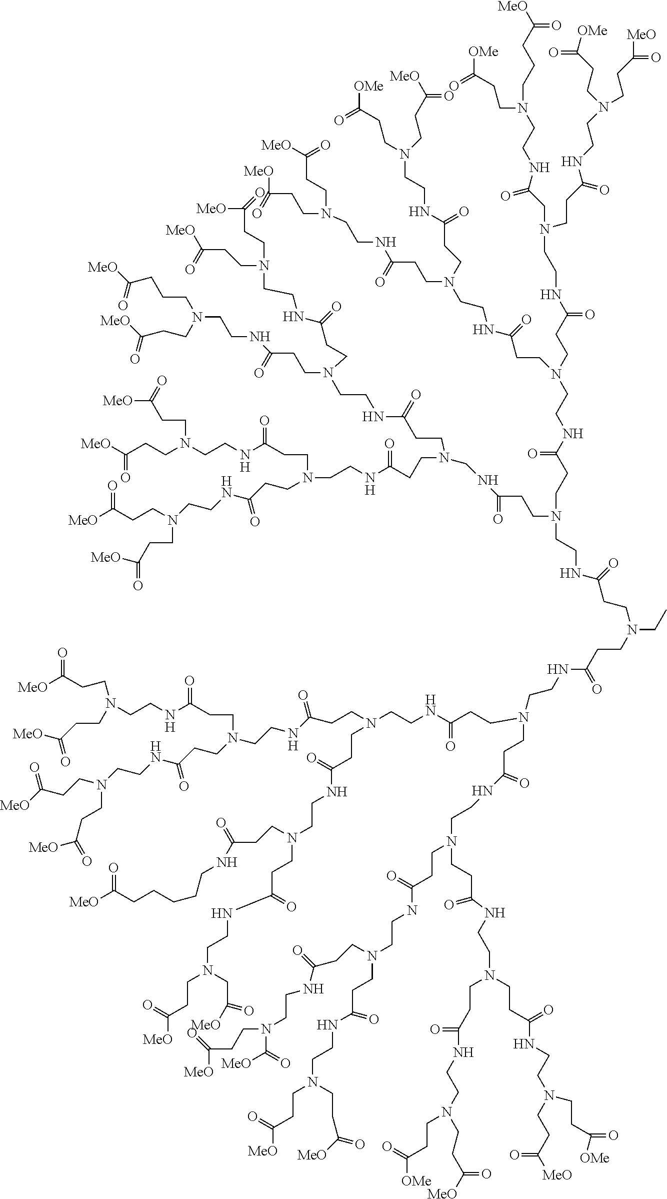

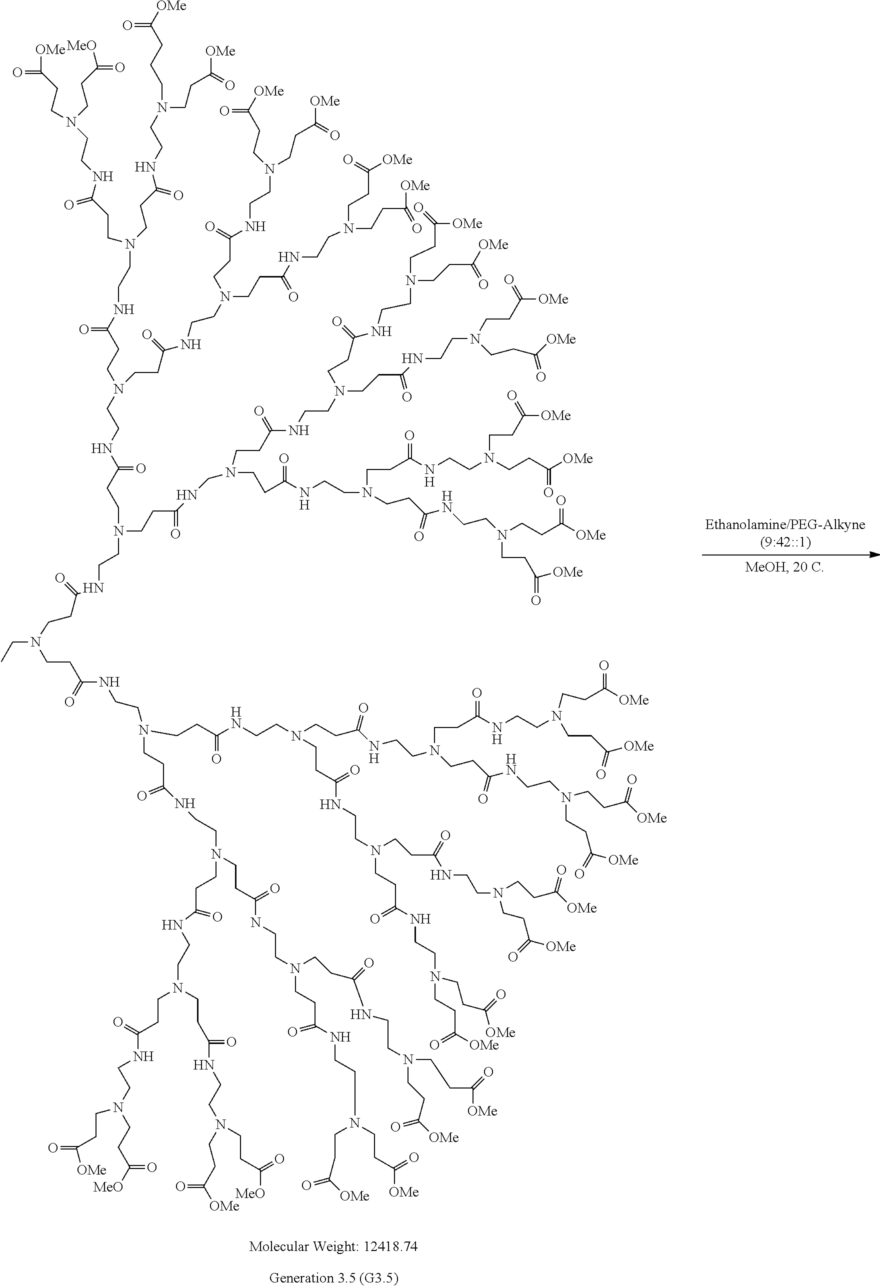

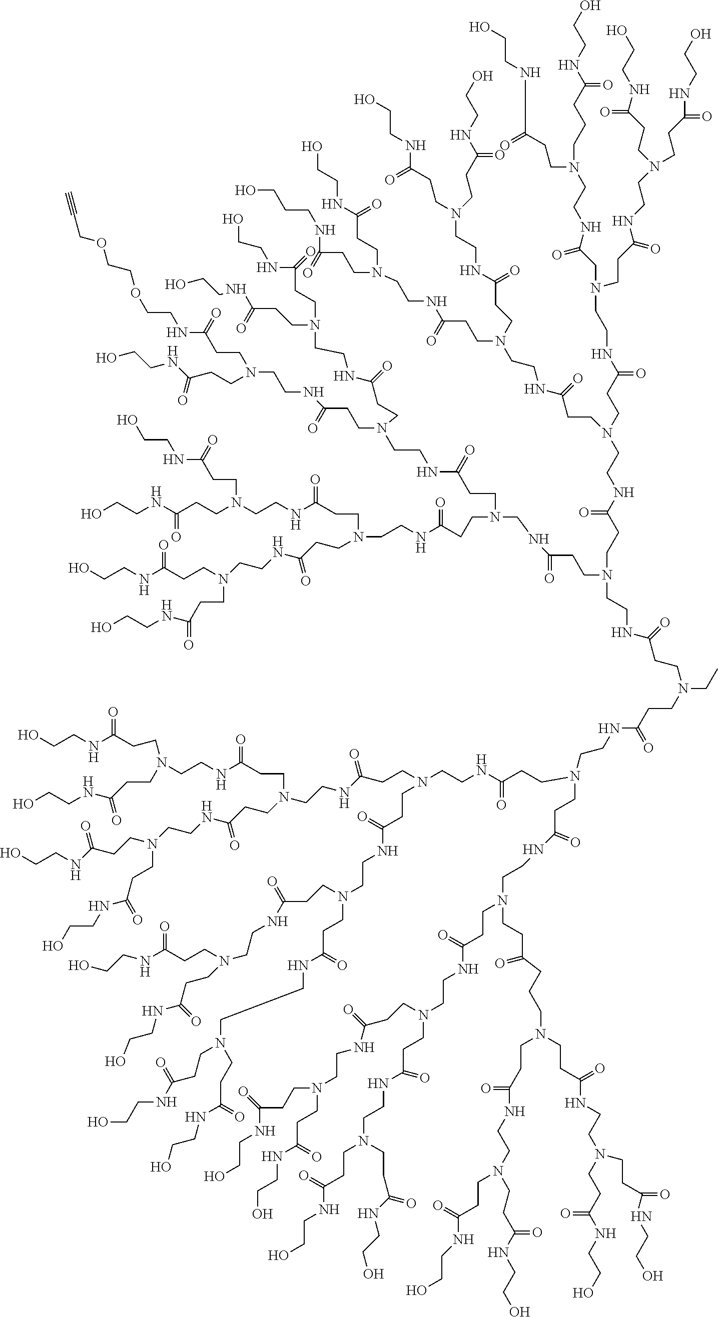

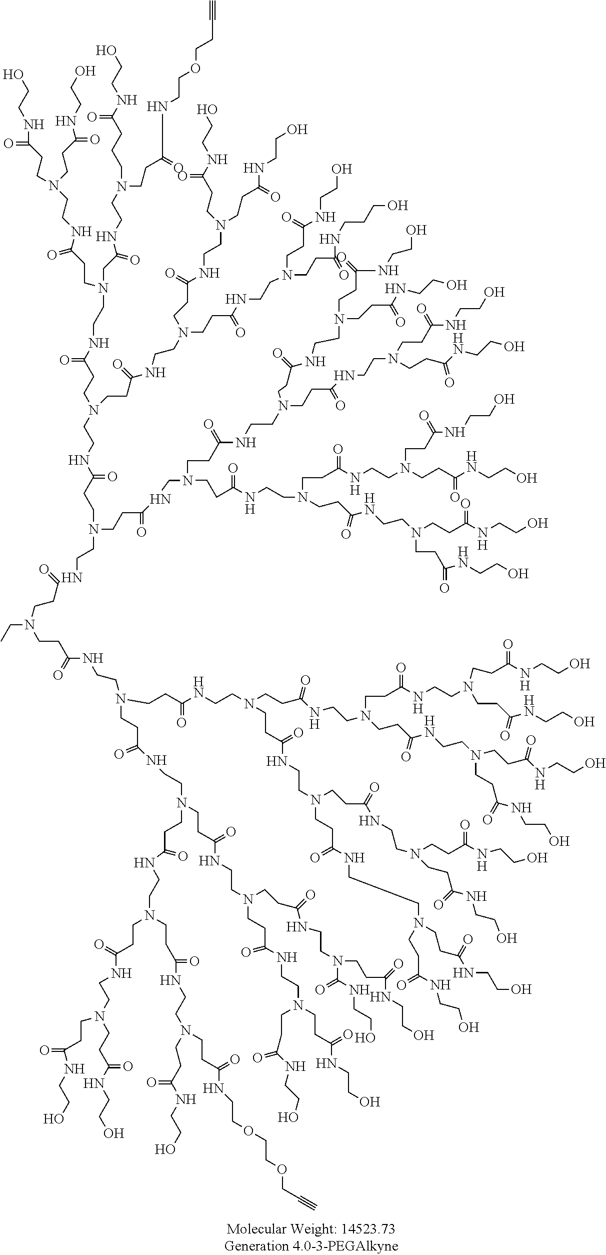

Based on 5 g validation run results, the process was scaled-up further to produce 40 g dendrimer functionalized with n=3 PEG-alkyne groups. A 1-Liter jacketed vessel was charged with a 9.42:1 molar ratio of ethanolamine and PEG-Alkyne followed by methanol. The vessel was purged with N₂(g) and cooled to 0° C. A 10 wt % of PAMAM G3.5 in methanol was then added over 2 hours to the reactor. The solution was then stirred for 2 hours at 0° C. before warming to 20° C. The solution was then allowed to stir at 20° C. for 6 days. After this time, the solution was diluted to half the concentration with water and subjected to purification by ultrafiltration providing the product as an aqueous solution. The solution was frozen, and water removed by lyophilization providing the product as a light-yellow foam. A small sample of the foam was taken for analysis by ¹H-NMR and ¹³C-NMR to determine the degree of alkyne functionalization using Method A and B described above. The remaining solid was dissolved in methanol to generate a 15.3 wt % methanolic solution (LOT #DP07-82-2). A summary of results for the scale-up is provided in Table 38.

TABLE 38
Summary of results for experiment DP07-82-1 (LOT# DP07-82-2)

| | |
|---|---|
| Lot number | DP07-82-2 |
| PAMAM G4 methanol | 300.4 |
| solution (g) | |
| Potency (% w/w) | 15.3% |
| Yield (g) | 46.0 |
| Yield (%) | 79% |
| Polydispersity (M_{w}/Mₙ) | 1.034 |
| Average # of PEG-alkyne | 3.02 |
| arms (from Method A and B) | |
| —OH/—NH ratio | 0.46 |
| Residual ethanolamine (dry | Below LOD |
| basis, GC) | |
| Residual PEG-Alkyne (dry | Below LOD |
| basis, GC) | |
| ¹³C-NMR | |

Scale-Up to Produce 160-Gram PAMAM G4.0 with 10.0 Alkyne Arms (DP07-85-1)

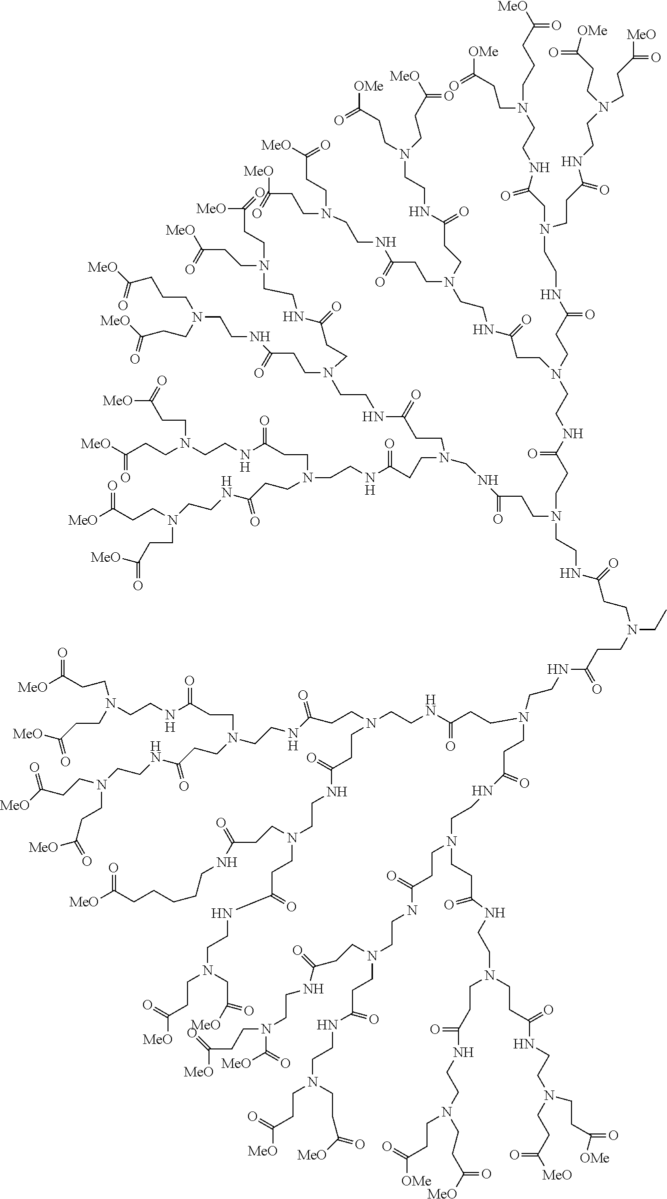

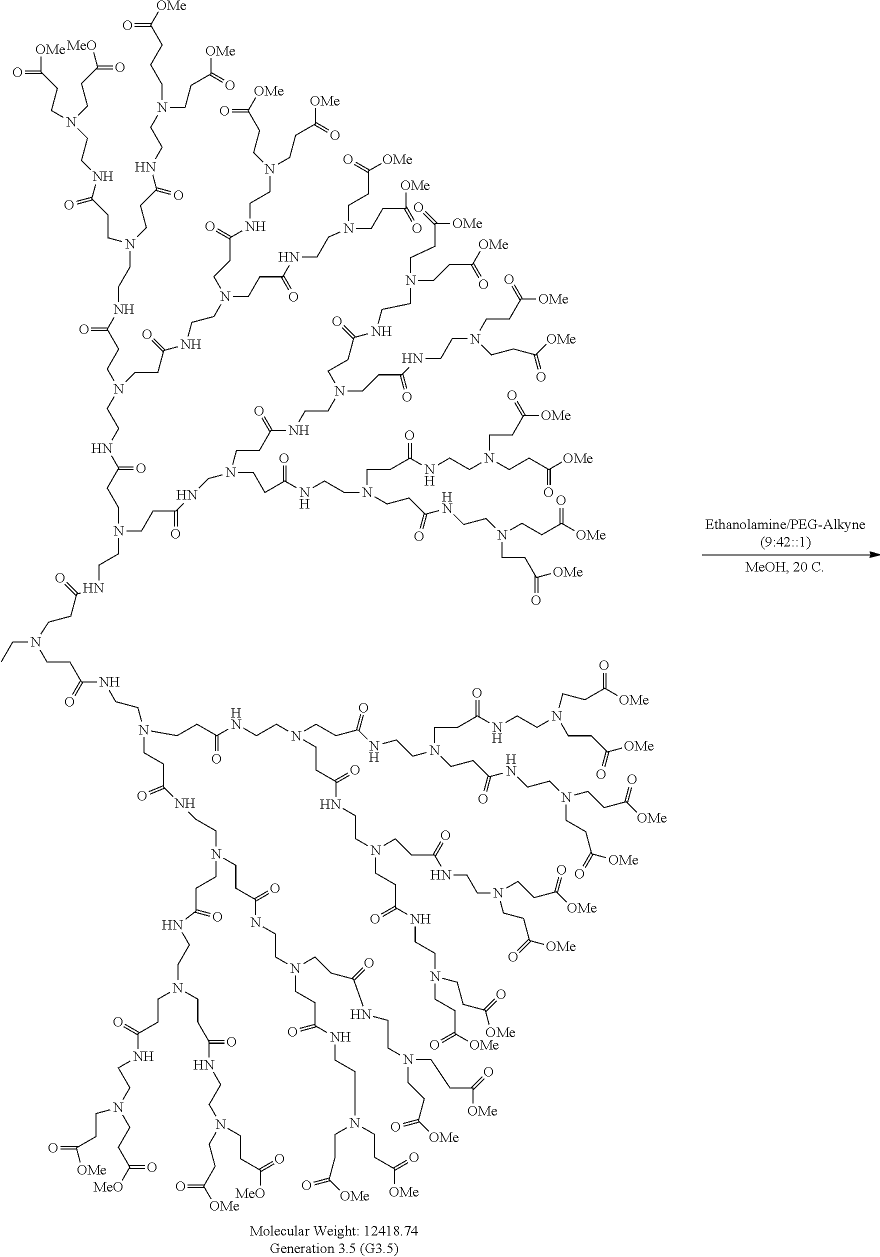

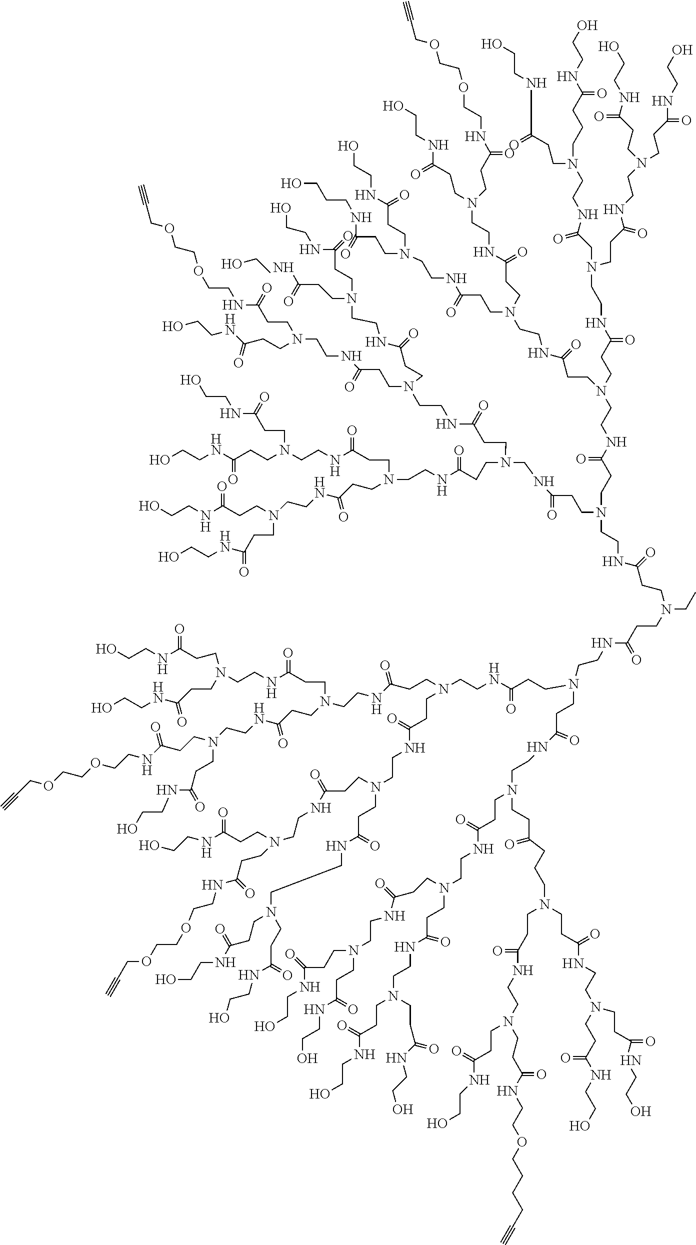

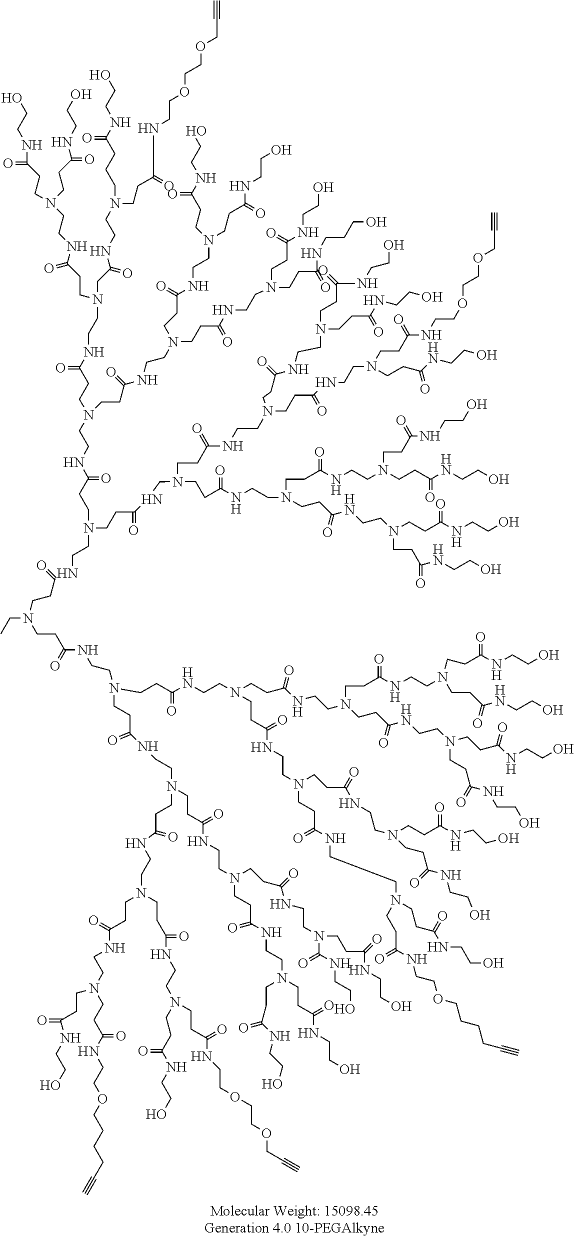

Similarly, as described above the synthesis of PAMAM G4.0 was scaled-up to produce dendrimer functionalized with n=10 PEG-alkyne. A 5-Liter jacketed vessel was charged with a 2.23:1 molar ratio of ethanolamine and PEG-Alkyne followed by methanol. The vessel was purged with N₂(g) and cooled to 0° C. A 10 wt % of PAMAM G3.5 in methanol was then added over 2 hours to the reactor. The solution was then stirred for 2 hours at 0° C. before warming to 20° C. The solution was then allowed to stir at 20° C. for 6 days. After this time, the solution was diluted to half the concentration with water and subjected to purification by ultrafiltration. The solution was frozen, and water removed by lyophilization providing the product as a light-yellow foam. A small sample of the foam was taken for analysis by ¹H-NMR and ¹³C-NMR to determine the degree of alkyne functionalization using Method A and B described in Example 4. The remaining solid was dissolved in methanol to generate a 15.3 wt % methanolic solution (LOT #DP07-85-3). A summary of results for the scale-up is provided in Table 39.

TABLE 39
Summary of results for experiment DP07-85-1 (LOT# DP07-85-3)

| | |
|---|---|
| Lot number | DP07-85-3 |
| PAMAM G4 methanol | 1469.6 |
| solution (g) | |
| Potency (% w/w) | 15.3% |
| Yield (g) | 224.85 |
| Yield (%) | 98% |
| Polydispersity (M_{w}/Mₙ) | 1.032 |
| Average # of PEG-alkyne | 10.01 |
| arms (from Method A and B) | |
| —OH/—NH ratio | 0.34 |
| Residual ethanolamine (dry | 0.1% |
| basis, GC) | |
| Residual PEG-Alkyne (dry | 0.1% |
| basis, GC) | |
| ¹³C-NMR | |

### Conclusion

A process for producing PAMAM G4-OH-Alkyne₃ in >40 g quantities and PAMAM G4-OH-Alkyne₁₀ >224 g was successfully developed. Quality attributes of the products are within the expected control range.

### Experimental

### PAMAM G4-OH-Alkyne₃ Synthesis—Lot DP07-82-2

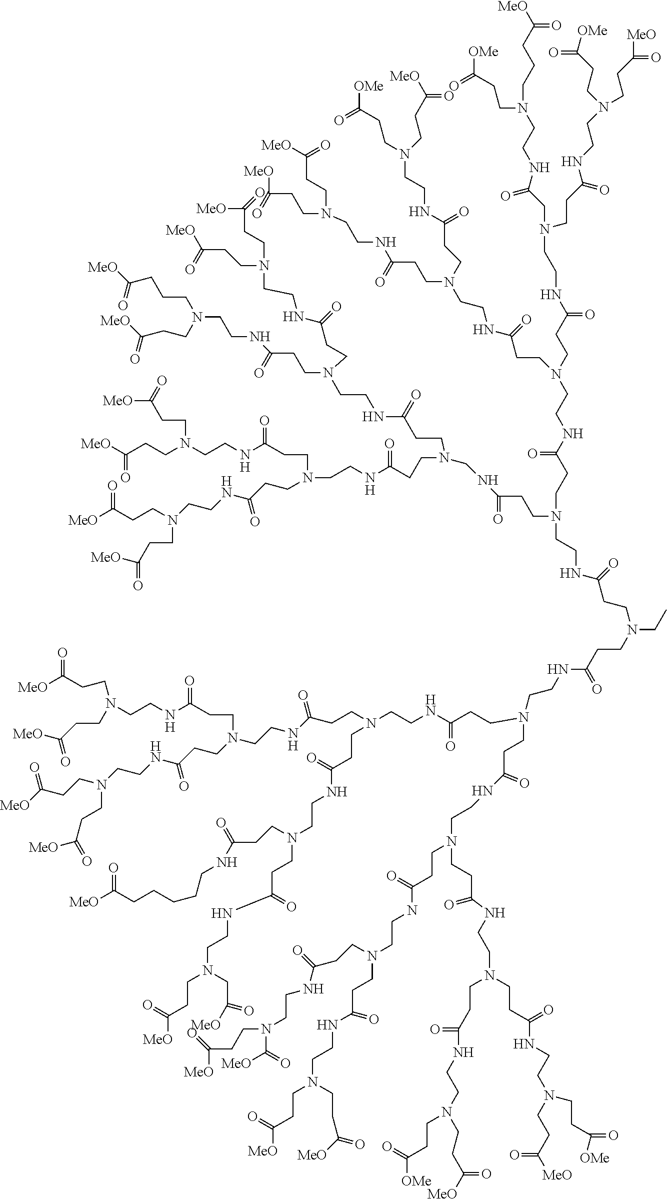

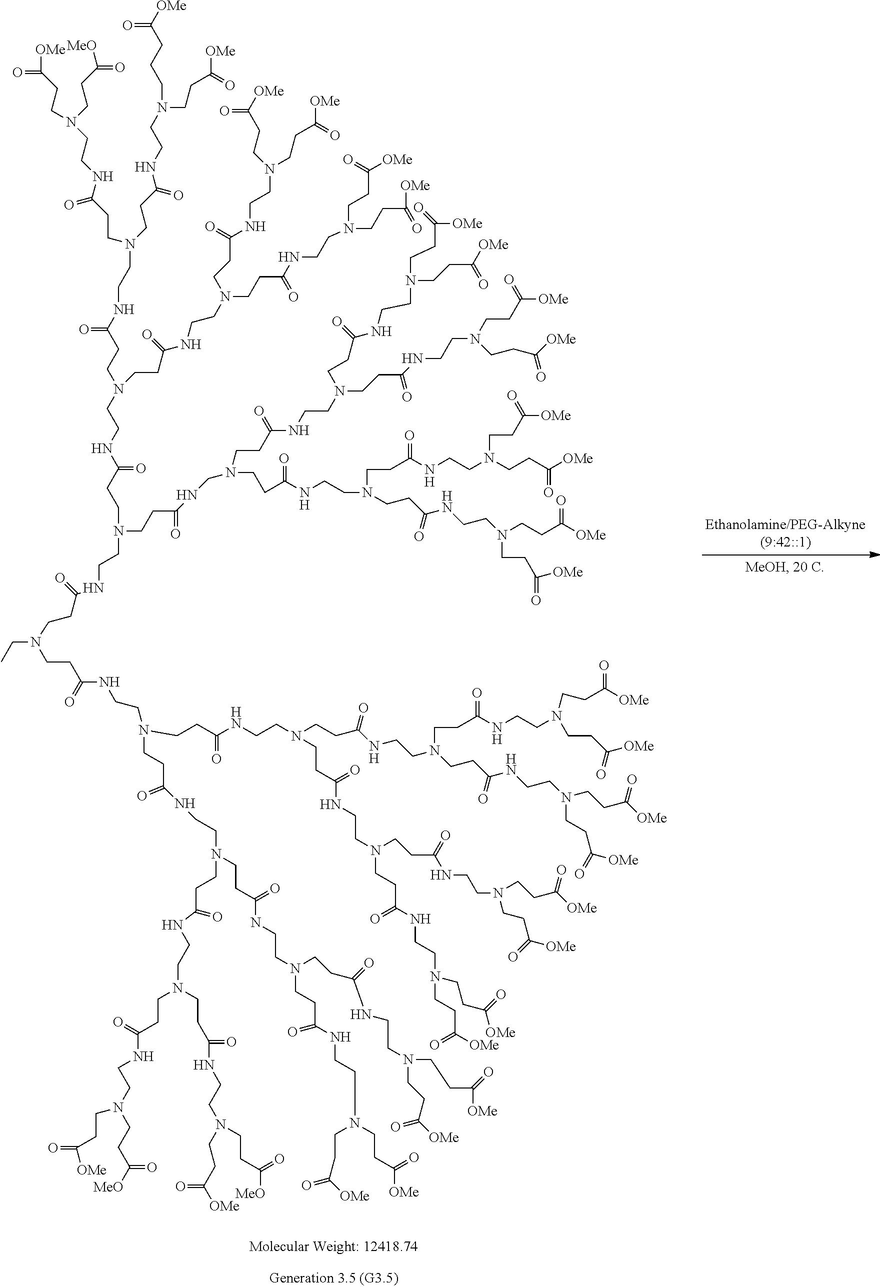

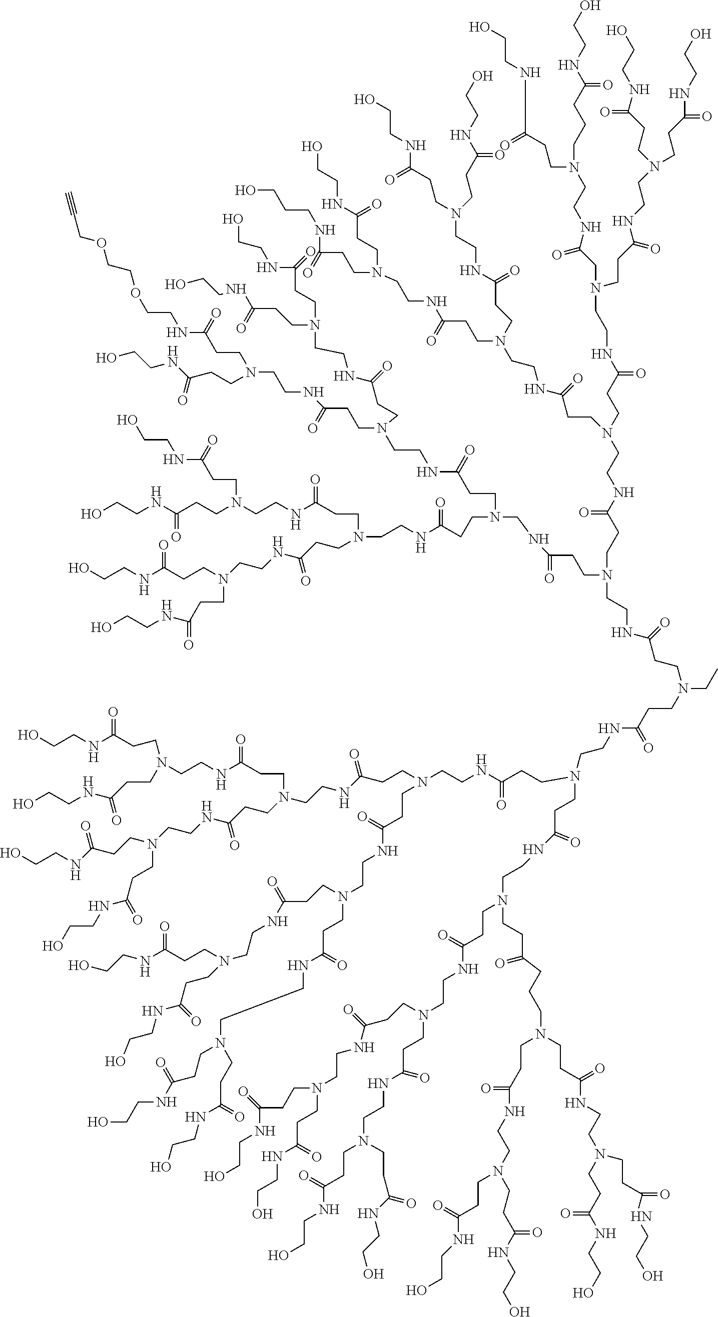

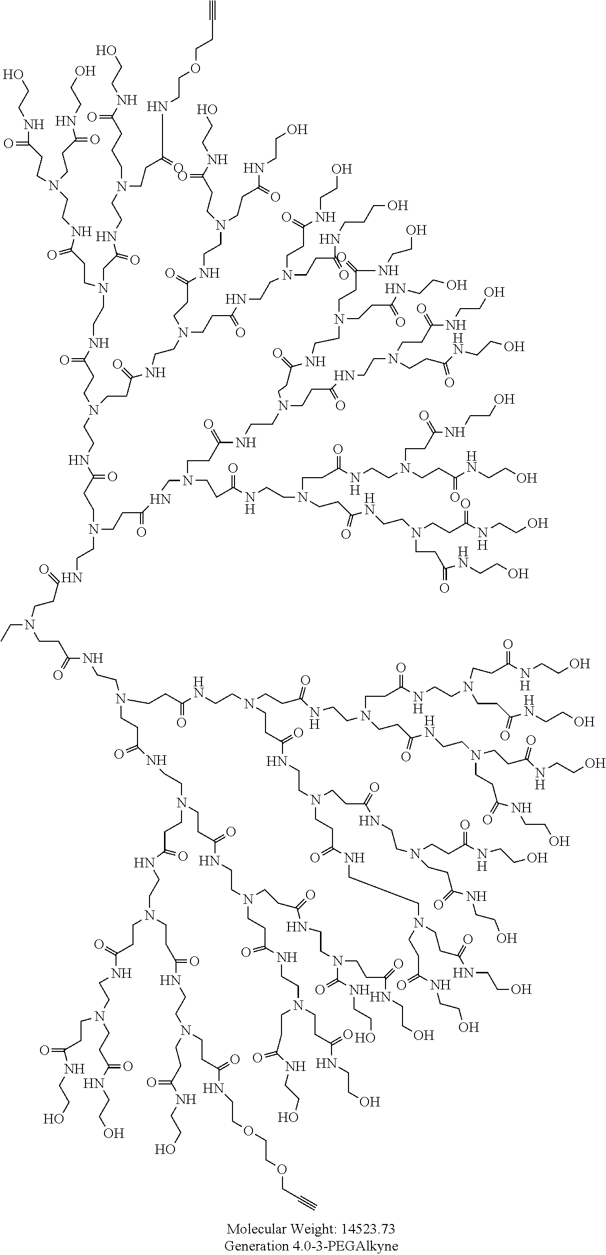

| | MW | Potency | Density | Amount | Amount | | |
|---|---|---|---|---|---|---|---|
| Reagent | (g/mol) | (% w/w) | (g/mL) | (g) | (mL) | mmol | Eq/vol |
| Amine solution | | | | 536.07 | | 6441.96 | 1600 |
| Ethanolamine | 61.08 | 100.00 | 1.01 | 355.74 | | 5823.73 | |
| 2-[2-(2- | 143.19 | 96.5% | 1.00 | 91.74 | | 618.23 | |
| Propynyloxy)ethoxylethylamine | | | | | | | |
| MeOH | | 100.00 | 0.79 | 88.60 | 111.87 | | 0.25 |
| PAMAM G3.5 solution (10 wt %) | | | | 500.01 | | | |
| PAMAM G3.5 solution (29.4 | 12418.72 | 29.40 | 0.80 | 170.07 | 212.59 | 4.026 | 1 |
| wt %) | | | | | | | |
| MeOH | | 100.00 | 0.79 | 329.94 | 416.59 | | |

A 1-Liter jacketed vessel was charged with a 9.42:1 molar ratio of ethanolamine and PEG-Alkyne (Ambeed, Lot #100902012-00223 0BFY) followed by methanol. The vessel was purged with N₂(g) and cooled to 0° C. A 10 wt % of PAMAM G3.5 (Dentritech, lot #0121-02-E3.5-LD-A) in methanol was then added over 2 hours to the reactor. The solution was then stirred for 2 hours at 0° C. before warming to 20° C. The solution was then allowed to stir at 20° C. for 6 days. After this time, the solution was diluted to half the concentration with water and subjected to purification by ultrafiltration providing the product as an aqueous solution. The solution was frozen, and water removed by lyophilization providing the product as a light-yellow foam. A small sample of the foam was taken for analysis by ¹H-NMR and C-NMR to determine the degree of alkyne functionalization using Method A and B described in Example 4. The remaining solid was dissolved in methanol to generate a 15.3 wt % methanolic solution (LOT #DP07-82-2). A summary of results for the scale-up is provided in Table 40.

TABLE 40
Summary of results for experiment DP07-82-1 (LOT# DP07-82-2)

| | |
|---|---|
| Lot number | DP07-82-2 |
| PAMAM G4 methanol | 300.4 |
| solution (g) | |
| Potency (% w/w) | 15.3% |
| Yield (g) | 46.0 |
| Yield (%) | 79% |
| Polydispersity (M_{w}/Mₙ) | 1.034 |
| Average # of PEG-alkyne | 3.02 |
| arms (from Method A and B) | |
| —OH/—NH ratio | 0.46 |
| Residual ethanolamine (dry | Below LOD |
| basis, GC) | |
| Residual PEG-Alkyne (dry | Below LOD |
| basis, GC) | |
| ¹³C-NMR | |

### PAMAM G4-OH-Alkyne₁₀ Synthesis—Lot DP07-85-3

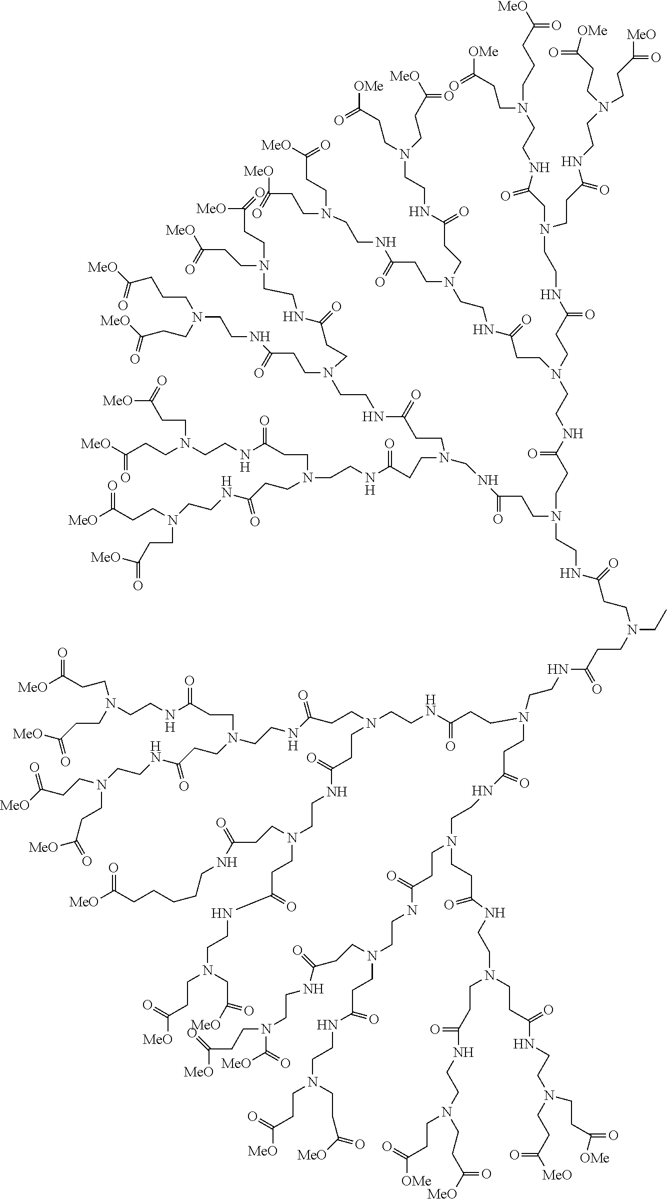

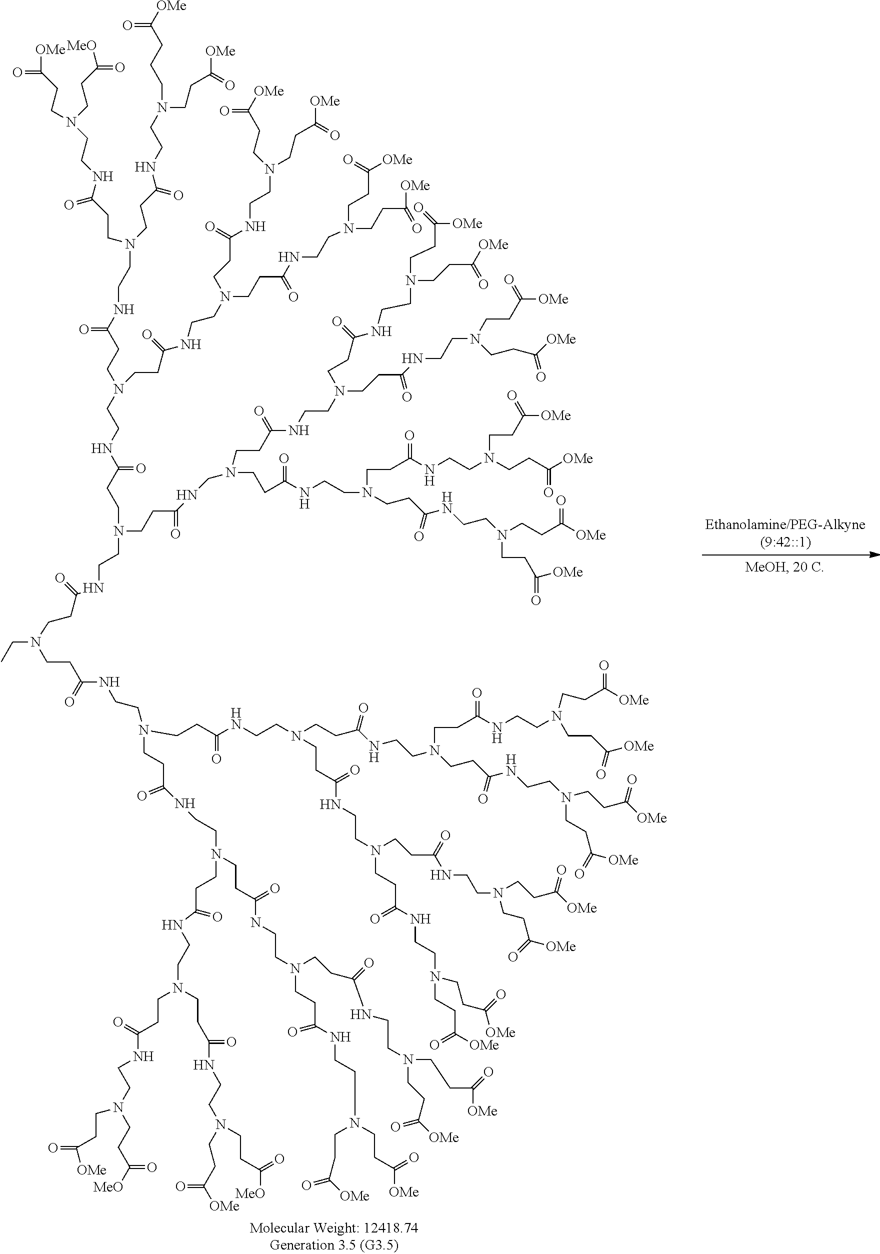

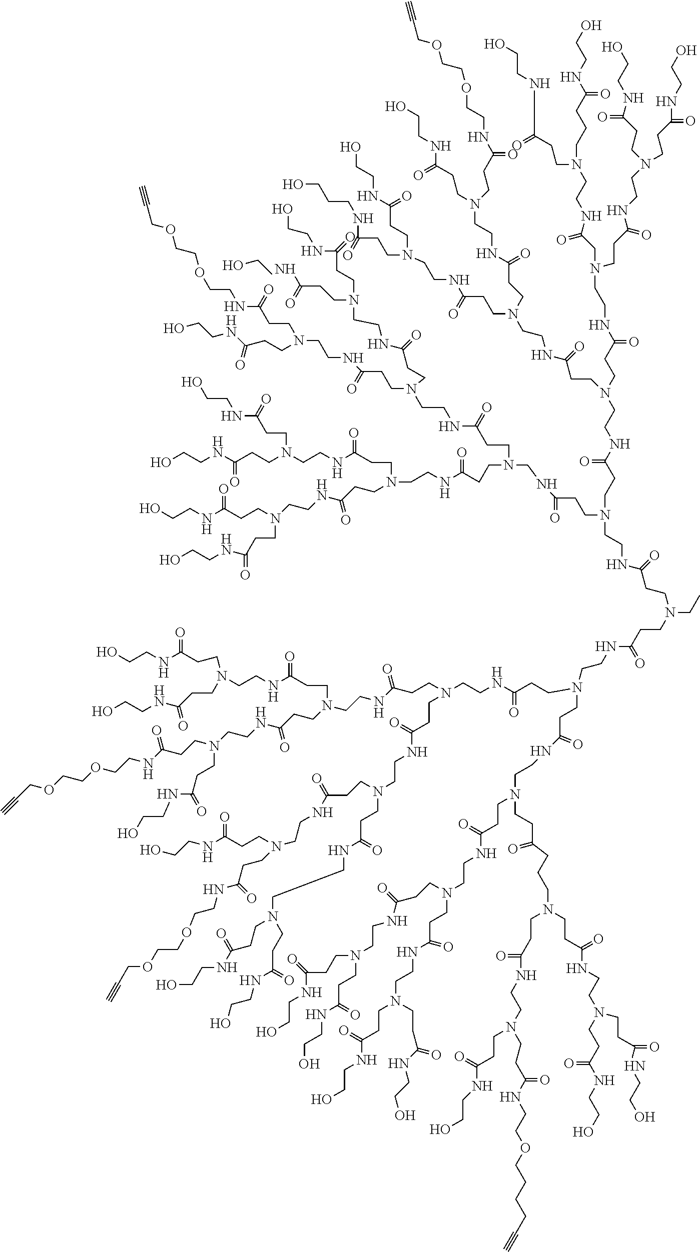

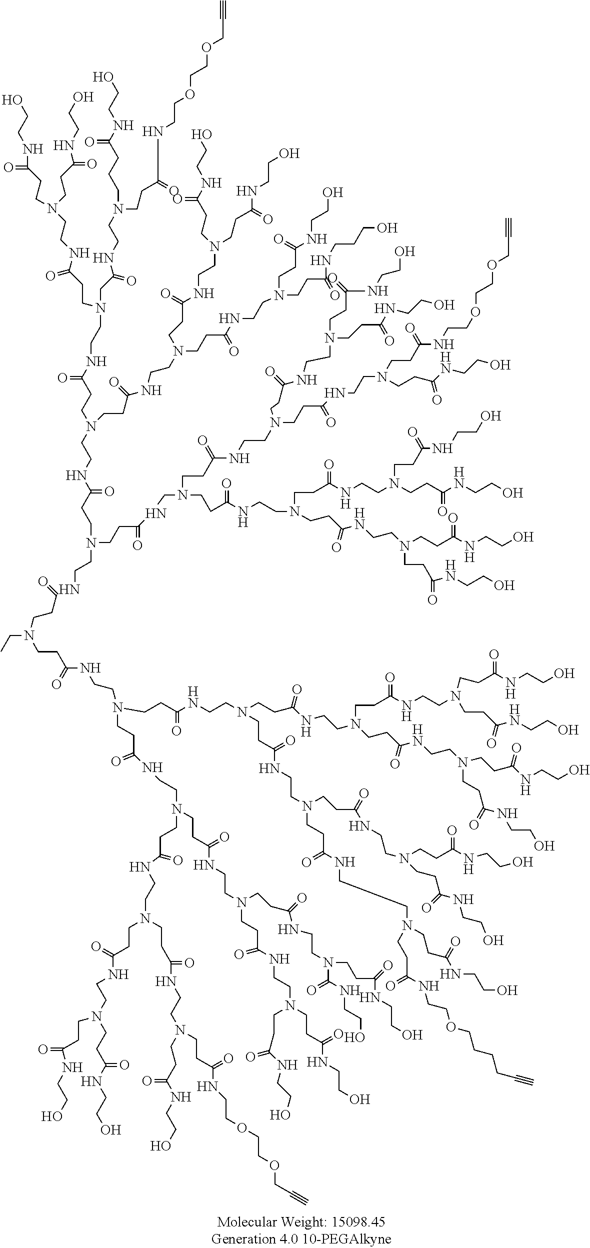

| | MW | Potency | Density | Amount | Amount | | |
|---|---|---|---|---|---|---|---|
| Reagent | (g/mol) | (% w/w) | (g/mL) | (g) | (mL) | mmol | Eq/vol |
| Amine solution | | | | 2558.94 | | 24242.1 | 1600 |
| | | | | | | 1 | |
| Ethanolamine | 61.084 | 100.00 | 1.0117 | 1022.35 | | 16736.81 | |
| 2-[2-(2- | 143.19 | 97% | 1.00 | 1113.66 | | 7505.30 | |
| Propynyloxy)ethoxylethylamine | | | | | | | |
| MeOH | | 100.00 | 0.792 | 422.93 | 534.00 | | 0.25 |
| PAMAM G3.5 solution (10 | | | | 1881.60 | | | |
| wt %) | | | | | | | |
| PAMAM G3.5 solution (29.4 | 12418.72 | 29.40 | 0.8 | 640.00 | 800.00 | 15.151 | 1 |
| wt %) | | | | | | | |
| MeOH | | 100.00 | 0.792 | 1241.60 | 1567.68 | | |

A 5-Liter jacketed vessel was charged with a 2.23:1 molar ratio of ethanolamine and PEG-Alkyne (Ambeed, Lot #100902012-00223 0BFY) followed by methanol. The vessel was purged with N₂(g) and cooled to 0° C. A 10 wt % of PAMAM G3.5 (Dentritech, lot #0121-02-E3.5-LD-A) in methanol was then added over 2 hours to the reactor. The solution was then stirred for 2 hours at 0° C. before warming to 20° C. The solution was then allowed to stir at 20° C. for 6 days. After this time, the solution was diluted to half the concentration with water and subjected to purification by ultrafiltration. The solution was frozen, and water removed by lyophilization providing the product as a light-yellow foam. A small sample of the foam was taken for analysis by H-NMR and ¹³C-NMR to determine the degree of alkyne functionalization using Method A and B described above. The remaining solid was dissolved in methanol to generate a 15.3 wt % methanolic solution (LOT #DP07-85-3). A summary of results for the scale-up is provided in Table 41.

TABLE 41
Summary of results for experiment DP07-85-1 (LOT# DP07-85-3)

| | |
|---|---|
| Lot number | DP07-85-3 |
| PAMAM G4 methanol | 1469.6 |
| solution (g) | |
| Potency (% w/w) | 15.3% |
| Yield (g) | 224.85 |
| Yield (%) | 98% |
| Polydispersity (M_{w}/Mₙ) | 1.032 |
| Average # of PEG-alkyne | 10.01 |
| arms (from Method A and B) | |
| —OH/—NH ratio | 0.34 |
| Residual ethanolamine (dry | 0.1% |
| basis, GC) | |
| Residual PEG-Alkyne (dry | 0.1% |
| basis, GC) | |
| ¹³C-NMR | |

### Analytical Methods

### GC Method (ASV003F)

### A. Chromatographic Conditions

Column: Agilent CP-Volamine (Part number CP7447)
Inlet Temperature: 250° C.
Inlet Pressure: 14.3 psi
Inlet Total flow: 37.3 mL/min
Inlet Split Ratio: 10:1
Injection Volume: 5 μL
Column Flowrate: 3.1 mL/min
FID Temperature: 300° C.
FID H₂ Flow: 40.0 mL/min
FID Air Flow: 360.0 mL/min
FID Makeup Flow: 25 mL/min
FID Lit Offset: 0.5 pA
Run Time: 20.33 min

Oven Temperature Gradient

| Rate (° C./min) | Temperature (° C.) | Hold time | Run time |
|---|---|---|---|
| N/A | 65 | 4 | 4 |
| 15 | 100 | 3 | 9.33 |
| 30 | 250 | 6 | 20.33 |

### D. Calculation

$$Weight \% = ( \frac{{A}_{c} - {A}_{b}}{{R}_{a}} ) / {C}_{s} \times 100$$

$${A}_{b} = Total area counts from the blank \\ {A}_{c} = Area counts of component in sample injection \\ {R}_{a} = Response factor of component \\ {C}_{s} = Concentration of sample$$

For determining impurities using this method, standards of 14.928 and 10.332 mg/mL of ethanolamine and PEG alkyne in isopropyl alcohol respectively were prepared and injected onto the GC. This gave a retention time of ethanolamine around 6.88 minutes while the PEG alkyne had a retention time of 14.1 minutes. These solutions were combined in a 1:1 ratio. This combined solution was used as a stock solution for ½ serial dilutions using isopropyl alcohol reaching concentrations of 0.007 and 0.005 mg for Ethanolamine and PEG alkyne respectively. The results of the calibration are shown below in Table 42.

TABLE 42
GC Calibration data for ethanolamine and PEG-Alkyne

| Concentration of | Concentration of PEG | ethanolamine | PEG-alkyne |
|---|---|---|---|
| Ethanolamine (mg/mL) | alkyne (mg/mL) | signal area | signal area |
| 14.928 | 10.332 | 22543.5 | 22341 |
| 7.4640 | 5.1660 | 11862.6 | 11688.7 |
| 3.7320 | 2.5830 | 5755.5 | 5656.6 |
| 1.8660 | 1.2915 | 2962 | 2966 |
| 0.93300 | 0.64575 | 1423.5 | 1486.3 |
| 0.46650 | 0.32288 | 708.2 | 758.3 |
| 0.23325 | 0.16144 | 323.2 | 360.1 |
| 0.11663 | 0.080719 | 153.4 | 177.7 |
| 0.058313 | 0.040359 | 72.9 | 91 |
| 0.029156 | 0.020180 | 30.5 | 42.3 |
| 0.014578 | 0.010090 | 13 | 21 |
| 0.0072891 | 0.0050449 | 4.7 | 11.5 |

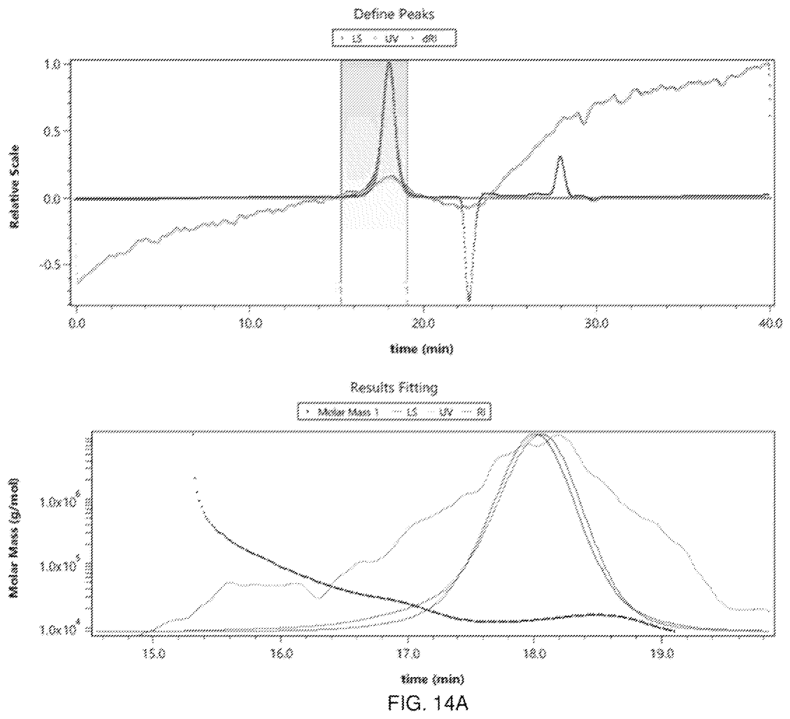

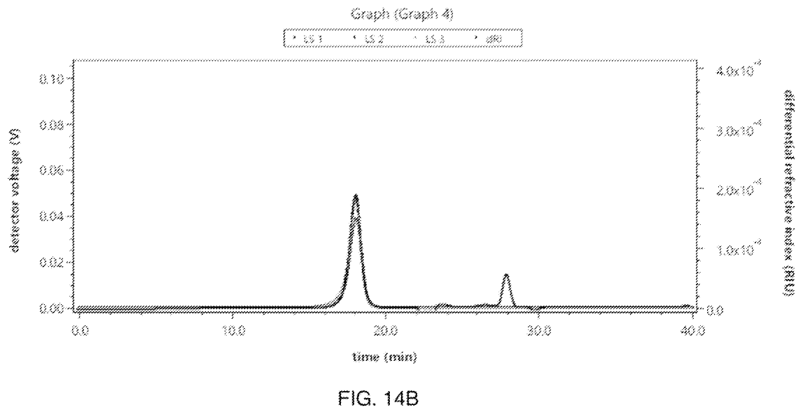

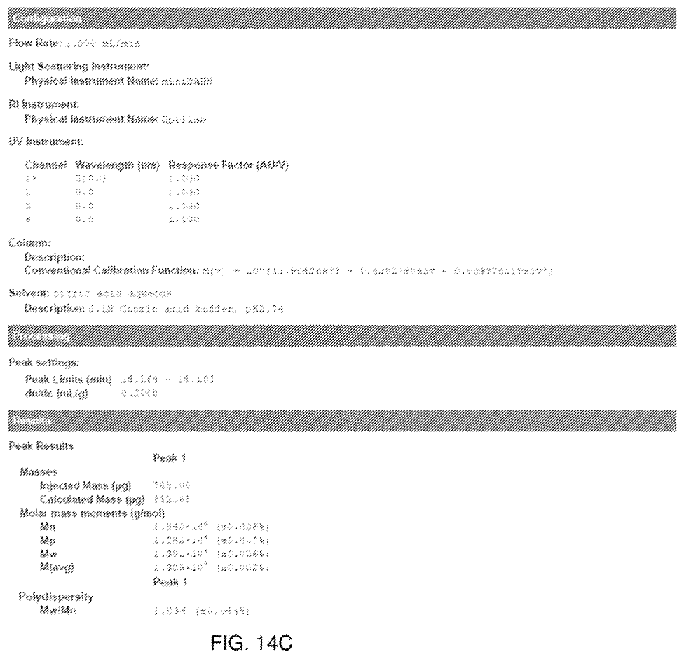

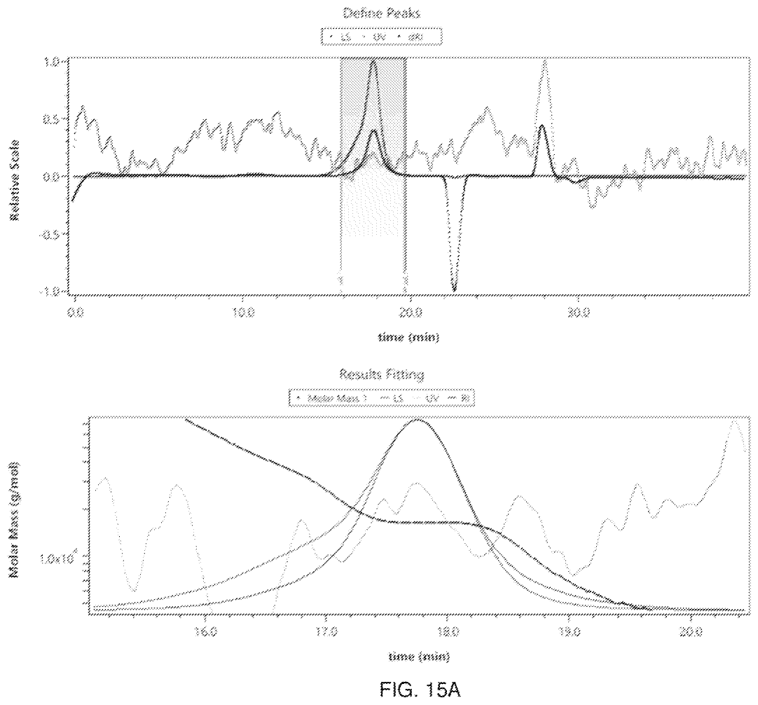

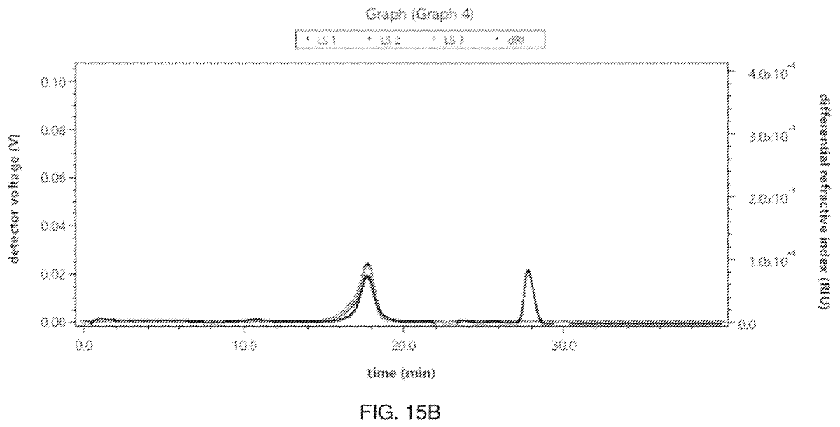

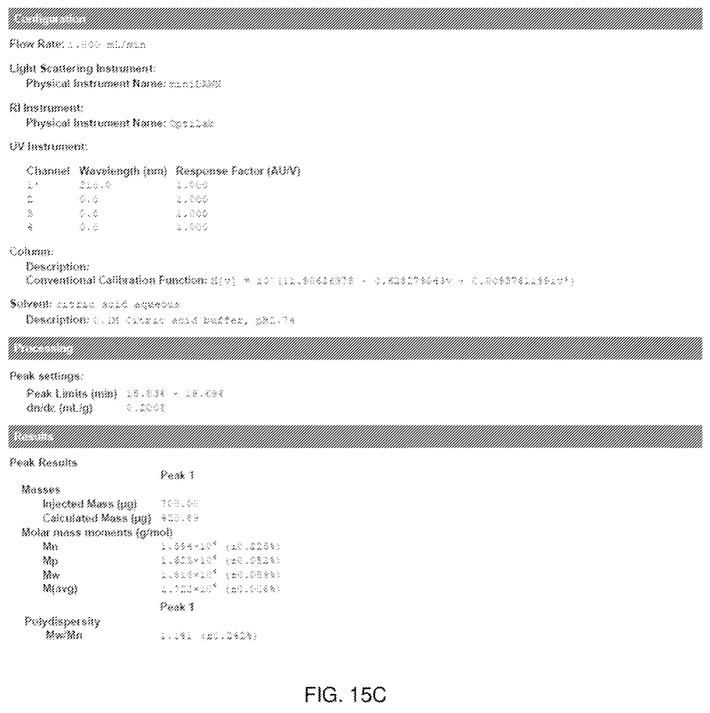

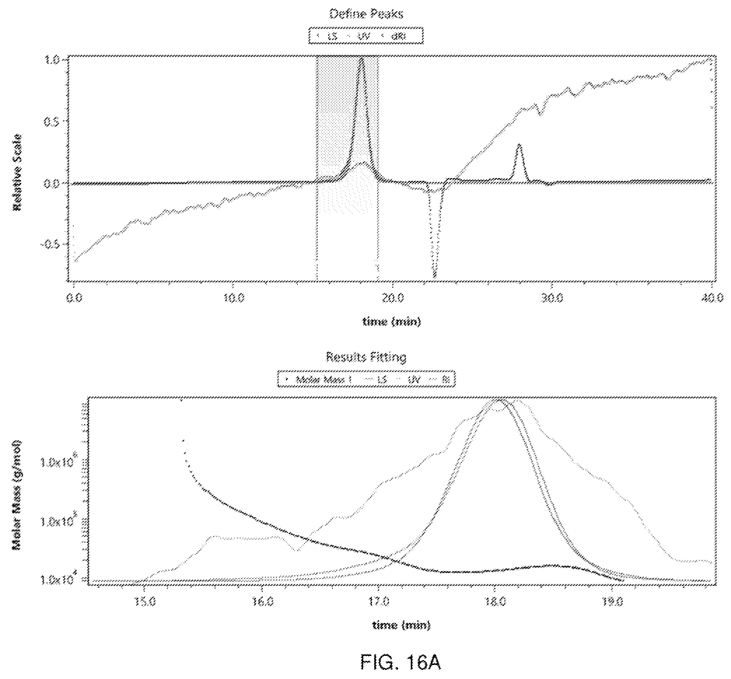

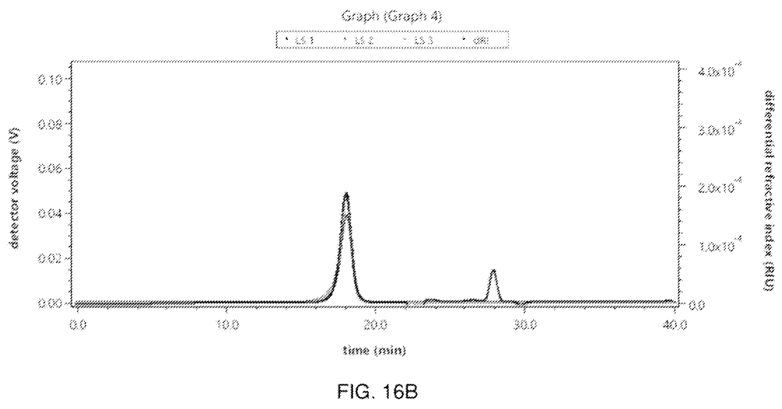

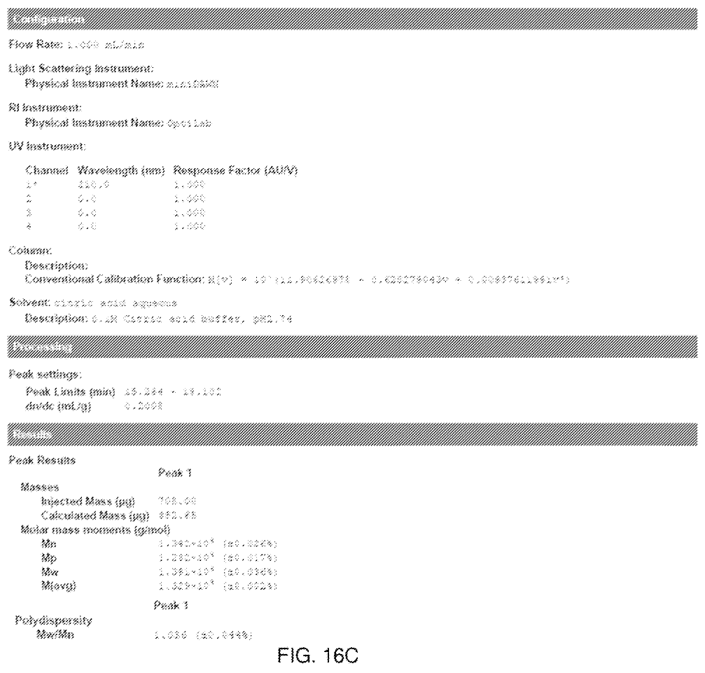

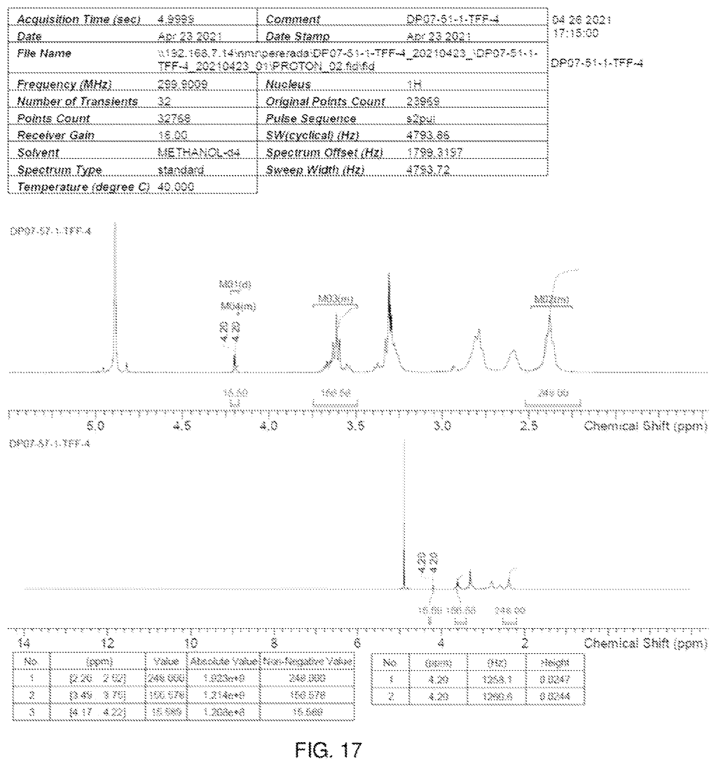

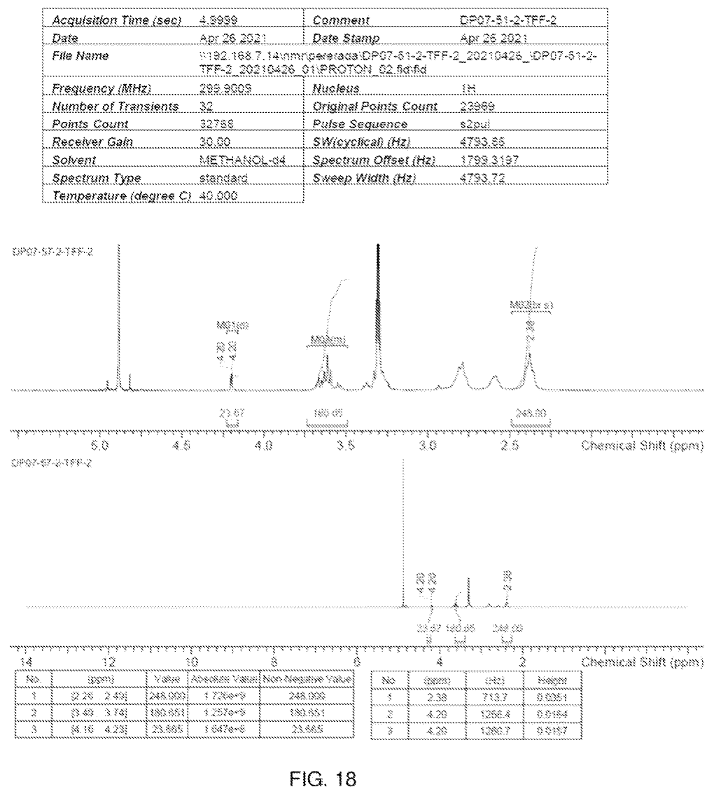

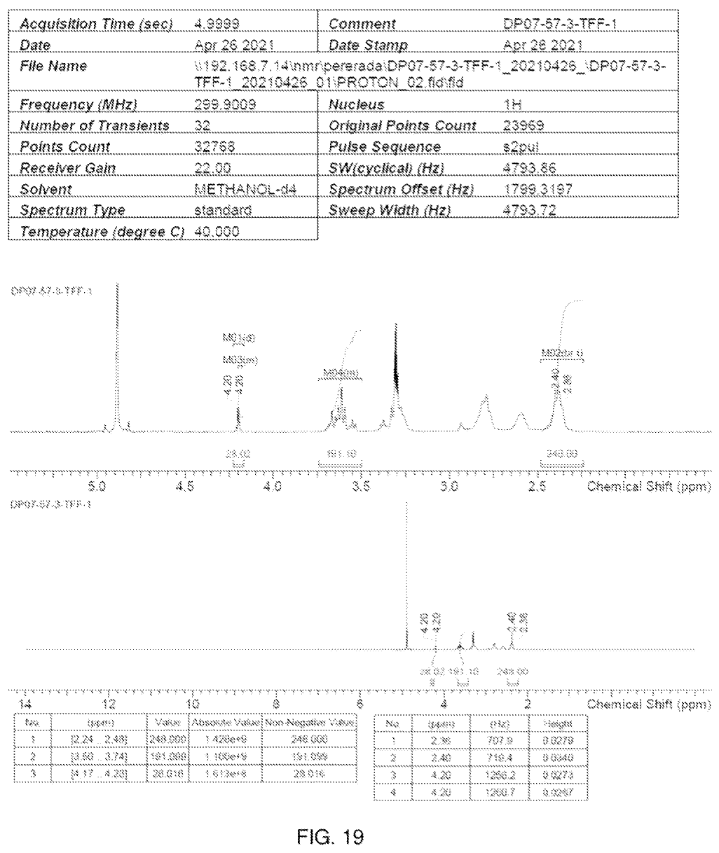

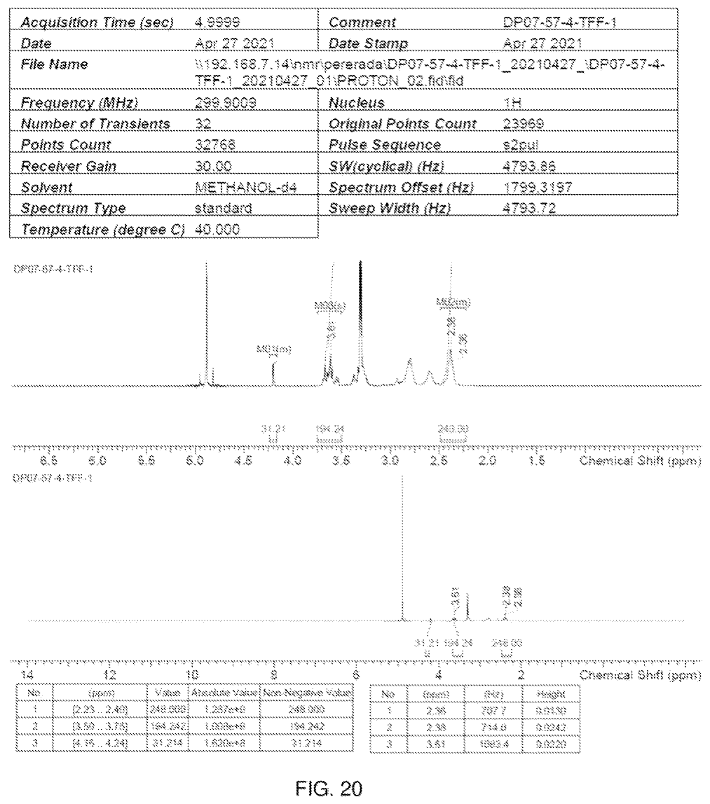

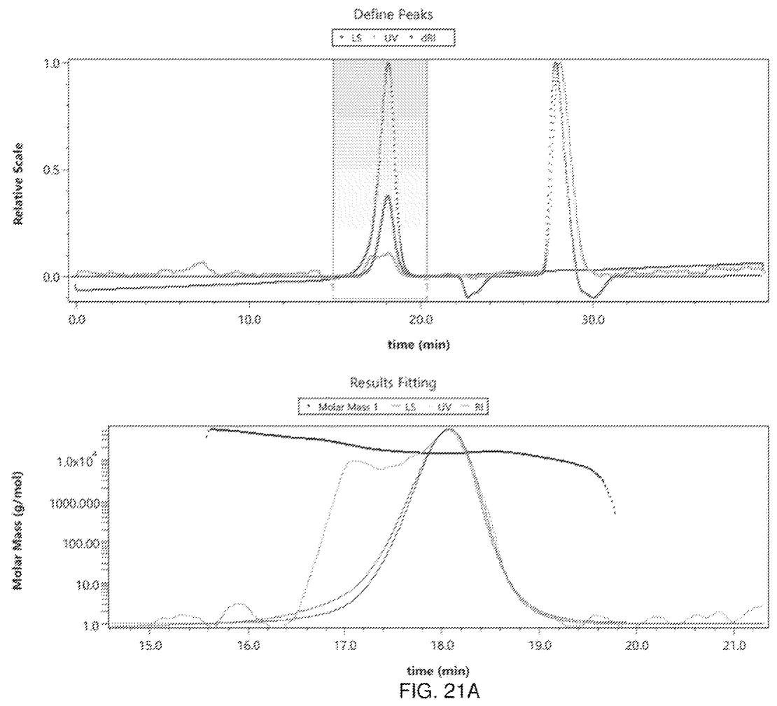

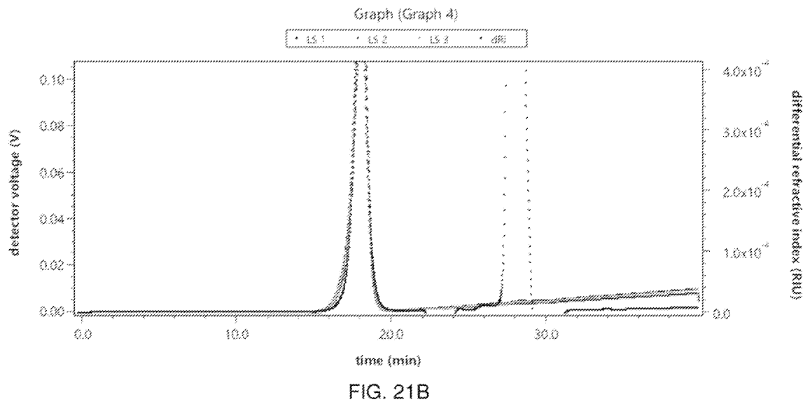

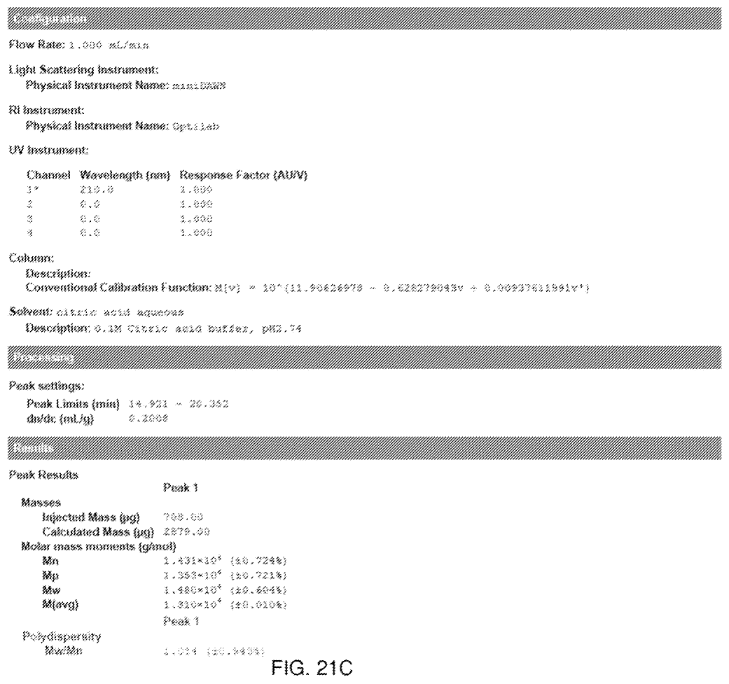

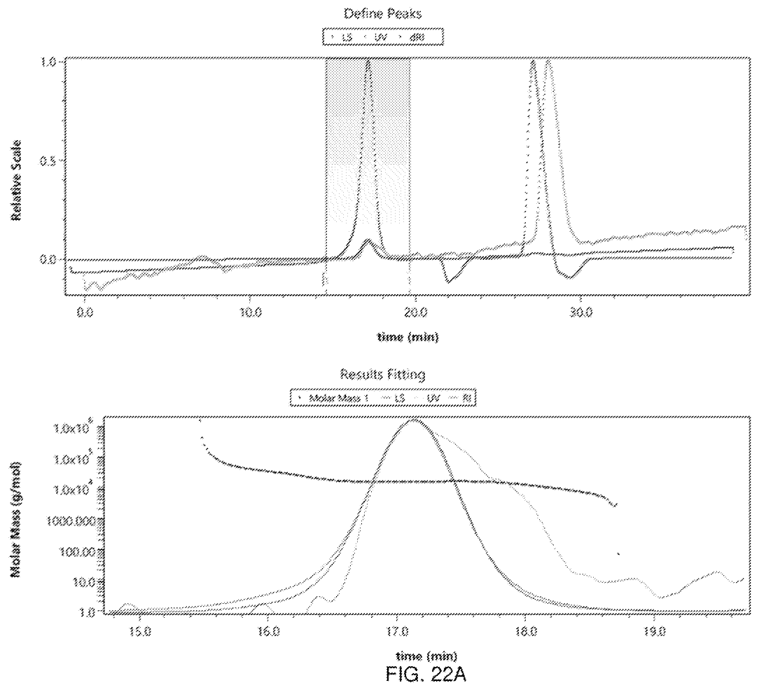

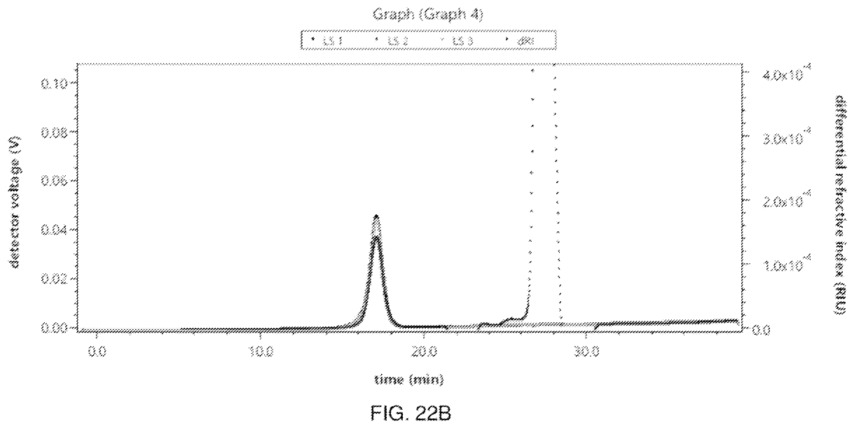

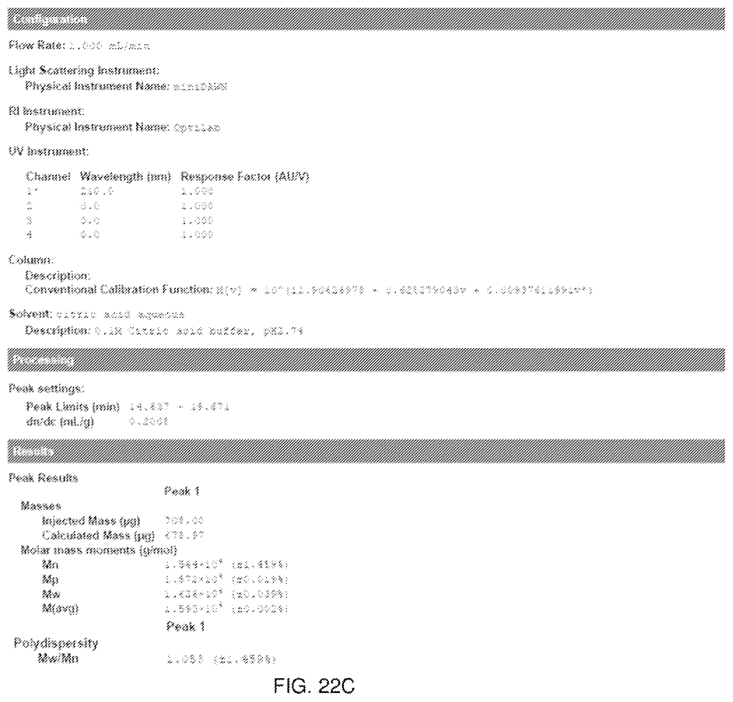

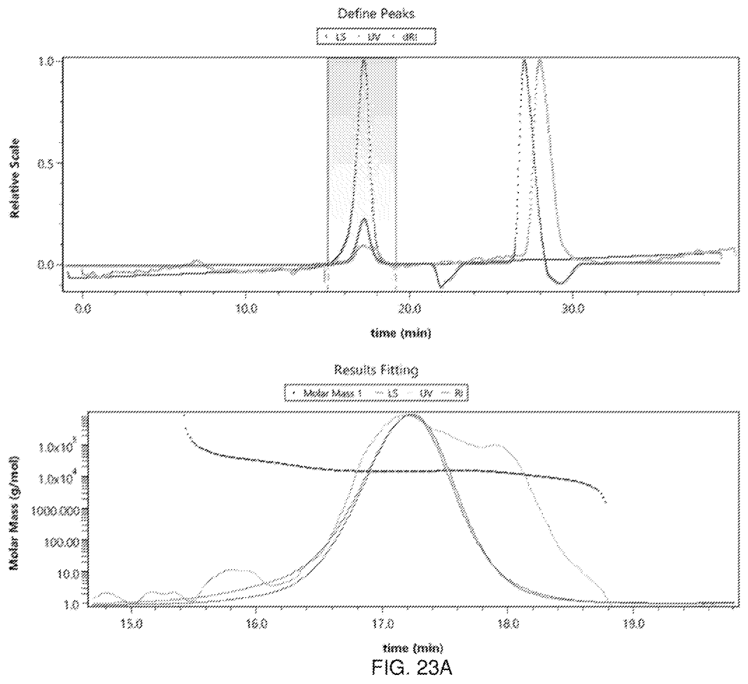

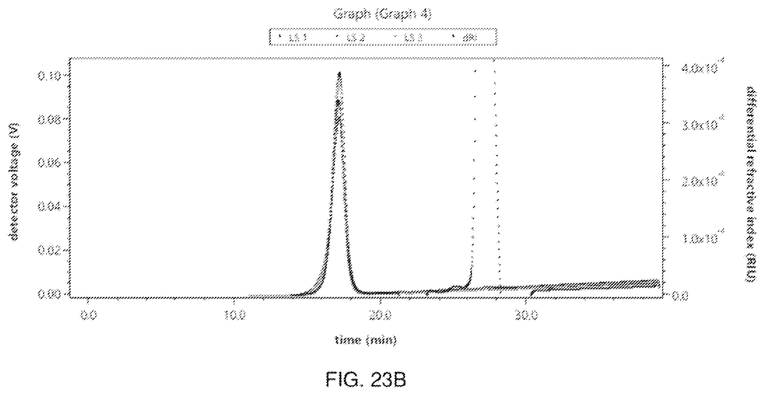

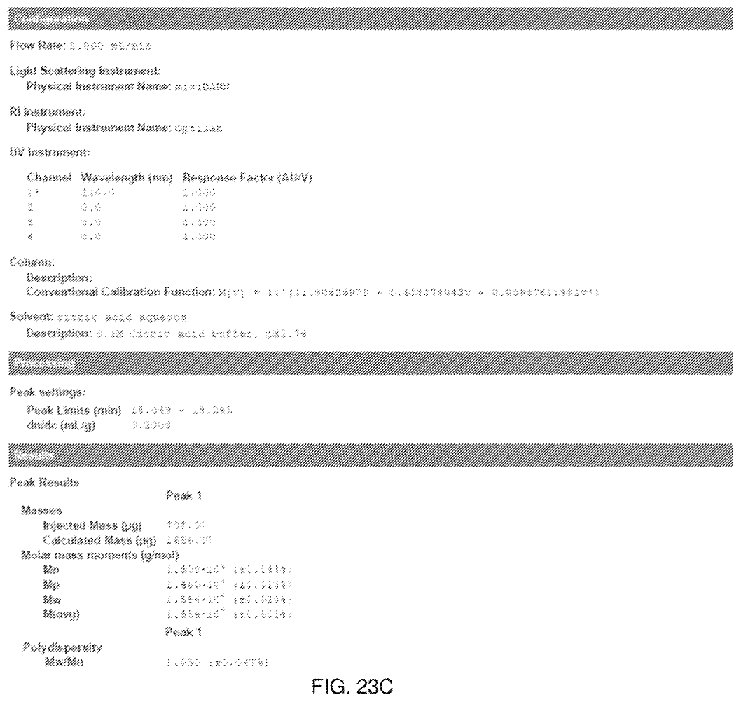

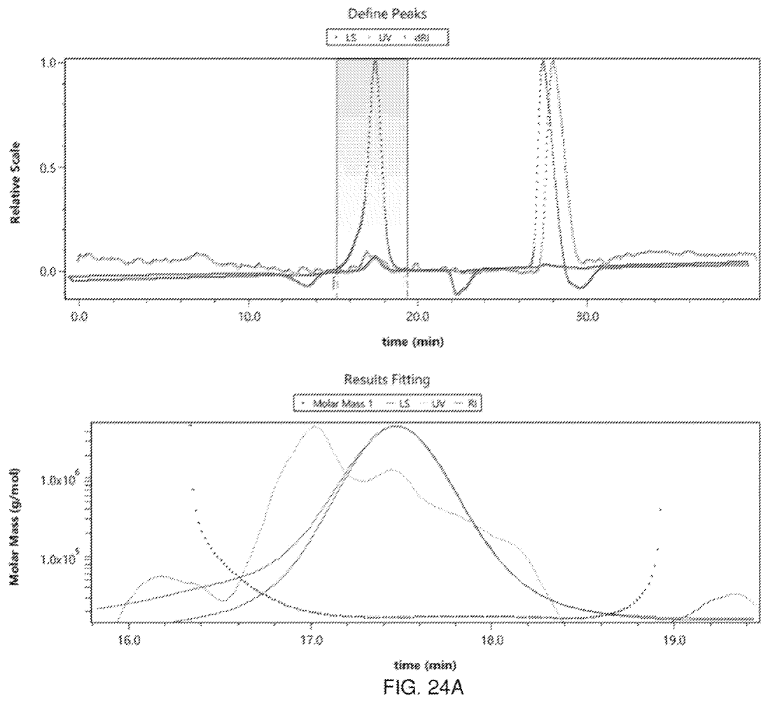

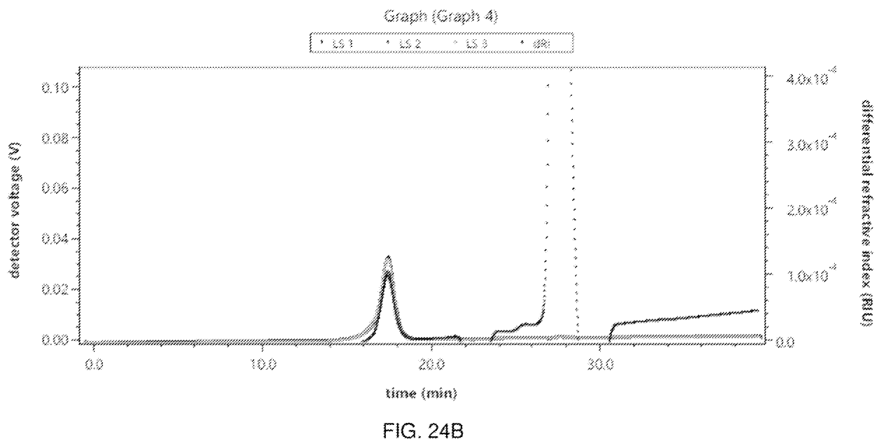

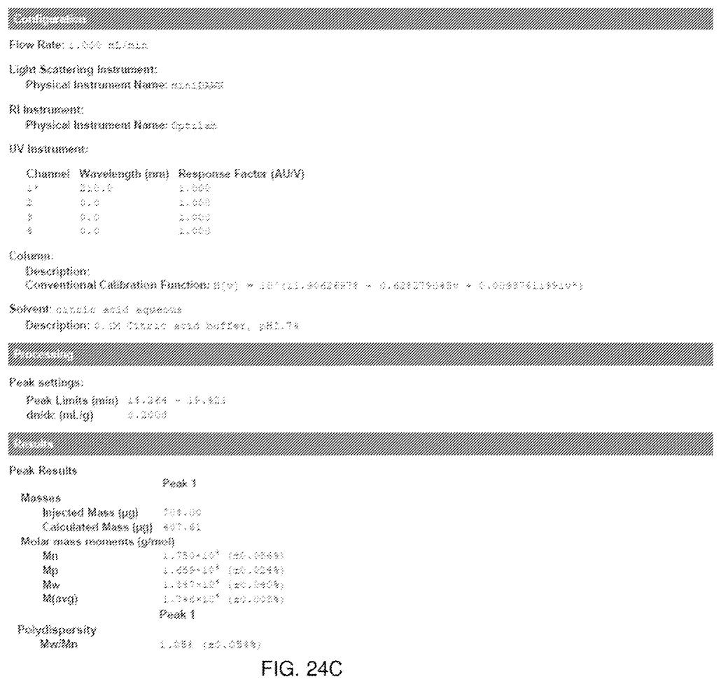

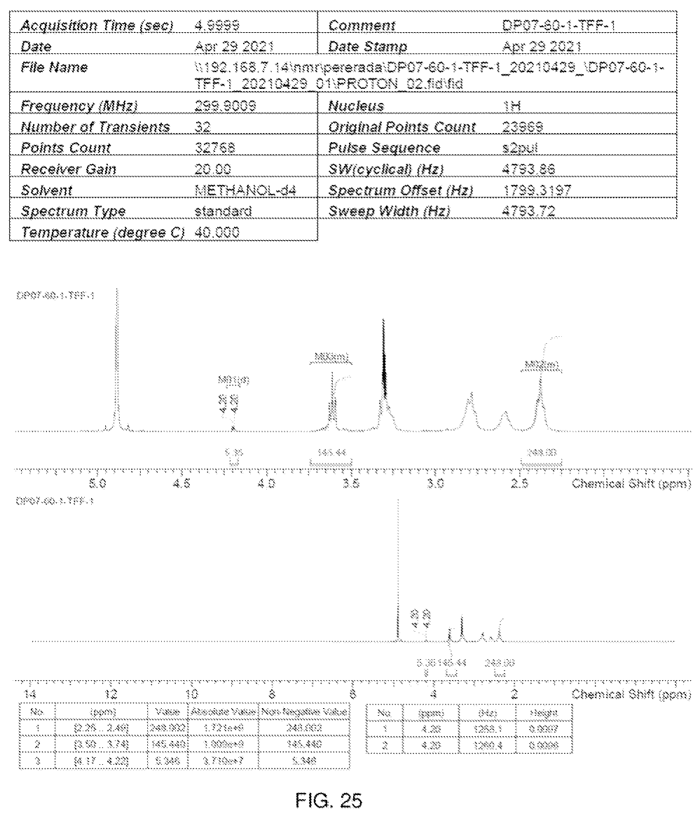

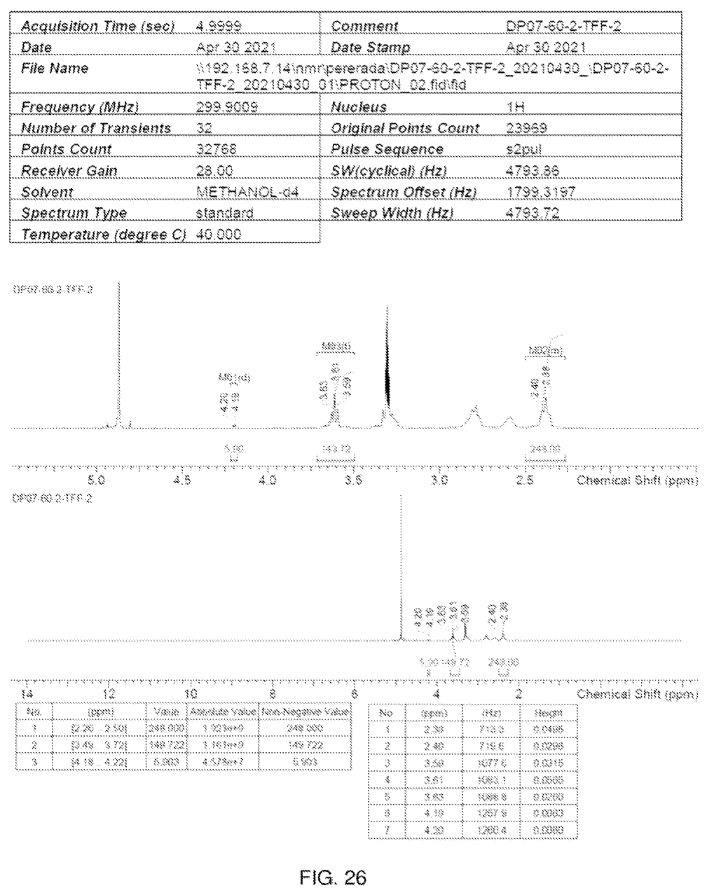

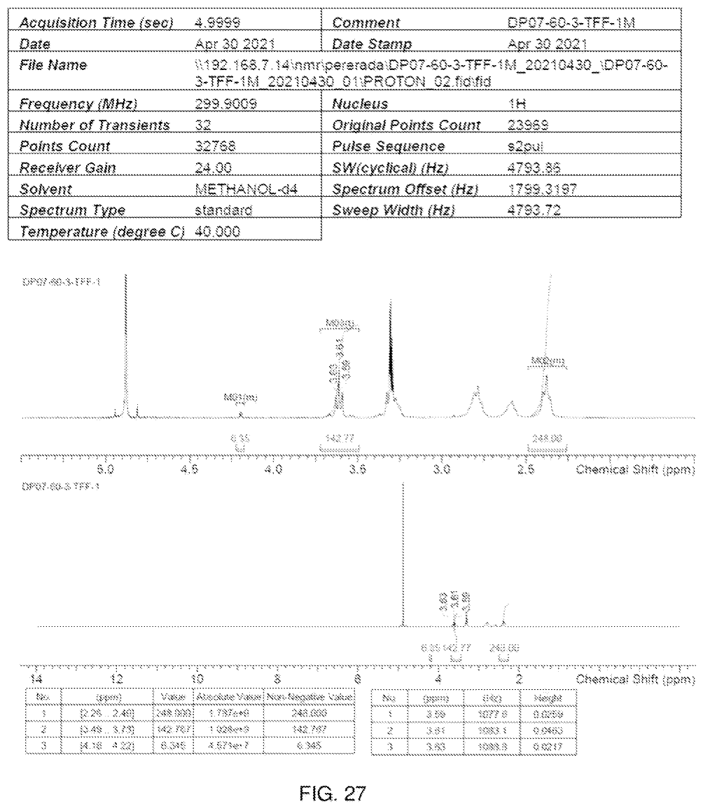

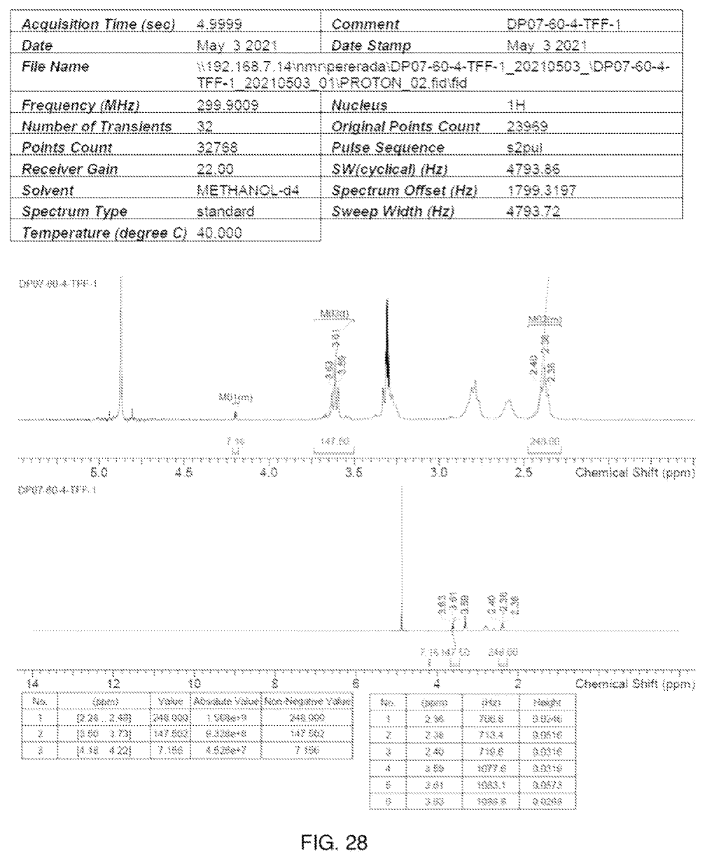

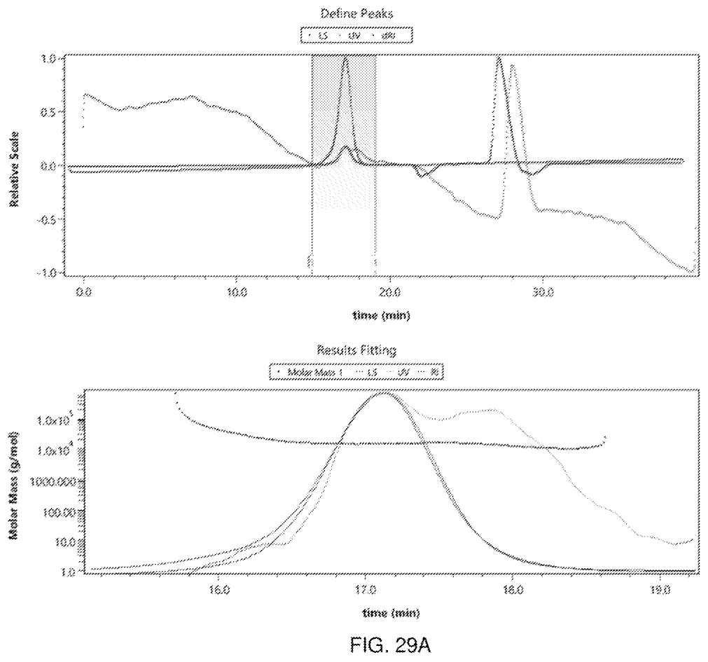

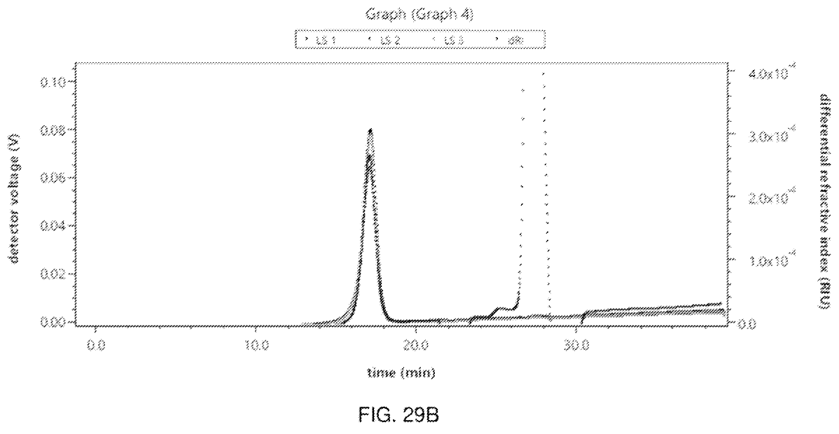

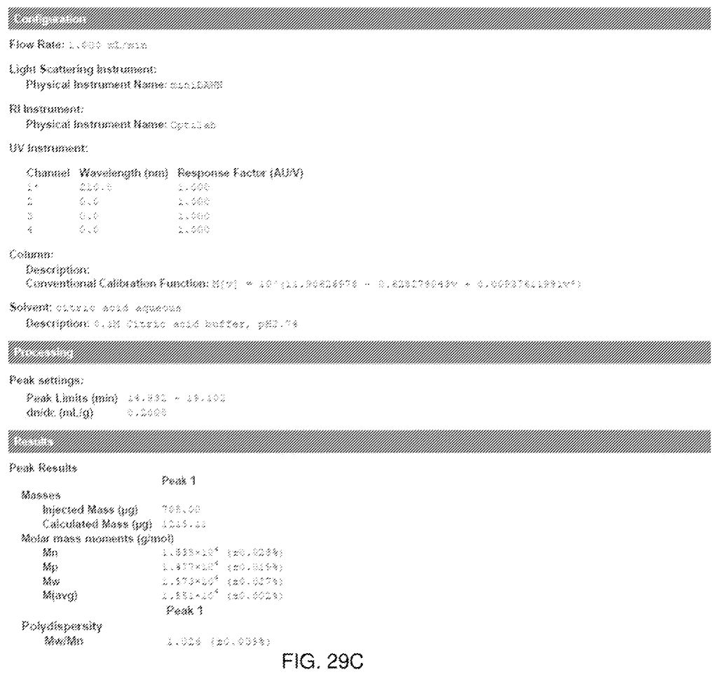

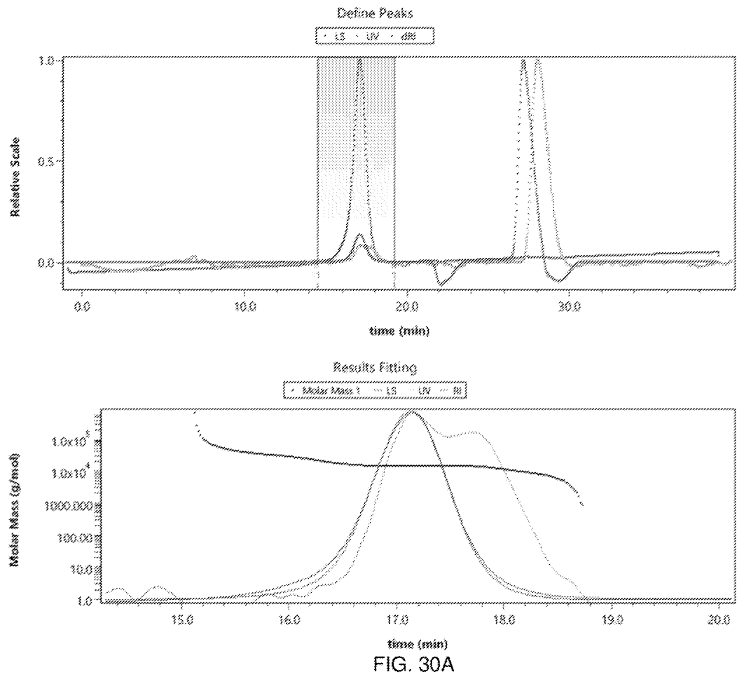

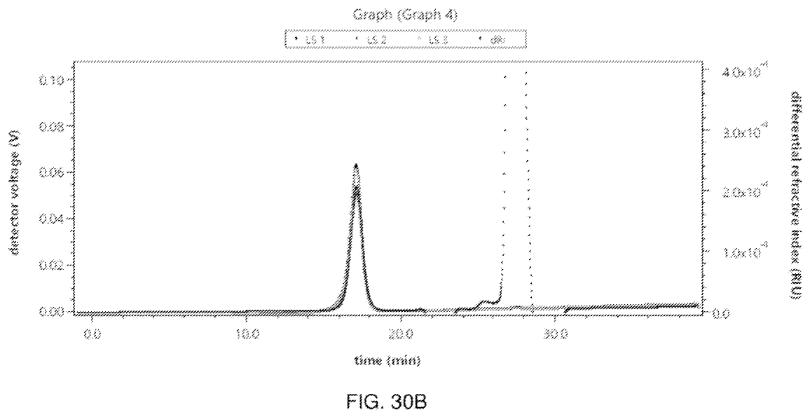

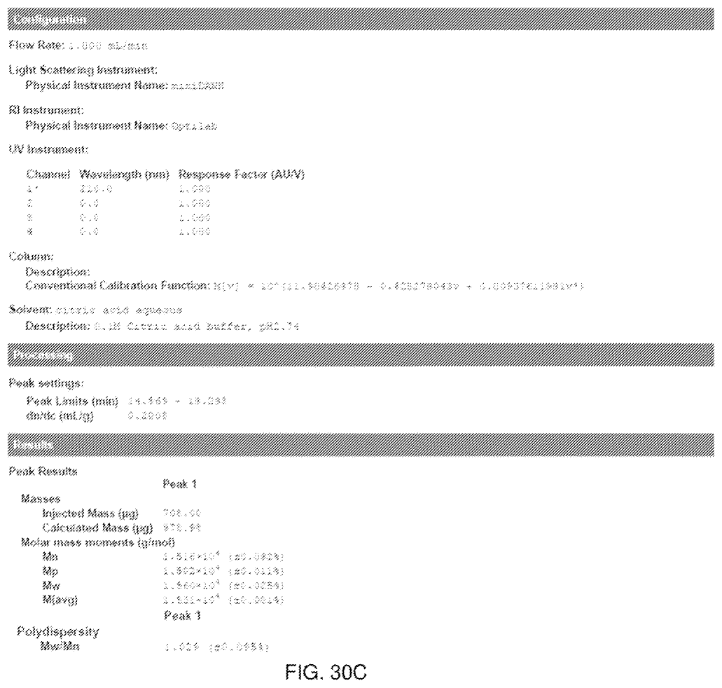

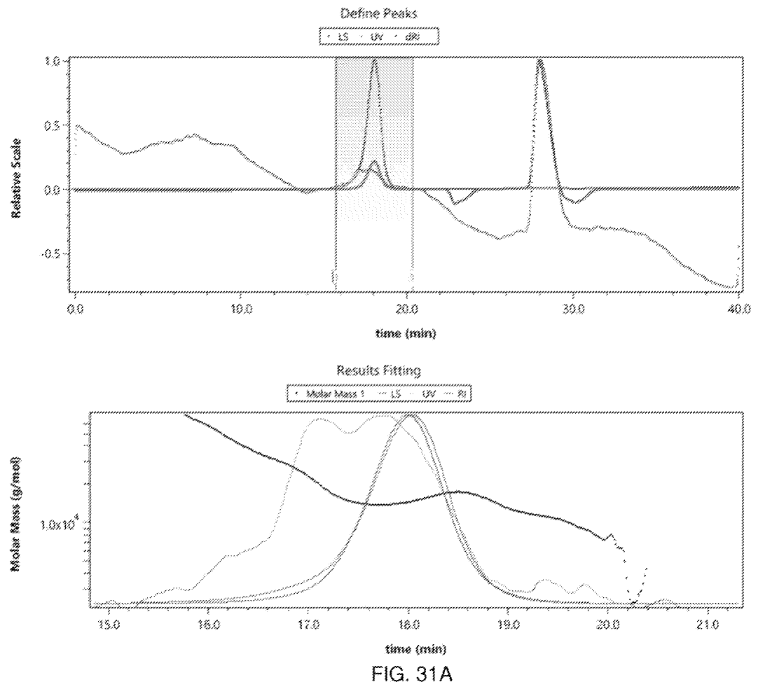

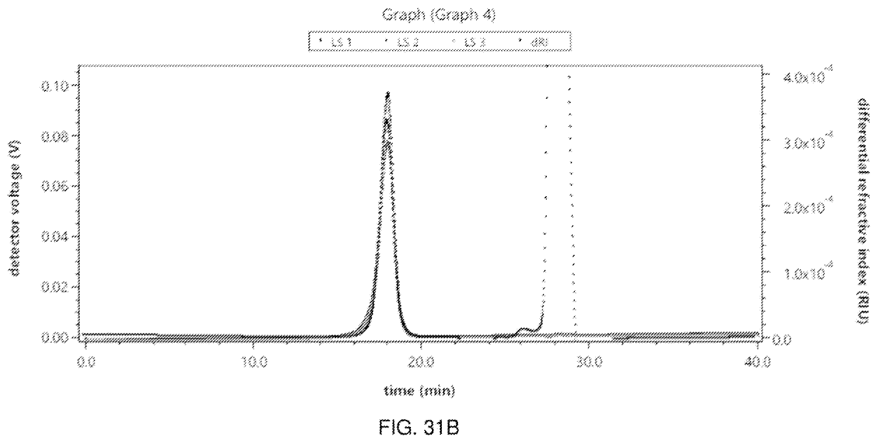

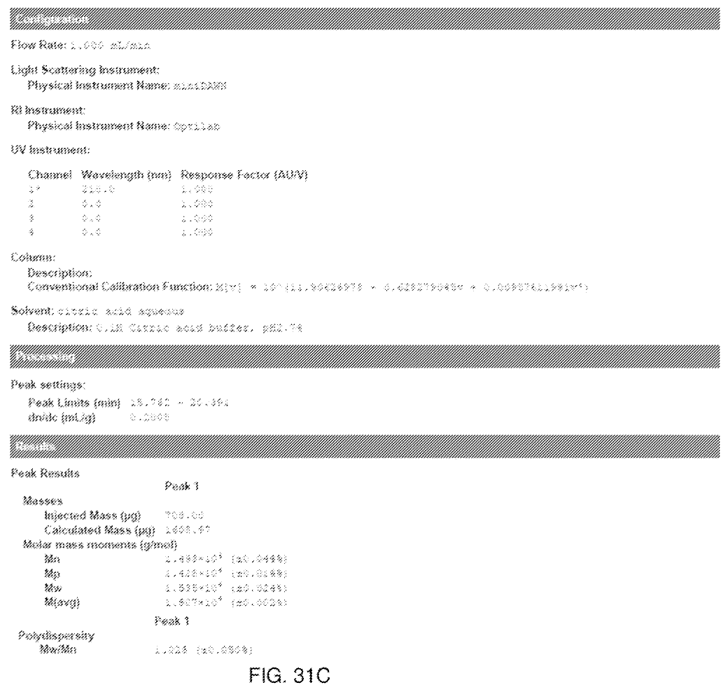

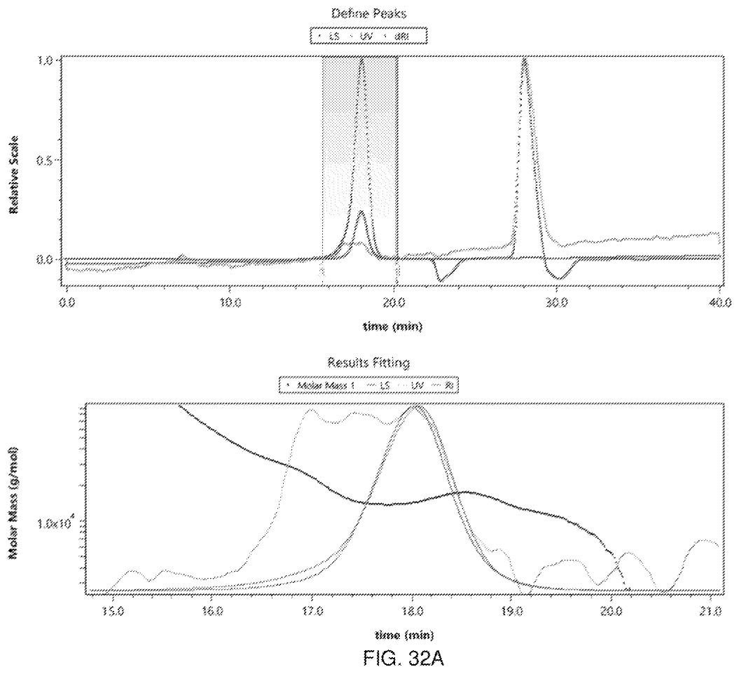

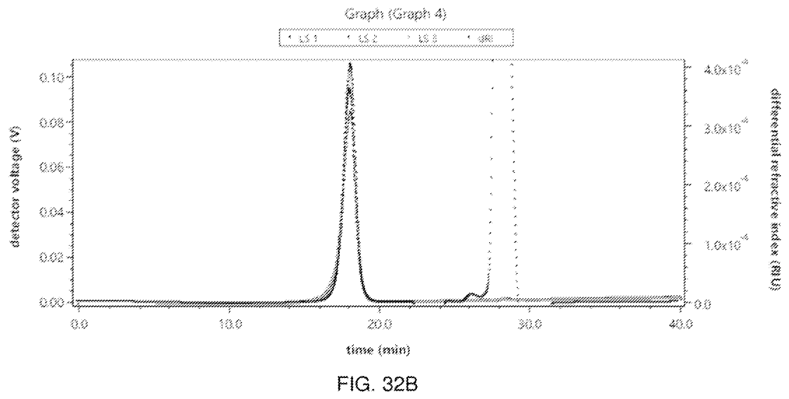

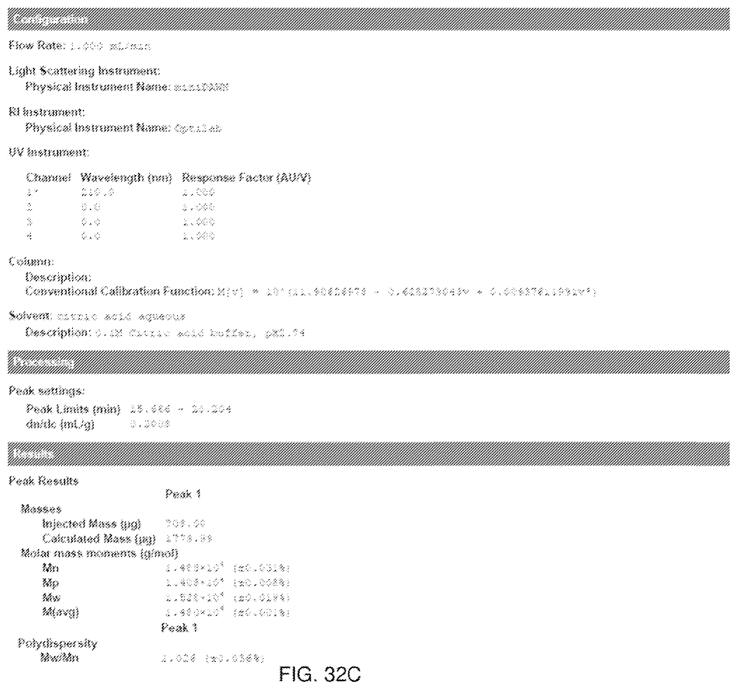

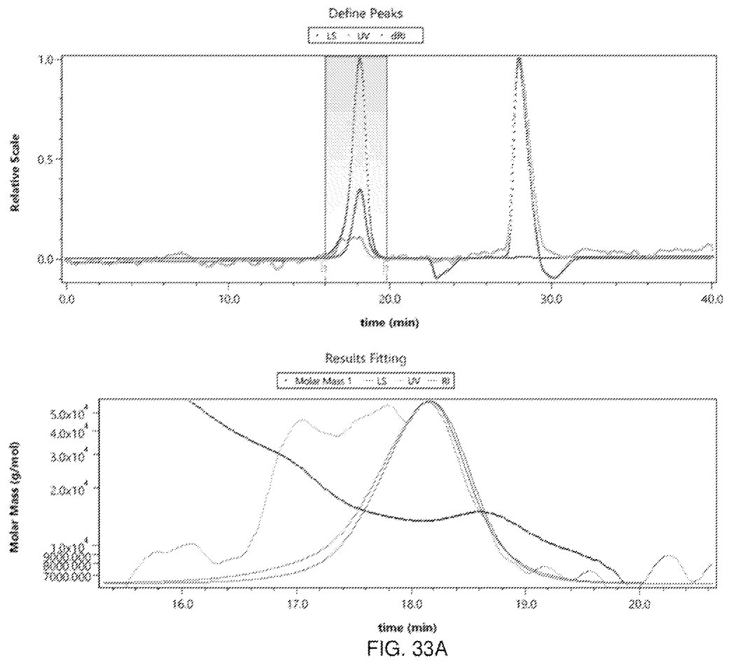

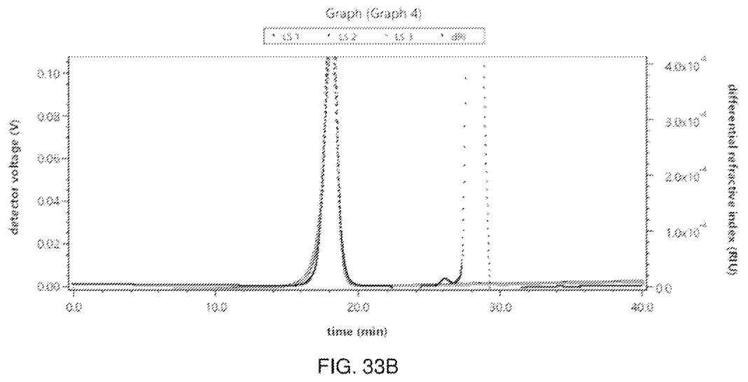

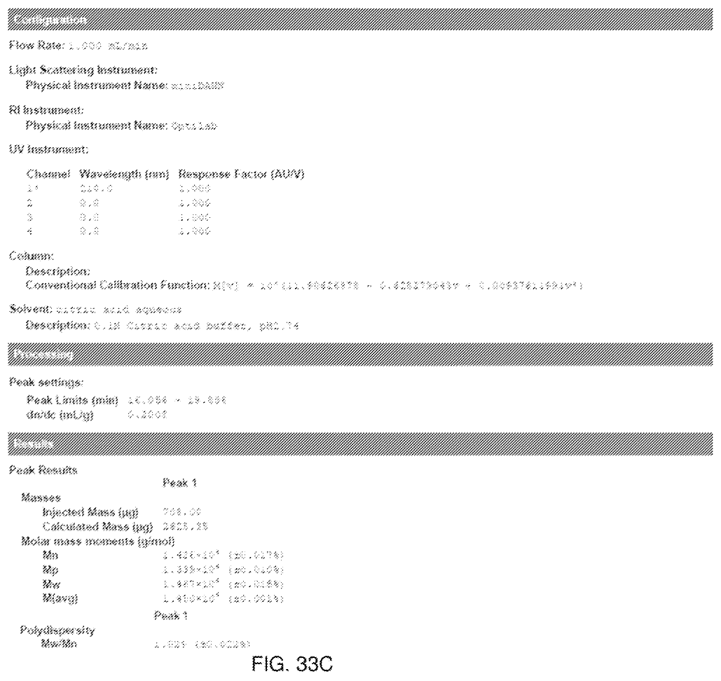

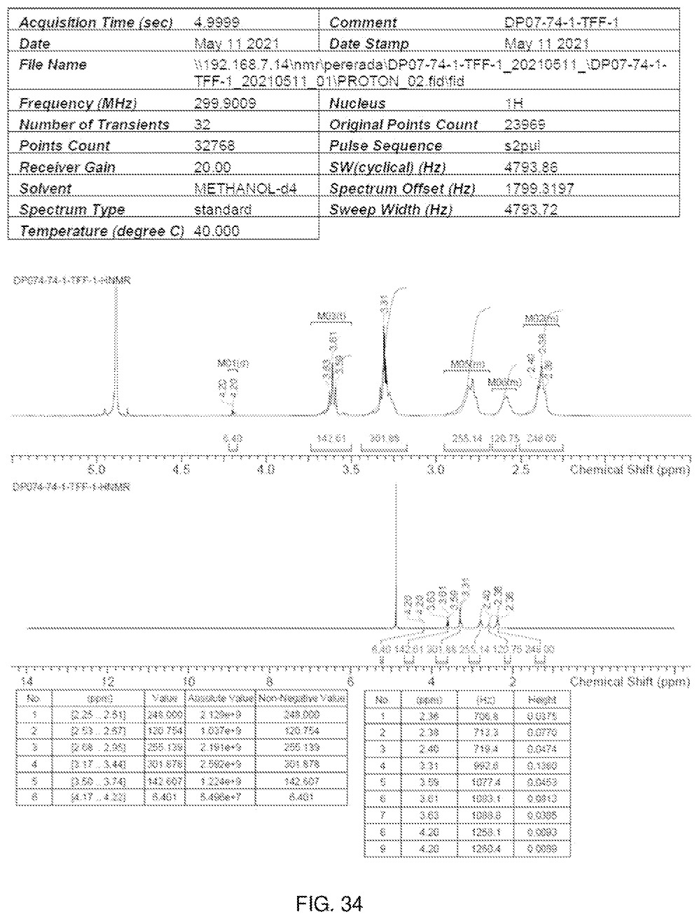

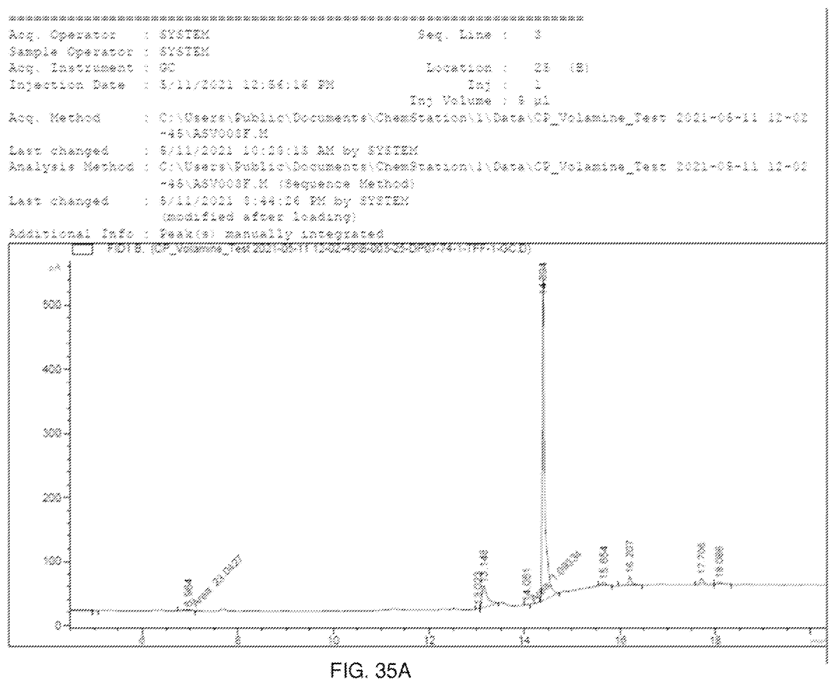

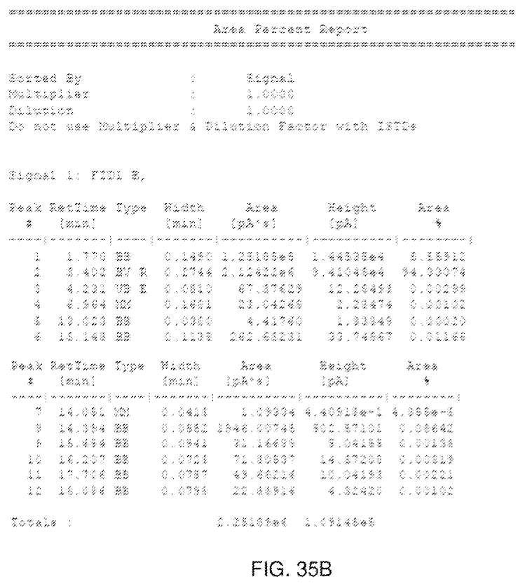

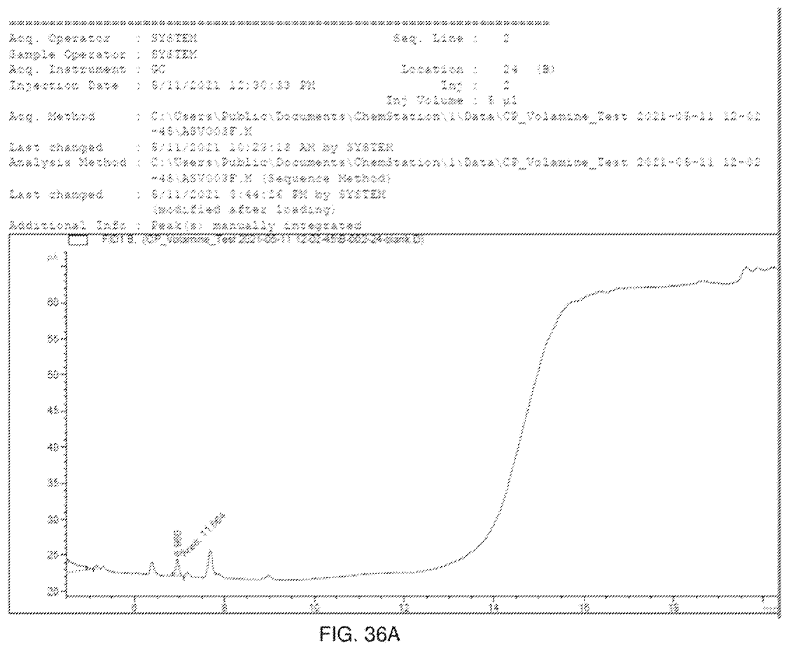

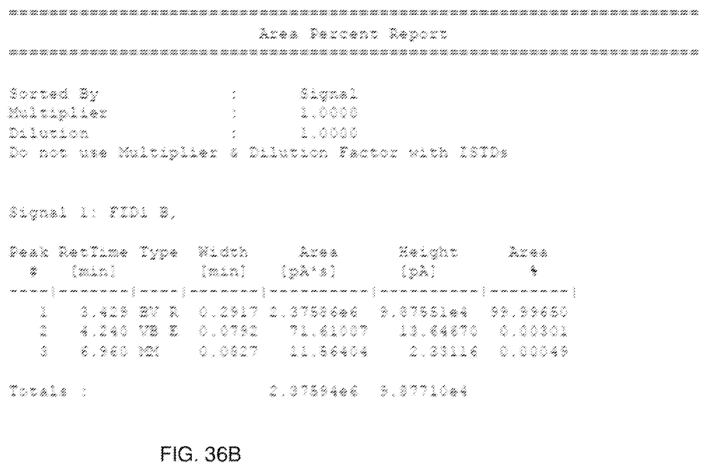

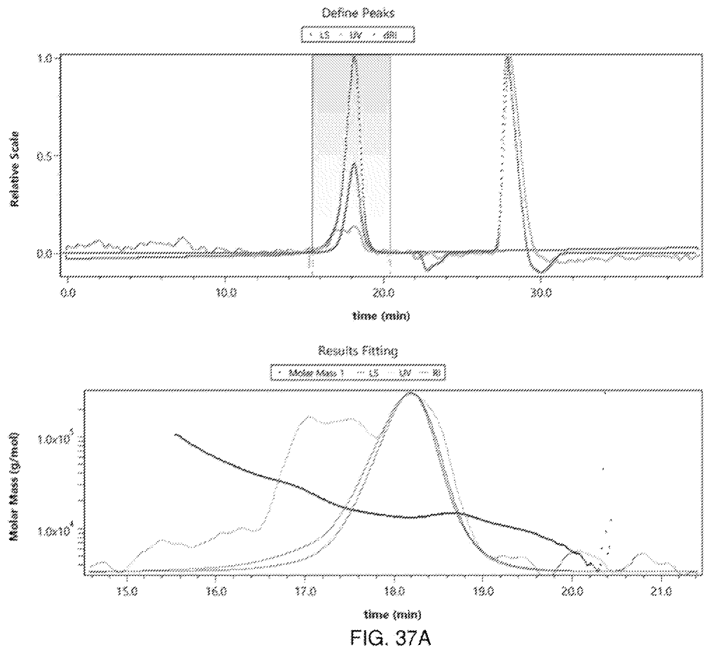

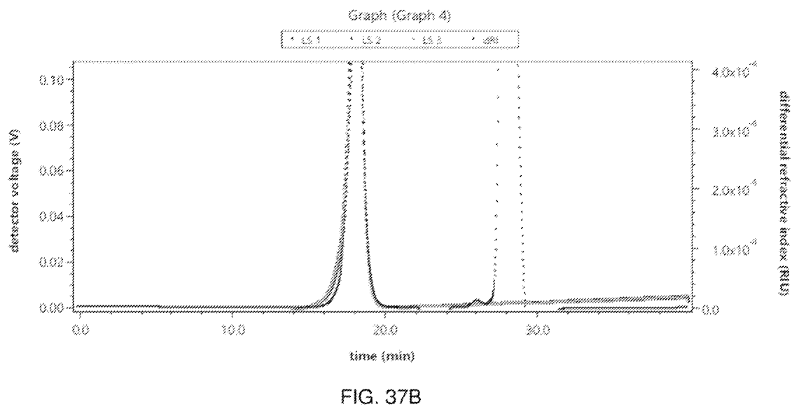

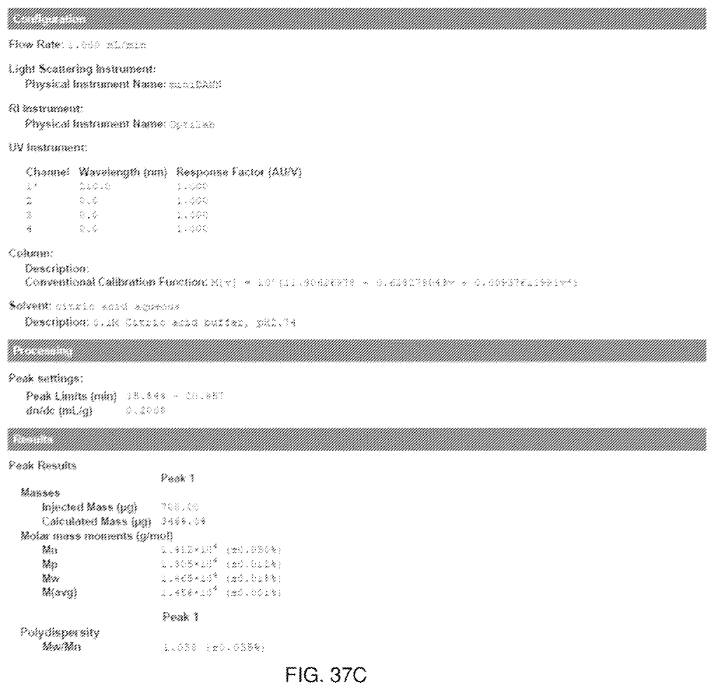

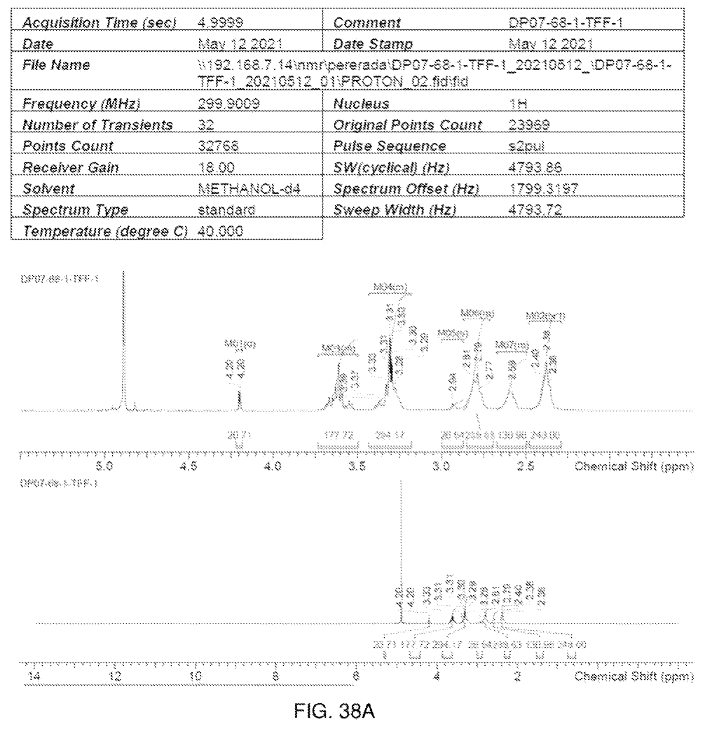

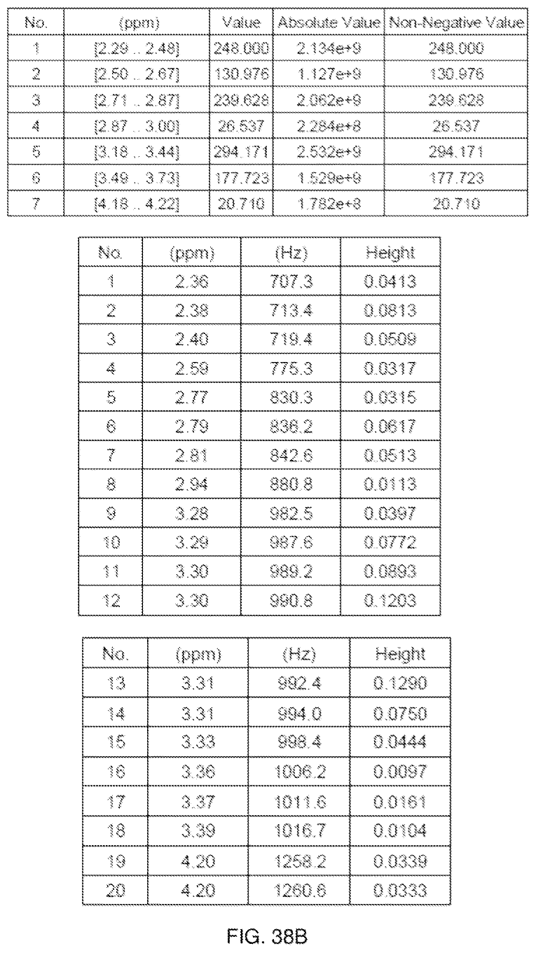

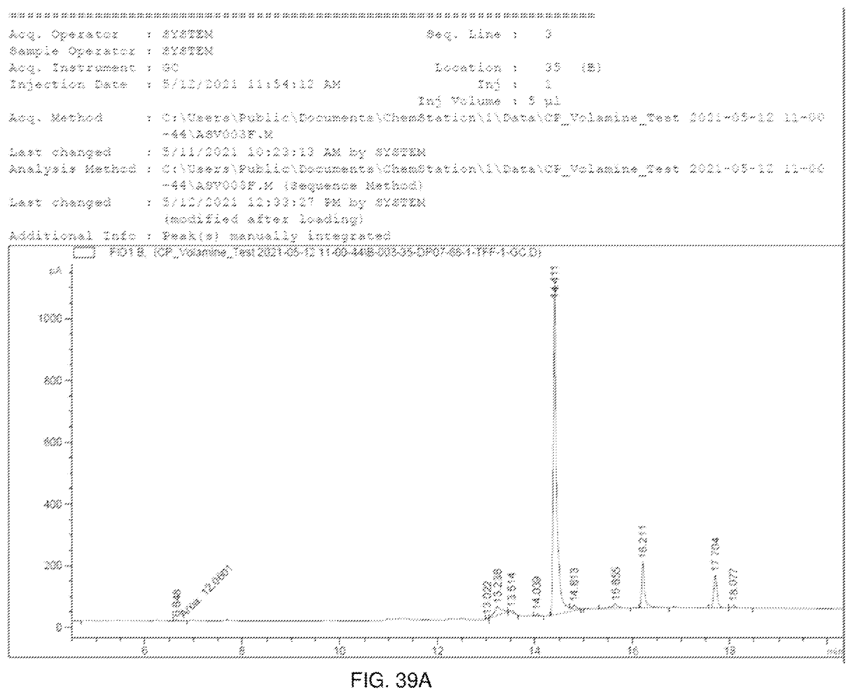

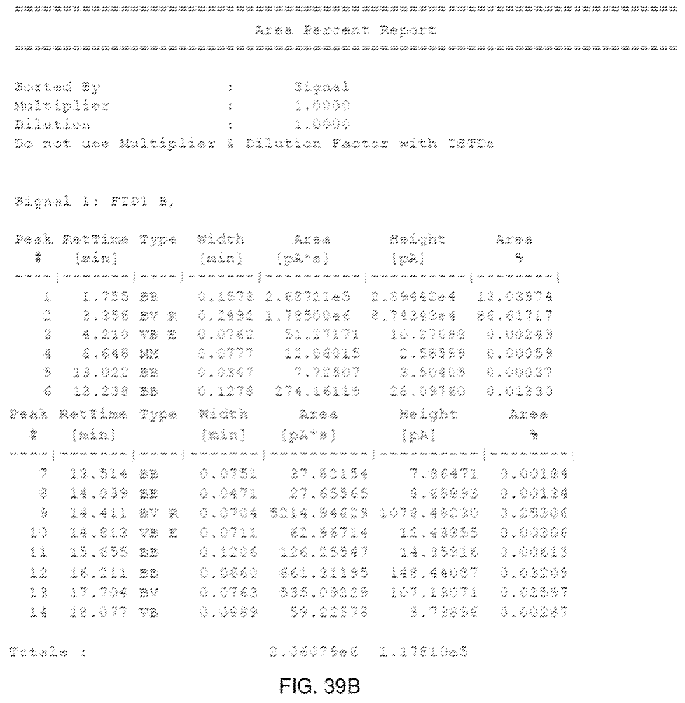

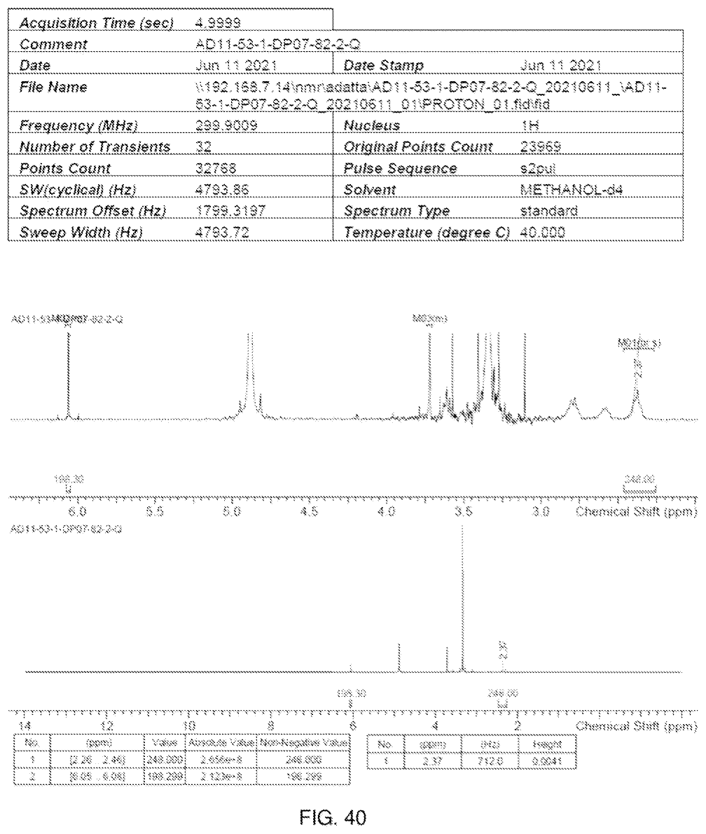

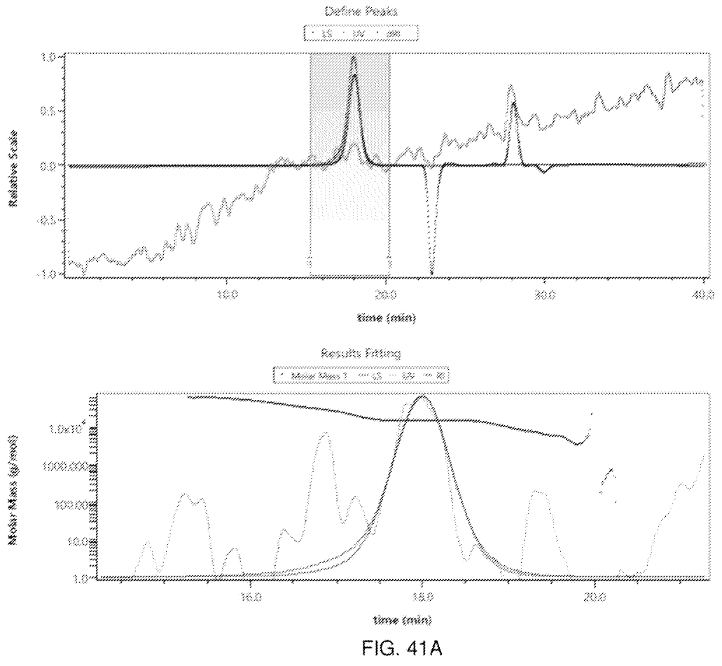

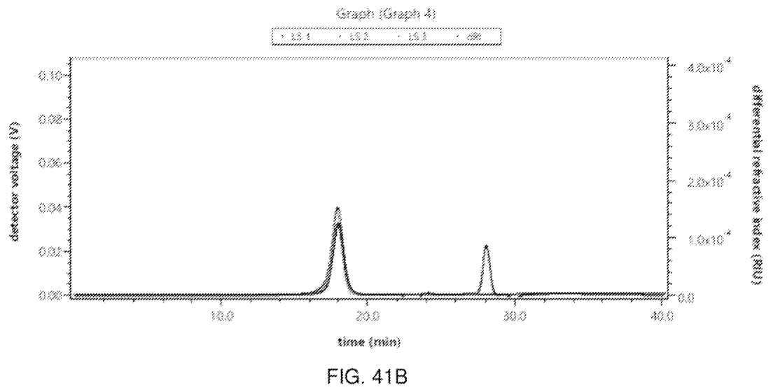

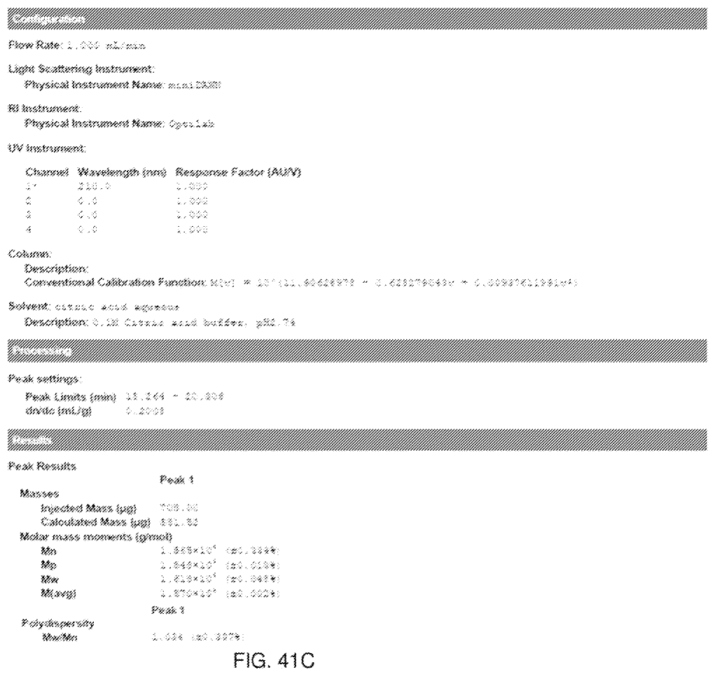

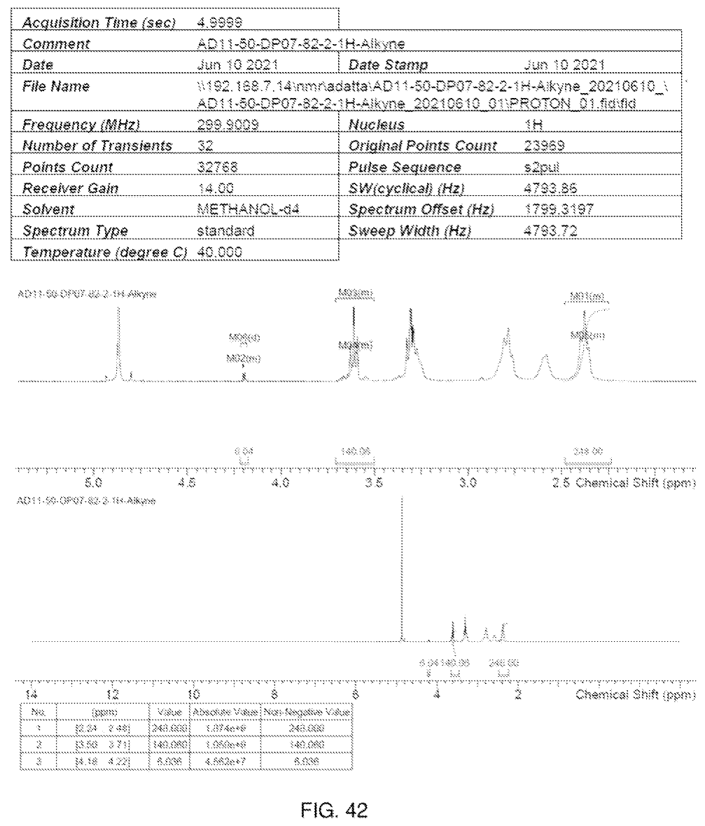

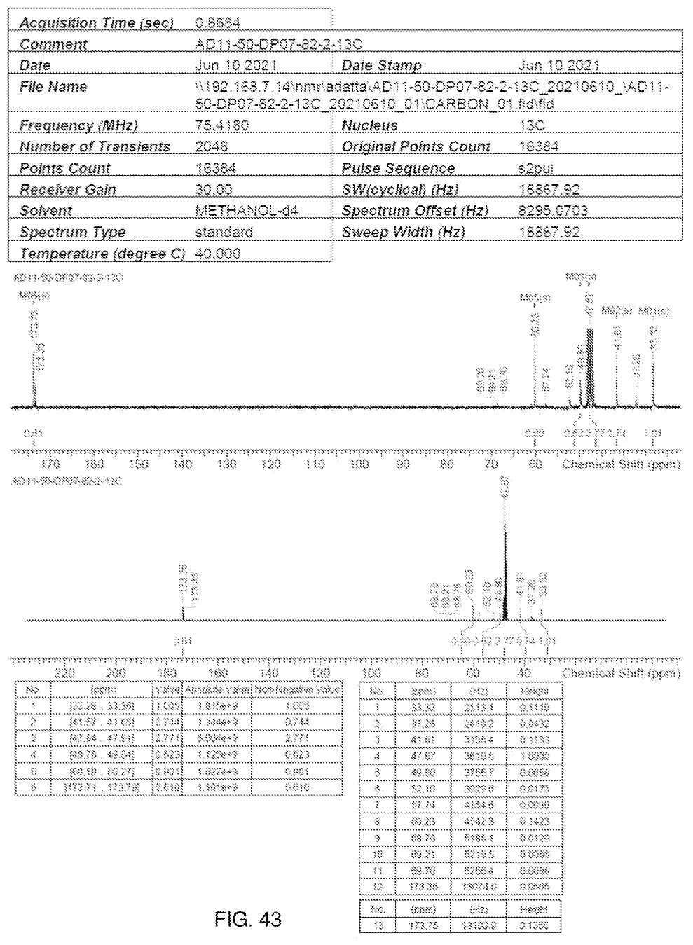

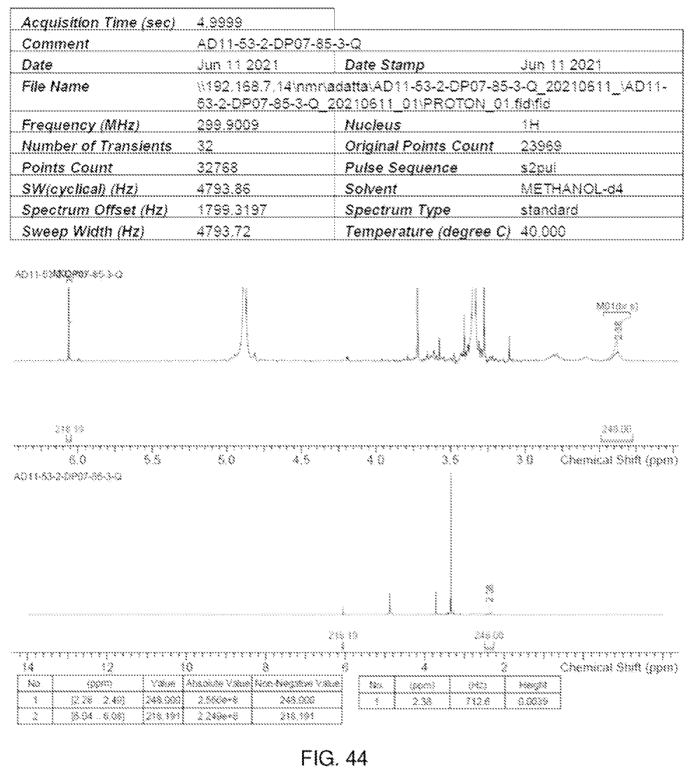

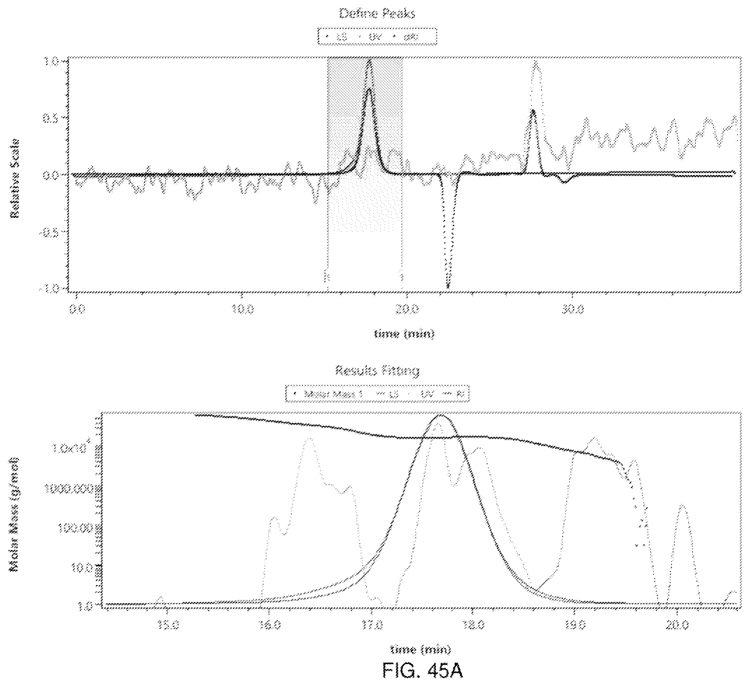

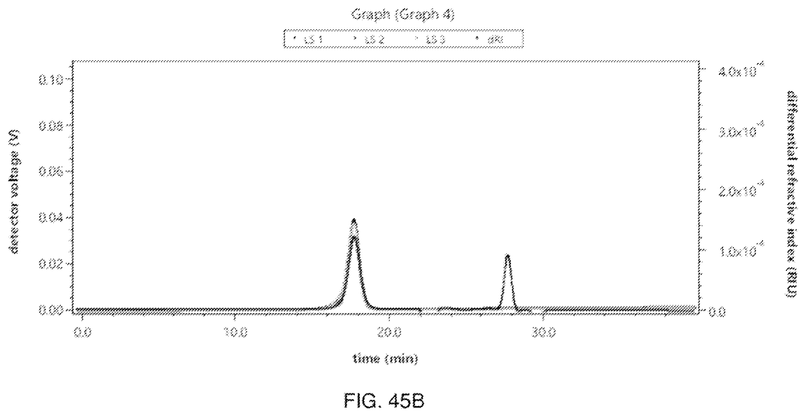

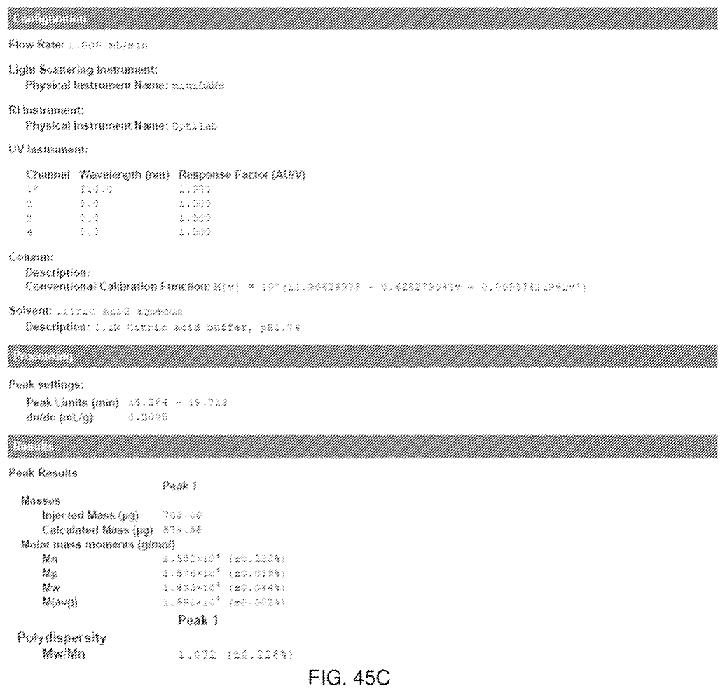

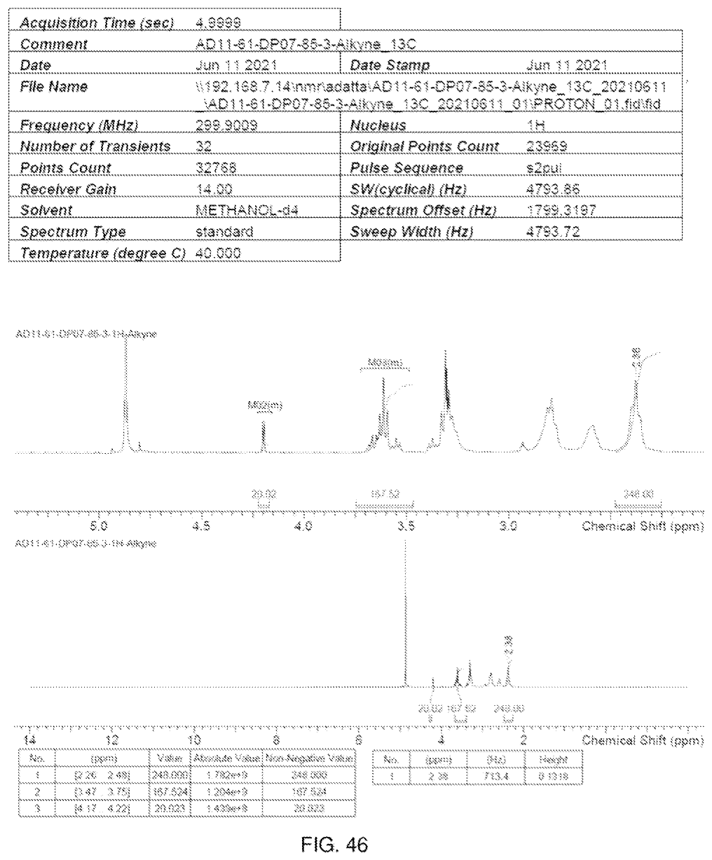

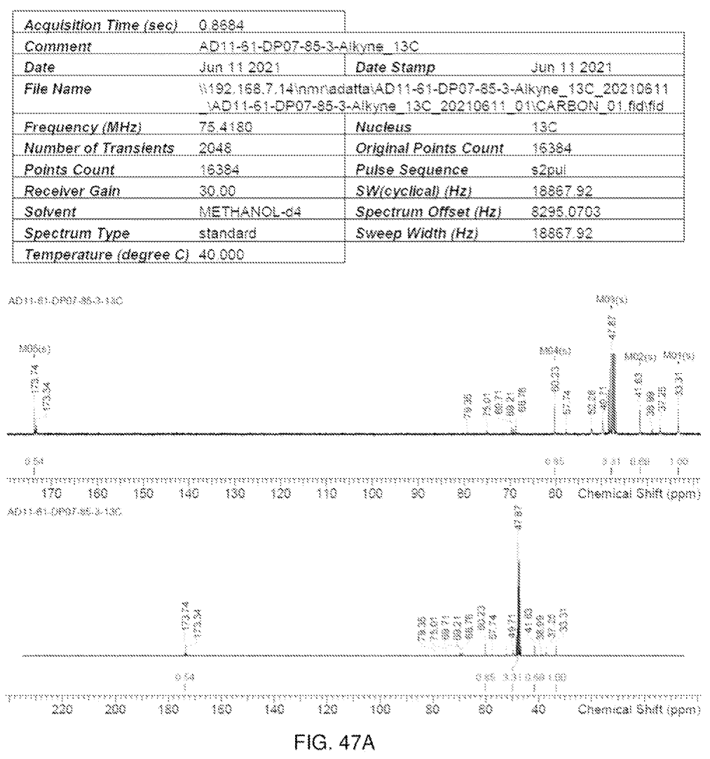

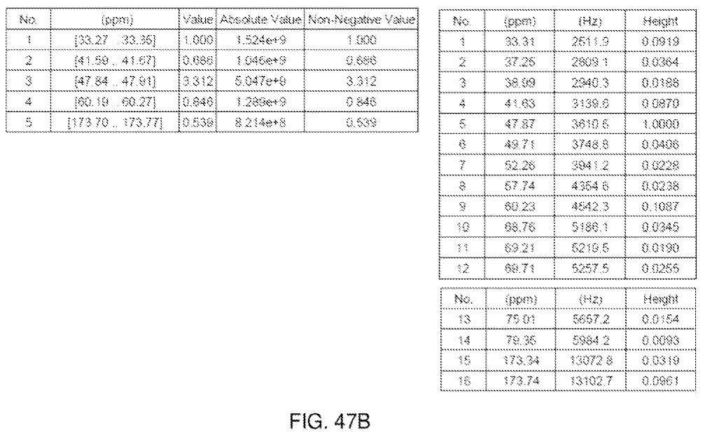

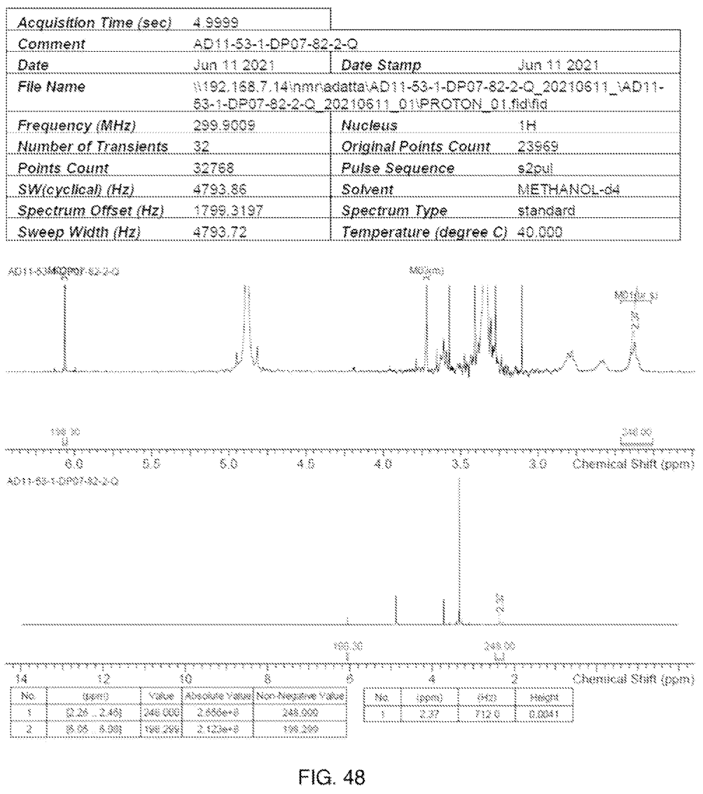

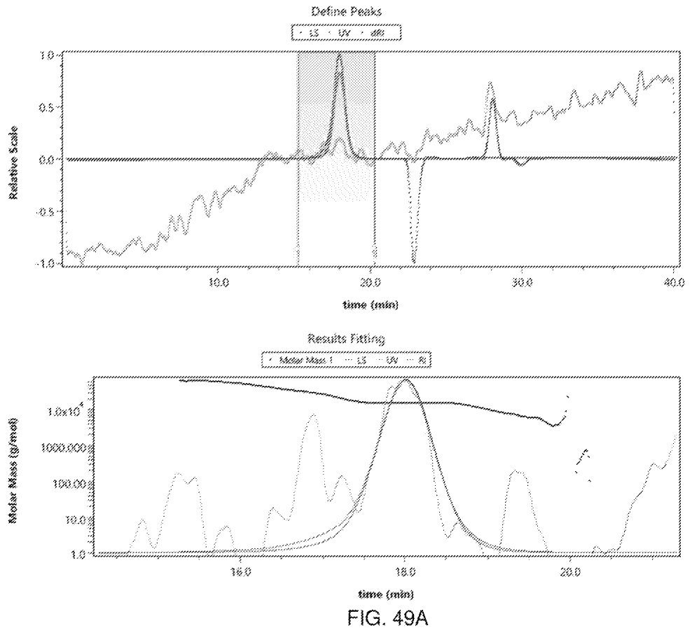

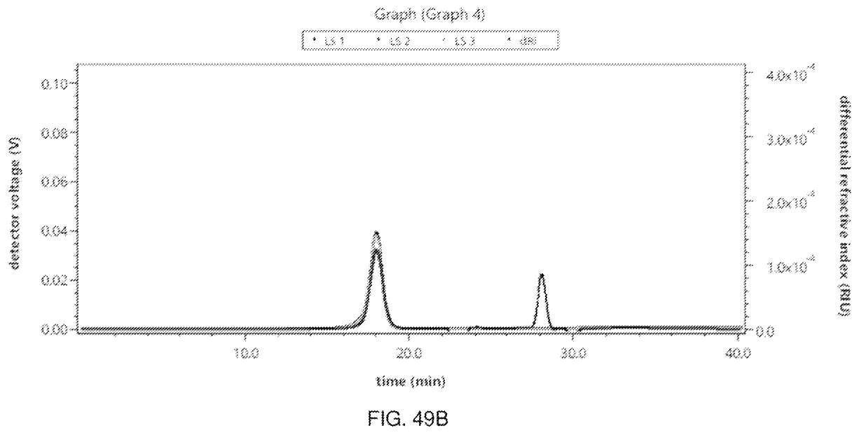

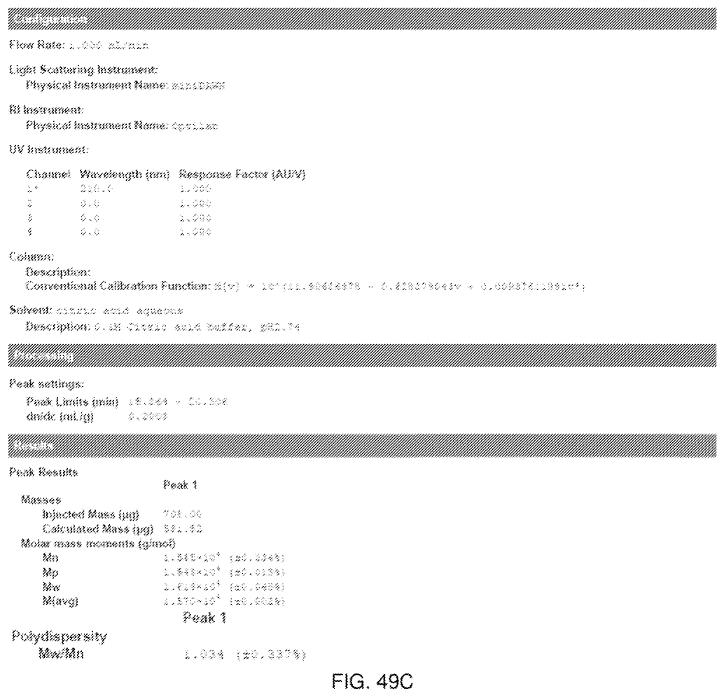

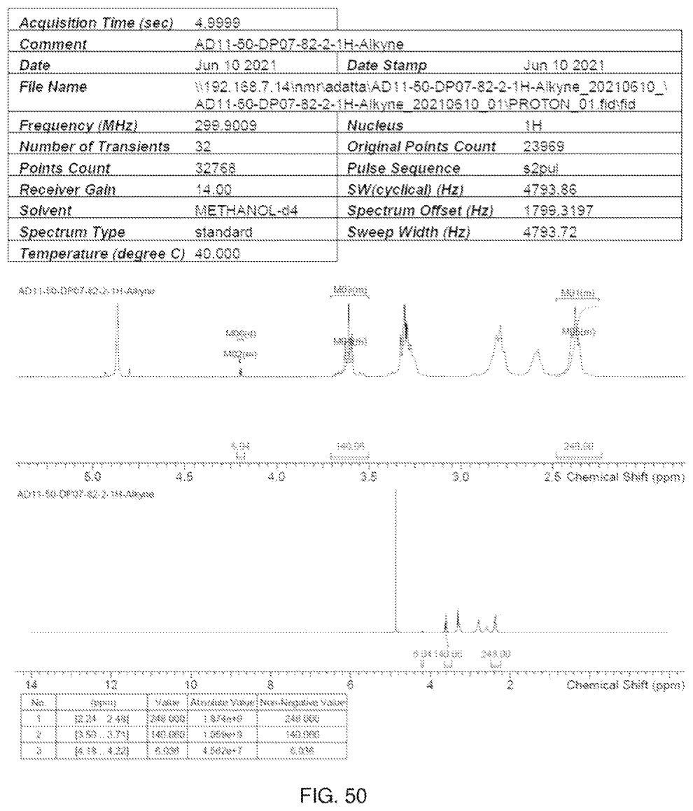

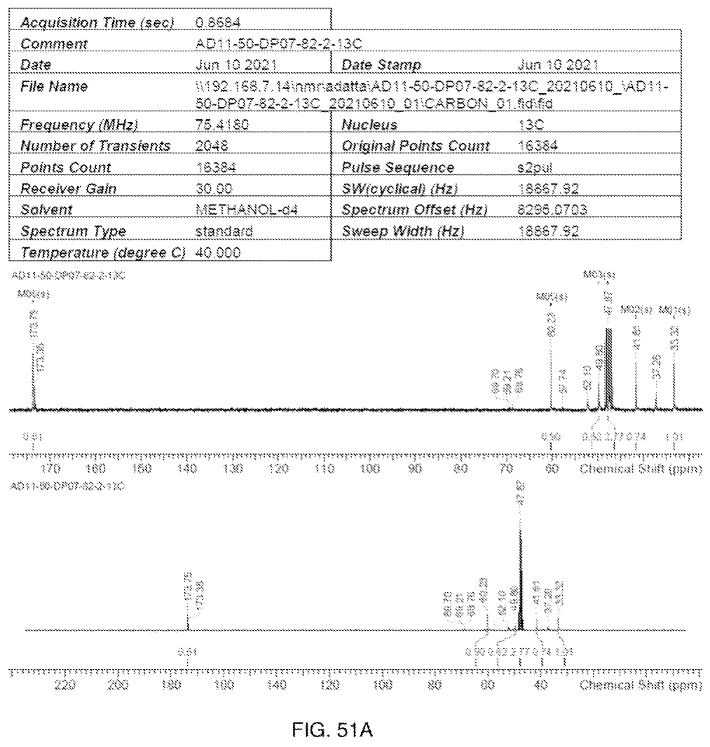

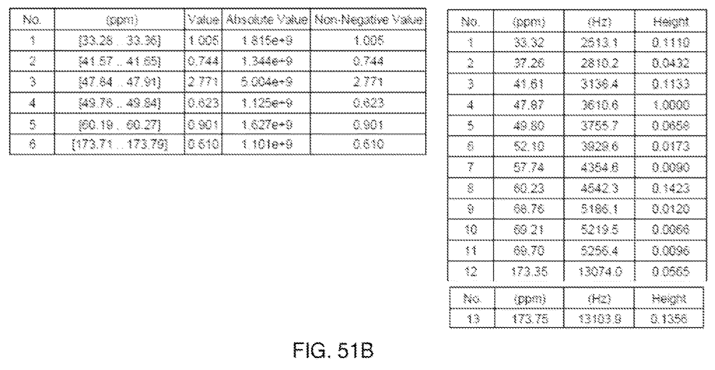

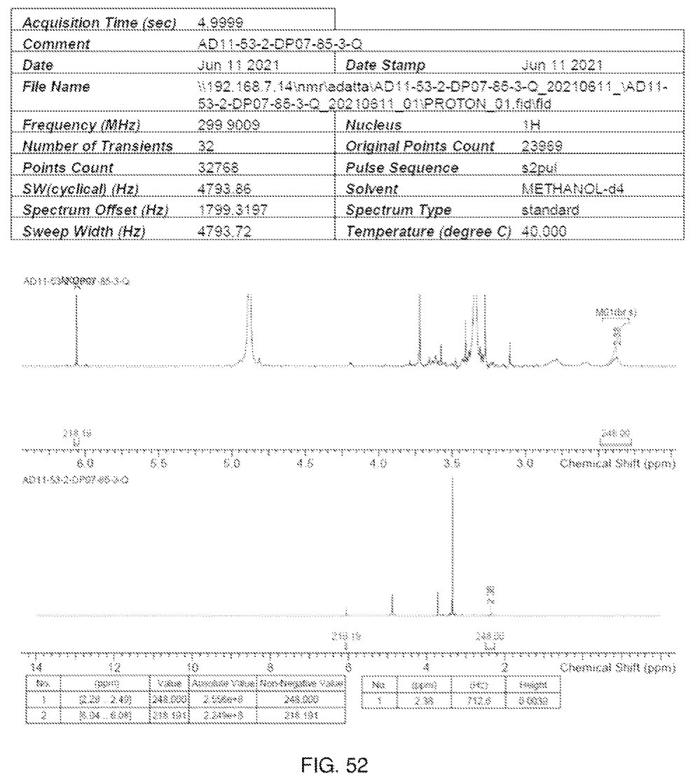

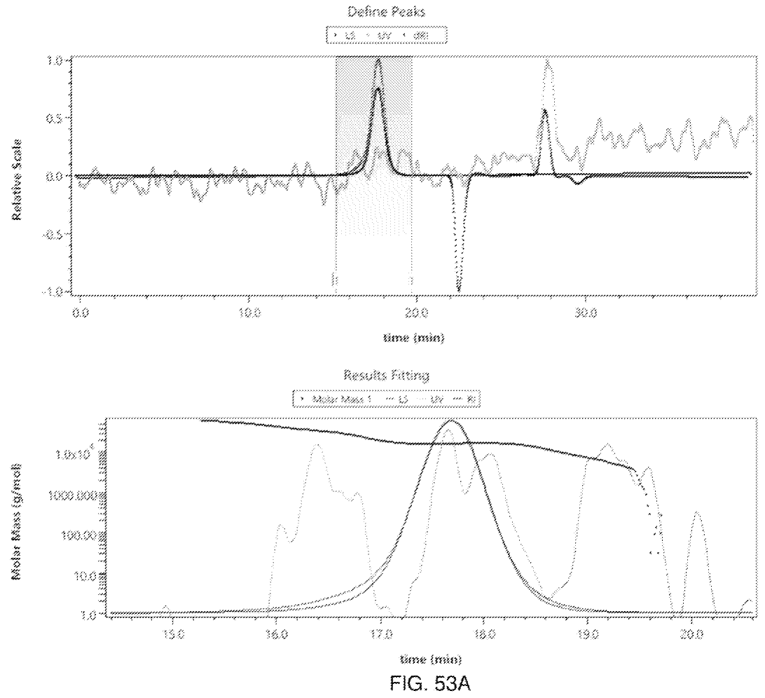

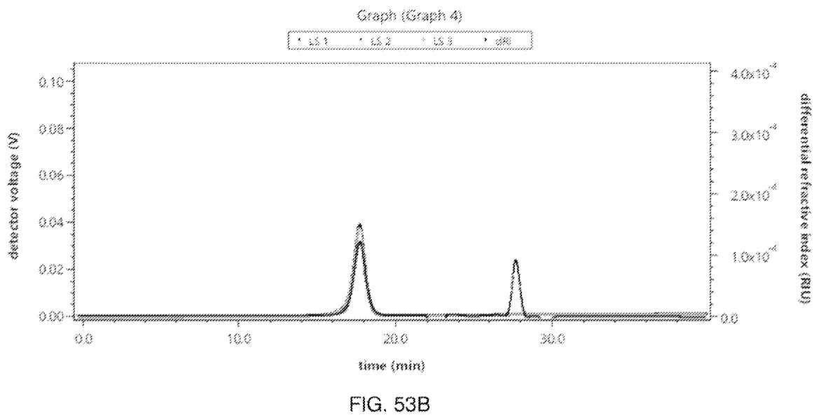

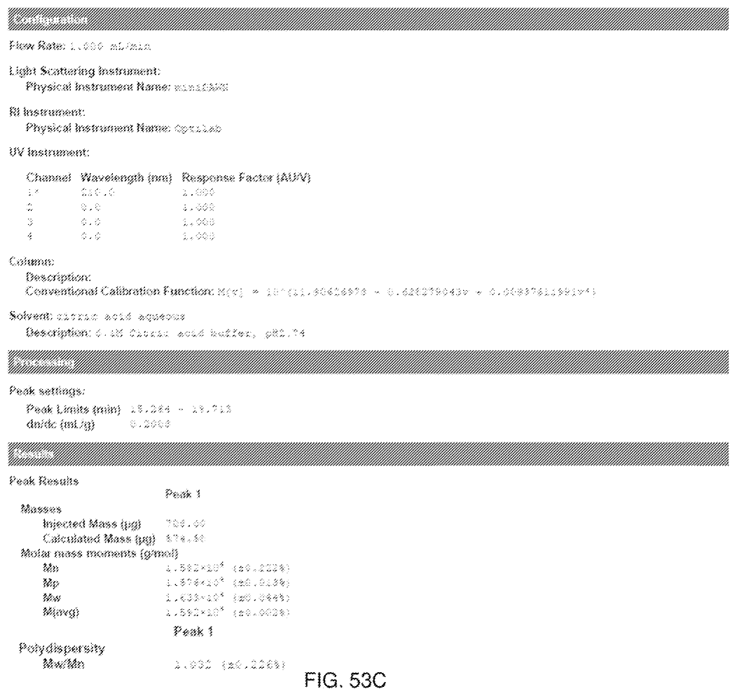

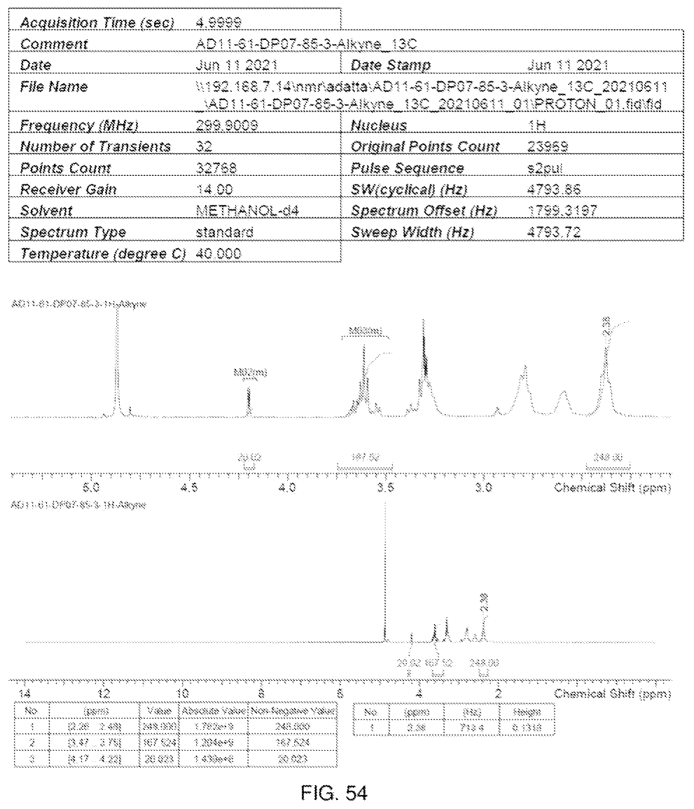

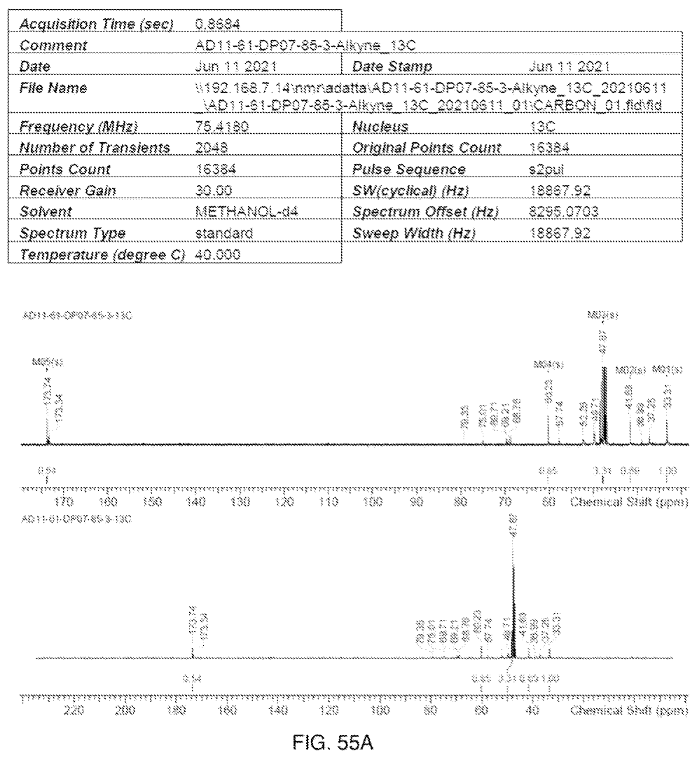

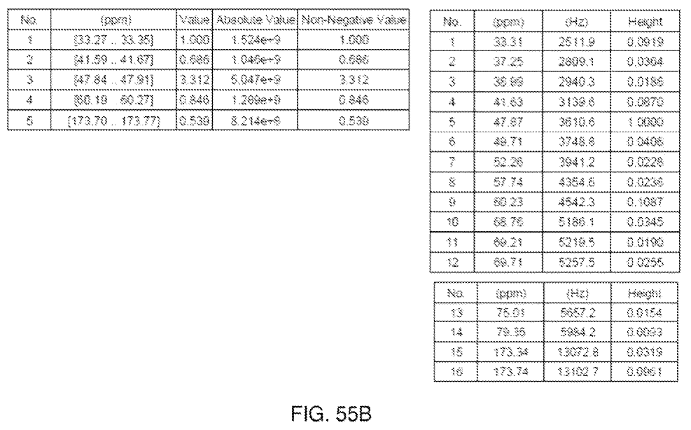

Using the data from the serial dilution described above, calibration curves were made (FIG. 13).

The calibration curve for ethanolamine was used to calculate the concentration of ethanolamine in the sample. The signal for PEG-Alkyne was below the LOD so the lowest value reached in the calibration was reported as the concentration.

### SEC-MALLS Method

### A. Reagents

1. Water: HPLC grade
2. Citric acid: ACS reagent, ≥99.5%

### B. Solutions

1. Diluent: Water, 0.1 M Citric acid, pH 2.7
2. Mobile phase:
   A. Water, 0.1 M Citric acid, pH 2.7
3. Sample Solutions: the sample solution at a target concentration of approximately 2 mg/mL.
4. Blank: Diluent.

### C. Chromatographic Conditions

Column: TosoHaas TSK-Gel Guard PHW 0662 (75 mm×7.5 mm, 12 μm), G2000 PW 05761 (300 mm×7.5 mm, 10 μm, 125 Å), G 3000 PW 05762 (300 mm×7.5 mm, 10 μm, 200 Å), G4000 PW (300 mm×7.5 mm, 17 μm, 500 Å) Temperature: 25° C.
Flow Rate: 1 mL/min
Detection: A: Wyatt Optilab differential refractive index detector operating at 658 nm
   B: Wyatt miniDAWN multi-angle laser light scattering detector with 120 mW laser operating at 658 nm
Injection Volume: 100 μL
Run Time: 40 min

### Example 6. Exemplary PAMAM Dendrimers Conjugated to Drugs

Schemes 1-19 below show exemplary reactions for functionalizing a PAMAM dendrimer (e.g., PAMAM carboxy methyl functionalized dendrimer), with a linker attached to a functional group R, where R is amine, alkyne, acetylene, COOH, hydroxy, bromo, DBCO, thiols, alkene, aldehyde, hydroxyl, sulphonate, nitrile. In Schemes 1-19, the polyethylene glycol (PEG) linker can be replaced with linkers such as

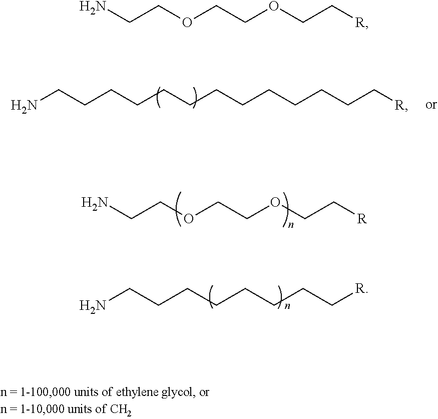

Exemplary reaction partners in step 1 of Schemes 1-19 include:

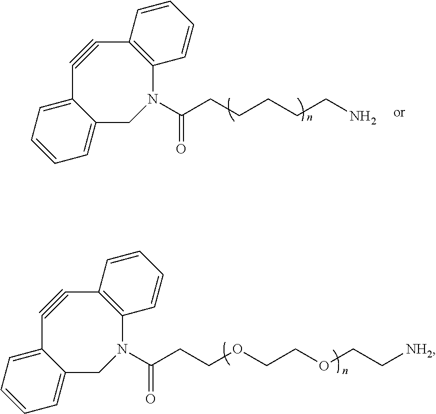

where linker length can vary from 1-100,000 units of CH₂ and ethylene glycol (PEG), and the linker can include PEG linkers with different molecular weights.

Other exemplary linkers are:

### Drug Linkers

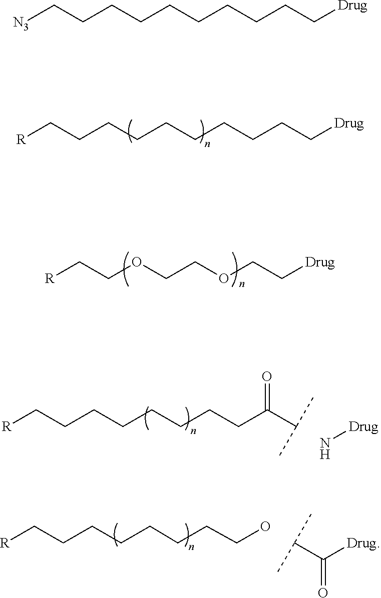

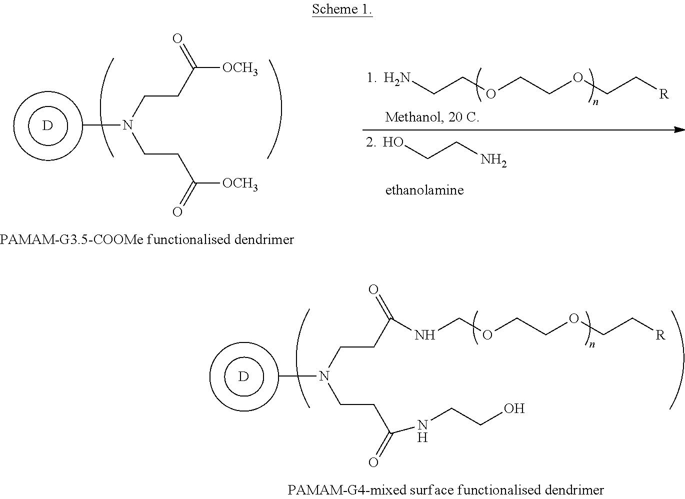

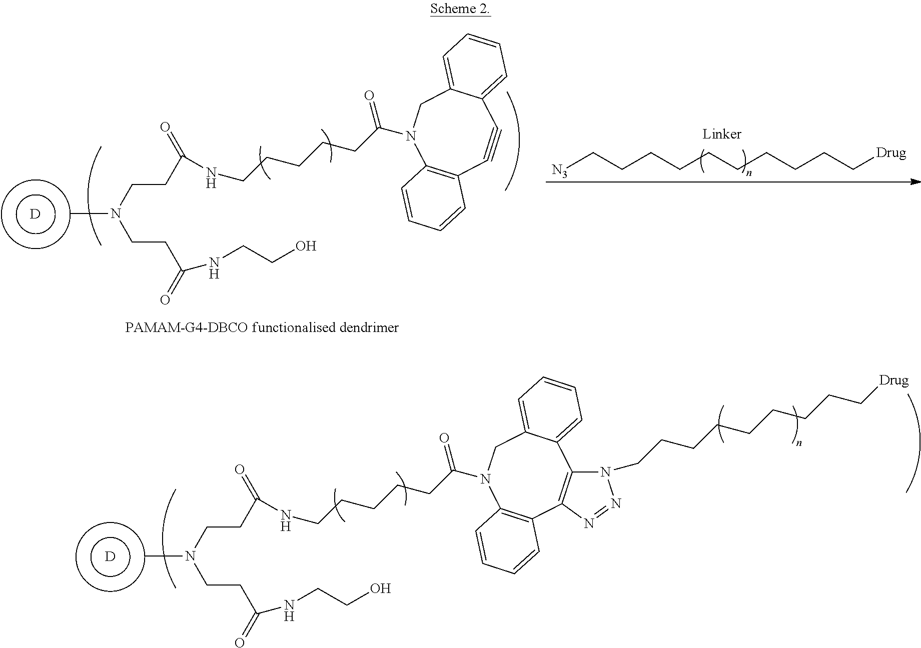

Other types of click reactions for functionalizing a dendrimer include the following (shown in Schemes 3-19):

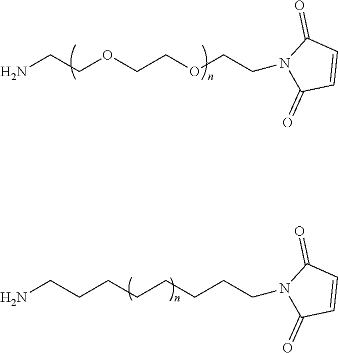

Thiol maleimide click:

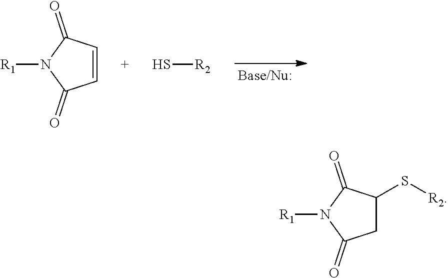

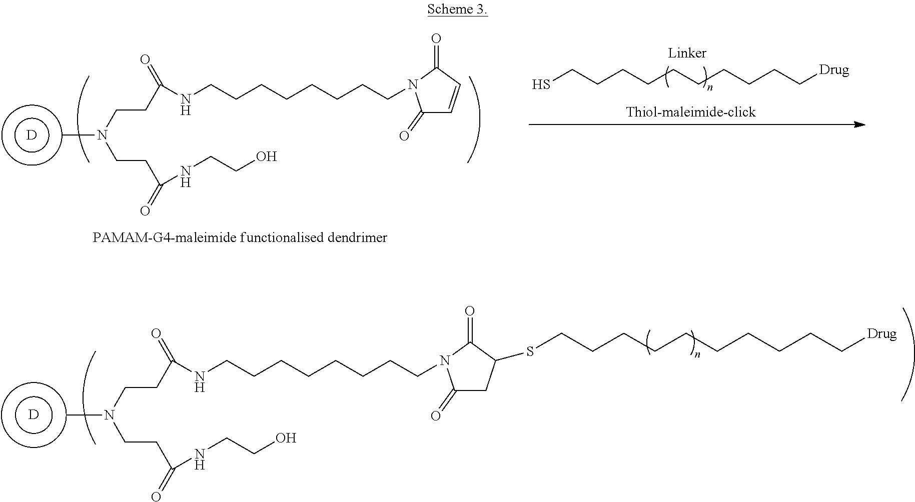

### Tetrazine Ligation:

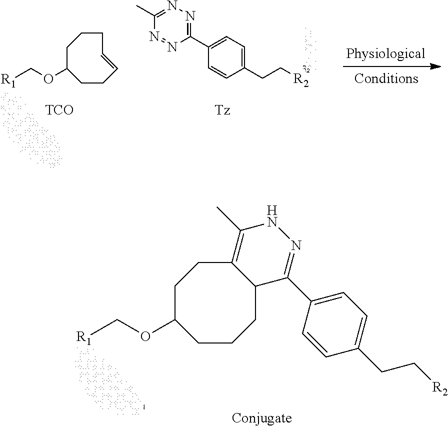

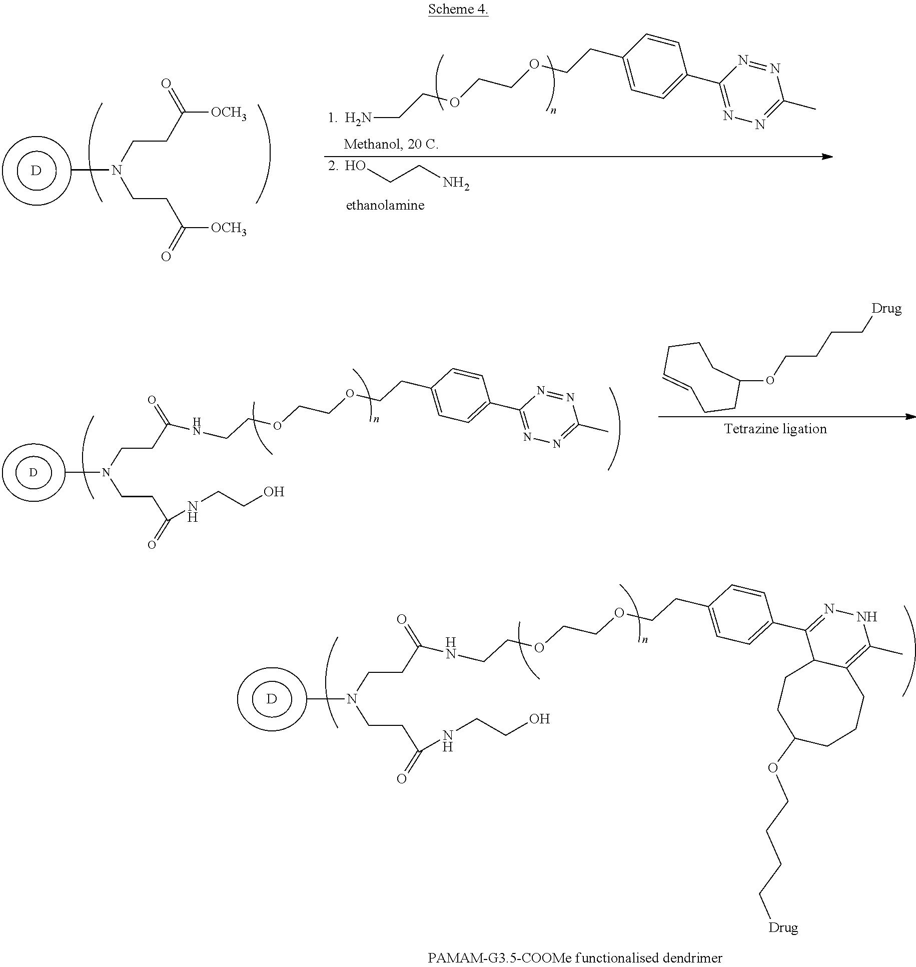

Isoxazole Formation from Alkyne and Oxime:

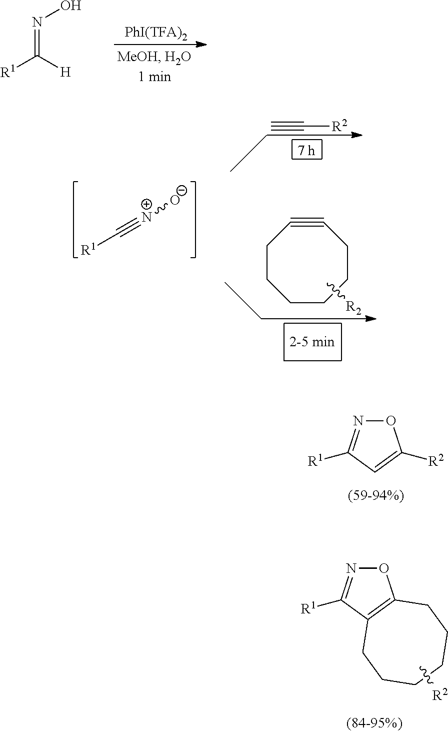

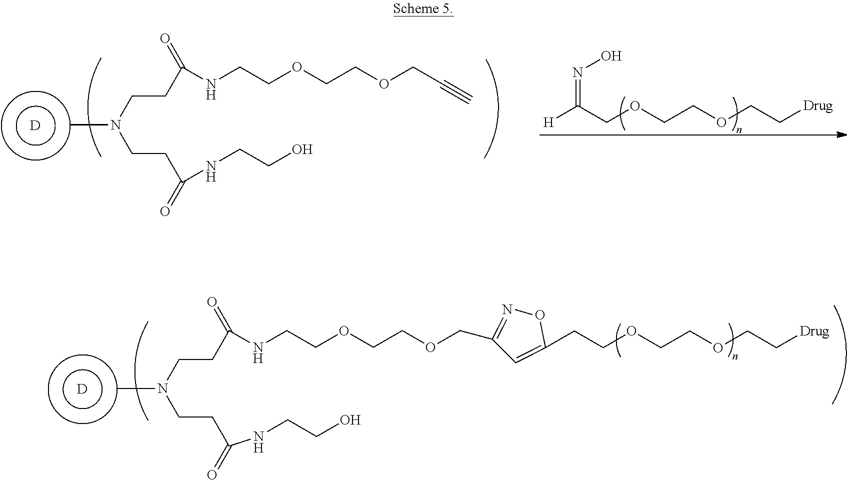

### Thiol-yne:

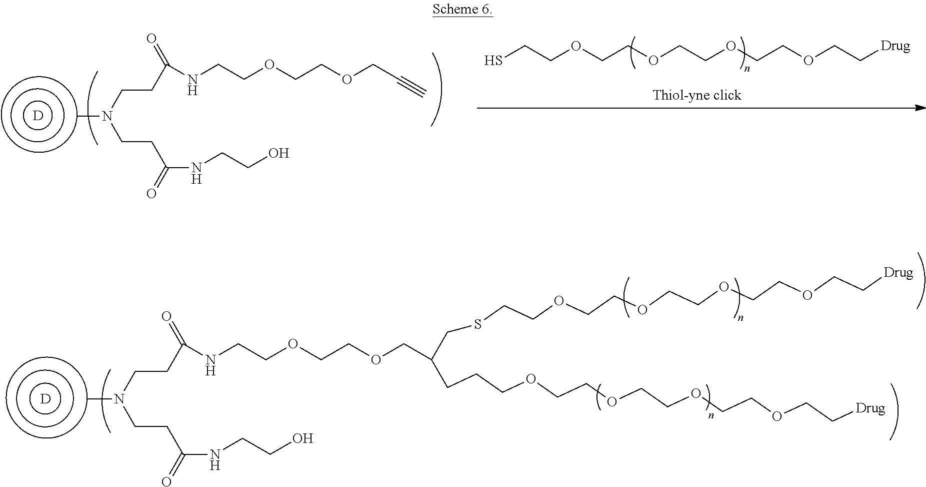

### Thiol-ene:

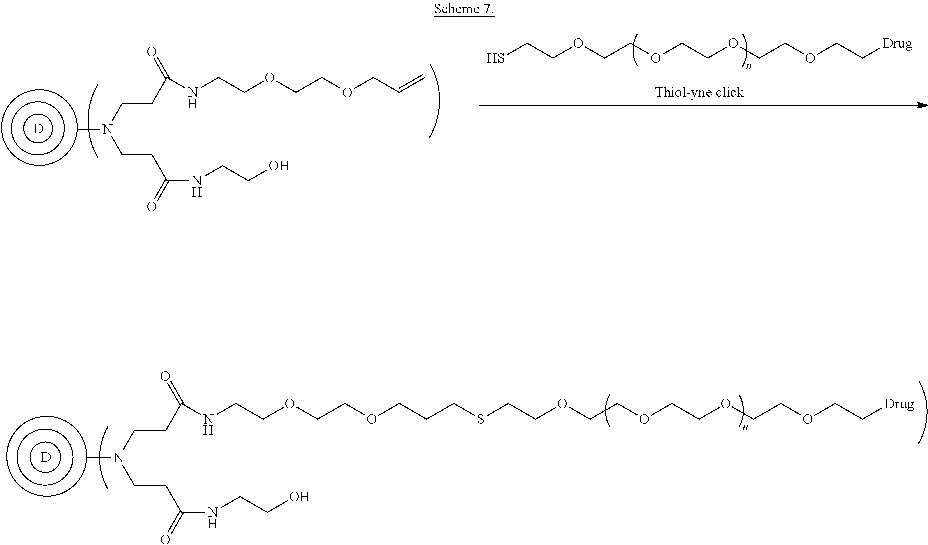

### Thiol-Michael Click:

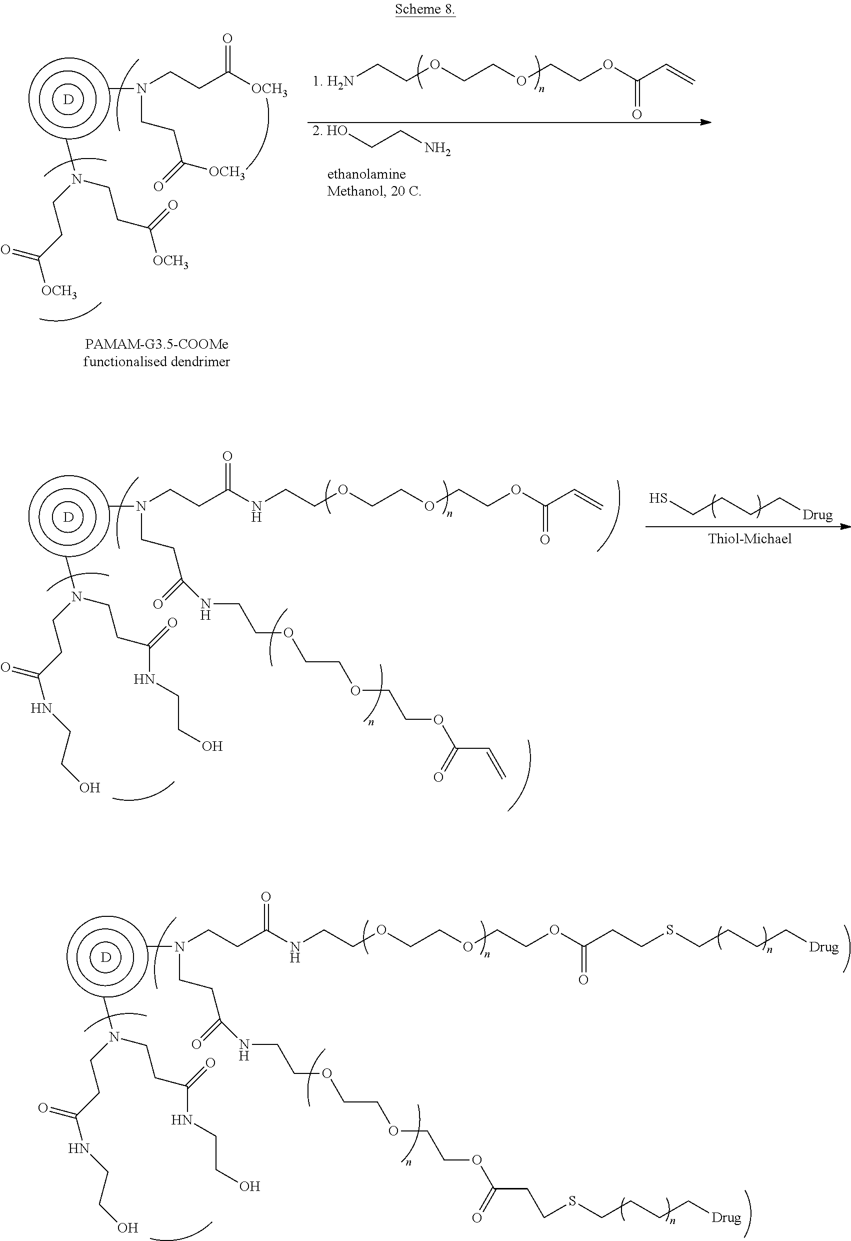

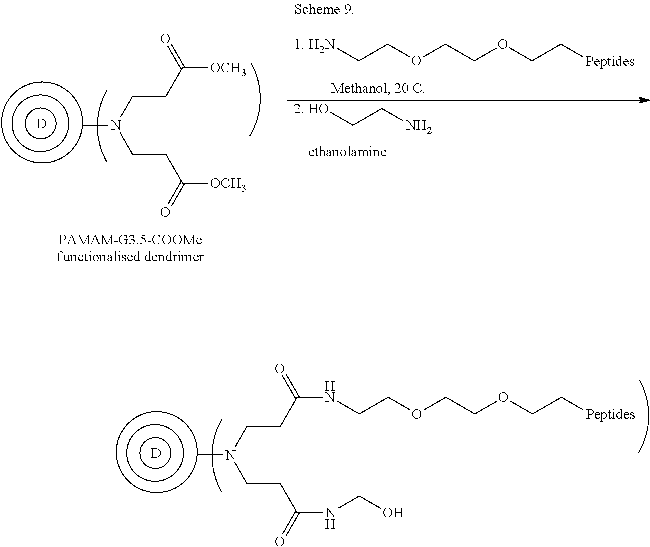

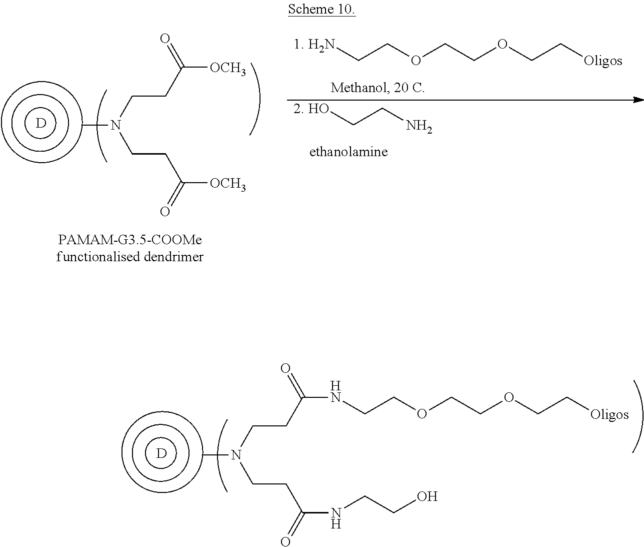

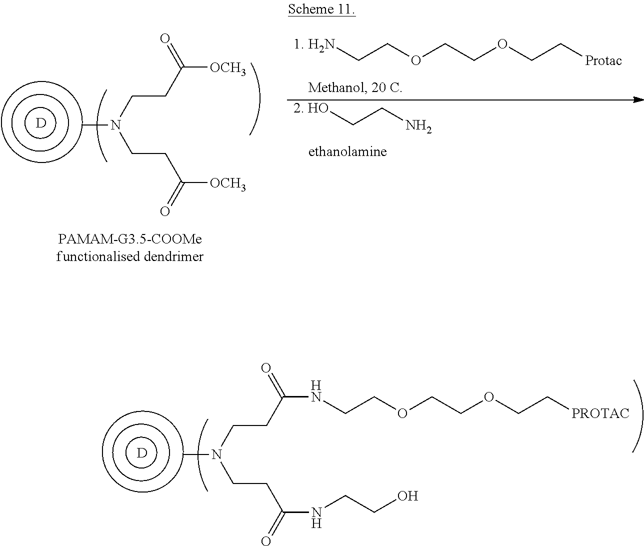

3 different moieties on one dendrimer in one pot using methodology:

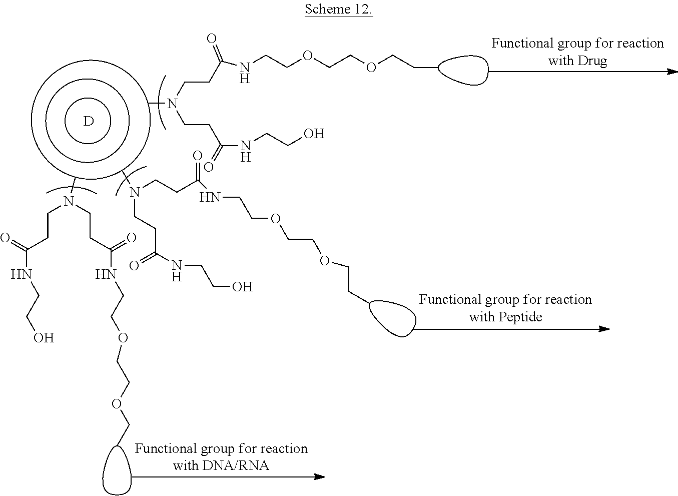

Two drugs on one dendrimer in one pot using methodology:

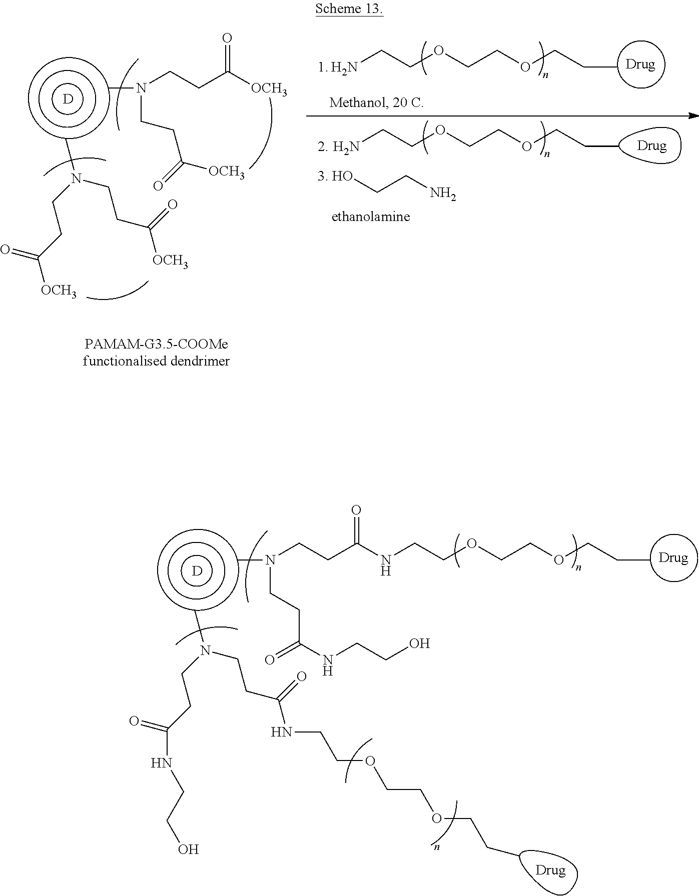

One drug and antibody on one dendrimer:

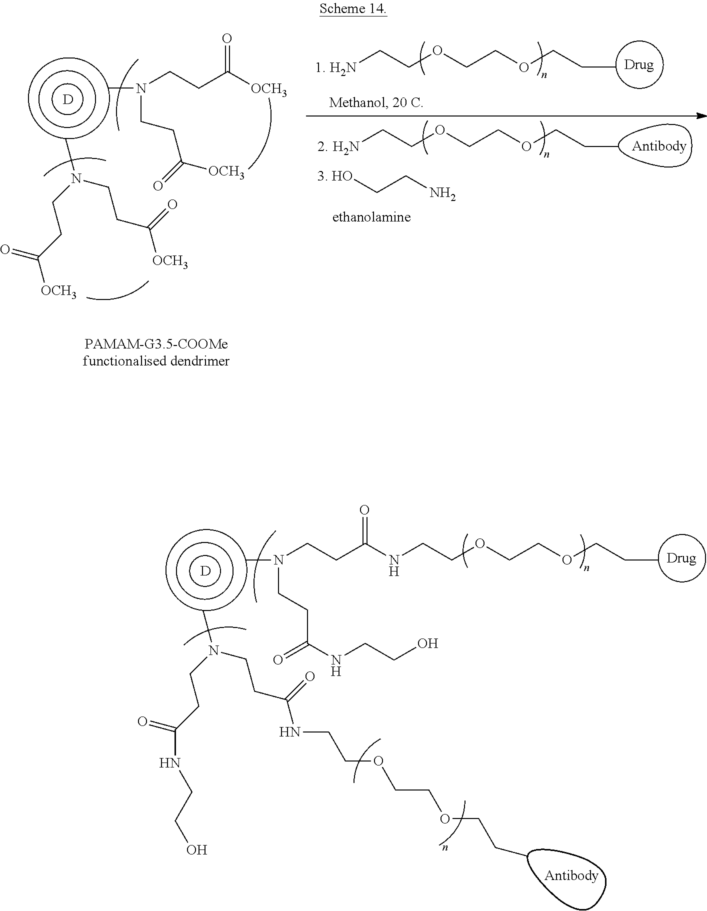

One antibody and oligonucleotide on one dendrimer:

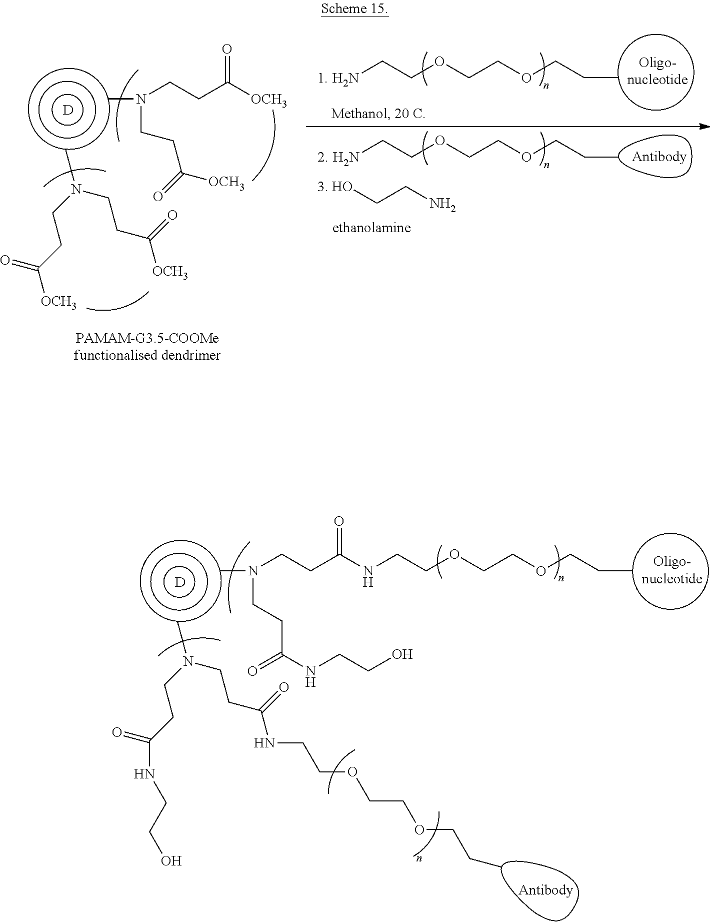

Peptide and Drug on one dendrimer in one pot:

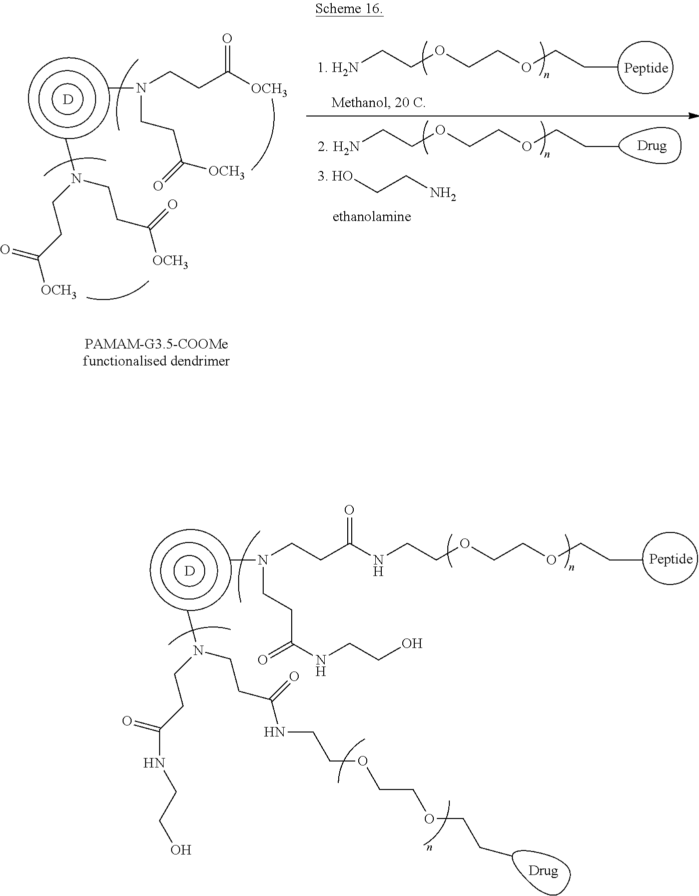

Targeting Peptide and PROTAC on one dendrimer in one pot:

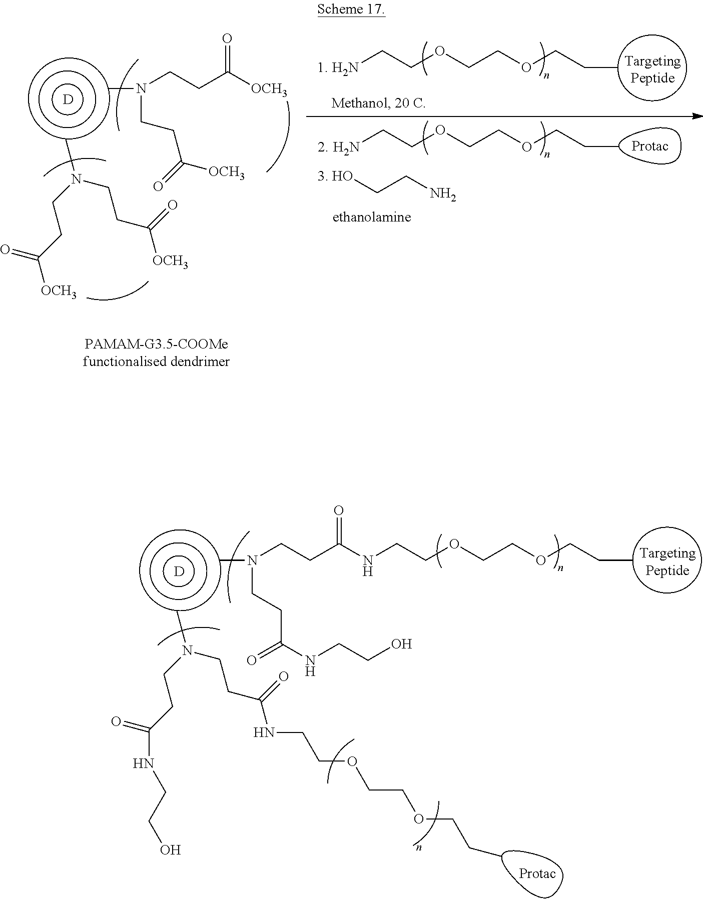

Drug and RNA or DNA on one dendrimer:

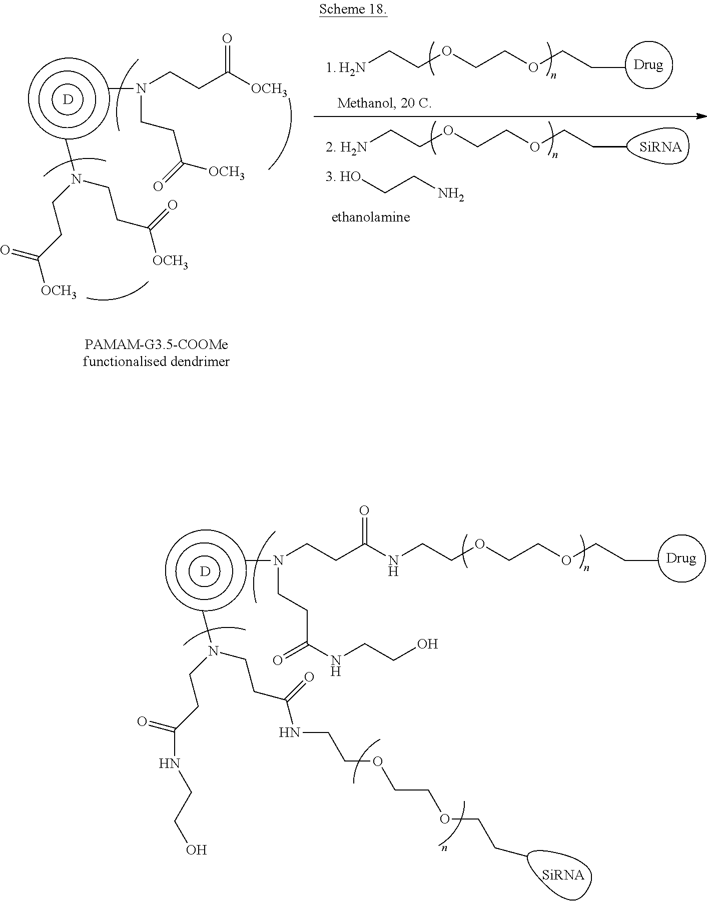

3 different drugs on one dendrimer in one pot:

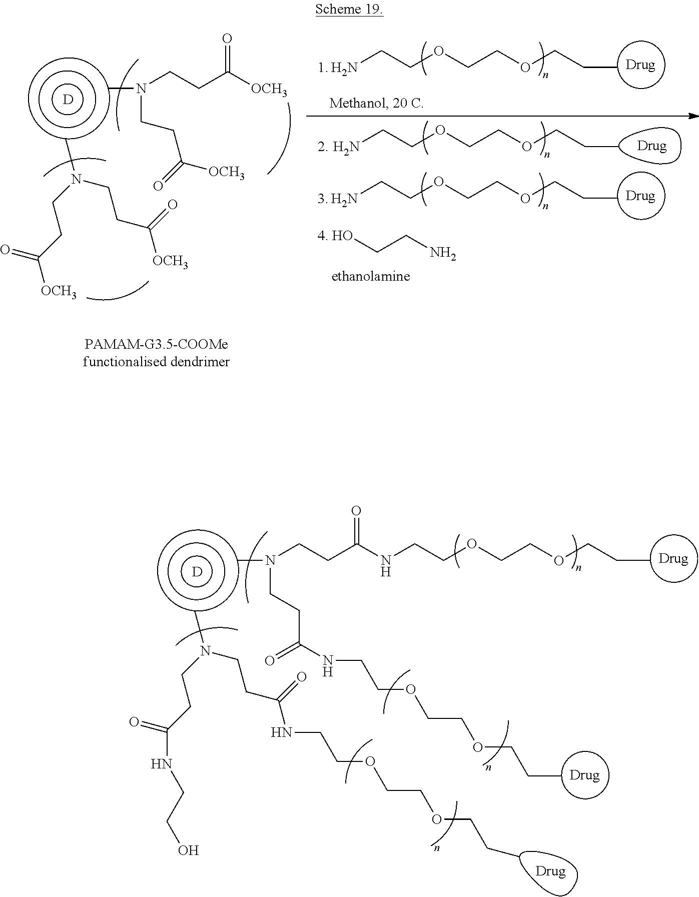

### Example 7. Conjugates of Dendrimer-PAK1 Inhibitor

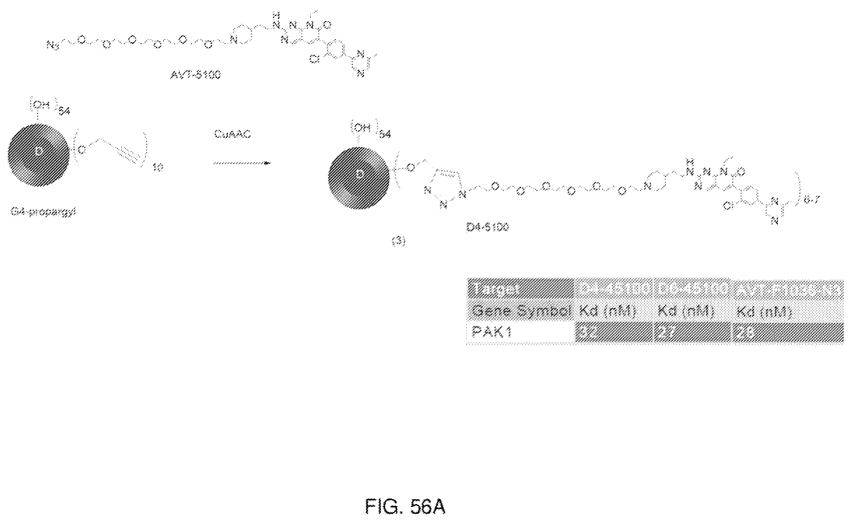

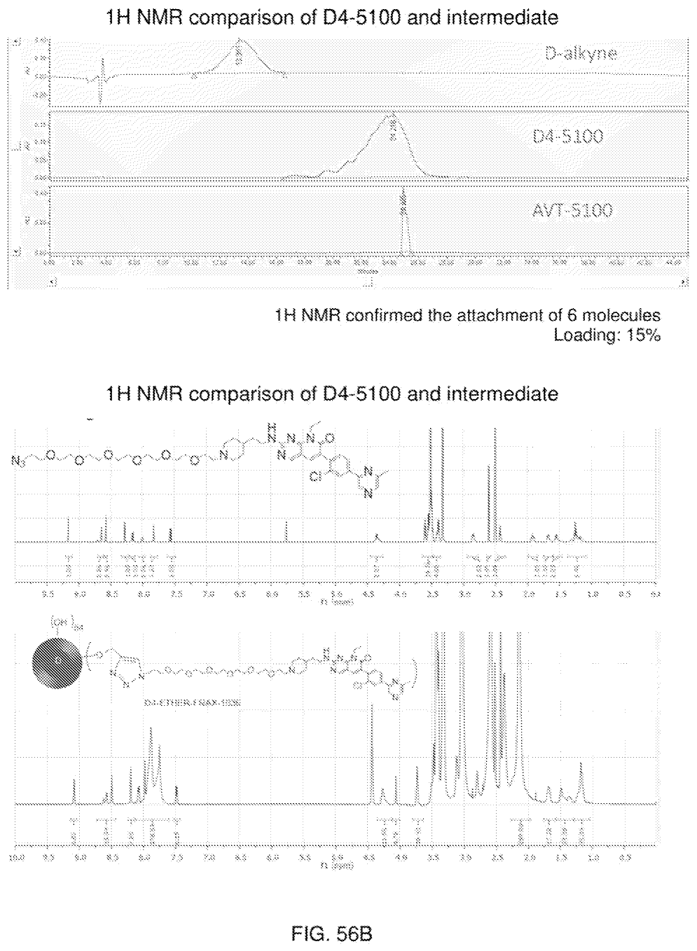

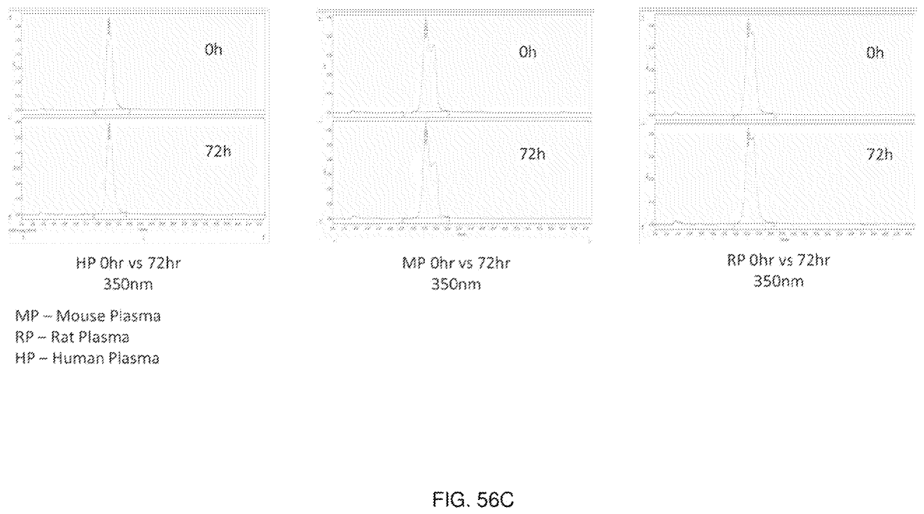

Dendrimers conjugated to PAK1 inhibitor (Frax-1036 analogue) were synthesized and characterized (FIGS. 56A-56C). FIG. 56A shows an example synthetic scheme for preparing a dendrimer-PAK1 conjugate, D4-5100 (D4-Frax-1036 analogue). PAK1 kinome binding for PAK1 was evaluated, and the results are shown in FIG. 56A (inset table). FIGS. 56B-56C show results from the characterization of D4-5100. D4-5100 was determined to be >99% pure by HPLC (FIG. 56B, top panels), and ¹H NMR confirmed the attachment of 6 molecules (FIG. 56B, bottom panels), with loading determined to be 15%. D4-5100 was further evaluated by plasma stability studies in human plasma (FIG. 56C, left panel), mouse plasma (FIG. 56C, middle panel), and rat plasma (FIG. 56C, right panel), which demonstrated that D4-5100 appeared stable in various plasma sources for 72 hours.

### Example 8. Conjugates of Dendrimer-MEK Inhibitor

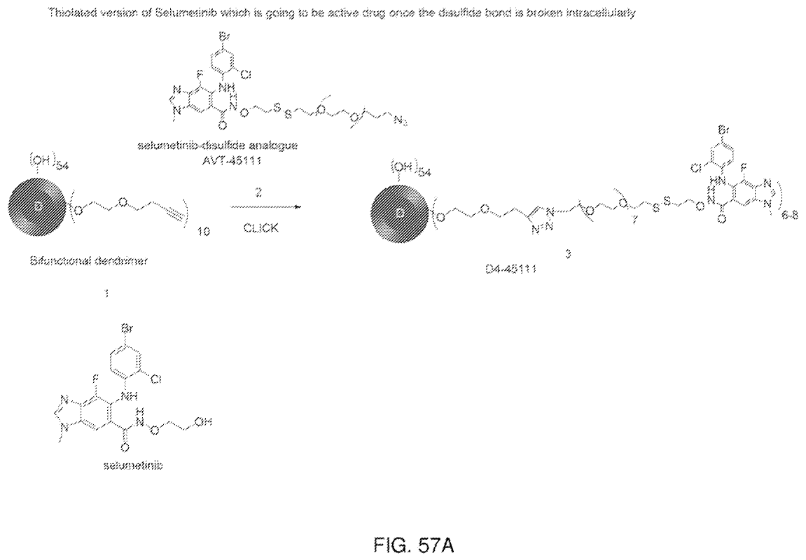

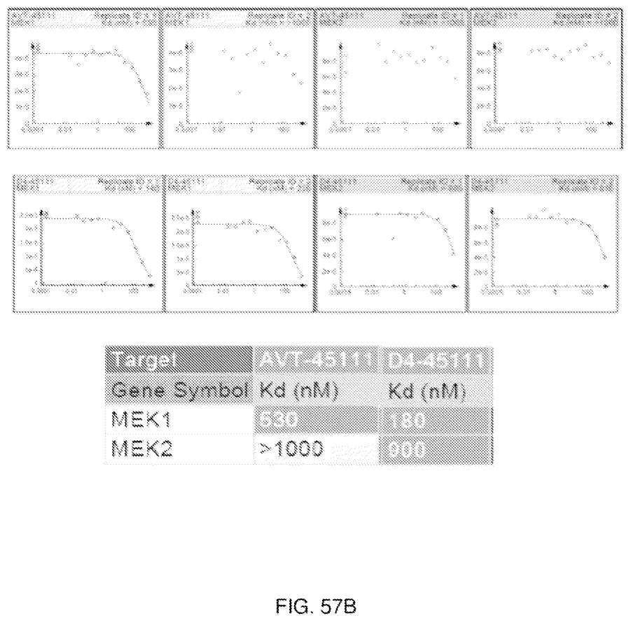

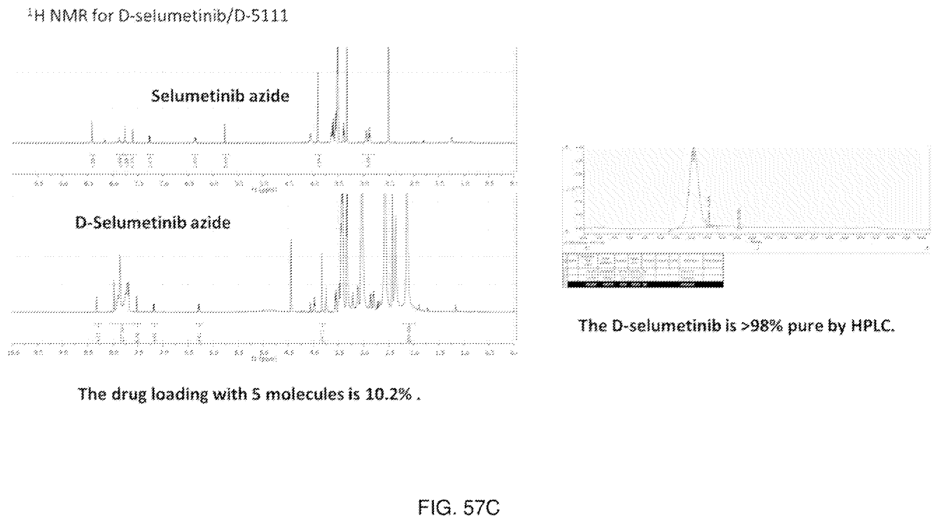

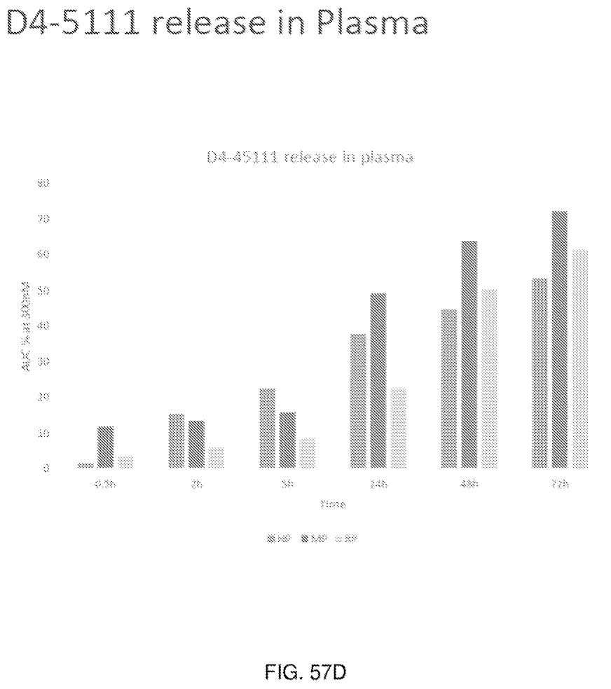

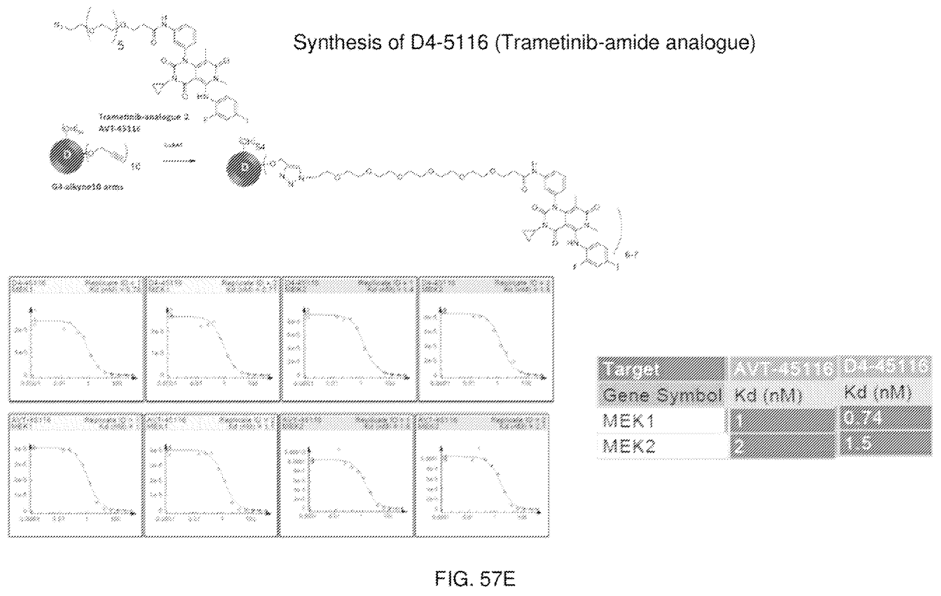

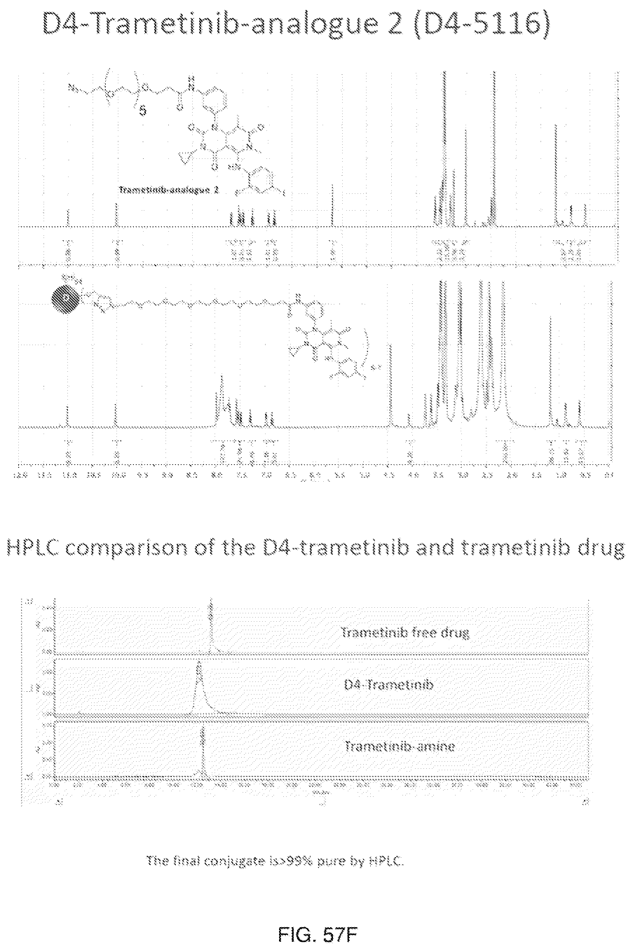

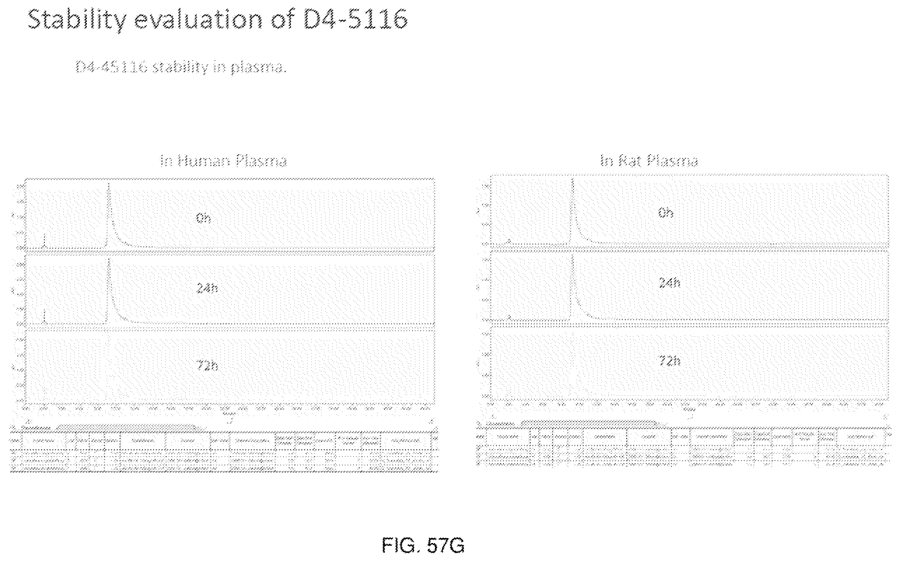

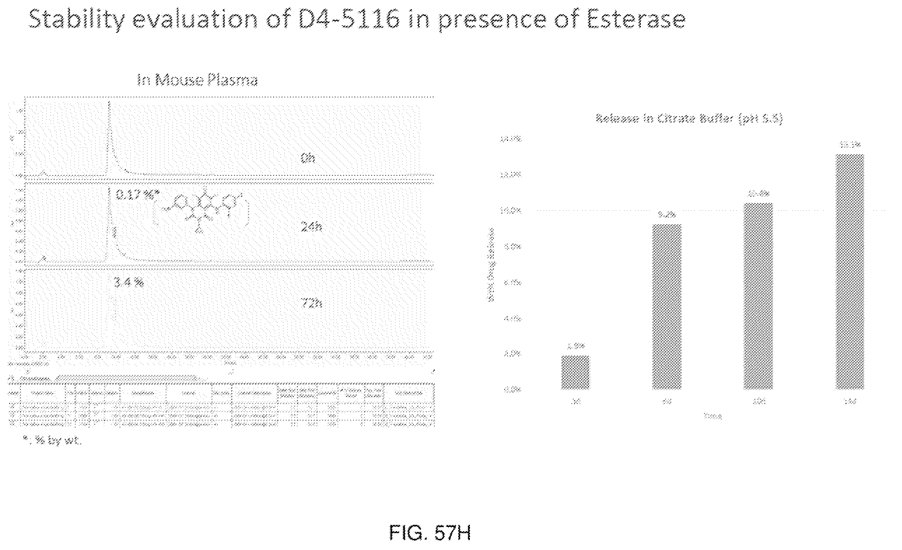

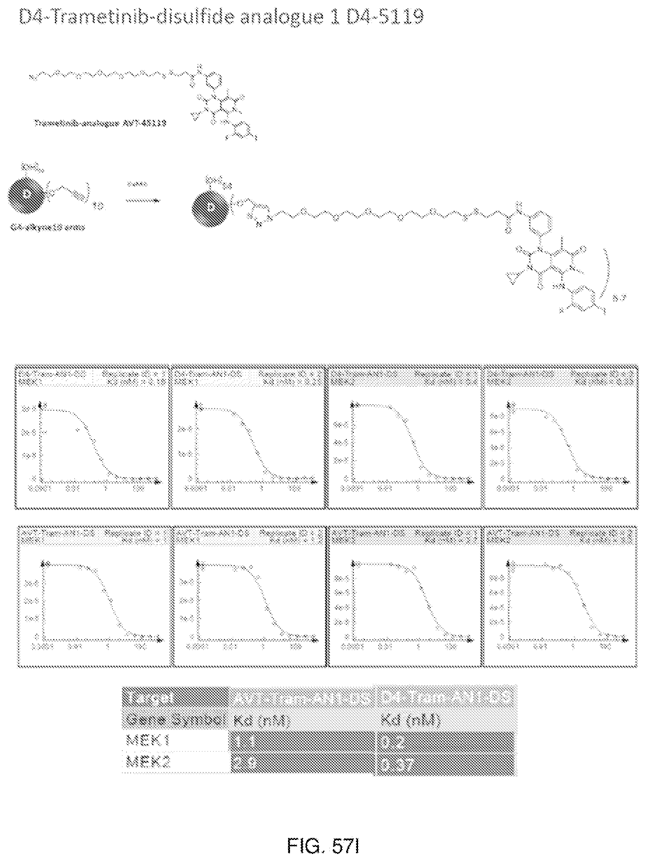

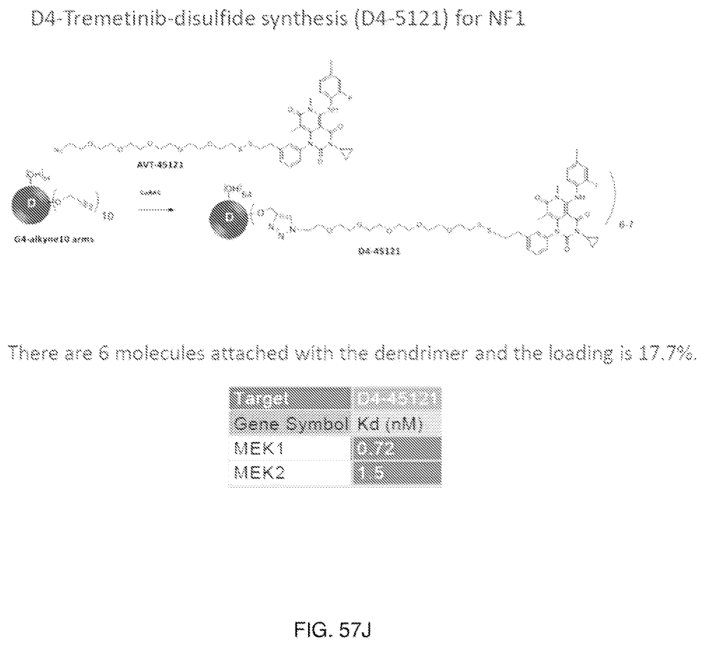

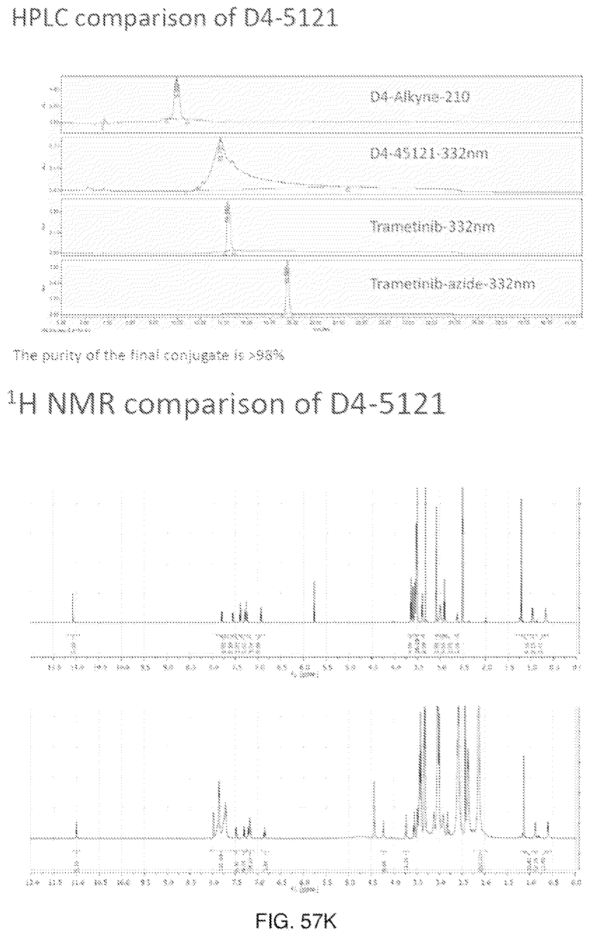

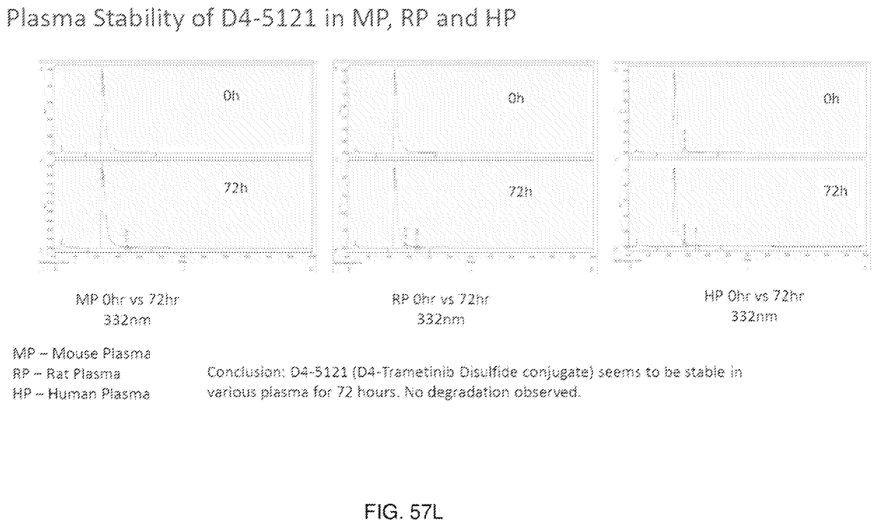

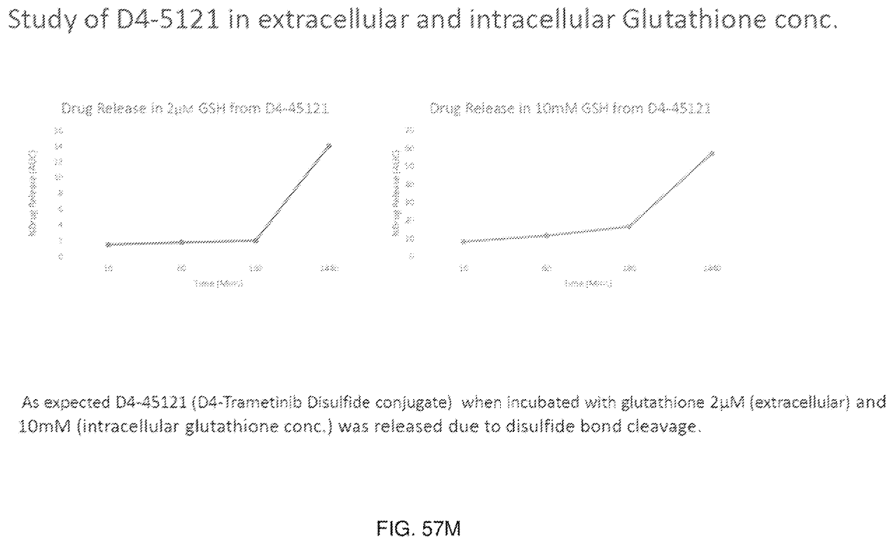

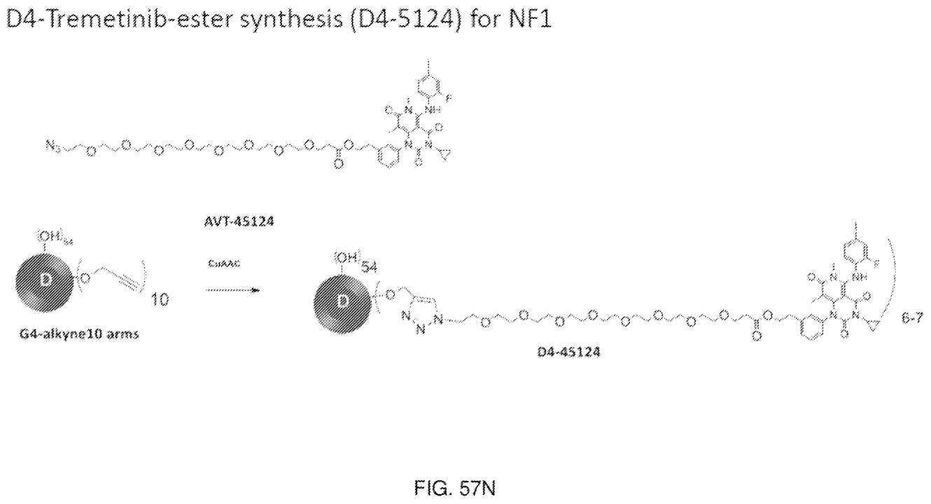

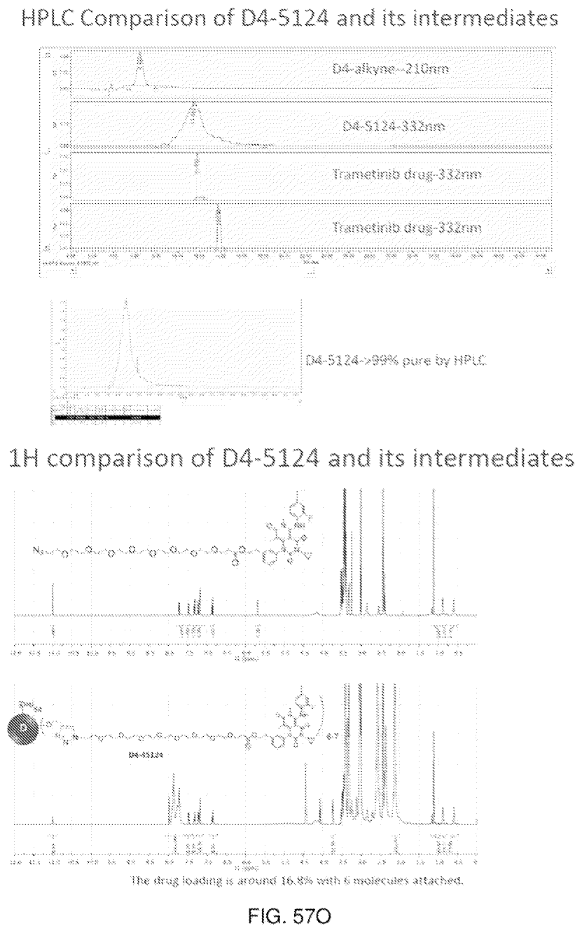

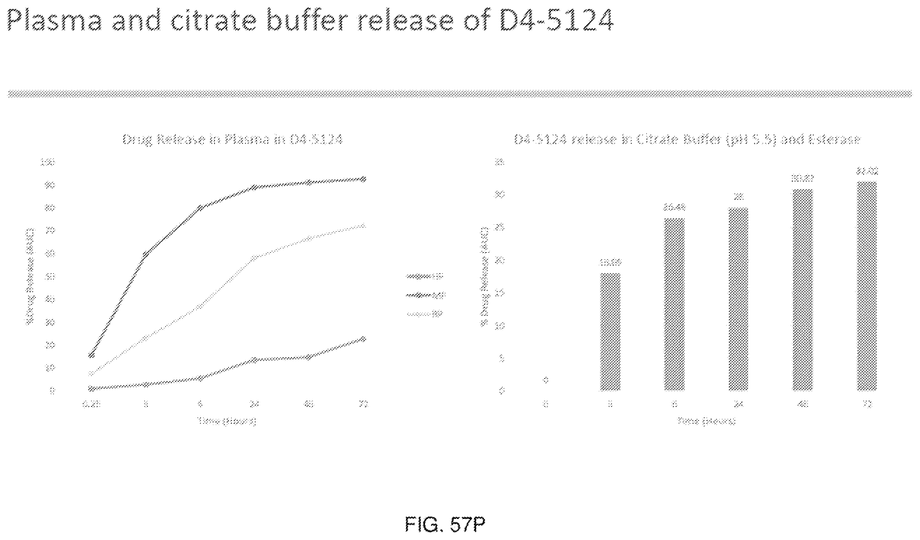

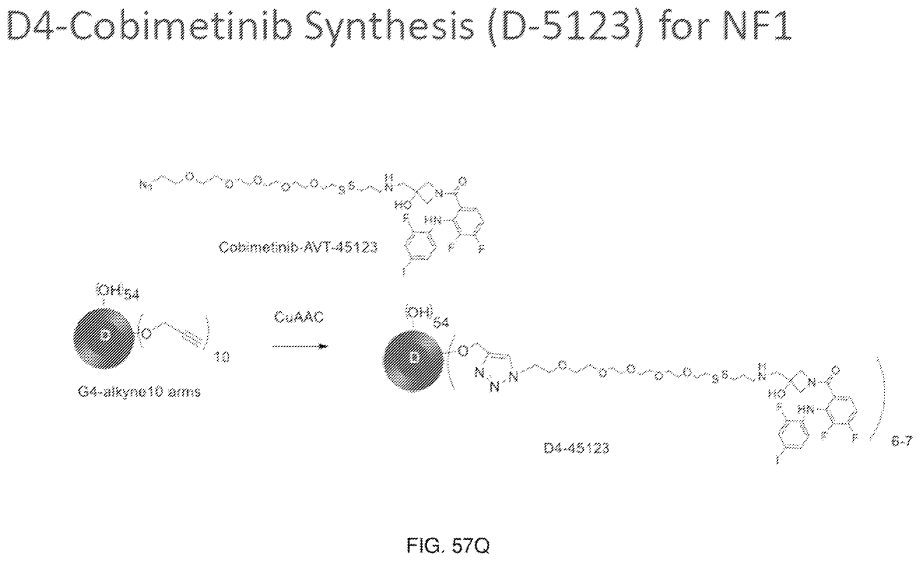

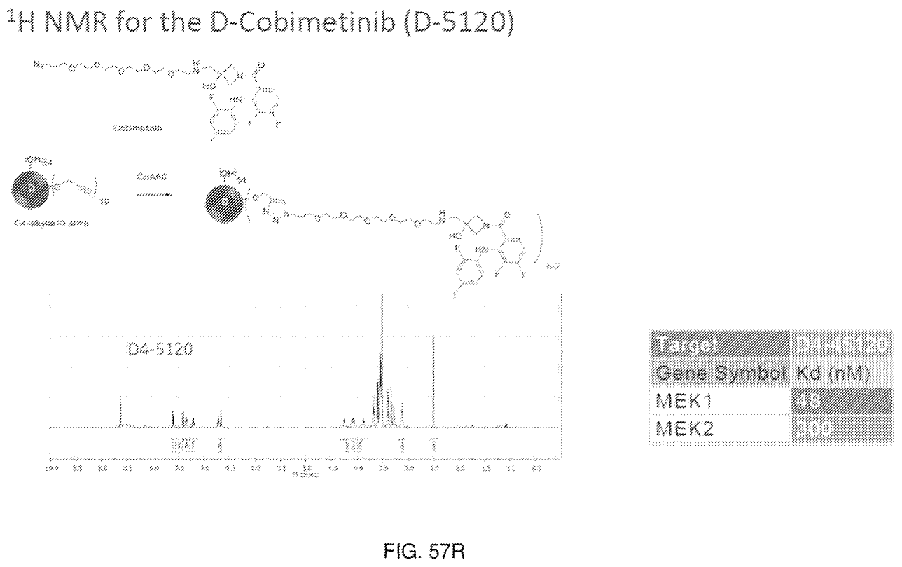

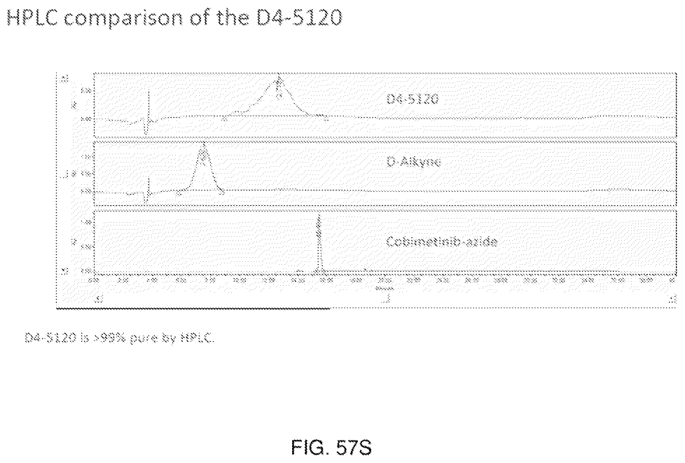

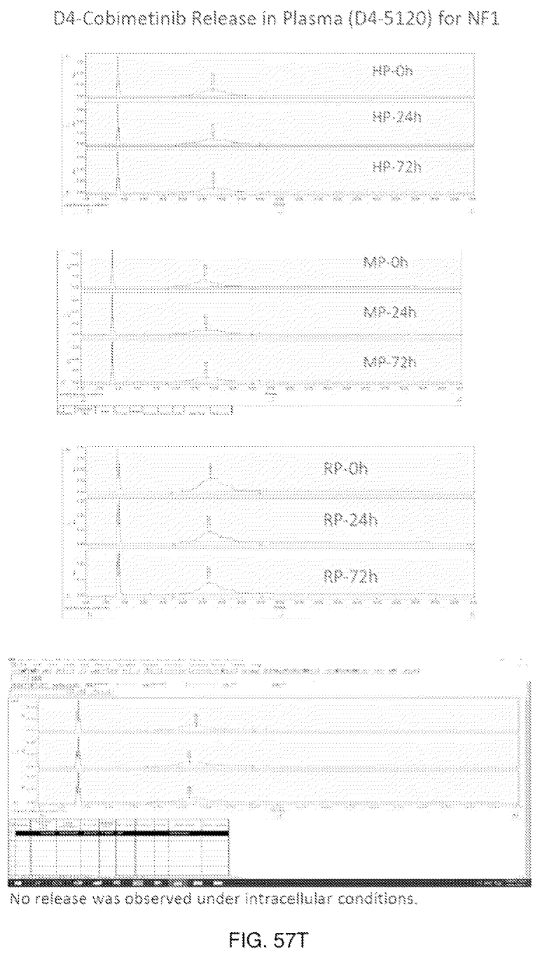

Dendrimers conjugated to MEK inhibitors (selumetinib, trametinib, or cobimetinib) were synthesized and characterized (FIGS. 57A-57T).

FIG. 57A shows an example synthetic scheme for preparing a dendrimer-selumetinib conjugate, D4-5111 (D4-selumetinib). FIGS. 57B-57D show results from characterization of D4-5111.

FIGS. 57E-57H show an example synthetic scheme for preparing a dendrimer-trametinib conjugate, D4-5116 (D4-trametinib-amide analogue), and results from characterization of D4-5116. FIG. 57I shows an example synthetic scheme for preparing a dendrimer-trametinib conjugate, D4-5119 (D4-trametinib-disulfide analogue), and results from characterization of D4-5119. FIGS. 57J-57M show an example synthetic scheme for preparing a dendrimer-trametinib conjugate, D4-5121 (D4-trametinib-disulfide), and results from characterization of D4-5121. FIGS. 57N-57P show an example synthetic scheme for preparing a dendrimer-trametinib conjugate, D4-5124 (D4-trametinib-ester), and results from characterization of D4-5124.

FIG. 57Q shows an example synthetic scheme for preparing a dendrimer-cobimetinib conjugate, D4-5123. FIGS. 57R-57T show an example synthetic scheme for preparing a dendrimer-cobimetinib conjugate, D4-5120, and results from characterization of D4-5120.

### Example 9. Conjugates of Dendrimer-Receptor Tyrosine Kinase Inhibitor

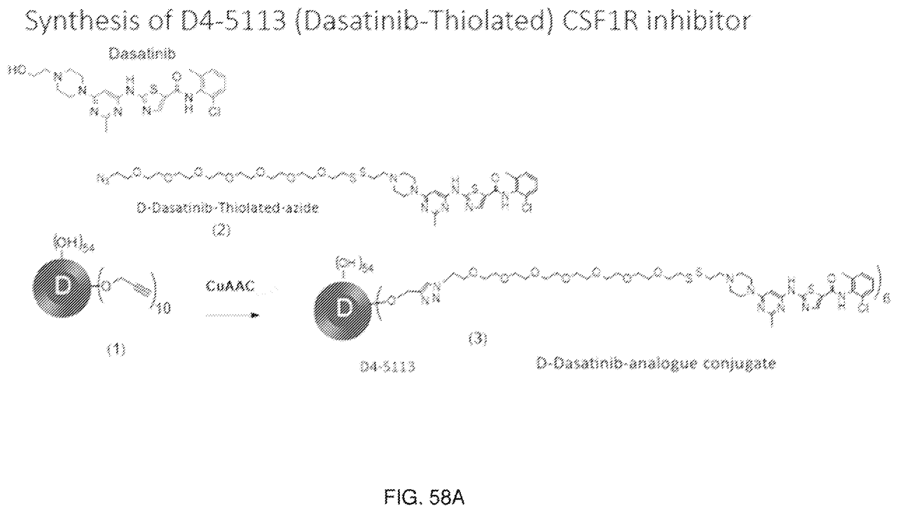

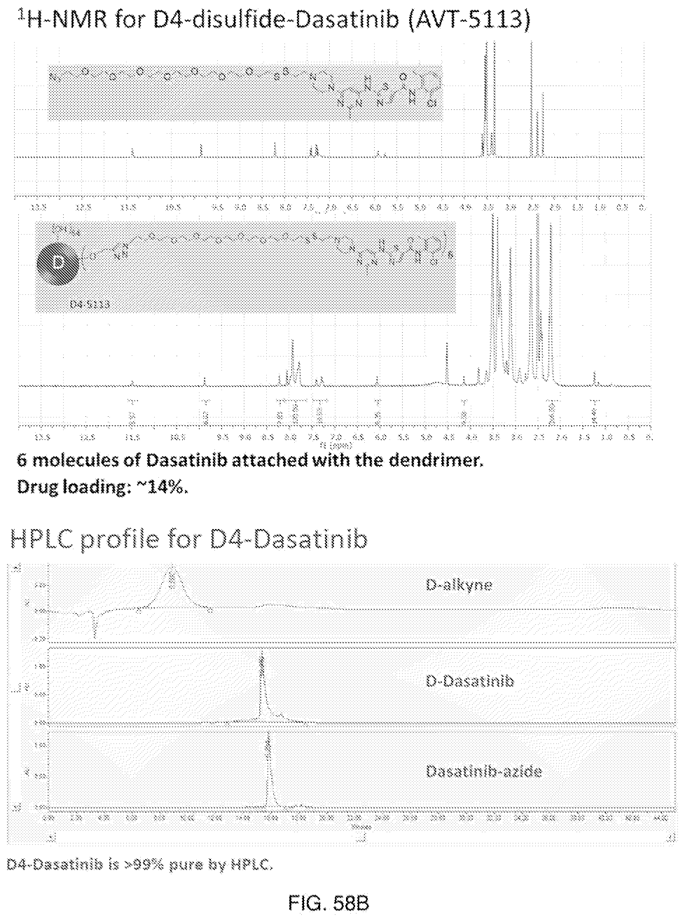

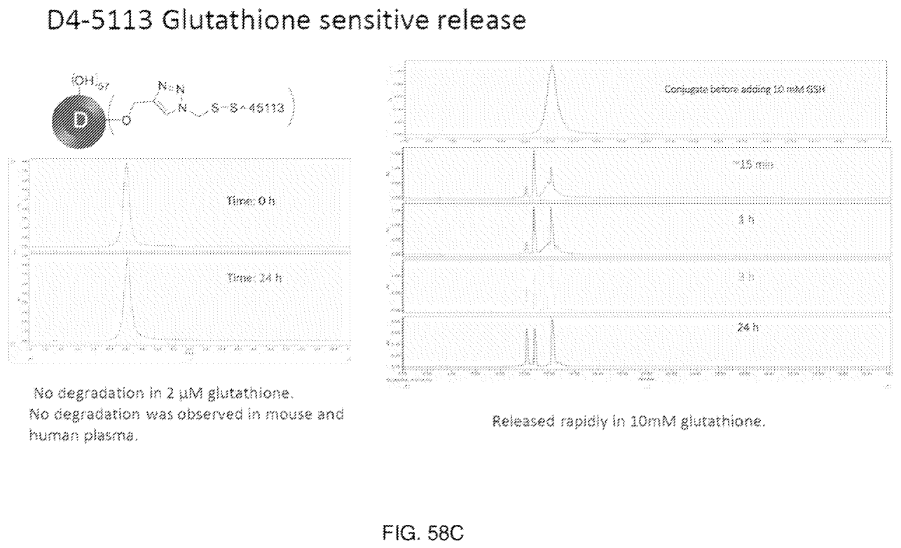

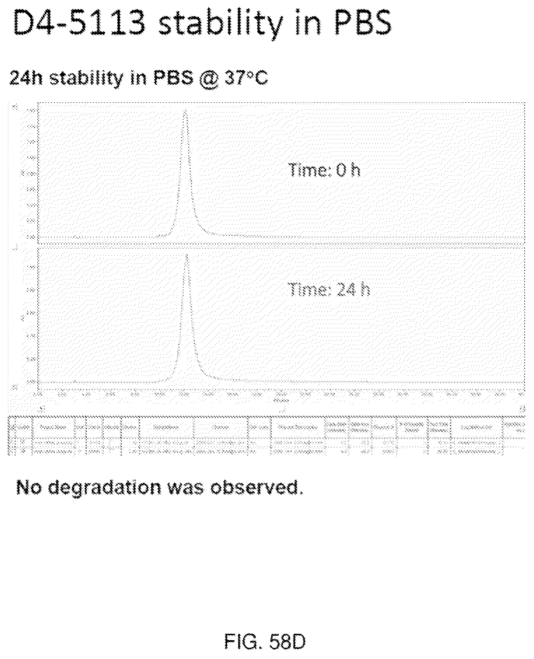

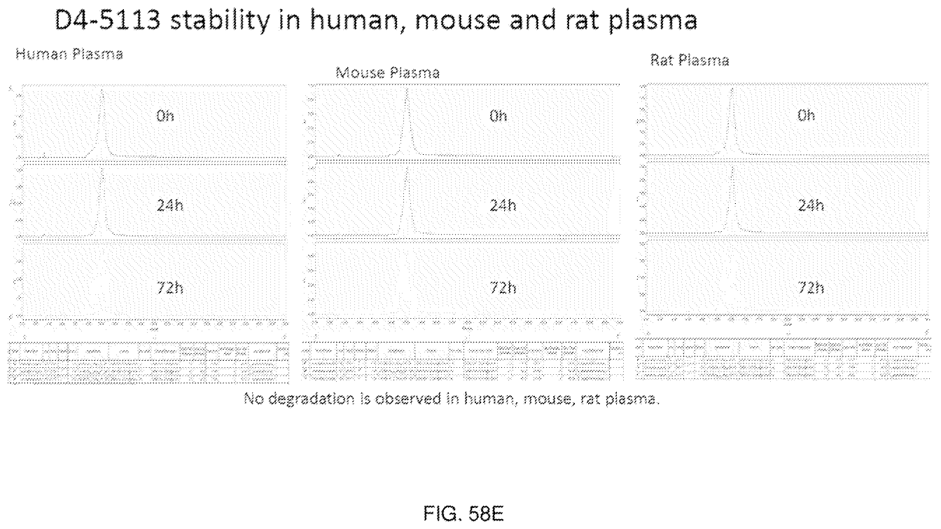

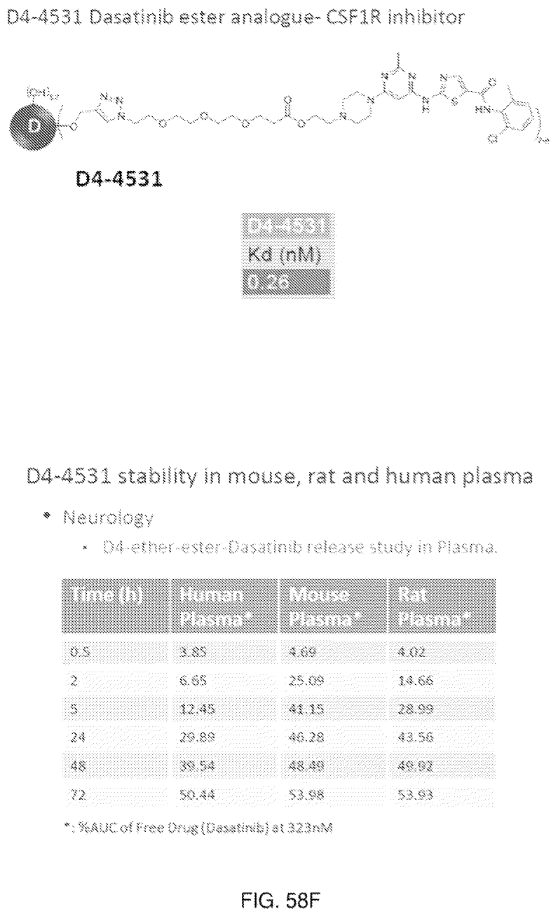

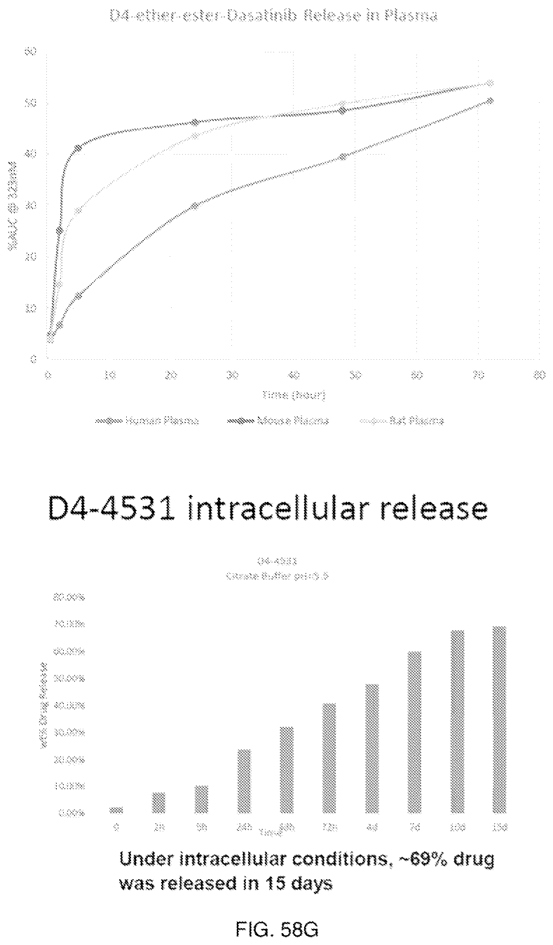

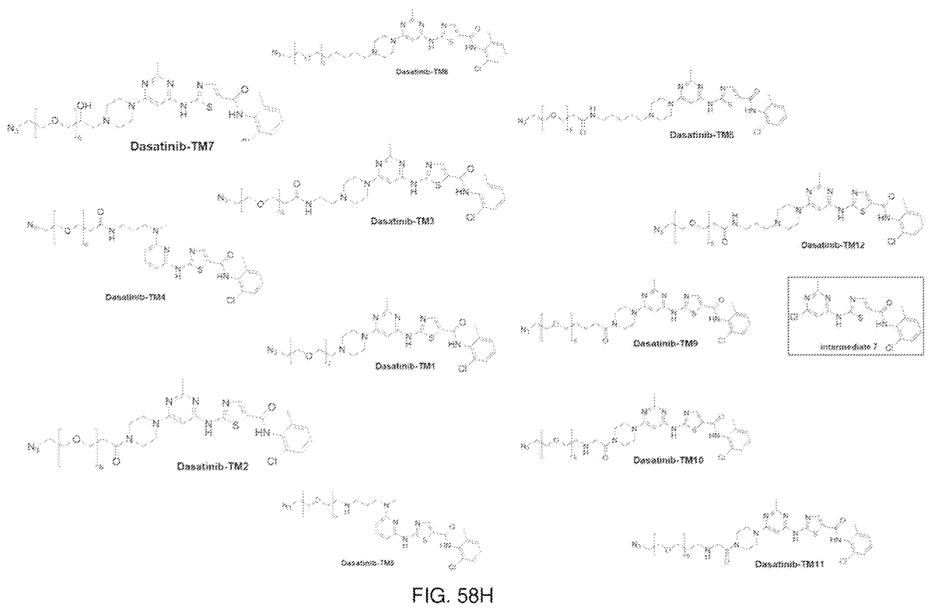

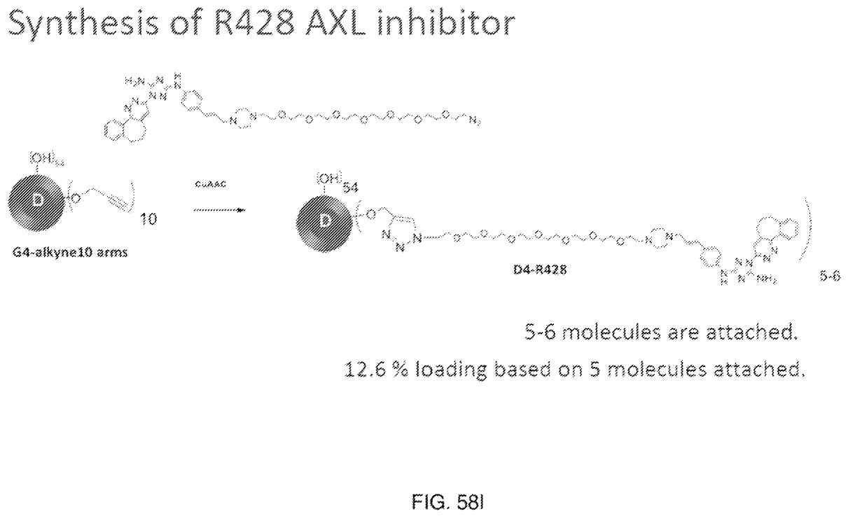

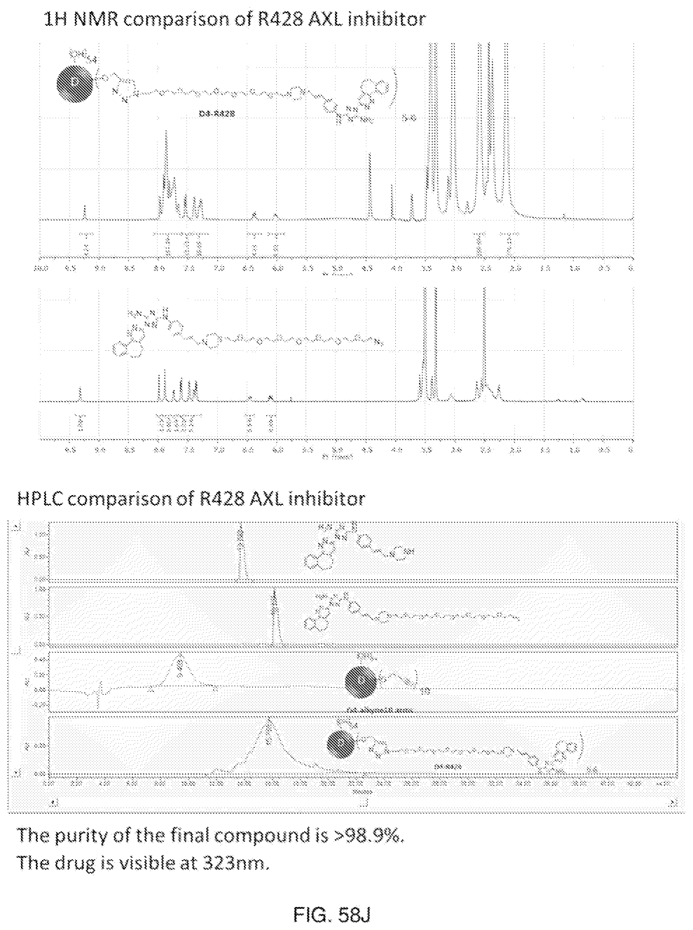

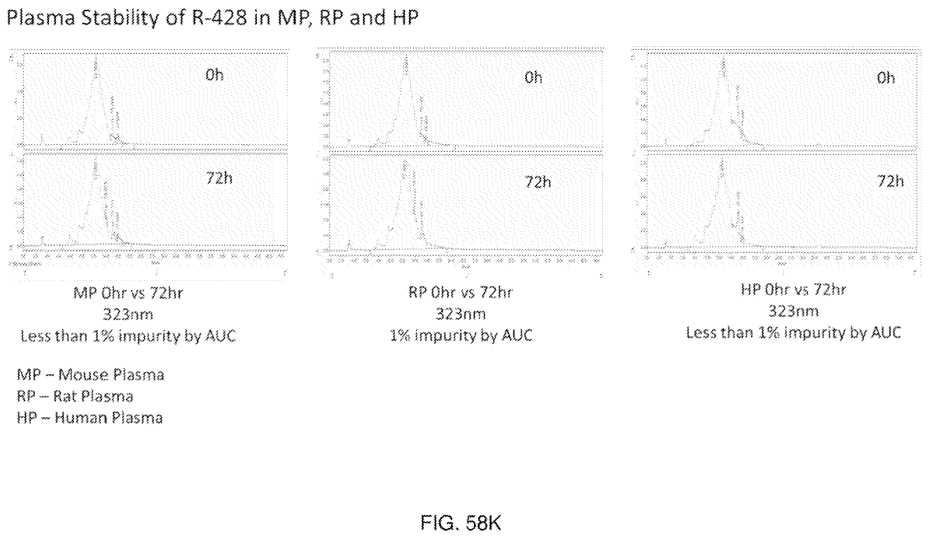

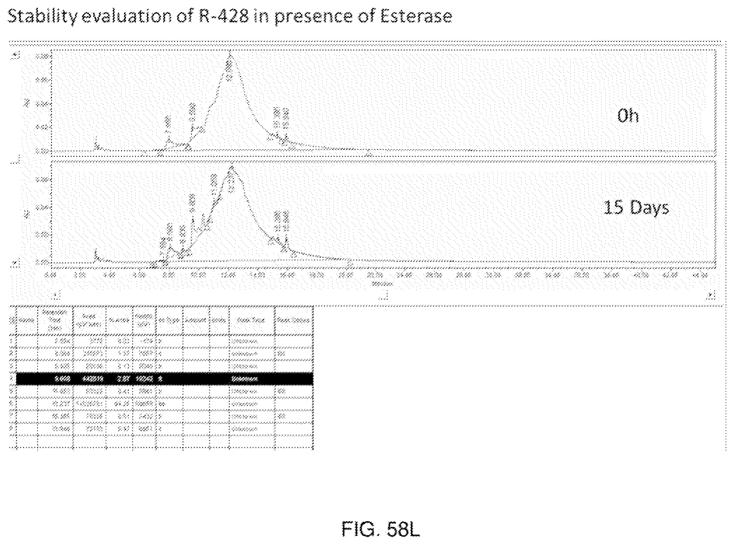

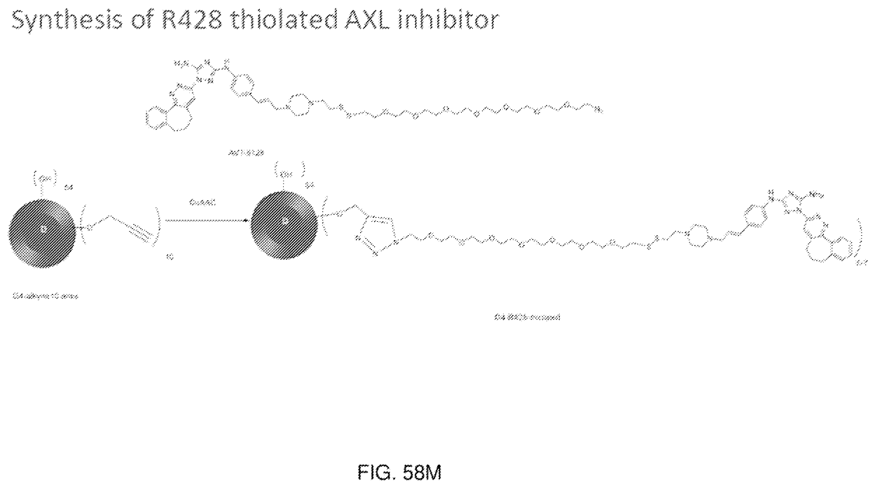

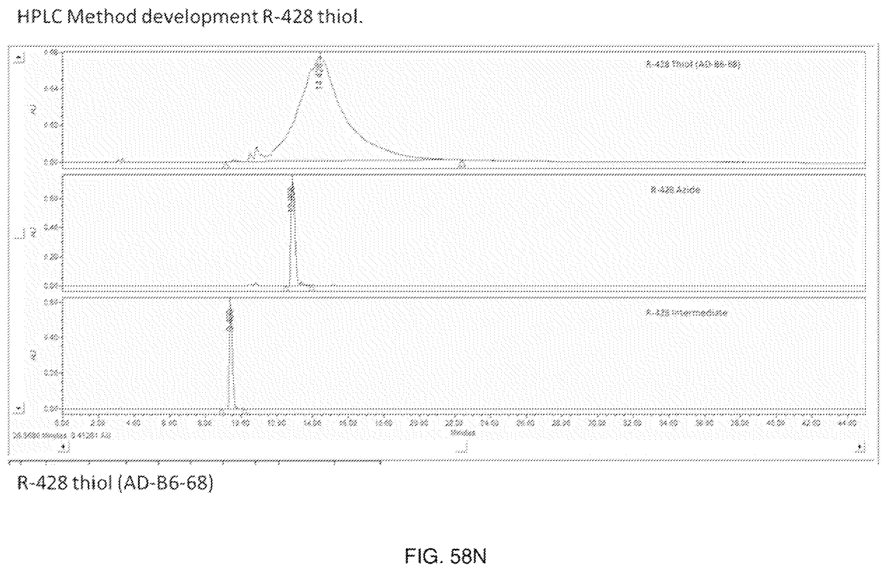

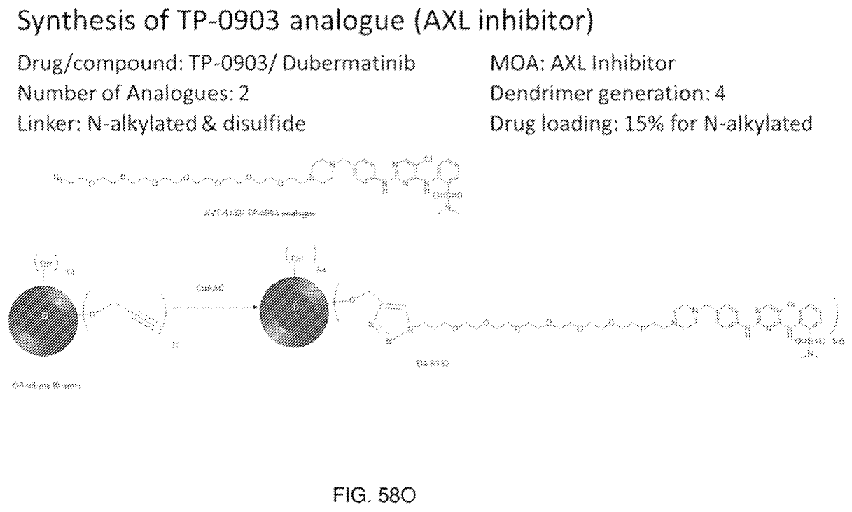

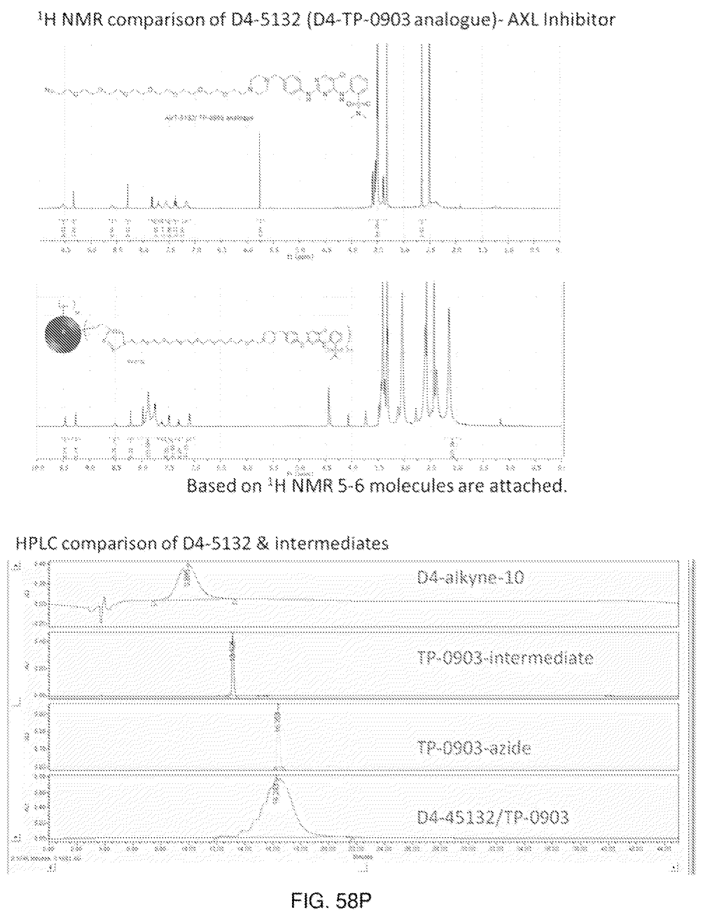

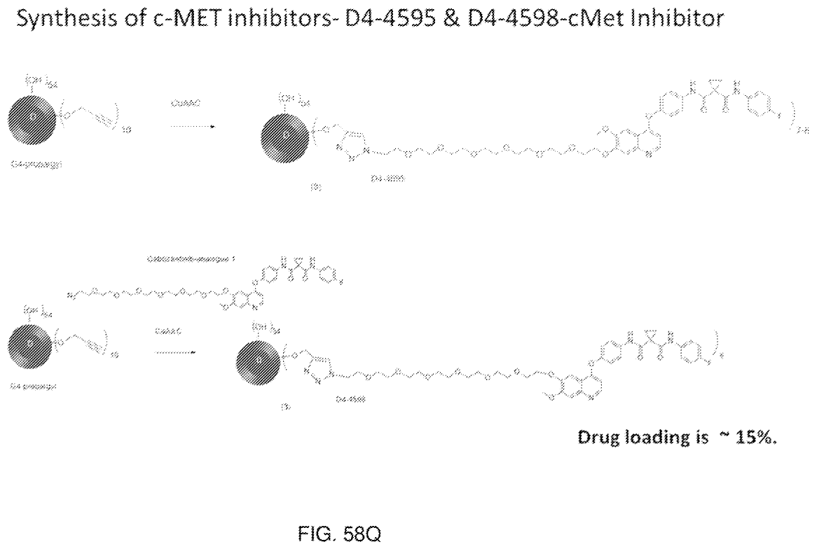

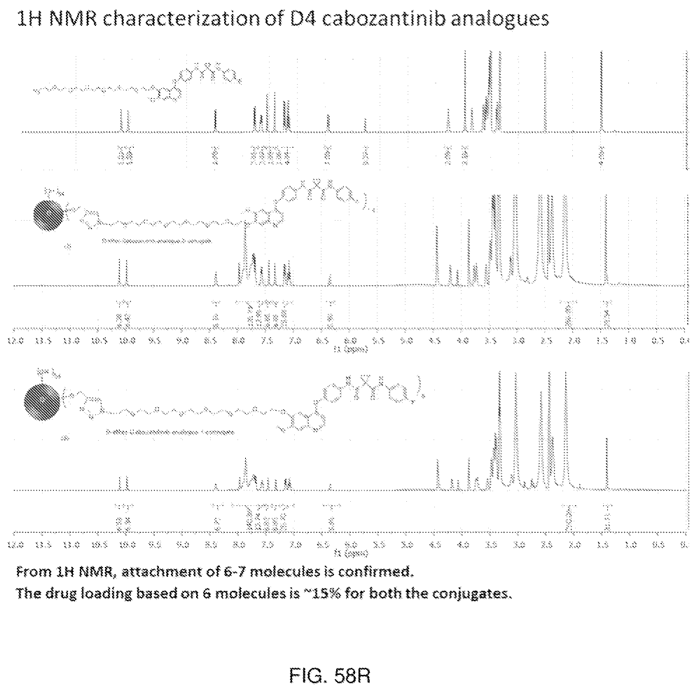

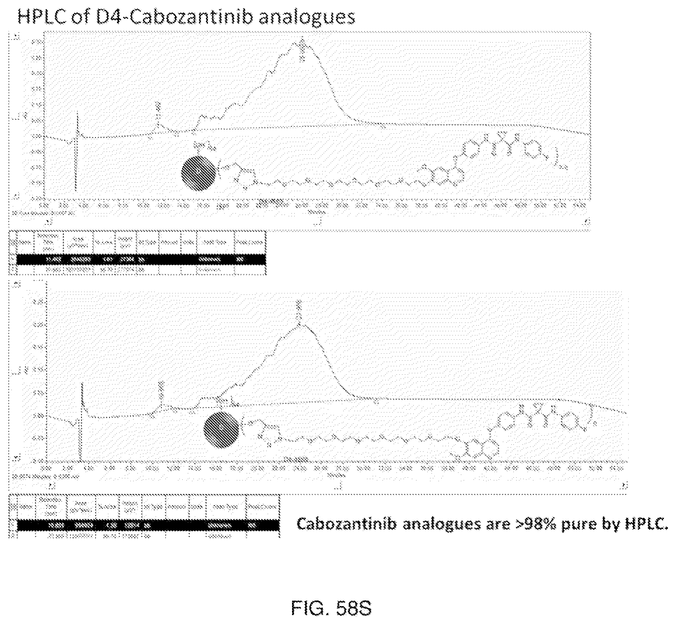

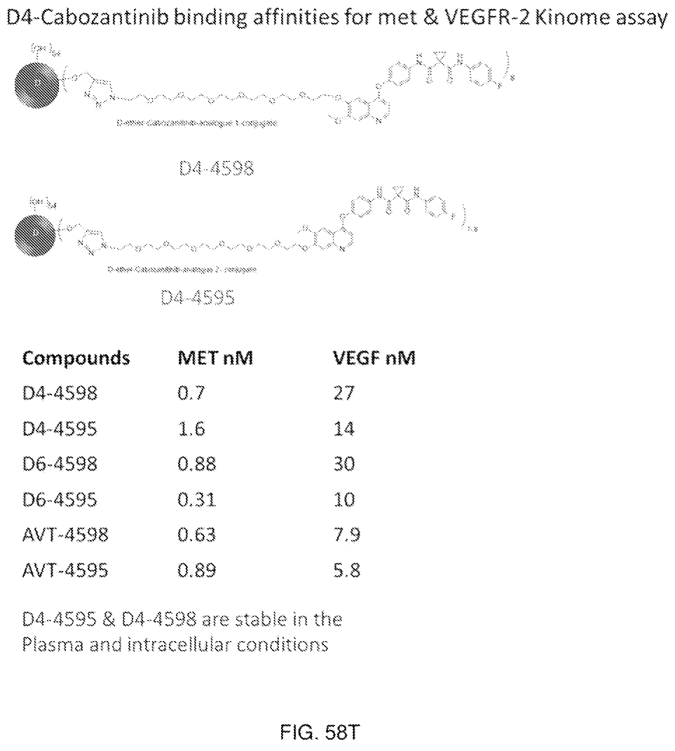

Dendrimers conjugated to receptor tyrosine kinase inhibitors (dasatinib, bemcentinib (R428), dubermatinib (TP-0903), or cabozantinib) were synthesized and characterized (FIGS. 58A-58T).

FIG. 58A shows an example synthetic scheme for preparing a dendrimer-dasatinib conjugate, D4-5113 (D4-dasatinib-thiolated). FIGS. 58B-58E show results from characterization of D4-5113. FIGS. 58F-58G show a chemical structure and results from characterization of a synthesized dendrimer-dasatinib conjugate, D4-4531 (D4-dasatinib ester analogue). FIG. 58H shows example chemical structures of dasatinib analogues for chemical conjugation via terminal azide groups.

FIG. 58I shows an example synthetic scheme for preparing a dendrimer-bemcentinib (R428) conjugate, D4-R428. FIGS. 58J-58L show results from characterization of D4-R428. FIG. 58M shows an example synthetic scheme for preparing a dendrimer-bemcentinib (R428) conjugate, D4-R428-thiolated. FIG. 58N shows results from characterization of D4-R428-thiolated.

FIG. 58O shows an example synthetic scheme for preparing a dendrimer-dubermatinib (TP-0903) conjugate, D4-5132 (D4-TP-0903 analogue). FIG. 58P shows results from characterization of D4-5132.

FIG. 58Q shows example synthetic schemes for preparing a dendrimer-cabozantinib conjugates, D4-4595 and D4-4598. FIGS. 58R-58T show results from characterization of D4-4595 and D4-4598.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

1-176. (canceled)

177. A composition comprising a carrier and functionalized dendrimers of Formula (I-A): 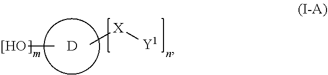
wherein:
D is a dendrimer selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof;
X is NH;
Y¹ is optionally substituted alkylene, optionally substituted alkenylene, optionally substituted alkynylene, or a covalent bond;
m is an integer from 16 to 4096, inclusive; and
n is an integer from 1 to 100, inclusive,
wherein the polydispersity value of the functionalized dendrimers in the composition is less than or equal to 1.10.

178. A method of synthesizing a functionalized dendrimer of Formula (I-A): 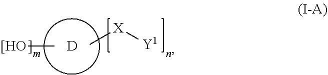
wherein:
D is a dendrimer selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof;
X is NH;
Y¹ is optionally substituted alkylene, optionally substituted alkenylene, optionally substituted alkynylene, or a covalent bond;
m is an integer from 16 to 4096, inclusive; and
n is an integer from 1 to 100, inclusive;
the method comprising:
Reacting a dendrimer of Formula (II-A): 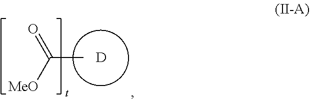
under suitable conditions to form the functionalized dendrimer of Formula (I-A);
wherein:
D is a dendrimer selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof;
t is an integer from 16 to 4096, inclusive;
with one or more amines, wherein each amine is of the formula H₂NR¹,
wherein R¹ is optionally substituted alkylene, optionally substituted alkenylene, optionally substituted alkynylene, or a covalent bond.

179. The method of claim 178, wherein n is 1, 2, 3, 4, or 10.

180. The method of claim 178, wherein m is 61 or 54.

181. The method of claim 178, wherein at least one instance of R¹ is optionally substituted alkylene.

182. The method of claim 178, wherein at least one instance of the one or more amines is a compound of Formula (A): 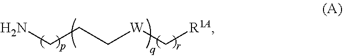
wherein:
R^{1A} is halogen, optionally substituted acyl, optionally substituted alkenylene, optionally substituted alkynylene, optionally substituted acetylene, optionally substituted carbocyclyl, optionally substituted heterocyclyl, optionally substituted aryl, optionally substituted heteroaryl, —CH(═N)(OH)R^{D1}, —CN, —NO₂, —OR^{D1}, —N(R^{D1}a)₂, —SO₂OR^{D1}, or —SR^{D1}, wherein R^{D1} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group when attached to an oxygen atom, or a sulfur protecting group when attached to a sulfur atom;
wherein each occurrence of R^{D1a} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring;
W is —O— or —CH₂—, as valency permits;
p is 0, 1, 2, or 3;
q is an integer between 1-100,000, inclusive; and
r is 0, 1, 2, 3, 4, 5, or 6.

183. The method of claim 182, wherein:
W is —O— and q is an integer between 1-100,000, inclusive; or
W is —CH₂— and q is an integer between 1-10,000, inclusive.

184. The method of claim 178, wherein at least one instance of the one or more amines is a PEG-alkyne of formula: 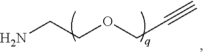
wherein q is 1, 2, 3, 4, 5, or 6.

185. The method of claim 178, wherein at least one instance of the one or more amines is ethanolamine.

186. The method of claim 178, wherein D is PAMAM.

187. The method of claim 178, wherein the dendrimer of Formula (II-A) is of formula:

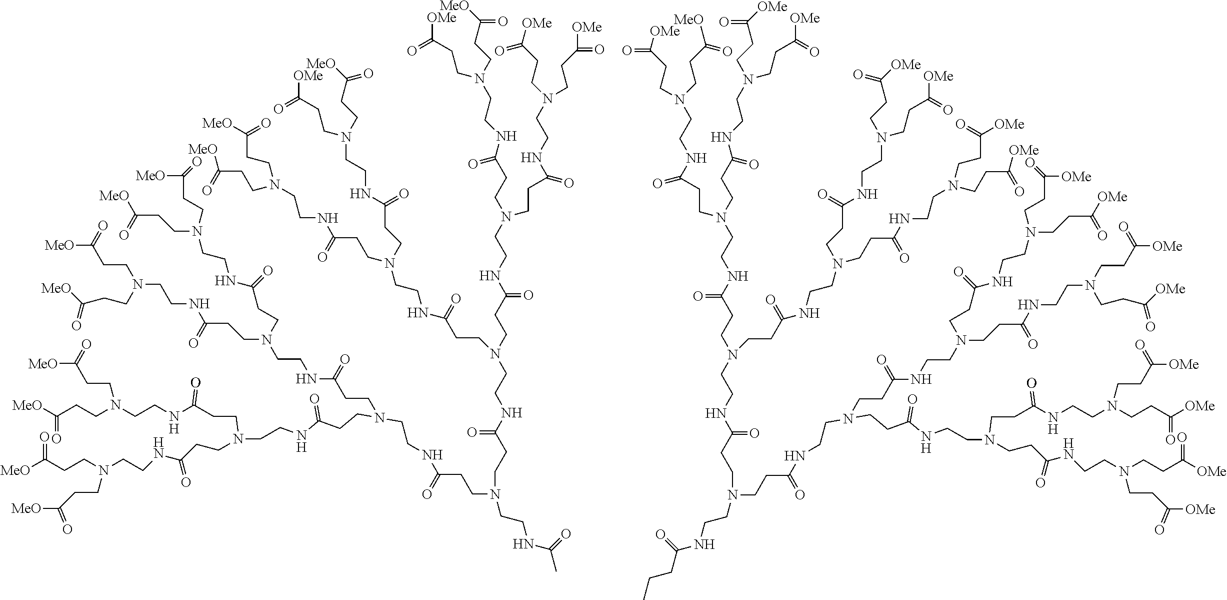

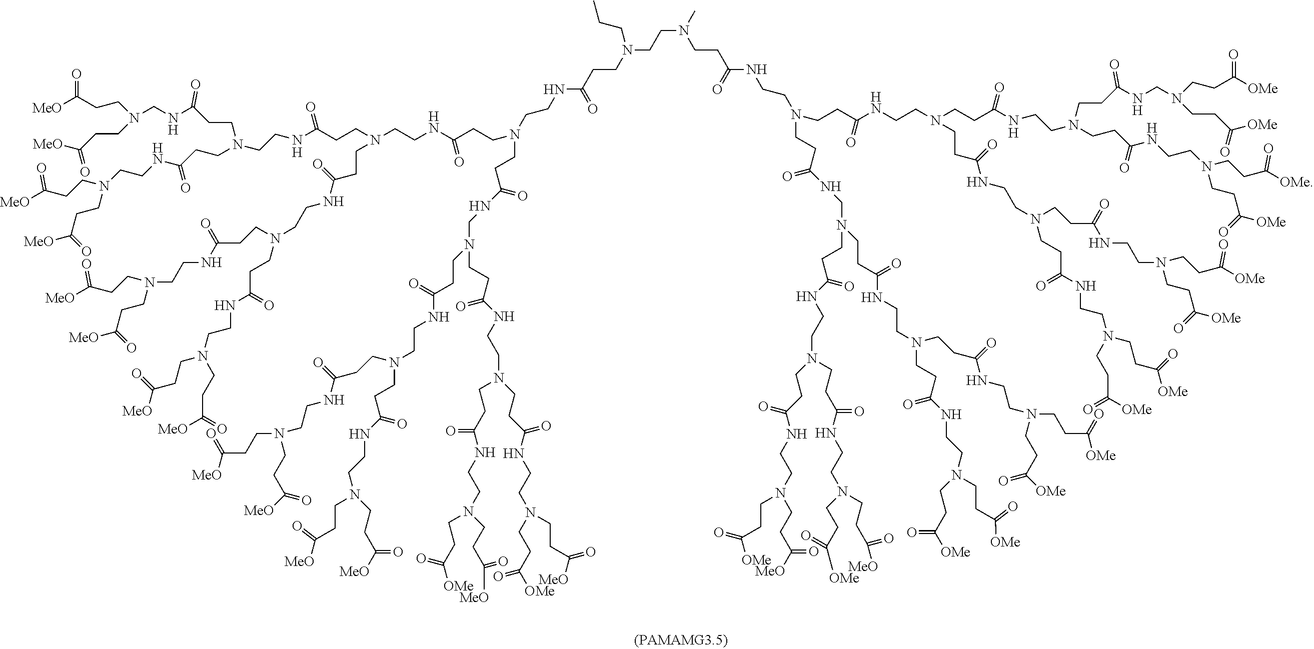

188. The method of claim 178, wherein the suitable conditions comprise a protic solvent and reacting at approximately 19° C. to approximately 23° C.

189. The method of claim 178, wherein the functionalized dendrimer of Formula (I-A) is of formula:

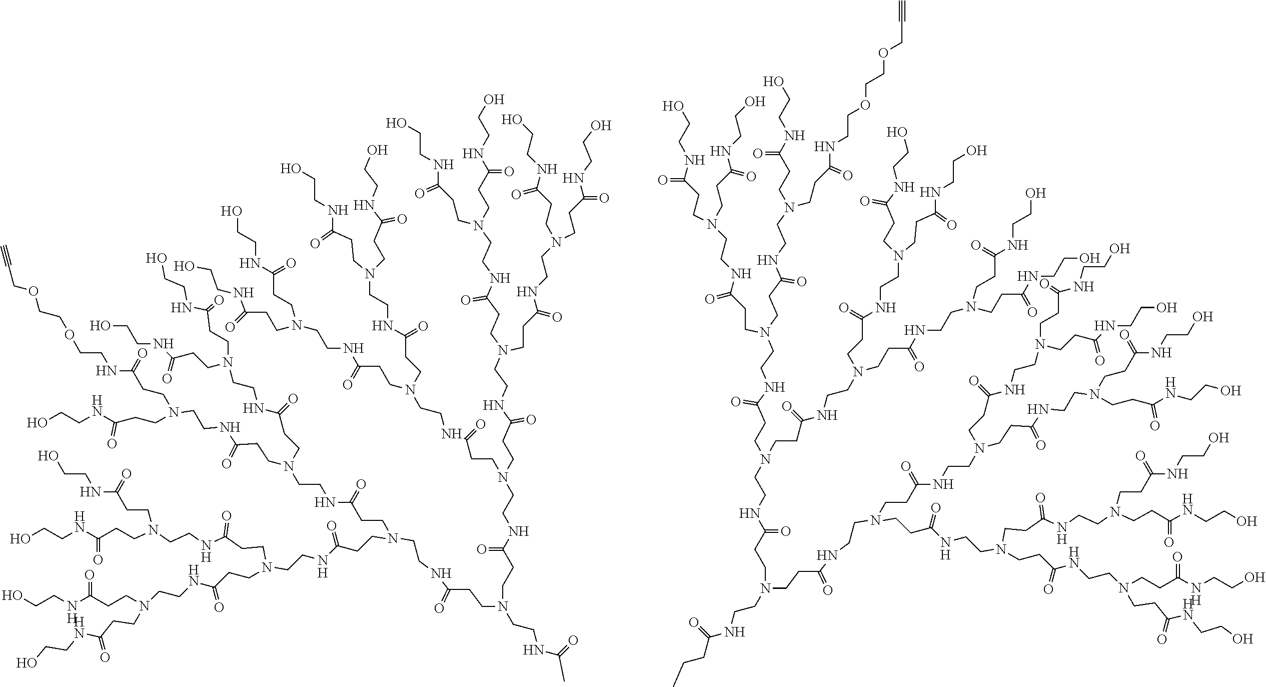

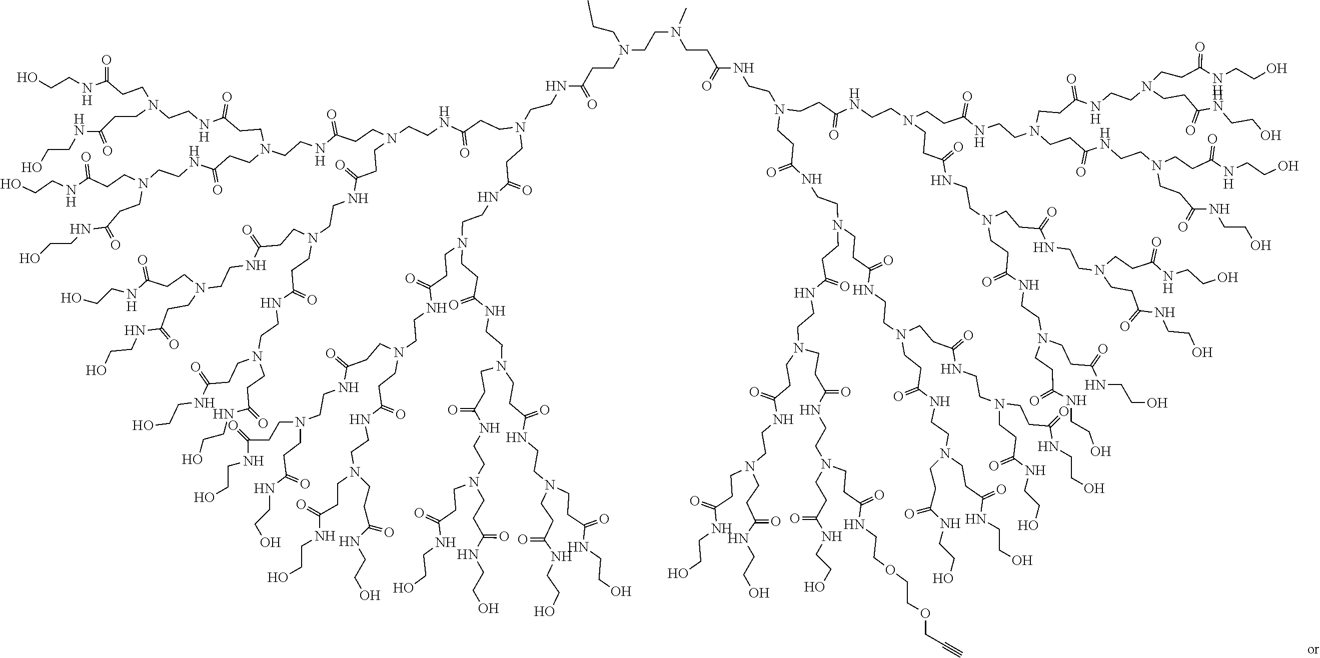

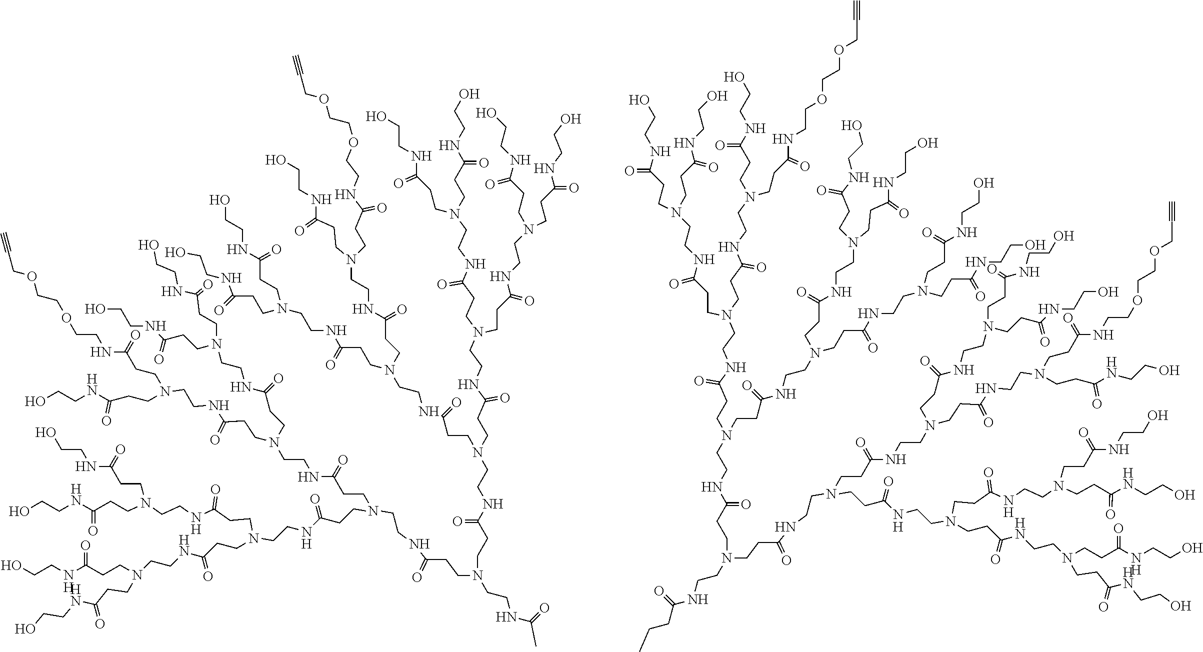

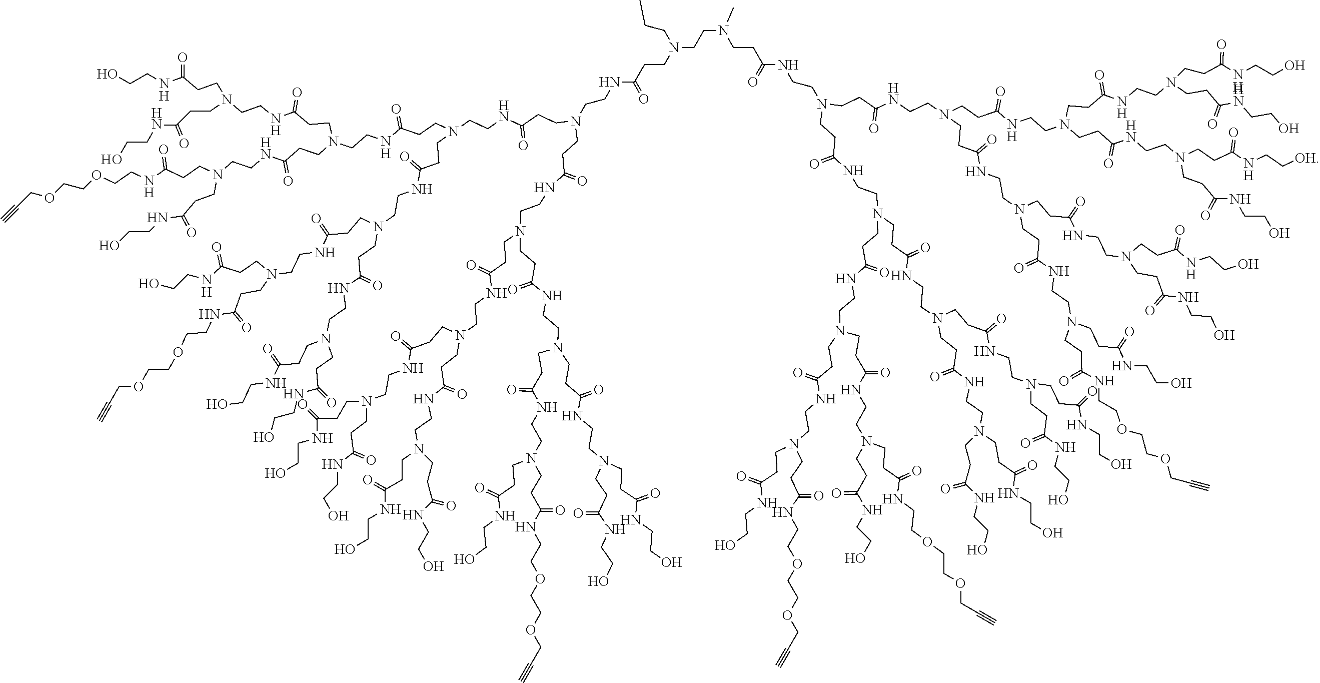

190. The method of claim 178, further comprising reacting with a compound of Formula (B): 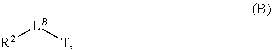
wherein:
R² is halogen, optionally substituted acyl, optionally substituted alkenyl, optionally substituted alkynyl, optionally substituted acetyl, optionally substituted carbocyclyl, optionally substituted heterocyclyl, optionally substituted aryl, optionally substituted heteroaryl, —N₃, —CH(═N)(OH)R^{D1}, —CN, —NO₂, —OR^{D1}, —N(R^{D1}a)₂, —SO₂OR^{D1}, or —SR^{D1}, wherein R^{D1} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group when attached to an oxygen atom, or a sulfur protecting group when attached to a sulfur atom;
wherein each occurrence of R^{D1a} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring;
provided that R^{1A} and R² are reaction partners;
L^{B} is an alkylene linker, wherein one or more chain atoms of the hydrocarbon chain are independently replaced with amide, ester, hydroxamate, ether, carbonate, carbamate, hydrazone, thioether, thioester, disulfide, orthoester, urethane, or oxime moiety, or a cyclic moiety; and
T is a therapeutic agent.

191. A dendrimer conjugate of Formula (I): 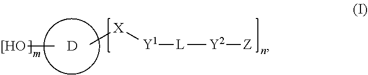
wherein:
D is a dendrimer selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof;
X is O or NH;
Y¹ is optionally substituted alkylene, optionally substituted alkenylene, optionally substituted alkynylene, or a covalent bond;
Y² is selected from the group consisting of secondary amides, tertiary amides, sulfonamide, secondary carbamates, tertiary carbamates, carbonates, ureas, carbinols, disulfides, hydrazones, hydrazides, ethers, carbonyls, and combinations thereof;
Z is a therapeutic agent or an imaging agent;
L is a linker comprising a polymer and at least one moiety selected from the group consisting of 1,2,3-triazolyl, 4,5-dihydro-1,2,3-triazolyl, isoxazolyl, 4,5-dihydroisoxazolyl, and 1,4-dihydropyridazyl;
m is an integer from 16 to 4096, inclusive; and
n is an integer from 1 to 100, inclusive.

192. A dendrimer conjugate of Formula (II): 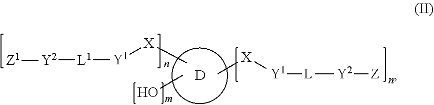
wherein:
D is a dendrimer selected from the group consisting of poly(amidoamine) (PAMAM) polymers, polypropylamine (POPAM) polymers, polyethylenimine polymers, polylysine polymers, polyester polymers, iptycene polymers, aliphatic poly(ether) polymers, aromatic polyether polymers, 2,2-bis(hydroxymethyl)propionic acid (bis-MPA) polymers, and combinations thereof;
each instance of X is independently O or NH;
each instance of Y¹ is independently optionally substituted alkylene, optionally substituted alkenylene, optionally substituted alkynylene, or a covalent bond;
each instance of Y² is independently selected from the group consisting of secondary amides, tertiary amides, sulfonamide, secondary carbamates, tertiary carbamates, carbonates, ureas, carbinols, disulfides, hydrazones, hydrazides, ethers, carbonyls, and combinations thereof;
Z¹ and Z² are independently therapeutic agents, targeting agents, or imaging agents, with the proviso that Z¹ and Z² are different;
L¹ and L² are independently linkers comprising a polymer and at least one moiety selected from the group consisting of 1,2,3-triazolyl, 4,5-dihydro-1,2,3-triazolyl, isoxazolyl, 4,5-dihydroisoxazolyl, and 1,4-dihydropyridazyl;
m is an integer from 16 to 4096, inclusive; and
each instance of n is independently an integer from 1 to 100, inclusive.

193. A composition of a therapeutic compound comprising a dendrimer conjugated to a therapeutic agent through a terminal ether or amide bond, wherein the dendrimer comprises a high-density of terminal hydroxyl groups optionally substituted with the therapeutic agent, wherein the therapeutic compound is 10-20% by mass of therapeutic agent.

194. A method of treating or imaging a disease or disorder of the brain or central nervous system in a subject in need thereof, the method comprising:
administering to the subject the composition of claim 193 in an amount effective to treat or image the disease or disorder of the brain or central nervous system in the subject.

195. A method of treating or imaging a disease or disorder of the eye in a subject in need thereof, the method comprising:
administering to the subject the composition of claim 193 in an amount effective to treat or image the disease or disorder of the eye in the subject.

196. A method of treating or imaging a proliferative disease in a subject in need thereof, the method comprising:
administering to the subject the composition of claim 193 in an amount effective to treat or image the proliferative disease in the subject.
